(19) United States
(12) Patent Application Publication
(10) Pub. No.: US 2024/0228957 A9
(43) Pub. Date: Jul. 11, 2024

(54) METHODS FOR CULTURING CELLS

(71) Applicant: Lyell Immunopharma, Inc., South San Francisco, CA (US)

(72) Inventors: Suman Kumar VODNALA, San Mateo, CA (US); Yogin PATEL, South San Francisco, CA (US); Nicholas P. RESTIFO, South San Francisco, CA (US); Robert Langland EIL, South San Francisco, CA (US)

(73) Assignee: Lyell Immunopharma, Inc., South San Francisco, CA (US)

(21) Appl. No.: 18/548,006

(22) PCT Filed: Feb. 24, 2022
(86) PCT No.: PCT/US2022/017774

§ 371 Date: Aug. 25, 2023

Related U.S. Application Data
(60) Provisional application No. 63/273,141, filed on Oct. 28, 2021.
(60) Provisional application No. 63/181,224, filed on Apr. 28, 2021.
(60) Provisional application No. 63/167,606, filed on Mar. 29, 2021.
(60) Provisional application No. 63/167,608, filed on Mar. 29, 2021.
(60) Provisional application No. 63/165,021, filed on Mar. 23, 2021.
(60) Provisional application No. 63/165,042, filed on Mar. 23, 2021.
(60) Provisional application No. 63/153,905, filed on Feb. 25, 2021.
(60) Provisional application No. 63/153,920, filed on Feb. 25, 2021.

(51) Int. Cl. C12N 5/078; A61P 35/00; C12N 15/86

(52) U.S. Cl. CPC C12N 5/0634; A61P 35/00; C12N 15/86; C12N 2500/12; C12N 2500/14; C12N 2500/34; C12N 2501/2302; C12N 2501/2307; C12N 2501/2315; C12N 2501/2321; C12N 2510/00

(57) ABSTRACT

The preset disclosure provides methods of culturing cells, e.g., pluripotent cells, multipotent cells, and/or immune cells (e.g., T cells and/or NK cells) in a medium comprising at least about 5 mM potassium ion, wherein the medium is not hypertonic. In some aspects, the medium is hypotonic. In some aspects, the methods disclosed herein increases the number of less-differentiated cells in the population of cells. In some aspects, the cultured cells are engineered, e.g., to comprise a chimeric antigen receptor or an engineered T cell receptor. In some aspects, the cells are administered to a subject in need thereof.

### CROSS-REFERENCE TO RELATED APPLICATIONS

This application claims priority to and benefit of U.S. Provisional Application Nos. 63/153,905 filed on Feb. 25, 2021; 63/153,920 filed on Feb. 25, 2021; 63/165,021 filed on Mar. 23, 2021; 63/165,042 filed on Mar. 23, 2021; 63/167,606 filed on Mar. 29, 2021; 63/167,608 filed on Mar. 29, 2021; 63/181,224 filed on Apr. 28, 2021; 63/273,141 filed Oct. 28, 2021; each of which is incorporated by reference herein in its entirety.

### FIELD

The present disclosure relates to methods of culturing cells, e.g., pluripotent, multipotent, and/or immune cells (e.g., T cells and/or NK cells). In some aspects, the methods disclosed herein promote enrichment of less-differentiated cells and/or undifferentiated cells in culture. Cells cultured using the methods disclosed herein can be used for various cell therapies, including but not limited to chimeric antigen receptor (CAR) T cell therapy and TCR T cell therapy including neoantigen directed-T cell therapies.

### BACKGROUND

Cancer immunotherapy relies on harnessing T cells—the immune system's primary killers of infected and diseased cells—to attack and kill tumor cells. However, there is an important stumbling block for immunotherapy: T cells' ability to kill can fade, a phenomenon often referred to as exhaustion or terminal differentiation of T cells. Immune checkpoint blockade, ex vivo-expanded Tumor-Infiltrating Lymphocytes (TILs) therapy, chimeric antigen receptor (CAR) T cell therapy, and T cell receptor-engineered (TCR) T cell therapy are treatments that make use of functionally active T cells isolated from patients and require highly functional T cells in order to be effective. These T cells are engineered and expanded ex vivo to recognize antigens on target cancer cells. T cell therapies have not been consistently effective at curing solid cancers, in part because the T cells lose their ability to proliferate or kill over time.

One means of overcoming T cell exhaustion is to selectively administer T cells having a less-differentiated state. For example, T memory stem cells (T_{SCM}) persist for a greater period in patients following administration than do more differentiated T central memory (T_{CM}) or T effector memory (T_{EM}) cells, and T_{SCM} elicit a more pronounced and prolonged effect on tumor size than more differentiated cells. However, many adoptive cell therapy (ACT) cell preparations comprise an ill-defined mix of immune cells at various states of differentiation, which are ineffective at eradicating solid tumors. To be curative, T cells products with enhanced self-renewing stem/effector properties are needed. As such, there remains a need in the art for methods of efficiently enriching for less differentiated and/or naïve T cells from a mixed population of isolated T cells.

### BRIEF SUMMARY

Certain aspects of the present disclosure are directed to a method of culturing immune cells and/or stem cells ex vivo or in vitro comprising placing immune cells and/or stem cells in a medium comprising potassium ion at a concentration higher than 40 mM, wherein the medium is not hypertonic. Certain aspects of the present disclosure are directed to a method of preparing a population of immune cells and/or stem cells comprising placing immune cells and/or stem cells into a medium comprising potassium ion at a concentration higher than 40 mM, wherein the medium is not hypertonic.

In some aspects, the medium further comprises one or more cytokines. In some aspects, the one or more cytokines comprise Interleukin-2 (IL-2), Interleukin-7 (IL-7), Interleukin-21 (IL-21), Interleukin-15 (IL-15), or any combination thereof.

Certain aspects of the present disclosure are directed to a method of increasing a number or percentage of undifferentiated or less differentiated cells ex vivo or in vitro comprising culturing immune cells and/or stem cells in a medium comprising potassium ion at a concentration of higher than 40 mM, wherein the medium comprises IL-2, but does not comprise IL-7 and IL-15. In some aspects, the immune cells and/or stem cells after the culturing comprises a higher number and/or percentage of undifferentiated or less differentiated immune cells and/or stem cells compared to immune cells and/or stem cells that are cultured in a medium comprising IL-2, IL-7, and IL-15.

Certain aspects of the present disclosure are directed to a method of increasing a number and/or percentage of undifferentiated or less differentiated immune cells and/or stem cells ex vivo or in vitro comprising placing immune cells and/or stem cells in a medium comprising potassium ion at a concentration higher than 40 mM, wherein the medium comprises IL-7 and IL-21.

Certain aspects of the present disclosure are directed to a method of increasing a number or percentage of undifferentiated or less differentiated immune cells and/or stem cells ex vivo or in vitro comprising placing the immune cells and/or stem cells in a medium comprising potassium ion at a concentration higher than 40 mM, wherein the medium comprises IL-15 and IL-21 or wherein the medium comprises IL-2, IL-7 and IL-15.

In some aspects, the cells comprise immune cells. In some aspects, the immune cells comprise T cells, TILs, NK cells, TILs, Tregs, or any combination thereof. In some aspects, the cells comprise stem cells. In some aspects, the cells express chimeric antigen receptor (CAR). In some aspects, the cells express an engineered T cell receptor (TCR), which may be an engineered TCR. In some aspects, the medium is hypotonic. In some aspects, the medium further comprises sodium ion, e.g., NaCl, calcium ion, glucose, and/or any combination thereof.

In some aspects, the medium further comprises a cell expansion agent. In some aspects, the cell expansion agent comprises a GSK3B inhibitor, an ACLY inhibitor, a PI3K inhibitor, an AKT inhibitor, or any combination thereof. In some aspects, the PI3K inhibitor is selected from LY294002, pictilisib, CAL101, IC87114, or any combination thereof. In some aspects, the AKT inhibitor is selected from MK2206, A443654, AKTi-VIII, or any combination thereof. In some aspects, the medium is capable of: increasing the number and/or percentage of less differentiated and/or undifferentiated cells; increasing transduction efficiency; increasing stem-like immune cells; increasing in vivo viability; increasing cell potency; preventing cell exhaustion; or any combination thereof, e.g., in the final cell product as compared to the starting cell population.

In some aspects, the concentration of potassium ion is at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM. In some aspects, the concentration of potassium ion is about 45 mM to about 110 mM, about 45 mM to about 100 mM, about 45 mM to about 90 mM, about 45 mM to about 80 mM, about 45 mM to about 70 mM, about 45 mM to about 60 mM, about 45 mM to about 50 mM, about 50 mM to about 110 mM, about 50 mM to about 100 mM, about 50 mM to about 90 mM, about 50 mM to about 80 mM, about 50 mM to about 70 mM, about 60 mM to about 110 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 110 mM, about 80 mM to about 100 mM, about 80 mM to about 90 mM, about 90 mM to about 110 mM, about 90 mM to about 100 mM, or about 100 mM to about 110 mM. In some aspects, the concentration of potassium ion is about 50 mM to about 90 mM. In some aspects, the concentration of potassium ion is about 50 mM to about 80 mM.

In some aspects, the medium has an osmolality lower than about 280 mOsm/L. In some aspects, the medium has an osmolality between about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L. In some aspects, the medium has an osmolality of about 100 mOsm/L to about 280 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L.

In some aspects, the medium has an osmolality of about 236 mOsm/L. In some aspects, the medium has an osmolality of about 250 mOsm/L. In some aspects, the medium has an osmolality of about 255 mOsm/L. In some aspects, the medium has an osmolality of about 260 mOsm/L.

In some aspects, the medium is isotonic. In some aspects, the medium has an osmolality of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L. In some aspects, the medium has an osmolality of about 285 mOsm/L, about 290 mOsm/L, about 295 mOsm/L, about 300 mOsm/L, about 305 mOsm/L, about 310 mOsm/L, or about 315 mOsm/L.

In some aspects, the medium further comprises sodium ion. In some aspects, the concentration of the sodium ion is from about 25 mM to about 100 mM. In some aspects, the concentration of the sodium ion is from about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In some aspects, the concentration of the sodium ion is about 30 mM, about 35 mM, about 40 mM, about 45 mM, about 50 mM, about 55 mM, about 60 mM, about 65 mM, about 70 mM, about 75 mM, or about 80 mM.

In some aspects, the concentration of the sodium ion is about 55 mM. In some aspects, the concentration of the sodium ion is about 60 mM. In some aspects, the concentration of the sodium ion is about 65 mM. In some aspects, the concentration of the sodium ion is about 70 mM. In some aspects, the concentration of the sodium ion is about 75 mM. In some aspects, the concentration of the sodium ion is about 80 to about 85 mM. In some aspects, the concentration of the sodium ion is about 80 to about 90 mM.

In some aspects, the medium further comprises glucose. In some aspects, the concentration of glucose is more than about 10 mM. In some aspects, the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM. In some aspects, the concentration of glucose is about 15.4 mM, about 15.9 mM, about 16.3 mM, about 16.8 mM, about 17.2 mM, or about 17.7 mM.

In some aspects, the medium further comprises calcium ion. In some aspects, the concentration of calcium ion is more than about 0.4 mM. In some aspects, the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM. In some aspects, the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

In some aspects, the medium comprises about 50 mM potassium ion and (i) about 80.5 mM sodium ion; (ii) about 17.7 mM glucose; (iii) about 1.8 mM calcium ion; or (iv) any combination of (i)-(iii). In some aspects, the medium has an osmolality of about 254.7 mOsmol.

In some aspects, the medium comprises about 55 mM potassium ion and (i) about 76 mM sodium ion; (ii) about 17.2 mM glucose; (iii) about 1.7 mM calcium ion; or (iv) any combination of (i)-(iii). In some aspects, the medium has an osmolality of about 255.2 mOsmol.

In some aspects, the medium comprises about 60 mM potassium ion and (i) about 72.2 mM sodium ion; (ii) about 16.8 mM glucose; (iii) about 1.6 mM calcium ion; or (iv) any combination of (i)-(iii). In some aspects, the medium has an osmolality of about 257.2 mOsmol.

In some aspects, the medium comprises about 65 mM potassium ion and (i) about 67.6 mM sodium ion; (ii) about 16.3 mM glucose; (iii) about 1.5 mM calcium ion; or (iv) any combination of (i)-(iii). In some aspects, the medium has an osmolality of about 257.5 mOsmol.

In some aspects, the medium comprises about 70 mM potassium ion and (i) about 63.9 mM sodium ion; (ii) about 15.9 mM glucose; (iii) about 1.4 mM calcium ion; or (iv) any combination of (i)-(iii). In some aspects, the medium has an osmolality of about 259.7 mOsmol.

In some aspects, the medium comprises about 75 mM potassium ion and (i) about 59.3 mM sodium ion; (ii) about 15.4 mM glucose; (iii) about 1.3 mM calcium ion; or (iv) any combination of (i)-(iii). In some aspects, the medium has an osmolality of about 260 mOsmol.

In some aspects, the medium comprises about 80 mM potassium ion and (i) about 55.6 mM sodium ion; (ii) about 15 mM glucose; (iii) about 1.2 mM calcium ion; or (iv) any combination of (i)-(iii). In some aspects, the medium has an osmolality of about 262.26 mOsmol.

In some aspects, the immune cells are CD3+, CD45RO−, CCR7+, CD45RA+, CD62L+, CD27+, CD28+, or TCF7+, or any combination thereof, following culture.

In some aspects, the concentration of IL-2 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

In some aspects, the concentration of IL-2 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-2 is about 1.0 ng/mL. In some aspects, the concentration of IL-2 is about 10 ng/mL.

In some aspects, the concentration of IL-21 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-21 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-21 is about 1.0 ng/mL. In some aspects, the concentration of IL-21 is about 10 ng/mL.

In some aspects, the concentration of IL-7 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-7 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-7 is about 1.0 ng/mL. In some aspects, the concentration of IL-7 is about 10 ng/mL.

In some aspects, the concentration of IL-15 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-15 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-15 is about 1.0 ng/mL. In some aspects, the concentration of IL-15 is about 10 ng/mL.

In some aspects, the immune cells and/or stem cells comprises a chimeric antigen receptor, an engineered T cell receptor, or any combination thereof.

In some aspects, the immune cells and/or stem cells are administered to a human subject following culture.

In some aspects, the cells are further transduced with a vector. In some aspects, the vector comprises a transgene encoding a chimeric antigen receptor (CAR), a T cell receptor (TCR), or a TCR mimic. In some aspects, the CAR targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof. In some aspects, the TCR targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin Bi, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LTLRA2, CD300LF, CLEC12A, BST2, EMR2, LY75, GPC3, FCRL5, IGLL1, CD2, CD3P, CD4, CD5, CD7, the extracellular portion of the APRIL protein, or any combinations thereof. In some aspects, the vector is a retroviral vector, a lentiviral vector, an adeno-associated virus (AAV), an adenovirus, an AAV hybrid virus, a baculovirus, or any combination thereof Certain aspects of the present disclosure are directed to a population of cells prepared by a method disclosed herein. Certain aspects of the present disclosure are directed to a population of cells comprising at least about 5%, at least about 10%, at least about 15%, at least about 20%, at least about 25%, at least about 30%, at least about 35%, at least about 40%, at least about 45%, at least about 50%, at least about 60%, or at least about 70% of the cells are CD3+, CD45RO−, CCR7+, CD45RA+, CD62L+, CD27+, CD28+, and TCF7+, wherein the cells are cultured according to a method disclosed herein. Certain aspects of the present disclosure are directed to a cell culture medium useful for a method disclosed herein. Certain aspects of the present disclosure are directed to a cell culture medium comprising (i) immune cells or stem cells as disclosed herein and (ii) a potassium ion at a concentration higher than 40 mM, wherein the culture medium is not hypertonic.

In some aspects, the concentration of the potassium ion is at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM. In some aspects, the concentration of the potassium ion is about 50 mM, about 60 mM, about 70 mM, or about 80 mM. In some aspects, the culture medium further comprises one or more cytokines. In some aspects, the one or more cytokines are IL-2, IL-7, IL-15, IL-21, or any combination thereof. Certain aspects of the present disclosure are directed to a method of treating a disease or condition in a subject in need thereof comprising administering a population of cells disclosed herein to the subject. In some aspects, the disease or condition comprises a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

Certain aspects of the present disclosure are directed to a method of expanding immune cells obtained from a human subject comprising culturing the immune cells in initial expansion media, wherein the initial expansion media are hyperkalemic. In some aspects, the method comprises culturing the immune cells in secondary expansion media, after culturing the cells in the initial expansion media. In some aspects, the second expansion media are hyperkalemic.

Certain aspects of the present disclosure are directed to a method of expanding immune cells obtained from a human subject comprising culturing the immune cells (a) in initial expansion media and (b) in secondary expansion media, wherein the initial expansion media or the secondary expansion media are hyperkalemic. In some aspects, the secondary expansion media are hyperkalemic, and wherein the initial expansion media is not hyperkalemic.

In some aspects, the method further comprises culturing the immune cells in third (or final) expansion media. In some aspects, the third expansion media are hyperkalemic. In some aspects, the initial expansion media and the secondary expansion media are hyperkalemic and the third expansion media are not hyperkalemic.

In some aspects, the initial expansion media further comprises IL-2, IL-21, or both. In some aspects, the initial expansion media further comprises a T cell supplement, a serum replacement, glutamine, a glutamine substitute (e.g., Glutamax (L-alanine-L-glutamine)), non-essential amino acids, an antibiotics (e.g., Penicillin, Streptomycin, or both), an anti-fungal agent (e.g., FUNGIN™), and/or sodium pyruvate. In some aspects, the immune cells are cultured in the initial expansion media for up to about six days, about seven days, about eight days, about nine days, or about 10 days.

In some aspects, the immune cells are stimulated with a CD3 agonist, a CD28 agnostic, or both in the secondary expansion media. In some aspects, the immune cells are further stimulated with a CD27 ligand in the secondary expansion media. In some aspects, the immune cells are further stimulated with a 4-1BB ligand in the secondary expansion media. In some aspects, the immune cells are cultured for at least about 20 days, at least about 21 days, at least about 22 days, at least about 23 days, at least about 24 days, at least about 25 days, or at least about 26 days, after stimulation with a CD3 agonist, a CD28 agonist, and/or a CD27 ligand.

In some aspects, the immune cells are stimulated with a CD3 agonist, a CD28 agonist, a CD27 ligand, and/or a 4-1BB ligand in the third expansion media. In some aspects, the third expansion media are not hyperkalemic. In some aspects, the immune cells are cultured for at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, or at least about 43 days.

Some aspects of the present disclosure are directed to a method of increasing tumor reactive immune cells comprising: (a) culturing immune cells in initial expansion media, which are hyperkalemic and optionally comprise IL-2 and/or IL-21, up to about seven to 14 days; (b) culturing the immune cells in secondary expansion media, which are hyperkalemic after adding a CD3 agonist, a CD28 agonist, a CD27 ligand, a 4-1BB ligand, or any combination thereof for about 20 days to about 25 days, e.g., about 21 days to about 24 days; (c) culturing the immune cells in third expansion media, which are not hyperkalemic, after adding a CD3 agonist, a CD28 agonist, a CD27 ligand, a 4-1BB ligand, or any combination thereof for about 30 days to about 50 days, e.g., about 34 days to about 45 days.

Some aspects of the present disclosure are directed to an immune cell that expresses one or more stem-like markers and one or more effector-like markers. In some aspects, the stem-like markers comprise CD45RA+, CD62L+, CCR7+, CD27+, CD28+, BACH2+, LEF1+, TCF7+, or any combination thereof. In some aspects the stem-like markers comprise CD45RA+, CD62L+, CCR7+, TCF7+, or any combination thereof. In some aspects the stem-like markers comprise CD45RA+, CD45RO^{Ow}, CD62L+, CCR7+, TCF7+, or any combination thereof. In some aspects, the effector-like markers comprise pSTAT5+, STAT5+, pSTAT3+, STAT3+, or any combination thereof Some aspects of the present disclosure are directed to a population of engineered cells comprising an immune cell disclosed herein. In some aspects, at least about 40%, at least about 50%, at least about 60%, at least about 70%, at least about 80%, at least about 85%, at least about 90%, at least about 95%, at least about 99%, or about 100% of the engineered cells comprise the immune cell. Some aspects of the present disclosure are directed to a pharmaceutical composition comprising the immune cell or the population of engineered cells and a pharmaceutically acceptable carrier. Some aspects of the present disclosure are directed to a method of treating a disease or condition in a subject in need thereof comprising administering the immune cell, the population of engineered cells, or the pharmaceutical composition to the subject. In some aspects, the disease or condition is a cancer. Some aspects of the present disclosure are directed to a method of preparing the immune cell, comprising culturing the immune cell in culture medium under suitable conditions.

Some aspects of the present disclosure are directed to a population of human T cells, wherein at least about 5% of the T cells in the population of T cells have a stem-like phenotype. In some aspects, at least about 10%, at least about 15%, at least about 20%, at least about 25%, at least about 30%, at least about 35%, at least about 40%, at least about 45%, or at least about 50% of the T cells in the population of T cells have a stem-like phenotype. In some aspects, the T cells having a stem-like phenotype are TCF7⁺. In some aspects, the T cells having a stem-like phenotype are CD3⁺, CD45RO⁻, CCR7⁺, CD45RA⁺, CD62L+, CD27⁺, CD28⁺, and TCF7⁺. In some aspects, the T cells having a stem-like phenotype are CD39− and CD69−. In some aspects, the T cells are CD8⁺ T cells.

Some aspects of the present disclosure are directed to a pharmaceutical composition comprising a population of human T cells disclosed herein, and a pharmaceutically acceptable carrier. Some aspects of the present disclosure are directed to a method of killing target cells, comprising contacting the target cells with a population of human T cells disclosed herein or a pharmaceutical composition disclosed herein under conditions that allow killing of the target cells by the T cells. Some aspects of the present disclosure are directed to a method of treating a patient in need thereof, comprising administering a population of human T cells disclosed herein or a pharmaceutical composition disclosed herein to the patient. Some aspects of the present disclosure are directed to a method of inducing an anti-tumor immune response in a patient, comprising administering a population of human T cells disclosed herein or a pharmaceutical composition disclosed herein to the patient. Some aspects of the present disclosure are directed to use of a population of human T cells disclosed herein or a pharmaceutical composition disclosed herein for the manufacture of a medicament for treating a patient in need thereof in the method disclosed herein.

In some aspects, after the administration the size of a tumor (tumor size) in the patient is decreased compared to a reference tumor size. In some aspects, the reference tumor size comprises: (i) the tumor size before the administration, (ii) the tumor size in a corresponding subject that did not receive the administration, or (iii) both (i) and (ii). In some aspects, compared to the reference tumor size, the tumor size is decreased by at least about 5%, at least about 10%, at least about 20%, at least about 30%, at least about 40%, at least about 50%, at least about 60%, at least about 70%, at least about 80%, at least about 90% or about 100%.

In some aspects, after the administration the duration of survival of the subject is increased compared to a reference duration of survival. In some aspects, the reference duration of survival comprises the duration of survival of a corresponding subject that did not receive the administration. In some aspects, compared to the reference duration of survival, the duration of survival is by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more.

In some aspects, the immune cells express increased IL-2 following culture, as compared to immune cells cultured in control medium. In some aspects, the immune cells express at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold more IL-2 as compared to immune cells cultured in control medium.

In some aspects, the immune cells express at least about 20 pg/ml, at least about 25 pg/ml, at least about 50 pg/ml, at least about 75 pg/ml, at least about 100 pg/ml, at least about 150 pg/ml, at least about 200 pg/ml, at least about 250 pg/ml, at least about 300 pg/ml, at least about 350 pg/ml, at least about 400 pg/ml, at least about 450 pg/ml, at least about 500 pg/ml, at least about 600 pg/ml, at least about 700 pg/ml, at least about 800 pg/ml, at least about 900 pg/ml, at least about 1000 pg/ml, at least about 1250 pg/ml, at least about 1500 pg/ml, at least about 1750 pg/ml, at least about 2000 pg/ml, at least about 2500 pg/ml, at least about 3000 pg/ml, at least about 3500 pg/ml, at least about 4000 pg/ml, at least about 4500 pg/ml, or at least about 5000 pg/ml IL-2. In some aspects, the immune cells express increased IL-2 following a single stimulation. In some aspects, the immune cells express increased IL-2 following at least two serial stimulations, and least three serial stimulations, and least four serial stimulations, or at least five serial stimulations.

In some aspects, the immune cells further express c-Jun.

In some aspects, the immune cells exhibit increased cytotoxic activity as compared to immune cells cultured in control medium. In some aspects, immune cell cytotoxicity is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold more IL-2 as compared to immune cells cultured in control medium.

In some aspects, the immune cells exhibit increased persistence as compared to immune cells cultured in control medium. In some aspects, the immune cells exhibit cytotoxic activity following repeated contact with target cells for at least about 5 days, at least about 7 days, at least about 10 days, at least about 12 days, or at least about 14 days.

### ASPECTS

Aspect 1. A method of culturing tumor infiltrating lymphocytes (TILs) ex vivo or in vitro comprising placing a heterogeneous population of TILs in a metabolic reprogramming medium (“MRM”) comprising potassium ion at a concentration of about 30 mM to about 100 mM.

Aspect 2. The method of aspect 1, wherein the heterogeneous population of TILs is enriched in CD8⁺ TILs after being placed in the MRM.

Aspect 3. A method of increasing a number or percentage of CD8⁺ TILs ex vivo or in vitro comprising culturing a heterogeneous population of TILs in an MRM comprising potassium ion at a concentration of about 30 mM to about 100 mM.

Aspect 4. A method of preparing a CD8⁺-enriched population of TILs, comprising culturing a heterogeneous population of TILs ex vivo or in vitro in an MRM comprising potassium ion at a concentration of about 30 mM to about 100 mM.

Aspect 5. The method of any one of aspects 1 to 4, wherein the heterogeneous population of TILs comprises CD4⁺ TILs and CD8⁺ TILs.

Aspect 6. The method of any one of aspects 1 to 5, wherein the heterogeneous population of TILs is obtained from one or more tumor sample obtained from a subject.

Aspect 7. The method of aspect 6, wherein the tumor sample subjected to an initial TIL culture.

Aspect 8. The method of aspect 7, wherein the initial TIL culture comprises culturing the tumor sample in the MRM.

Aspect 9. The method of aspect 8, wherein the MRM further comprises IL-2 during the initial TIL culture.

Aspect 10. The method of aspect 9, wherein the MRM further comprises IL-7, IL-15, IL-21, or any combination thereof during the initial TIL culture.

Aspect 11. The method of any one of aspects 8 to 10, wherein the MRM comprises IL-2 and IL-21 during the initial TIL culture.

Aspect 12. The method of any one of aspects 7 to 11, wherein the initial TL culture lasts at least about 14-19 days.

Aspect 13. The method of any one of aspects 7 to 12, wherein the initial TIL culture lasts at least about 14 days.

Aspect 14. The method of any one of aspects 7 to 13, wherein the proportion of CD8⁺ TILs to non-CD8⁺ TILs is increased following the initial TIL culture, as compared to the proportion of CD8⁺ TILs to non-CD8⁺ TILs prior to the initial TIL culture.

Aspect 15. The method of any one of aspects 7 to 14, wherein the TILs are stimulated following the initial TIL culture.

Aspect 16. The method of aspect 15, wherein the TILs are stimulated by culturing the TILs with a CD3 agonist and/or a CD28 agonist.

Aspect 17. The method of any one of aspects 6 to 16, wherein the tumor sample comprises a tumor biopsy.

Aspect 18. The method of any one of aspects 6 to 17, wherein the tumor sample is fragmented prior to culturing.

Aspect 19. The method of any one of aspects 1 to 18, wherein following culture of the heterogeneous population of TILs, at least about 300%, at least about 355%, at least about 40%, at least about 45%, at least about 50%, at least about 55%, at least about 60%, at least about 65%, at least about 70%, at least about 75%, or at least about 80% of the TILs in the population are CD8⁺ TILs.

Aspect 20. The method of any one of aspects 1 to 19, wherein following culture of the heterogeneous population of TILs, at least about 50% of the TILs in the population are CD8⁺ TILs.

Aspect 21. The method of any one of aspects 1 to 20, wherein the MRM further comprises sodium ion, calcium ion, glucose, and any combination thereof.

Aspect 22. The method of any one of aspects 1 to 21 wherein the MRM further comprises a cell expansion agent.

Aspect 23. The method of aspect 22, wherein the cell expansion agent comprises a GSK3B inhibitor, an ACLY inhibitor, a PI3K inhibitor, an AKT inhibitor, or any combination thereof.

Aspect 24. The method of aspect 23, wherein the PI3K inhibitor comprises LY294002, pictilisib, CAL101, IC87114, or any combination thereof.

Aspect 25. The method of aspect 24, wherein the AKT inhibitor comprises MK2206, A443654, AKTi-VIII, or any combination thereof.

Aspect 26. The method of any one of aspects 3 to 25, wherein the concentration of potassium ion is at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM.

Aspect 27. The method of any one of aspects 3 to 26, wherein the concentration of potassium ion is about 30 mM to about 100 mM, about 30 mM to about 90 mM, about 30 mM to about 80 mM, about 30 mM to about 70 mM, about 30 mM to about 60 mM, about 30 mM to about 50 mM, about 40 mM to about 100 mM, about 40 mM to about 90 mM, about 40 mM to about 80 mM, about 40 mM to about 70 mM, about 40 mM to about 60 mM, or about 40 mM to about 50 mM.

Aspect 28. The method of any one of aspects 1 to 27, wherein the concentration of potassium ion is about 40 mM to about 90 mM.

Aspect 29. The method of any one of aspects 1 to 28, wherein the concentration of potassium ion is about 50 mM to about 90 mM.

Aspect 30. The method of any one of aspects 1 to 29, wherein the concentration of potassium ion is about 50 mM to about 80 mM.

Aspect 31. The method of any one of aspects 1 to 30, wherein the MRM further comprises sodium ion.

Aspect 32. The method of aspect 31, wherein the concentration of the sodium ion is from about 25 mM to about 100 mM.

Aspect 33. The method of aspect 31 or 32, wherein the concentration of the sodium ion is from about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM.

Aspect 34. The method of any one of aspects 31 to 33, wherein the concentration of the sodium ion is about 30 mM, about 35 mM, about 40 mM, about 45 mM, about 50 mM, about 55 mM, about 60 mM, about 65 mM, about 70 mM, about 75 mM, or about 80 mM.

Aspect 35. The method of any one of aspects 31 to 34, wherein the concentration of the sodium ion is about 55 mM.

Aspect 36. The method of any one of aspects 31 to 35, wherein the concentration of the sodium ion is about 60 mM.

Aspect 37. The method of any one of aspects 31 to 36, wherein the concentration of the sodium ion is about 65 mM.

Aspect 38. The method of any one of aspects 1 to 37, wherein the MRM further comprises glucose.

Aspect 39. The method of aspect 38, wherein the concentration of glucose is more than about 10 mM.

Aspect 40. The method of aspect 38 or 39, wherein the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM.

Aspect 41. The method of any one of aspects 38 to 40, wherein the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM.

Aspect 42. The method of any one of aspects 1 to 41, wherein the MRM further comprises calcium ion.

Aspect 43. The method of aspect 42, wherein the concentration of calcium ion is more than about 0.4 mM.

Aspect 44. The method of aspect 42 or 43, wherein the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

Aspect 45. The method of any one of aspects 42 to 44, wherein the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

Aspect 46. The method of any one of aspects 1 to 45, wherein the MRM comprises about 40 mM to about 90 mM potassium ion and (i) about 40 mM to about 80 mM sodium ion; (ii) about 10 mM to about 24 mM glucose; (iii) about 0.5 mM to about 2.8 mM calcium ion; or (iv) any combination of (i)-(iii).

Aspect 47. A method of expanding TILs obtained from a human subject comprising: (a) culturing the TILs in an initial TIL culture media; (b) culturing the TILs in a secondary TIL culture media; (c) culturing the TILs in a third (or final) TIL culture media, wherein the initial TIL culture media, the secondary TIL expansion media, and/or the third TIL expansion media are MRM.

Aspect 48. The method of aspect 47, wherein the initial TIL culture media and the secondary TIL expansion media are hyperkalemic and the third TIL expansion media are not hyperkalemic.

Aspect 49. The method of aspect 47 or 48, wherein the initial TIL culture media further comprises IL-2.

Aspect 50. The method of aspect 49, wherein the initial TIL culture media further comprises IL-21.

Aspect 51. The method of any one of aspects 47 to 50, wherein the initial TIL culture media further comprises a T cell supplement, a serum replacement, glutamine, a glutamine substitute (e.g., Glutamax (L-alanine-L-glutamine)), non-essential amino acids, an antibiotics (e.g., Penicillin, Streptomycin, or both), an anti-fungal agent (e.g., FUNGIN™) and/or sodium pyruvate.

Aspect 52. The method of any one of aspects 47 to 51, wherein the TILs are cultured in the initial TIL culture media for at least about 1 week, at least about 2 weeks, or at least about 3 weeks.

Aspect 53. The method of any one of aspects 47 to 52, wherein the TILs are cultured in the initial TIL culture media until cell yield in the initial culture reaches at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶ cells per fragment.

Aspect 54. The method of any one of aspects 47 to 53, wherein the TILs are stimulated with a CD3 agonist, a CD28 agonist, or both in or prior to the secondary TIL culture media in (b).

Aspect 55. The method of any aspects 47 to 54, wherein the TILs are further stimulated with a CD27 agonist in or prior to the secondary TIL culture media.

Aspect 56. The method of any one of aspects 47 to 55, wherein the TILs are further stimulated with a 4-1BB agonist in or prior to the secondary TIL culture media.

Aspect 57. The method of any one of aspects 53 to 56, wherein the TILs are cultured for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, or at least about 26 days, after the stimulation.

Aspect 58. The method of any one of aspects 47 to 57, wherein the TILs are cultured in the secondary culture media until cell yield reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells.

Aspect 59. The method of any one of aspects 47 to 58, wherein the TILs are stimulated with a CD3 agonist, a CD28 agonist, a CD27 agonist, and/or a 4-1BB agonist in the third TIL culture media.

Aspect 60. The method of aspect 59, wherein the third TIL culture media is not hyperkalemic.

Aspect 61. The method of aspect 59 or 60, wherein the TILs are cultured in the third TIL culture media for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days.

Aspect 62. A method of increasing tumor reactive TILs comprising: (a) culturing one or more tumor fragments in initial TIL culture media, which are hyperkalemic and comprise IL-2 and optionally IL-21, up to about 14 to 19 days thereby obtaining TILs from the tumor fragment; (b) culturing the TTLs in a secondary TIL culture media, which are hyperkalemic, after adding (i) a CD3 agonist and (ii) a CD28 agonist, a CD27 agonist, a 4-1BB agonist, or any combination thereof, for about 7 to at least about 14 days; (c) culturing the TILs in a third TIL culture media, which are not hyperkalemic, after adding (i) a CD3 agonist and (ii) a CD28 agonist, a CD27 agonist, a 4-1BB agonist, or any combination thereof, for about 14 days to at least about 21 days.

Aspect 63. The method of any one of aspects 1 to 62, wherein the TTLs exhibit increased expression of TCF7 following culture in the MRM, relative to TCF7 expression in a population of TILs following culture in a control medium that is not hyperkalemic.

Aspect 64. The method of any one of aspects 1 to 63, wherein the population of TILs comprises an increased proportion of CD8⁺ CD62L+ TTLs following culture in the MRM, relative to the proportion of CD8⁺ CD62L⁺ TILs following culture in a control medium that is not hyperkalemic.

Aspect 65. The method of any one of aspects 1 to 64, wherein the population of TILs comprises an increased proportion of CD8⁺ PD1V TILs following culture in the MRM, relative to the proportion of CD8⁺ PD1V TILs following culture in a control medium that is not hyperkalemic.

Aspect 66. A composition of immune cells, comprising one or more CD8⁺ TIL cultured according to the method of any one aspects 1 to 65.

Aspect 67. The composition of aspect 66, wherein at least about 30%, at least about 35%, at least about 40%, at least about 45%, at least about 50%, at least about 55%, at least about 60%, at least about 65%, at least about 70%, at least about 75%, or at least about 80% of the immune cells are CD8⁺ TILs.

Aspect 68. A composition comprising a population of immune cells, wherein at least about 30%, at least about 35%, at least about 40%, at least about 45%, at least about 50%, at least about 55%, at least about 60%, at least about 65%, at least about 70%, at least about 75%, or at least about 80% of the immune cells are CD8⁺ TILs.

Aspect 69. The composition of any one of aspects 66 to 68, wherein at least about 50% of the cells are CD8⁺ TILs.

Aspect 70. The composition of any one of aspects 66 to 69, wherein the cells exhibit increased expression of TCF7 following culture in the MRM, relative to TCF7 expression in a population of immune cells following culture in a control medium that is not hyperkalemic.

Aspect 71. The composition of any one of aspects 66 to 70, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/CD62L⁺ TTLs.

Aspect 72. The composition of any one of aspects 66 to 71, wherein at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺.

Aspect 73. The composition of any one of aspects 66 to 72, wherein at least about 10%, at least about 15%, at least about 20%, at least about 25%, at least about 30%, at least about 35%, at least about 40%, or at least about 50% of the CD8⁺ TILs obtained at the end of the initial TIL culture are CD39⁺.

Aspect 74. The composition of any one of aspects 66 to 73, wherein at least about 10%, at least about 15%, at least about 20%, at least about 25%, at least about 30%, at least about 35%, at least about 40%, or at least about 50% of the CD8⁺ TILs are CD27⁺.

Aspect 75. The composition of any one of aspects 66 to 74, wherein at least about 10%, at least about 15%, at least about 20%, at least about 25%, at least about 30%, at least about 35%, at least about 40%, or at least about 50% of the CD8⁺ TILs are CD28⁺.

Aspect 76. The composition of any one of aspects 66 to 75, wherein at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺ CD39⁺.

Aspect 77. The composition of any one of aspects 66 to 76, wherein at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺ CD27⁺.

Aspect 78. The composition of any one of aspects 66 to 77, wherein at least about 10%, at least about 15%, at least about 20%, at least about 25%, at least about 30%, at least about 35%, at least about 40%, or at least about 50% of the CD8⁺ TILs are CD27⁺ CD62L⁺.

Aspect 79. The composition of any one of aspects 66 to 78, wherein at least about 10%, at least about 15%, at least about 20%, at least about 25%, at least about 30%, at least about 35%, at least about 40%, or at least about 50% of the CD8+ TTLs obtained at the end of the initial TIL culture are CD27⁺ CD28⁺ CD103′ PD1V TCF7⁺.

Aspect 80. The composition of any one of aspects 66 to 79, comprising at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ cells.

Aspect 81. The composition of any one of aspects 66 to 80, comprising at least at least about 1×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ CD8⁺ cells.

Aspect 82. A method of treating a cancer in a subject in need thereof, comprising administering a population of TTLs to the subject, wherein the population of TILs are cultured according to the method of any one of aspects 1 to 65.

Aspect 83. The method of aspect 82, wherein the population of TILs is enriched for CD8⁺ TTLs.

Aspect 84. The method of aspect 82 or 83, wherein at least about 30%, at least about 35%, at least about 40%, at least about 45%, at least about 50%, at least about 55%, at least about 60%, at least about 65%, at least about 70%, at least about 75%, or at least about 80% of the TILs in the population of TTLs are CD8⁺ TTLs.

Aspect 85. The method of any one of aspects 82 to 84, wherein at least about 50% of the TILs in the population of TTLs are CD8⁺ TTLs.

Aspect 86. A method treating a cancer in a subject in need thereof, comprising administering to a subject the composition of any one of aspects 66 to 71.

Aspect 87. The method of any one of aspects 82 to 86, wherein the cancer comprises a solid tumor.

Aspect 88. The method of any one of aspects 82 to 87, wherein the cancer comprises a solid tumor derived from a melanoma, a colon cancer, a lung cancer, a cervical cancer, a gastrointestinal cancer, a breast cancer, a prostate cancer, a liver cancer, bone cancer, a pancreatic cancer, a small cell carcinoma of the head and neck, lung squamous cell carcinoma, lung adenocarcinoma, pancreatic adenocarcinoma, head and neck squamous cell carcinoma, testicular germ cell tumors, stomach adenocarcinoma, skin cutaneous melanoma, mesothelioma, kidney renal clear cell carcinoma, cervical squamous cell carcinoma and endocervical adenocarcinoma, esophageal carcinoma, bladder urothelial carcinoma, breast invasive carcinoma, kidney renal papillary cell carcinoma, colon adenocarcinoma, and any combination thereof.

Aspect 89. The method of any one of aspects 82 to 88, comprising administering at least about 2×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10¹⁰, or at least about 10×10¹⁰, or at least about 15×10¹⁰, or at least about 20×10¹⁰, or at least about 25×10¹⁰, or at least about 30×10¹⁰ cells to the subject.

Aspect 90. The method of any one of aspects 82 to 89, comprising administering at least about 1×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10⁹ CD8⁺ cells to the subject.

Aspect 91. The method of any one of aspects 82 to 90, further comprising administering a checkpoint inhibitor.

Aspect 92. The method of aspect 91, wherein the checkpoint inhibitor is administered to the subject after administering the population of cells.

Aspect 93. The method of aspect 91 or 92, wherein the checkpoint inhibitor comprises a CTLA-4 antagonist, a PD1 antagonist, a TIM-3 antagonist, or a combination thereof.

Aspect 94. The method of any one of aspects 91 to 93, wherein the checkpoint inhibitor comprises an anti-CTLA-4 antibody, an anti-PD1 antibody, an anti-PD-L1 antibody, an anti-TIM-3 antibody, or a combination thereof.

Aspect 95. The method of any one of aspects 92 to 94, further comprising administering a checkpoint activator.

Aspect 96. The method of any one of aspects 90 to 95, wherein the checkpoint inhibitor is administered to the subject after administering the population of TILs.

Aspect 97. The method of aspect 95 or 96, wherein the checkpoint activator comprises an OX40 agonist, a LAG-3 agonist, a 4-1BB (CD137) agonist, a GITR agonist, a TIM3 agonist, or a combination thereof.

Aspect 98. The method of aspect 95 or 96, wherein the checkpoint activator comprises an anti-OX40 antibody, an anti-LAG-3 antibody, an anti-CD137 antibody, an anti-GITR antibody, an anti-TIM3 antibody, or a combination thereof.

Aspect 99. The method of any one of aspects 92 to 98, further comprising administering a cytokine.

Aspect 100. The method of aspect 99, wherein the cytokine is administered to the subject after administering the population of TILs.

Aspect 101. The method of aspect 99 or 100, wherein the cytokine is IL-2.

Aspect 102. The method of any one of aspects 99 to 101, further comprising administering a lymphodepleting therapy to the subject prior to administering the population of cells.

Aspect 103. The method of aspect 102, wherein the lymphodepleting therapy comprises cyclophosphamide, fludarabine, or both cyclophosphamide and fludarabine.

Aspect B1. A method of increasing the yield of human immune cells and/or stem cells during ex vivo or in vitro culturing while increasing stemness of the human immune cells and/or stem cells comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 mM and 140 mM.

Aspect B2. A method of preparing a population of human immune cells and/or stem cells for immunotherapy comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 mM and 140 mM.

Aspect B3. A method of increasing stemness of human immune cells during ex vivo or in vitro culturing for immunotherapy comprising culturing human immune cells in a medium comprising potassium ion at a concentration between 40 mM and 80 mM and NaCl at a concentration between 30 mM and 100 mM, wherein the total concentration of potassium ion and NaCl is between 110 mM and 140 mM.

Aspect B4. The method of any one of Aspects B1 to 3, wherein the total concentration of potassium ion and NaCl is between 120 mM and 140 mM.

Aspect B5. The method of any one of Aspects B1 to 4, wherein the concentration of potassium ion is selected from the group consisting of about 40 mM, about 45 mM, about 50 mM, about 55 mM, about 60 mM, about 65 mM, about 70 mM, about 75 mM, and about 80 mM.

Aspect B6. The method of any one of Aspects B1 to 8, wherein the concentration of potassium ion is between 50 mM and 60 mM.

Aspect B7. The method of any one of Aspects Bi to 6, wherein the concentration of potassium ion is about 50 mM.

Aspect B8. The method of any one of Aspects B1 to 7, wherein the concentration of potassium ion is about 50 mM, and the concentration of NaCl is less than 90 mM.

Aspect B9. The method of any one of Aspects Bi to 6, wherein the concentration of potassium ion is about 55 mM.

Aspect B10. The method of any one of Aspects B1 to 6 and 9, wherein the concentration of potassium ion is about 55 mM, and the concentration of NaCl is less than 85 mM.

Aspect B11. The method of any one of Aspects B1 to 6, wherein the concentration of potassium ion is about 60 mM.

Aspect B12. The method of any one of Aspects B1 to 6 and 11, wherein the concentration of potassium ion is about 60 mM, and the concentration of NaCl is less than 80 mM.

Aspect B13. The method of any one of Aspects B1 to 5, wherein the concentration of potassium ion is between 60 mM and 70 mM.

Aspect B14. The method of Aspect B13, wherein the concentration of potassium ion is about 65 mM.

Aspect B15. The method of Aspect B13 or 14, wherein the concentration of potassium ion is about 65 mM, and the concentration of NaCl is less than 75 mM.

Aspect B16. The method of Aspect B13, wherein the concentration of potassium ion is about 70 mM.

Aspect B17. The method of Aspect B13 or 16, wherein the concentration of potassium ion is about 70 mM, and the concentration of NaCl is less than 70 mM.

Aspect B18. The method of Aspect B13, wherein the concentration of potassium ion is about 75 mM.

Aspect B19. The method of Aspect B13 or 18, wherein the concentration of potassium ion is about 75 mM, and the concentration of NaCl is less than 65 mM.

Aspect B20. The method of any one of Aspects B1 to 19, wherein the medium further comprises one or more cytokines.

Aspect B21. The method of Aspect B20, wherein the one or more cytokines comprise Interleukin-2 (IL-2), Interleukin-7 (IL-2), Interleukin-21 (IL-21), Interleukin-15 (IL-15), or any combination thereof.

Aspect B22. The method of Aspect B20, wherein the one or more cytokines comprise IL-2, IL-7, and IL-15.

Aspect B23. The method of any one of Aspects Bi to 22, wherein the cells comprise human immune cells.

Aspect B24. The method of Aspect B23, wherein the human immune cells comprise T cells, TILs, NK cells, TILs, Tregs, or any combination thereof.

Aspect B25. The method of any one of Aspects Bi to 22, wherein the cells comprise human stem cells.

Aspect B26. The method of any one of Aspects B1 to 25, wherein the cells express a chimeric antigen receptor (CAR).

Aspect B27. The method of any one of Aspects Bi to 25, wherein the cells express an engineered T cell receptor (TCR).

Aspect B28. The method of any one of Aspects B1 to 27, wherein the medium further comprises calcium ion, glucose, or any combination thereof.

Aspect B29. The method of any one of Aspects B1 to 28, wherein the medium further comprises a cell expansion agent.

Aspect B30. The method of Aspect B29, wherein the cell expansion agent comprises a GSK3B inhibitor, an ACLY inhibitor, a PI3K inhibitor, an AKT inhibitor, or any combination thereof.

Aspect B31. The method of Aspect B27, wherein the PI3K inhibitor is selected from LY294002, pictilisib, CAL101, IC87114, and any combination thereof.

Aspect B32. The method of Aspect B30, wherein the AKT inhibitor is selected from MK2206, A443654, AKTi-VIII, and any combination thereof.

Aspect B33. The method of any one of Aspects B1 to 32, wherein the medium is capable of: a. increasing the number and/or percentage of less differentiated and/or undifferentiated cells; b. increasing transduction efficiency; c. increasing stem-like immune cells; d. increasing in vivo viability; e. increasing cell potency; f. preventing cell exhaustion; or g. any combination thereof; in the final cell product as compared to the starting cell population.

Aspect B34. The method of any one of Aspects B1 to 33, wherein the medium further comprises glucose.

Aspect B35. The method of Aspect B34, wherein the concentration of glucose is more than about 10 mM.

Aspect B36. The method of Aspect B34 or 35, wherein the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM.

Aspect B37. The method of any one of Aspects B34 to 36, wherein the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM.

Aspect B38. The method of any one of Aspects B34 to 37, wherein the concentration of glucose is about 15.4 mM, about 15.9 mM, about 16.3 mM, about 16.8 mM, about 17.2 mM, or about 17.7 mM.

Aspect B39. The method of any one of Aspects B1 to 38, wherein the medium further comprises calcium ion.

Aspect B40. The method of Aspect B39, wherein the concentration of calcium ion is more than about 0.4 mM.

Aspect B41. The method of Aspect B39 or 40, wherein the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

Aspect B42. The method of any one of Aspects B39 to 41, wherein the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

Aspect B43. The method of any one of Aspects B1 to 42, wherein the medium comprises about 50 mM potassium ion and (i) about 80.5 mM NaCl; (ii) about 17.7 mM glucose; (iii) about 1.8 mM calcium ion; or (iv) any combination of (i)-(iii).

Aspect B44. The method of any one of Aspects B1 to 42, wherein the medium comprises about 55 mM potassium ion and (i) about 76 mM NaCl; (ii) about 17.2 mM glucose; (iii) about 1.7 mM calcium ion; or (iv) any combination of (i)-(iii).

Aspect B45. The method of any one of Aspects B1 to 42, wherein the medium comprises about 60 mM potassium ion and (i) about 72.2 mM NaCl; (ii) about 16.8 mM glucose; (iii) about 1.6 mM calcium ion; or (iv) any combination of (i)-(iii).

Aspect B46. The method of any one of Aspects B1 to 42, wherein the medium comprises about 65 mM potassium ion and (i) about 67.6 mM NaCl; (ii) about 16.3 mM glucose; (iii) about 1.5 mM calcium ion; or (iv) any combination of (i)-(iii).

Aspect B47. The method of any one of Aspects B1 to 42, wherein the medium comprises about 70 mM potassium ion and (i) about 63.9 mM NaCl; (ii) about 15.9 mM glucose; (iii) about 1.4 mM calcium ion; or (iv) any combination of (i)-(iii).

Aspect B48. The method of any one of Aspects B1 to 42, wherein the medium comprises about 75 mM potassium ion and (i) about 59.3 mM NaCl; (ii) about 15.4 mM glucose; (iii) about 1.3 mM calcium ion; or (iv) any combination of (i)-(iii).

Aspect B49. The method of any one of Aspects B1 to 42, wherein the medium comprises about 80 mM potassium ion and (i) about 55.6 mM NaCl; (ii) about 15 mM glucose; (iii) about 1.2 mM calcium ion; or (iv) any combination of (i)-(iii).

Aspect B50. The method of any one of Aspects B1 to 49, wherein the immune cells are CD3⁺, CD45RO⁻, CCR7⁺, CD45RA⁺, CD62L⁺CD27⁺, CD28⁺, or TCF7⁺, or any combination thereof, following culture.

Aspect B51. The method of any one of Aspects B1 to 50, wherein the medium comprises IL-2 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

Aspect B52. The method of Aspect B51, wherein the concentration of IL-2 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

Aspect B53. The method of Aspect B51 or 52, wherein the concentration of IL-2 is about 1.0 ng/mL.

Aspect B54. The method of Aspect B51 or 52, wherein the concentration of IL-2 is about 10 ng/mL.

Aspect B55. The method of any one of Aspects B1 to 54, wherein the medium comprises IL-21 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

Aspect B56. The method of Aspect B55, wherein the concentration of IL-21 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

Aspect B57. The method of Aspect B55 or 56, wherein the concentration of IL-21 is about 1.0 ng/mL.

Aspect B58. The method of Aspect B55 or 56, wherein the concentration of IL-21 is about 10 ng/mL.

Aspect B59. The method of any one of Aspects B1 to 58, wherein the medium comprises IL-7 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

Aspect B60. The method of Aspect B56, wherein the concentration of IL-7 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

Aspect B61. The method of Aspect B59 or 60, wherein the concentration of IL-7 is about 1.0 ng/mL.

Aspect B62. The method of Aspect B59 or 60, wherein the concentration of IL-7 is about 10 ng/mL.

Aspect B63. The method of any one of Aspects B1 to 62, wherein the medium comprises IL-15 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

Aspect B64. The method of Aspect B63, wherein the concentration of IL-15 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

Aspect B65. The method of Aspect B63 or 64, wherein the concentration of IL-15 is about 1.0 ng/mL.

Aspect B66. The method of Aspect B63 or 64, wherein the concentration of IL-15 is about 10 ng/mL.

Aspect B67. The method of any one of Aspects B1 to 66, wherein the immune cells and/or stem cells comprise a chimeric antigen receptor (CAR), an engineered T cell receptor, or any combination thereof.

Aspect B68. The method of any one of Aspects B1 to 67, wherein the immune cells and/or stem cells are administered to a human subject following culture.

Aspect B69. The method of any one of Aspects B1 to 68, wherein the cells are further transduced with a vector.

Aspect B70. The method of Aspect B69, wherein the vector comprises a transgene encoding a chimeric antigen receptor (CAR), a T cell receptor (TCR), or a TCR mimic.

Aspect B71. The method of any one of Aspects B26 and 67 to 70, wherein the CAR targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof.

Aspect B72. The method of any one of Aspects B27 and 67 to 70, wherein the TCR targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p⁵³ mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof.

Aspect B73. The method of any one of Aspects B69 to 72, wherein the vector is a retroviral vector, a lentiviral vector, an adeno-associated virus (AAV), an adenovirus, an AAV hybrid virus, a baculovirus, or any combination thereof.

Aspect B74. The method of any one of Aspects B1 to 73, wherein the potassium ion comprises potassium chloride.

Aspect B75. A population of cells prepared by the method of any one of Aspects Bi to 74.

Aspect B76. A population of cells comprising at least about 5%, at least about 10%, at least about 15%, at least about 20%, at least about 25%, at least about 30%, at least about 35%, at least about 40%, at least about 45%, at least about 50%, at least about 60%, or at least about 70% of the cells are CD3+, CD45RO−, CCR7+, CD45RA+, CD62L+, CD27+, CD28+, and TCF7+, wherein the cells are cultured according to the method of any one of Aspects B1 to 74.

Aspect B77. A cell culture medium useful for the method of any one of Aspects Bi to 74.

Aspect B78. A cell culture medium comprising (i) a potassium ion at a concentration higher than 40 mM and (ii) NaCl at a concentration of less than 100 mM.

Aspect B79. The culture medium of Aspect 78, wherein the concentration of the potassium ion is at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM.

Aspect B80. The culture medium of Aspect B78 or 79, comprising: a. about 45 mM potassium ion and less than 95 mM NaCl, b. about 50 mM potassium ion and less than 90 mM NaCl, c. about 55 mM potassium ion and less than 85 mM NaCl, d. about 60 mM potassium ion and less than 80 mM NaCl, e. about 65 mM potassium ion and less than 75 mM NaCl, f. about 70 mM potassium ion and less than 70 mM NaCl, g. about 75 mM potassium ion and less than 65 mM NaCl, h. about 80 mM potassium ion and less than 60 mM NaCl, i. about 85 mM potassium ion and less than 55 mM NaCl, or j. about 90 mM potassium ion and less than 50 mM NaCl.

Aspect B81. The culture medium of any one of Aspects B78 to 90, which further comprises one or more cytokines.

Aspect B82. The culture medium of Aspect B91, wherein the one or more cytokines are selected from the group consisting of IL-2, IL-7, IL-15, IL-21, and any combination thereof.

Aspect B83. The culture medium of any one of Aspects B78 to 82, wherein the potassium ion comprises potassium chloride.

Aspect B84. A method of treating a disease or condition in a subject in need thereof comprising administering the population of cells of Aspect B75 or 76 to the subject.

Aspect B85. The method of Aspect B84, wherein the disease or condition comprises a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof

### BRIEF DESCRIPTION OF THE DRAWINGS/FIGURES

FIG. 1A shows stringent cell surface markers and transcription factors for FACS analysis that accurately define “stem cell-like” cells, e.g., T cells. FIG. 1B is an illustration of progressive differentiation of exemplary cells, e.g., T cells, annotated with the various phenotypic characteristics and biomarker expression profiles.

FIGS. 2A-2F are scatter plots showing the relative stem-like populations of cells (FIGS. 2A-2C) and the relative yields (FIGS. 2D-2F) at varying potassium concentrations, wherein the medium has a tonicity of 250 mOsmol (FIGS. 2A and 2D), 280 mOsmol (FIGS. 2B and 2E), or 320 mOsmol (FIGS. 2C and 2F) (circles: 50 mM potassium ion; squares: 60 mM potassium ion; triangles: 70 mM potassium ion; inverted triangles: 80 mM potassium ion; diamonds: 90 mM potassium ion; open circles: control). FIG. 2G is a line graph showing the relative stem-like populations following culture in control medium or hypotonic medium having a tonicity of 250 mOsmol. Tonicity of the media was calculated based on the concentrations of potassium and NaCl as shown in Table 2.

FIGS. 3A-3G show the effects of culturing T cells in elevated potassium in hypotonic culture conditions. FIG. 3A illustrates a T cell culture scheme consistent with some aspects disclosed herein. FIGS. 3B-3H show FACS analysis of expression of CD45RA CCR7 under various culture conditions. FIGS. 3I-3O show FACS analysis of expression of CD45RA and CD62L under various culture conditions. FIGS. 3O-3V show FACS analysis of expression of T cell persistence associated marker CD27 and CCR7⁺ under various culture conditions.

FIGS. 3W-3CC show FACS analysis of expression of TCF7 and CD39 under various culture conditions. FIGS. 3DD-3FF show FACs analysis of TCF7 expression and CD3. FIG. 3GG shows the percentage of TCF7⁺ cells from control and hypotonic modified media with elevated potassium culture conditions.

FIGS. 4A-4B show T cell yield and viability during in vitro expansion. FIG. 4A shows fold expansion (10⁶) of T cells cultured in control media and modified (hypotonic) media, i.e., media containing elevated potassium and NaCl such that the combination of the potassium ion and NaCl is less than 140 mM. FIG. 4B shows viability of T cells cultured in control media and modified (hypotonic) media, i.e., media containing elevated potassium and NaCl such that the combination of the potassium ion and NaCl is less than 140 mM.

FIGS. 5A-5R show hypotonic conditioning medium-enriched “stem cell-like” T cells. FIGS. 5A-5I show FACS analysis of “stem cell-like” markers from CD4 and CD8 T cells cultured in control media. FIGS. 5J-5R show FACS analysis of “stem cell-like” markers from CD4 and CD8 T cells cultured in modified (hypotonic) media, i.e., media containing elevated potassium and NaCl such that the combination of the potassium ion and NaCl is less than 140 mM.

FIG. 6 shows the effect of modified media on transduction efficiency. The modified (hypotonic) media have elevated potassium and NaCl such that the combination of the potassium ion and NaCl is less than 140 mM.

FIG. 7 shows the effect of elevated potassium in the modified media on T_{SCM} populations.

FIGS. 8A-8B are images of western blots illustrating the proteomic profile of T cells cultured in MRM, showing features of both stemness (FIG. 8A) and cells that are poised for effector function (FIG. 8B).

FIGS. 9A-9J show the effect of elevated potassium on the fate of effector functions of T_{SCM} populations. FIGS. 9A-9D show FACS plots showing enrichment of IL-2 expression in CCR7⁺ and reduction of IFN-γ in CD19-CAR engineered CD4 T cells. FIGS. 9E-9H show FACS plots showing enrichment of IL-2 expression in CCR7⁺ and reduction of IFN-γ in CD19-CAR engineered CD8 T cells. FIGS. 9I-9J shows a quantification of IL-2 (FIG. 91) and IFN-γ (FIG. 9J) expression in control and elevated potassium in hypotonic culture conditions.

FIGS. 10A-10D show the effect of cytokines on T_{SCM} populations in control (left panels) or in modified (hypotonic) media (right panels) with elevated potassium and hypotonic culture conditions. FIGS. 10A-10B show the effect of the combination of IL-2, IL-7, and IL-15. FIGS. 10C-10D show the effect of the combination of IL-7 and IL-21.

FIGS. 11A-11B show the number of T_{SCM} cells in CAR engineered products cultured in control and elevated potassium in hypotonic culture conditions in modified (hypotonic) media comprising elevated potassium with cytokine combinations. FIG. 11A shows the number of T_{SCM} cells in CAR engineered products from Donor 1. From left to right, cells were cultured with IL-2, IL-7, and IL-15; IL-2 (10 ng/mL); IL-2 and IL-21 (1 ng/mL); IL-7 and IL-21 (1 ng/ml); IL-15 and IL-21 (1 ng/mL); Potassium and IL-2, IL-7, and IL-15; Potassium and IL-2 (10 ng/mL); Potassium and IL-2 and IL-21 (1 ng/mL); Potassium and IL-7 and IL-21 (1 ng/mL); Potassium and IL-15 and IL-21 (1 ng/mL). FIG. 11B shows the number of T_{SCM} cells in CAR engineered products from Donor 1. From left to right, cells were cultured with IL-2, IL-7, and IL-15; IL-2 (10 ng/mL); IL-2 and IL-21 (1 ng/mL); IL-7 and IL-21 (1 ng/ml); IL-15 and IL-21 (1 ng/mL); Potassium and IL-2, IL-7, and IL-15; Potassium and IL-2 (10 ng/mL); Potassium and IL-2 and IL-21 (1 ng/mL); Potassium and IL-7 and IL-21 (1 ng/mL); Potassium and IL-15 and IL-21 (1 ng/mL).

FIG. 12A-12G is a schematic showing generally an exemplary process of culturing and expanding TILs from tumor fragments. FIGS. 12B-12C show exemplary processes comprising an initial expansion and a secondary expansion, wherein the TILs are optionally stimulated (e.g., according to the methods disclosed herein, e.g., by contacting the cells with 4-1BBL, TRANSACT™, anti-CD3 antibody, an antigen presenting cell, or any combination thereof) at the transition from the initial TIL culture to the secondary TIL expansion (FIGS. 12B-12C) and during the initial TIL culture (FIG. 12C). FIGS. 12D-12E show exemplary processes comprising an initial expansion, a secondary expansion, and a final expansion, wherein the TILs are optionally stimulated (i) at the transition from the initial TIL culture to the secondary TIL expansion (FIGS. 12D-12E); (ii) at the transition from the secondary TIL expansion to the final TIL expansion (FIGS. 12D-12E); and (iii) during the initial TIL culture (FIG. 12E). FIGS. 12F-12G show exemplary processes for generating young TILs, wherein the initial expansion and the secondary expansion are shorter in duration, e.g., 11 days (or less) for each expansion, and wherein the TTLs are optionally stimulated at the transition from the initial TIL culture to the secondary TIL expansion (FIGS. 12F-12G) and during the initial TIL culture (FIG. 12G).

FIGS. 13A-13B are graphical representations of FACS cell phenotyping of TILs after initial culture (day 14) in T cell conditioned media (CTS™ OPTIMIZER™, also referred to herein as “control media”; FIG. 13A) or metabolic reprogramming media (also referred to herein as “MRM”; FIG. 13B). FIGS. 13A and 13B show that culture in MRM produced TILs with enhanced expression of CD39 and PD1 (greater than 20%) as compared to TILs cultured in control media. FIG. 13C is a scatter plot showing the individual differences in the percentage of CD8⁺ cells obtained by culturing TILs from various tumor types in either control or MRM. Each of the linked points represent TILs obtained from the same sample such that the figure summarizes data from 13 patients. Asterisks indicate that the average percentage of CD8⁺ TTLs following culture in control media is significantly different than the average percentage of CD8⁺ TILs following culture in MRM. These data show that culturing TILs in MRM results in enrichment of CD8⁺ T cells as compared to culturing TTLs in control media.

FIGS. 14A-14E are graphical representations of FACS cell phenotyping based on expression of PD1 and CD27 of cultured CD4⁺ (FIGS. 14A-14B) and CD8⁺ (FIGS. 14C-14D) TILs following 14-day culture in control media (FIGS. 14A and 14C) or MRM (FIGS. 14B and 14D). FIG. 14C is a scatter plot showing the individual differences in the percentage of CD27⁺ PD1⁺ cells obtained by culturing TILs from various tumor types in either control or MRM. Each of the linked points represent TTLs obtained from the same sample such that FIG. 14C summarizes data from 9 patients. Asterisks indicate that the average percentage of CD27⁺ PD1V TTLs following culture in control media is significantly different than the average percentage of CD27⁺ PD1 TILs following culture in MRM. These data show that culturing TTLs in MRM results in enrichment of CD27⁺ PD1⁺ T cells as compared to culturing TTLs in control media.

FIG. 15 is a graphical representation illustrating the statistically significant difference in the percentages of CD27⁺ CD28⁺ cells obtained by culturing TILs from various tumor types in either control media or MRM after the initial culture (day 14). Each of the linked points represent TILs obtained from the same sample such that FIG. 15 summarizes data from 9 patients. These data show that culturing TILs in MRM results in enrichment of CD27⁺ CD28⁺ T cells as compared to TTLs cultured in control media.

FIGS. 16A-16B are graphical representations of FACS cell phenotyping of TILs cultured (day 14) in control media (FIG. 16A) or MRM (FIG. 16B), gated first by CD8 or CD4 expression, followed by CD28 and CD27 expression, followed by CD103 and CD27 expression, followed by PD1 and CD103 expression, and finally by TCF7 and CD27 expression. FIG. 16C is a graphical representation illustrating the mean fluorescence intensity (MFI) of TCF7T TILs following initial culture in control media (1) or MRM (2) (about day 14).

FIGS. 17A-17H are graphical representations of FACS cell phenotyping of TILs expanded in control media (FIGS. 17A-17D) or MRM (FIGS. 17E-17H) after the secondary expansion (about day 21-26), gated first by CD8 or CD4 expression (FIGS. 17A and 17E), CD28 and CD27 expression gated on CD8⁺ cells (FIGS. 17B and 17F), PD1 and CD27 expression gated on CD8⁺ cells (FIGS. 17C and 17G), and finally by TCF7 and CD39 expression gated on CD8⁺ cells (FIGS. 17D and 17H). FIGS. 17B-17D and 17F-17H are CD8⁺ cells.

FIGS. 18A-18H are graphical representations of FACS cell phenotyping of CD8⁺ TILs expanded by co-culture with mutant KRAS-pulsed dendritic cells in control media (FIGS. 18A-18D) or MRM (FIGS. 18E-18H) after the secondary expansion (about day 21), gated first by CD8 or CD4 expression (FIGS. 18A and 18E), followed by CD28 and CD27 expression gated on CD8⁺ cells (FIGS. 18B and 18F), followed by PD1 and CD27 expression gated on CD8⁺ cells (FIGS. 18C and 18G), and finally by TCF7 and CD8 expression gated on PD1⁺ only and CD27PD1⁺ cells (FIGS. 18D and 18H). FIGS. 18B-18D and 18F-18H are CD8⁺ cells.

FIGS. 19A-19H are graphical representations of FACS cell phenotyping of TILs expanded by co-culture with wild-type KRAS-pulsed dendritic cells in control media (FIGS. 19A-19D) or MRM (FIGS. 19E-19H) after the secondary expansion (about day 21), gated first by CD8 or CD4 expression (FIGS. 19A and 19E), followed by CD28 and CD27 expression gated on CD8⁺ cells (FIGS. 19B and 19F), followed by PD1 and CD27 expression gated on CD8⁺ cells (FIGS. 19C and 19G), and finally by TCF7 and CD8 expression gated on PD1⁺ only and CD27⁺ PD1⁺ cells (FIGS. 19D and 19H). FIGS. 19B-19D and 19F-19H are CD8⁺ cells.

FIGS. 20A-20B are graphical representations of FACS cell phenotyping of cultured TILs following secondary expansion (about day 21-26) in control media (FIG. 20A) or MRM (FIG. 20B).

FIG. 21 is a bar graph showing the fold-change (FC) in gene expression of IL-2, B2M, GZMB, IFNγ, and TCF7 in TILs cultured in control media or MRM after the secondary expansion (about day 21). Expression of each gene is normalized to the expression in TILs cultured in control media.

FIGS. 22A-22L are graphical representations of FACS cell sorting of CD4⁺ or CD8⁺ TILs cultured in control media (FIGS. 22A, 22B, 22E, and 22F) or MRM (FIGS. 22C, 22D, and 22G-22L) after secondary expansion (about day 21-26), gated by PD1 expression (FIGS. 22A-22D) or CD103 expression (FIGS. 22E-22H) and CD39 expression (FIGS. 22A-22H). FIGS. 22I-22L show gating of CD4⁺ TILs (FIGS. 221 and 22K) and CD8⁺ TILs (FIGS. 22J and 22L) gated on PD1 and CD39 expression (FIGS. 22I-22J) followed by CD45RO and CD103 expression (FIGS. 21K-21L).

FIG. 23 is a bar graph of the percent of cells expressing IL-2 and/or IFNγ, as indicated, illustrating the differentiation status of ROR1 chimeric antigen receptor (CAR) T cells cultured in control media or metabolic reprogramming media (MRM; hypotonic media (i.e., modified media containing elevated potassium and NaCl such that the combination of the potassium ion and NaCl is less than 140 mM)).

FIG. 24 is a graphical representations of sequential kill data for ROR1 CAR T cells co-cultured in control media (“ROR1 CAR”) or in MRM media.

FIGS. 25A-25B are bar graphs illustrating IL-2 secretion (FIG. 25A) and IFNγ secretion (FIG. 25B) for CAR-T cells co-cultured with H1975 cells in control media or MRM, as indicated.

FIG. 26 is a line graph showing ROR1 CAR T cell proliferation in co-culture with H1975 target cells in control media (“ROR1 CAR”) and MRM, as indicated.

FIG. 27 is a Uniform Manifold Approximation and Projection (UMAP) illustrating the single-cell gene expression of a multitude of genes. Each individual dot represents a single cell. Control cells are generally clustered at UMAP_1 values of greater than 0; and MRM cells are generally clustered at UMAP_1 values of less than 0, with a limited degree of overlap as projected as ‘0’ along the abscissa.

FIG. 28 is a bar graph showing the percentage of total human healthy cells (“H”), or diseased donor cells (“D1” and “D2”) in culture that have a less-differentiate phenotype following culture in control media (1) or MRM (2).

FIG. 29 is a scatter plot of differential peak analysis from bulk ATAC-seq (y-axis) and differential gene analysis from bulk RNA-seq (x-axis) illustrating the transcriptional and epigenetic differences of cells cultured in control media or MRM.

FIG. 30 is a diagram illustrating the significant differential peak in the promoter region of TCF7 in cells cultured in control media or MRM, as indicated.

FIGS. 31A-31C provide comparison of the anti-ROR1 (R12) CAR transduction efficiency in T cells cultured (or grown) in T_{CM} (i.e., control medium) and MRM. The T cells (includes both CD4+ and CD8+ T cells) were derived from different donors:donor #1 (FIG. 31A), donor #2 (FIG. 31B), and donor #3 (FIG. 31C). The different transduction conditions (or test groups) are as follows: (1) non-transduced T cells cultured in T_{CM}; (2) T cells transduced with control CD19t-R12 CAR (i.e., R12 CAR without c-Jun) and cultured in T_{CM}; (3) T cells transduced with c-Jun-R12 CAR (i.e., R12 CAR with c-Jun) and cultured in T_{CM}; (4) non-transduced T cells cultured in MRM; (5) T cells transduced with control CD19t-R12 CAR and cultured in MRM; and (6) T cells transduced with c-Jun-R12 CAR and cultured in MRM.

FIGS. 32A-32C show the percentage of CD4+(black bar) and CD8+(white bar) T cells present within the total transduced T cell population from the different test groups. The different test groups are the same as that described in FIGS. 31A-31C. The T cells that were transduced were derived from three different donors: donor #1 (FIG. 32A), donor #2 (FIG. 32B), and donor #3 (FIG. 32C).

FIGS. 33A-33C show the effect of MRM on c-Jun protein expression level (shown as median fluorescence intensity (MFI)) in transduced T cells from the different test groups. The different test groups are the same as that described in FIGS. 31A-31C. The T cells that were transduced were derived from three different donors: donor #1 (FIG. 33A), donor #2 (FIG. 33B), and donor #3 (FIG. 33C).

FIGS. 34A-34F provide comparison of the percentage of stem-like transduced CD4+ T cells (FIGS. 34A, 34B, and 34C—three different donors) and CD8⁺ T cells (FIGS. 34D, 34E, and 34F—from the three different donors) from the different test groups. The different test groups. The different test groups are the same as that described in FIGS. 31A-31C. As explained in Example 2, stem-like cells were identified as CD45RO⁻CCR7⁺ CD45RA⁺CD62L⁺CD27⁺ CD28⁺TCF7⁺.

FIGS. 35A-35J provide comparison of the expression of various surface markers on transduced T cells (derived from donor #1) from the different test groups. The different test groups are the same as that described in FIGS. 31A-31C. FIGS. 35A, 35B, 35C, 35D, and 35E show the percentage of anti-ROR1 (R12) CAR transduced CD4+ T cells that expressed CD39, LAG3, PD1, TIGIT, and TIM3, respectively. FIGS. 35F, 35G, 35H, 35I, and 35J show the percentage of anti-ROR1 CAR transduced CD8⁺ T cells that expressed CD39, LAG3, PD1, TIGIT, and TIM3, respectively.

FIGS. 36A-36J provide comparison of the expression of various surface markers on transduced T cells (derived from donor #2) from the different test groups. The different test groups are the same as that described in FIGS. 31A-31C. FIGS. 36A, 36B, 36C, 36D, and 36E show the percentage of anti-ROR1 (R12) CAR transduced CD4+ T cells that expressed CD39, LAG3, PD1, TIGIT, and TIM3, respectively. FIGS. 36F, 36G, 36H, 36I, and 36J show the percentage of anti-ROR1 CAR transduced CD8⁺ T cells that expressed CD39, LAG3, PD1, TIGIT, and TIM3, respectively.

FIGS. 37A-37J provide comparison of the expression of various surface markers on transduced T cells (derived from donor #3) from the different test groups. The different test groups are the same as that described in FIGS. 31A-31C. FIGS. 37A, 37B, 37C, 37D, and 37E show the percentage of anti-ROR1 (R12) CAR transduced CD4+ T cells that expressed CD39, LAG3, PD1, TIGIT, and TIM3, respectively. FIGS. 37F, 37G, 37H, 37I, and 37J show the percentage of anti-ROR1 (R12) CAR transduced CD8⁺ T cells that expressed CD39, LAG3, PD1, TIGIT, and TIM3, respectively.

FIGS. 38A-38C provide comparison of IL-2 production by T cells transduced and cultured in MRM or T_{CM} after primary antigen stimulation. The T cells that were transduced were derived from three different donors: donor #1 (FIG. 38A), donor #2 (FIG. 38B), and donor #3 (FIG. 38C). The different test groups are as follows: (1) T cells transduced with control CD19t-R12 CAR (i.e., R12 CAR without c-Jun) and cultured in T_{CM} (closed circle); (2) T cells transduced with c-Jun-R12 CAR (i.e., R12 CAR with c-Jun) and cultured in T_{CM} (closed square); (3) T cells transduced with control CD19t-R12 CAR and cultured in MRM (open circle); and (4) T cells transduced with c-Jun-R12 CAR and cultured in MRM (open square). The x-axis provides the effector:target (E:T) ratio (i.e. ratio of transduced T cells to target tumor cell).

FIGS. 39A-39C provide comparison of IFN-7 production by T cells transduced and cultured in MRM or T_{CM} after multiple rounds of antigen stimulation. As further provided in Example 3, the serial stimulation assay was terminated when the number of transduced T cells required to reseed the subsequent round was not achieved: (i) four rounds of antigen stimulation for donor #1 (FIG. 39A), (ii) three rounds of antigen stimulation for donor #2 (FIG. 39B), and (iii) two rounds of antigen stimulation for donor #3 (FIG. 39C). The different test groups are as follows: (1) T cells transduced with control CD19t-R12 CAR (i.e., R12 CAR without c-Jun) and cultured in T_{CM} (closed circle); (2) T cells transduced with c-Jun-R12 CAR (i.e., R12 CAR with c-Jun) and cultured in T_{CM} (closed square); (3) T cells transduced with control CD19t-R12 CAR and cultured in MRM (open circle); and (3) T cells transduced with c-Jun-R12 CAR and cultured in MRM (open square). The x-axis provides the effector:target (E:T) ratio (i.e. ratio of transduced T cells to target tumor cell).

FIGS. 40A-40F show the ability of the transduced CD8⁺ T cells to kill target tumor cells after multiple rounds of antigen stimulation. The T cells that were transduced were derived from three different donors: donor #1 (FIGS. 40B and 40E), donor #2 (FIGS. 40A and 40D), and donor #3 (FIGS. 40C and 40F). FIGS. 40A, 40B, and 40C provide results for CD8⁺ T cells transduced with either the control CD19t-R12 CAR (i.e., R12 CAR without c-Jun) (black bars) or the c-Jun-R12 CAR (i.e., R12 CAR with c-Jun) (white bars), and cultured in a control medium (i.e., T_{CM}). FIGS. 40E, 40F, and 40G provide results for CD8⁺ T cells transduced with either the control CD19t-R12 CAR (black bars) or the c-Jun-R12 CAR (white bars), and cultured in MRM. The x-axis provides the effector:target (E:T) ratio (i.e. ratio of transduced T cells to target tumor cell).

FIGS. 41A-41H are graphical representations, illustrating the expression of marker genes in NSCLC TILs expanded using a control process (FIGS. 41A-41D) or MRM (41E-41H). TILs expanded in MRM exhibited superior phenotypic characteristics as measured by CD8⁺ T cell fraction, low CD39/CD69 expression (FIGS. 41B and 41D), central memory (CD45RO+CD6L+; FIGS. 41C and 41G) and high CD27 expression (FIGS. 41D and 41H). Dashed line highlighted box indicates unfavorable phenotype and solid line highlighted box indicates favorable phenotype.

FIGS. 42A-42C are graphical representations, illustrating negative expression by CD8⁺ T cells of both CD39 and CD69 within the T cell compartment in TILs obtained from a melanoma (FIG. 42A), a NSCLC (FIG. 42B), or a colorectal cancer (FIG. 42C). Cultures were initiated from freshly supplied human tumor samples and cells were expanded under control or MRM conditions. After final rapid expansion process (REP), TILs were analyzed for negative expression by CD8⁺ T cells of both CD39 and CD69 within the T cell compartment. For each analysis, TILs expanded from melanoma (n=6 independent tumors), NSCLC (n=5 independent tumors) and colorectal cancer (n=11 independent tumors) were assessed. Statistical significance was measured by paired t test. *** p<0.001, * p<0.05.

### DETAILED DESCRIPTION

The present disclosure is directed to methods of culturing cells, cells prepared by the methods, and/or compositions or kits for the cell culturing methods. In some aspects, the disclosure provides methods of generating a population of immune cells, e.g., T cells, for an adoptive cell therapy (ACT), wherein the immune cells have a less differentiated state and retain the ability to proliferate. In some aspects, the immune cells have a less differentiated state and maintain the ability to target and kill tumor cells. In some aspects, the immune cells have a less differentiated state, retain the ability to proliferate, and maintain the ability to target and kill tumor cells. In some aspects, immune cells, e.g., T cells, cultured according to the methods disclosed herein, have increased efficacy in an ACT, as compared to immune cells cultured according to conventional methods. In some aspects, immune cells, e.g., T cells, cultured according to the methods disclosed herein, have increased persistence upon administration to a subject in an ACT, as compared to immune cells cultured according to conventional methods. Such increased persistence refers to the ability of the immune cell, e.g., T cell, to infiltrate and function in the tumor microenvironment, ability to resist exhaustion, and the persistence of stemness to ensure continued expansion and durability of response. In some aspects, immune cells, e.g. T cells, cultured according to the methods disclosed herein, are stem-like cells. Such cells are capable of self-renewal, proliferation and differentiation. In some aspects, immune cells, e.g. T cells, cultured according to the methods disclosed herein, are stem-like cells which also express effector-like markers. In some aspects, immune cells, e.g. T cells, cultured according to the methods disclosed herein, are stem-like cells which also maintain the ability to target and kill tumor cells.

The cell culturing methods of the present disclosure are capable of increasing multipotency and/or pluripotency of the cultured cells or increasing transduction efficiency when the cells are being transduced with a vector. In some aspects, the culturing methods are capable of reducing and/or preventing cell exhaustion when the cells are cultured and/or the cells are used in therapy in vivo. In some aspects, the culturing methods are also capable of increasing in vivo viability, in vivo persistence, in vivo effector function, or any combination thereof. In some aspects, the culturing methods disclosed herein are capable of enriching oligoclonal or polyclonal tumor reactive stem-like T-cells and/or CD8⁺ TILs. In some aspects, the culturing methods disclosed herein are capable of preserving clonal diversity of the TILs derived from cancer patients.

In some aspects, the disclosure is directed to methods of culturing cells, e.g., pluripotent, multipotent, and/or immune cells, comprising placing the cells in a medium comprising potassium at a concentration of at least about 5 mM (e.g., higher than 5 mM), wherein the medium is not hypertonic, e.g., hypotonic or isotonic. Some aspects of the present disclosure are directed to methods of culturing cells, e.g., pluripotent, multipotent, and/or immune cells, comprising placing the cells in a medium comprising potassium at a concentration higher than 40 mM, e.g., about 50 mM-80 mM. In some aspects, the cells are immune cells. In some aspects, the immune cells comprise T cells, tumor-infiltrating lymphocytes (TILs), natural killer (NK) cells, regulatory T (Treg) cells, or any combination thereof. In some aspects, the cells are multipotent cells. In some aspects, the cells are pluripotent cells. In some aspects, the pluripotent cells are induced pluripotent stem cells or embryonic stem cells. In some aspects, the cells are hematopoietic stem cells.

Some aspects of the present disclosure are directed to a method of increasing the yield of human immune cells and/or stem cells during ex vivo or in vitro culturing while increasing stemness of the human immune cells and/or stem cells comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of preparing a population of human immune cells and/or stem cells for immunotherapy comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of increasing stemness of human immune cells during ex vivo or in vitro culturing for immunotherapy comprising culturing human immune cells in a medium comprising potassium ion at a concentration between 40 mM and 80 mM and NaCl at a concentration between 30 mM and 100 mM, wherein the total concentration of potassium ion and NaCl is between 110 and 140 mM. In some aspects the human immune cells are T cells.

In some aspects, the disclosure is directed to methods of culturing TILs ex vivo or in vitro comprising culturing a heterogeneous population of TILs in a metabolic reprogramming medium, e.g., a hyperkalemic medium comprising potassium ion at a concentration higher than 40 mM, wherein the hyperkalemic medium is not hypertonic. In some aspects, the disclosure is directed to methods of increasing the number or percentage of CD8⁺ TILs ex vivo or in vitro comprising culturing a heterogeneous population of TILs in a metabolic reprogramming medium, e.g., a hyperkalemic medium comprising potassium ion at a concentration of at least 5 mM. In other aspects, the disclosure is directed to methods of preparing a CD8⁺-enriched population of tumor infiltrating lymphocytes (TILs), comprising culturing a heterogeneous population of TILs ex vivo or in vitro in a metabolic reprogramming medium, e.g., a hyperkalemic medium comprising potassium ion at a concentration of at least 5 mM.

In some aspects, the medium is hypotonic. In some aspects, the medium is isotonic. In certain aspects, the medium further comprises interleukin (IL)-2, IL-21, IL-7, IL-15, or any combination thereof. In some aspects, the medium comprises IL-2, IL-7 and IL-15. In some aspects, the medium comprises IL-2 and IL-21. In some aspects, the medium further comprises sodium ion, calcium ion, glucose, or any combination thereof.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). In addition, “or” is used mean an open list of the components in the list. For example, “wherein X comprises A or B” means X comprises A, X comprises B, X comprises A and B, or X comprises A or B and any other components.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, the term “pluripotent cell” refers to a self-replicating cell that is capable of differentiating into any cell type, e.g., in the human body.

As used herein, a “multipotent cell” refers to a self-replicating cell, which is capable of differentiating into more than one differentiated progeny cell.

The term “control media” as used herein refers to any media in comparison to the metabolic reprogramming media (“MRM”) disclosed herein. Control media can comprise the same components as the metabolic reprogramming media except certain ion concentrations, e.g., potassium ion. In some aspects, metabolic reprogramming media described herein are prepared from control media by adjusting one or more ion concentrations, e.g., potassium ion concentration, as described herein. In some aspects, control media comprise basal media, e.g., CTS™ OPTMIZER™. In some aspects, control media comprise AIM V, RPMI, or a mixture comprising AIM V and RPMI. In some aspects, control media comprise (i) 50% AIM V, (ii) 50% RPMI1640, (iii) 5% or 10% human serum, and (iv) IL-2. In some aspects, control media thus comprises one or more additional components, including, but not limited to, amino acids, glucose, glutamine, T cell stimulators, antibodies, substituents, etc. that are also added to the metabolic reprogramming media, but control media have certain ion concentrations different from the metabolic reprogramming media. Unless indicated otherwise, the terms “media” and “medium” can be used interchangeably.

As used herein, the term “immune cell” refers to a cell of the immune system. In some aspects, the immune cell is selected from a T lymphocyte (“T cell”), B lymphocyte (“B cell”), natural killer (NK) cell, macrophage, eosinophil, mast cell, dendritic cell or neutrophil). In some aspects, the immune cell is a tumor-infiltrating cell (TIL). As used herein, a “TIL” refers to T cell that has at least once entered into a tumor or is capable of entering a tumor, e.g., within the parenchyma of a tumor. In some aspects, the tumor is a solid tumor. In some aspects, the tumor is a liquid tumor, e.g., a hematopoietic cancer. TILs prepared by the present methods can have one or more properties that are the same as the naturally occurring TILs. In some aspects, TILs prepared by the present methods have one or more properties that are not present in the naturally occurring TILs. TILs can be obtained using any methods. In some aspects, the TILs are obtained from a tumor sample from a subject. In some aspects, the tumor sample, or a portion thereof, is cultured under conditions that promote evasion of the TILs from the tumor tissue, proliferation of the TILs, and/or expansion of the TILs. In some aspects, the medium used to promote evasion, proliferation, and/or expansion of the TILs is any metabolic reprogramming medium, e.g., hyperkalemic medium, disclosed herein.

TILs include, but are not limited to, CD8⁺ T cells (i.e. cytotoxic T cells), CD4+ T cells, B cells, and natural killer cells. TILs include both primary (e.g., obtained from a patient tissue sample) and secondary TILs (e.g., TIL cell populations that have been cultured, expanded or proliferated from primary TILs. In some aspects the TILs are genetically modified. In some aspects, the TIL is a CD8⁺ T cell. CD8⁺ TILs are generally considered to be the subpopulation of TILs responsible for destroying cancer cells. Conversely, CD4⁺ TILs are generally considered to act as suppressors of the immune response, which can limit the immune response against the tumor.

In some aspects, TILs can be defined biochemically using cell surface markers. TTLs can be generally categorized by expressing one or more of the following biomarkers: CD4, CD8, TCR αβ, CD27, CD28, CD56, CCR7, CD45RA, CD95, PD-1, and CD25. In some aspects, TILs can be defined functionally by their ability to infiltrate tumors and selectively kill the cancer cells.

Primary immune cells, including primary T cells, can be obtained from a number of tissue sources, including peripheral blood mononuclear cells (PBMCs), bone marrow, lymph node tissue, cord blood, thymus tissue, tissue from a site of infection, ascites, pleural effusion, spleen tissue, and/or tumor tissue. Leukocytes, including PBMCs, can be isolated from other blood cells by well-known techniques, e.g., FICOLL™ separation and leukapheresis. Leukapheresis products typically contain lymphocytes (including T and B cells), monocytes, granulocytes, and other nucleated white blood cells. T cells are further isolated from other leukocytes, for example, by centrifugation through a PERCOLL™ gradient or by counterflow centrifugal elutriation. A specific subpopulation of T cells, such as CD3⁺, CD25⁺, CD28⁺, CD4⁺, CD8⁺, CD45RA⁺, GITR⁺, and CD45RO⁺ T cells, can be further isolated by positive or negative selection techniques (e.g., using fluorescence-based or magnetic-based cell sorting). For example, T cells can be isolated by incubation with any of a variety of commercially available antibody-conjugated beads, such as DYNABEADS®, CELLECTION™, DETACHABEAD™ (Thermo Fisher) or MACS® cell separation products (Miltenyi Biotec), for a time period sufficient for positive selection of the desired T cells or negative selection for removal of unwanted cells.

In some instances, autologous T cells are obtained from a cancer patient directly following cancer treatment. It has been observed that following certain cancer treatments, in particular those that impair the immune system, the quality of T cells collected shortly after treatment can have an improved ability to expand ex vivo and/or to engraft after being engineered ex vivo.

Whether prior to or after genetic modification, T cells can be activated and expanded generally using methods as described, for example, in U.S. Pat. Nos. 5,858,358; 5,883,223; 6,352,694; 6,534,055; 6,797,514; 6,867,041; 6,692,964; 6,887,466; 6,905,680; 6,905,681; 6,905,874; 7,067,318; 7,144,575; 7,172,869; 7,175,843; 7,232,566; 7,572,631; and 10,786,533, each of which is expressly incorporated by reference herein in its entirety. Generally, T cells can be expanded in vitro or ex vivo by contact with a surface having attached thereto an agent that stimulates a CD3/TCR complex associated signal and a ligand that stimulates a costimulatory molecule on the surface of the T cells. In particular, T cell populations can be stimulated, such as by contact with an anti-CD3 antibody or antigen-binding fragment thereof, or an anti-CD3 antibody immobilized on a surface or by contact with a protein kinase C activator (e.g., bryostatins) in conjunction with a calcium ionophore. For co-stimulation of an accessory molecule on the surface of the T cells, a ligand that binds the accessory molecule can be used. For example, a population of T cells can be contacted with an anti-CD3 antibody and an anti-CD28 antibody under conditions appropriate for stimulating proliferation of the T cells. To stimulate proliferation of either CD4+ T cells or CD8⁺ T cells, an anti-CD3 antibody and an anti-CD28 antibody can be employed. In certain aspects, the T cells are activated and expanded by contacting with TRANSACT™, DYNABEADS®, or other known activation agents.

As used herein, a “population” of cells refers to a collection of more than one cell, e.g., a plurality of cells. In some aspects, the population of cells comprises more than one immune cell, e.g., a plurality of immune cells. In some aspects, the population of cells is comprises a heterogeneous mixture of cells, comprising multiple types of cells, e.g., a heterogeneous mixture of immune cells and non-immune cells. In some aspects, the population of cells comprises a plurality of T cells.

As used herein, the terms “T cell” and “T lymphocyte” are interchangeable and refer to any lymphocytes produced or processed by the thymus gland. Non-limiting classes of T cells include effector T cells (such as CD8⁺ T cells) and T helper (Th) cells (such as CD4⁺ T cells). In some aspects, the immune cell (e.g., T cell) is a Th17 cell. In some aspects, the immune cell is a Th2 cell. In some aspects, the immune cell (e.g., T cell) is a Tc17 cell. In some aspects, the immune cell is a Th17 cell. In some aspects, the immune cell (e.g., T cell) is a tumor-infiltrating cell (TIL). In some aspects, the immune cell (e.g., T cell) is a Treg cell.

As used herein, the term “memory” T cells refers to T cells that have previously encountered and responded to their cognate antigen (e.g., in vivo, in vitro, or ex vivo) or which have been stimulated with, e.g., an anti-CD3 antibody (e.g., in vitro or ex vivo). Immune cells having a “memory-like” phenotype upon secondary exposure, such memory T cells can reproduce to mount a faster and strong immune response than during the primary exposure. In some aspects, TILs having a “memory-like” phenotype, upon secondary exposure to antigen or stimulation, reproduce or proliferate to mount a faster and strong immune response than during the primary exposure. In some aspects, memory T cells comprise central memory T cells (T_{CM} cells), effector memory T cells (T_{EM} cells), tissue resident memory T cells (T_{RM} cells), stem cell-like memory T cells (T_{SCM} cells), or any combination thereof.

As used herein, the term “stem-like” or “stem cell-like” refers to a property or an ability of a cell to self-renew and has the multipotent capacity to generate and reconstitute the entire spectrum of memory and effector T cell subsets. In some aspects, a stem-like cell can be measured by specific markers expressed by the cell. In some aspects, those stem-like markers can be one or more of CD45RA+, CD62L+, CCR7+, CD27+, CD28+, BACH2+, LEF1+, and TCF7+. In some aspects, the stem-like cells can be identified by a transcriptome analysis, e.g., using stemness gene signatures disclosed herein. In some aspects, the effector-like marker comprises a marker disclosed in Krishna et al., Science 370:1328-34 (Dec. 11, 2020); and/or Galletti et al., Nature Immunology (October 2018), each of which is incorporated by reference herein in its entirety.

As used herein, the term “stem cell-like memory T cells,” “T memory stem cells,” or “T_{SCM} cells” refers to memory T cells that express CD95, CD45RA, CCR7, and CD62L and are endowed with the stem cell-like ability to self-renew and the multipotent capacity to reconstitute the entire spectrum of memory and effector T cell subsets.

As used herein, the term “central memory T cells” or “T_{CM} cells” refers to memory T cells that express CD45RO, CCR7, and CD62L. Central memory T cells are generally found within the lymph nodes and in peripheral circulation.

As used herein, the term “effector-like” or “effector cell-like” refers to tumor cell killing capacity and cytokine polyfunctionality, e.g., ability of a cell to produce inflammatory cytokines and/or cytotoxic molecules. In some aspects, an effector-like cell can be measured by specific markers expressed by the cell. In some aspects, those effector-like markers can be one or more of pSTAT5+, STAT5+, pSTAT3+, and STAT3⁺. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL1ORB, IL11, IL11RA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, TL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof. In some aspects, the effector-like cells can be identified by a transcriptome analysis. In some aspects, the effector-like marker comprises a marker disclosed in Kaech et al., Cell 111:837-51 (2002); Tripathi et al., J. Immunology 185:2116-24 (2010); and/or Johnnidis et al., Science Immunology 6:eabe3702 (Jan. 15, 2021), each of which is incorporated by reference herein in its entirety.

In some aspects, the effector-like cells are characterized using an effector-associated gene set described in Gattinoni, L., et al., Nat Med 17(10):1290-97 (2011). In some aspects, the gene signature for effector-like cells comprises one or more genes selected from MTCH2, RAB6C, KIAA0195, SETD2, C2orf24, NRD1, GNA13, COPA, SELT, TNIP1, CBFA2T2, LRP10, PRKC1, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, Clorf58, Cllorf63, C6orfl29, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMI, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEFlA, GCNT1, GABARAPLI, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSFlB, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEEl, PYHINI, F2R, PLD1, THBS1, CD58, FAS, NETO2, CXCR6, ST6GALNAC2, DUSP4, AUTS2, Clorf2l, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, KLRB1, and any combination thereof (see, e.g., Gattinoni, L., et al., Nat Med 17(10):1290-97 (2011).

As used herein, the term “effector memory T cells” or “T_{EM} cells” refers to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

As used herein, the term “tissue resident memory T cells” or “T_{RM} cells” refers to memory T cells that do not circulate and remain resident in peripheral tissues, such as the skin, lung, and the gastrointestinal tract. In certain aspects, tissue resident memory T cells are also effector memory T cells.

As used herein, the term “naïve T cells” or “T_{N} cells” refers to T cells that express CD45RA, CCR7, and CD62L, but which do not express CD95. As used herein, the term “naïve TILs” refers to TILs that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated T_{EFF} cell and an immune response.

As used herein, the term “fragmenting,” “fragment,” and “fragmented” describe processes for disrupting a tumor, including mechanical fragmentation methods such as crushing, slicing, dividing, and morcellating tumor tissue as well as any other methods for disrupting the physical structure of tumor tissue.

The term “culturing” as used herein refers to the controlled growth of cells ex vivo and/or in vitro. As used herein, “culturing” includes the growth of cells, e.g., T cells and/or NK cells, during cell expansion, or cell engineering (e.g., transduction with a construct for expressing a CAR, a TCR, or a TCRm). In some aspects, the cultured cells are obtained from a subject, e.g., a human subject. In some aspects, the cultured cells comprise T cells or NK cells obtained from a human subject. In some aspects, the cultured cells comprise immune cells obtained from a human subject. In some aspects, the cultured cells comprise one or more engineered immune cell disclosed herein. In some aspects, the T cells and/or NK cells are purified prior to the culture. In some aspects, the T cells and/or NK cells are tumor-infiltrating T cells and/or NK cells. In some aspects, the cultured cells comprise TILs obtained from a human subject. In some aspects, the culturing comprises placing a tumor sample or tumor fragment into a medium disclosed herein, wherein the medium promotes TIL evasion from the tumor sample and TIL expansion. In some aspects, the TILs are isolated or purified prior to the culture. In some aspects, the cell culturing is intended to expand the number of cultured cells, e.g., to increase proliferation of the cells.

As used herein, “cell engineering” refers to the targeted modification of a cell, e.g., a pluripotent cell, a multipotent cell, or an immune cell disclosed herein. In some aspects, the cell engineering comprises viral genetic engineering, non-viral genetic engineering, introduction of receptors to allow for tumor specific targeting (e.g., a TCR, TCRm, and/or a CAR), introduction of one or more endogenous genes that improve T cell function, introduction of one or more synthetic genes that improve T cell function, or any combination thereof.

“Expand” or “expansion,” as used herein in reference to immune cell culture, e.g., TIL culture, refers to the process of stimulating or activating the cells and culturing the cells. The expansion process can lead to an increase in the proportion or the total number of desired cells, e.g., an increase in the proportion or total number of TILs, in a population of cultured cells, after the cells are stimulated or activated and cultured. Expansion does not require that all cell types in a population of cultured cells are increased in number. Rather, in some aspects, only a subset of cells in a population of cultured cells are increased in number during expansion, while the number of other cell types may not change or may decrease.

As used herein, the term “yield” refers to the total number of cells following a culture method or a portion thereof In some aspects, the term “yield” refers to a particular population of cells, e.g., stem-like T cells in a population of T cells. The yield can be determined using any methods, including, but not limited to, estimating the yield based on a representative sample.

As used herein, the term “stemness,” “stem cell-like,” “stem-like,” or “less-differentiated” refers to a cell, e.g., a pluripotent cell, a multipotent cell, or an immune cell (e.g., a T cell and/or an NK cell), that expresses markers consistent with a more naïve phenotype. For example, a less differentiated T cell can express one or more marker characteristic of a T_{N} or a T_{SCM} cell. In some aspects, a “less-differentiated” or “stem-like” T cell expresses CD45RA, CCR7, and CD62L. In some aspects, a “less-differentiated” or “stem-like” T cell expresses CD45RA, CCR7, and CD62L, and is CD45RO^{low}. In some aspects, a “less-differentiated” or “stem-like” T cell expresses CD45RA, CCR7, and CD62L, and does not express CD45RO. In some aspects, a “less-differentiated” or “stem-like” T cell expresses CD45RA, CCR7, CD62L, and TCF7. In some aspects, the methods disclosed herein promote cells, e.g., a pluripotent cell, a multipotent cell, or an immune cell (e.g., a T cell and/or an NK cell), having a less-differentiated phenotype. In some aspects, the methods disclosed herein promote the growth and/or proliferation of cells, e.g., TILs, having a less-differentiated phenotype. Without being bound by any particular mechanism, in some aspects, the methods disclosed herein block, inhibit, or limit differentiation of less-differentiated cells, e.g., a pluripotent cell, a multipotent cell, or an immune cell (e.g., a T cell and/or an NK cell), resulting in an increased number of stem-like cells in culture. For example, it is generally thought that to effectively control tumors, adoptive transfer of less-differentiated immune cells, e.g., T cells, NK cells, and/or TIL, with a stem cell-like memory or central memory phenotype are preferred. See Gattinoni, L., et al., J. Clin. Invest. 115:1616-1626 (2005); Gattinoni, L., et al. Nat Med 15(7):808-814 (2009); Lynn, R. C., et al., Nature 576(7786): 293-300 (2019); Gattinoni, L., et al., J. Clin. Invest. 115:1616-1626 (2005); Gattinoni, L., et al. Nat Med 15(7):808-814 (2009); and Gattinoni, L., et al., Nat Med 17(10): 1290-1297 (2011).

Stemness is characterized by the capacity to self-renew, the multipotency, and the persistence of proliferative potential. In some aspects, stemness is characterized by a particular gene signature, e.g., a combined pattern of expression across a multitude of genes. In some aspects, the gene signature comprises one or more genes selected from ACTN1, DSC1, TSHZ2, MYB, LEF1, TIMD4, MAL, KRT73, SESN3, CDCA7L, LOC283174, TCF7, SLC16A10, LASS6, UBE2E2, IL7R, GCNT4, TAF4B, SULTIBI, SELP, KRT72, STXBP1, TCEA3, FCGBP, CXCR5, GPA33, NELL2, APBA2, SELL, VIPR1, FAM153B, PPFIBP2, FCER1G, GJB6, OCM2, GCET2, LRRN1, IL6ST, LRRC16A, IGSF9B, EFHA2, LOC129293, APP, PKIA, ZC3H12D, CHMP7, KIAA0748, SLC22A17, FLJ13197, NRCAM, C5orfl3, GIPC3, WNT7A, FAM117B, BENDS, LGMN, FAM63A, FAM153B, ARHGEF11, RBM11, RIC3, LDLRAP1, PELI1, PTK2, KCTD12, LMO7, CEP68, SDK2, MCOLN3, ZNF238, EDAR, FAM153C, FAAH2, BCL9, C17orf48, MAP1D, ZSWIM1, SORBS3, IL4R, SERPINF1, C16orf45, SPTBN1, KCNQ1, LDHB, BZW2, NBEA, GAL3ST4, CRTC3, MAP3K1, HLA-DOA, RAB43, SGTB, CNN3, CWH43, KLHL3, PIM2, RGMB, C16orf74, AEBP1, SNORD115-11, SNORD115-11, GRAP, and any combination thereof (see, e.g., Gattinoni et al., Nature Medicine 17(10):1290-97 (2011)). In some aspects, the gene signature comprises one or more gene selected from NOG, TIMD4, MYB, UBE2E2, FCER1G, HAVCR1, FCGBP, PPFIBP2, TPST1, ACTN1, IGF1R, KRT72, SLC16A10, GJB6, LRRN1, PRAGMIN, GIPC3, FLNB, ARRB1, SLC7A8, NUCB2, LRRC7, MYO15B, MAL, AEBP1, SDK2, BZW2, GAL3ST4, PITPNM2, ZNF496, FAM117B, C16orf74, TDRD6, TSPAN32, C18orf22, C3orf44, LOC129293, ZC3H12D, MLXIP, C7orfl0, STXBP1, KCNQ1, FLJ13197, LDLRAP1, RAB43, RIN3, SLC22A17, AGBL3, TCEA3, NCRNA00185, FAM153B, FAM153C, VIPR1, MMP19, HBS1L, EEF2K, SNORA5C, UBASH3A, FLJ43390, RP6-213H19.1, INPP5A, PIM2, TNFRSF10D, SNRK, LOC100128288, PIGV, LOC100129858, SPTBN1, PROS1, MMP28, HES1, CACHDI, NSUN5C, LEF1, TTTY14, SNORA54, HSF2, C16orf67, NSUN5B, KIAA1257, NRG2, CAD, TARBP1, STRADB, MT1F, TMEM41B, PDHX, KDM6B, LOC100288322, UXS1, LGMN, NANOS2, PYGB, RASGRP2, C14orf80, XPO6, SLC24A6, FAM113A, MRM1, FBXW8, NDUFS2, KCTD12, and any combination thereof (see, e.g., Gattinoni, L., et al., Nat Med 17(10): 1290-1297 (2011) or Galletti et al. Nat Immunol 21, 1552-1562 (2020)).

In the presence of prolonged antigen exposure, such as in many cancers, more differentiated immune cells, e.g., effector and effector memory T cells, often become exhausted and lose their anti-tumor function. Biomarkers, e.g., T cell markers, can be measured using any methods. In some aspects, T cells are identified using antibody-staining following by gated flow cytometry.

As used herein, the term “tonicity” refers to the measure of the effective osmotic pressure gradient across a cell membrane. Tonicity can be measured or calculated based on the level of potassium ion and sodium chloride (NaCl) in a solution. Herein, tonicity is calculated as the sum of the concentration of potassium ion (K+) and the concentration of sodium chloride (NaCl), multiplied by two. Tonicity can be expressed in terms of the osmolality of the solution, e.g., the media. As used herein, a solution, e.g., medium, is considered “isotonic” when the concentration of solutes in the media is equivalent to the concentration of solutes inside the cell. As used herein, an isotonic medium has a tonicity of about 280 mOsm/L (e.g., ([K+] +[NaCl])×2=280).

As used herein, a solution, e.g., a medium, is considered “hypotonic” if the concentration of solutes in the solution is lower than the concentration of solutes in the cell. As used herein, a hypotonic solution has a tonicity of less than 280 mOsm/L (e.g., ([K+]+[NaCl])×2<280). In some aspects, a hypotonic medium has a tonicity from at least about 210 mOsm/L to less than about 280 mOsm/L. In some aspects, a hypotonic medium has a tonicity from at least about 220 mOsm/L to less than about 280 mOsm/L. In some aspects, a hypotonic medium has a tonicity from at least about 230 mOsm/L to less than about 280 mOsm/L. In some aspects, a hypotonic medium has a tonicity from at least about 240 mOsm/L to less than about 280 mOsm/L. In some aspects, a hypotonic medium described herein has a tonicity of about 250 mOsm/L (e.g., ([K+]+[NaCl])×2=250).

As used herein, a solution, e.g., a medium, is considered “hypertonic” if the concentration of solutes in the solution is higher than the concentration of solutes in the cell. As used herein, a hypertonic solution has an osmolality of greater than 300 mOsm/L (e.g., ([K+]+[NaCl])×2>280). In some aspects, a hypertonic medium described herein has a tonicity of about 320 mOsm/L. In certain aspects, the tonicity of the solution, e.g., medium is adjusted by increasing or decreasing the concentration of one or more solute selected from potassium ions, sodium ions, glucose, and any combination thereof. In some aspects, the tonicity of the solution, e.g., medium is adjusted by increasing or decreasing the concentration of potassium ions and NaCl. In some aspects, the tonicity of a medium can be maintained by offsetting the increase of one solute with a decrease in a second solute. For example, increasing the concentration of potassium ion in a medium without changing the concentration of sodium ions can increase the tonicity of the medium. However, if the concentration of potassium ions is increased and the concentration of sodium ions is decreased, the tonicity of the original medium can be maintained. As used herein, the tonicity of a medium is defined by the sum of the potassium concentration and the NaCl concentration, multiplied by two. See, e.g., Table 2.

As used herein, the terms “potassium,” “potassium ion,” “potassium cation,” and “K+” are used interchangeably to refer to elemental potassium. Elemental potassium exists in solution as a positive ion. However, it would be readily apparent to a person of ordinary skill in the art that standard means of preparing a solution comprising potassium ion include diluting a potassium containing salt (e.g., KCl) into a solution. As such, a solution, e.g., a medium, comprising a molar (M) concentration of potassium ion, can be described as comprising an equal molar (M) concentration of a salt comprising potassium.

As used herein, the terms “sodium ion” and “sodium cation” are used interchangeably to refer to elemental sodium. Elemental sodium exists in solution as a monovalent cation. However, it would be readily apparent to a person of ordinary skill in the art that standard means of preparing a solution comprising sodium ion include diluting a sodium-containing salt (e.g., NaCl) into a solution. As such, a solution, e.g., a medium, comprising a molar (M) concentration of sodium ion, can be described as comprising an equal molar (M) concentration of a salt comprising sodium.

As used herein, the terms “calcium ion” and “calcium cation” are used interchangeably to refer to elemental calcium. Elemental calcium exists in solution as a divalent cation. However, it would be readily apparent to a person of ordinary skill in the art that standard means of preparing a solution comprising calcium ion include diluting a calcium-containing salt (e.g., CaCl₂)) into a solution. As such, a solution, e.g., a medium, comprising a molar (M) concentration of calcium ion, can be described as comprising an equal molar (M) concentration of a salt comprising calcium.

As used herein, the term “basal” media refers to any starting media that is supplemented with one or more of the additional elements disclosed herein, e.g., potassium, sodium, calcium, glucose, IL-2, IL-7, IL-15, IL-21, or any combination thereof. The basal media can be any media for culturing immune cells, e.g., T cells and/or NK cells. In some aspects, the basal media is selected from a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (11MDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™ XF, AIM V™, TEXMACS™ medium, TRANSACT™ TIL expansion medium, TIL rapid expansion protocol medium, and any combination thereof. In some aspects, the basal medium is serum free. In some aspects, the basal media comprises PRIME-XV T cell CDM. In some aspects, the basal media comprises OPTMIZER™. In some aspects, the basal media comprises OPTMIZER™ Pro. In some aspects, the basal media comprises X-VIVO™ 15 (LONZA). In some aspects, the basal media comprises IMMUNOCULT™. In some aspects, the basal media comprises Click's medium. In some aspects, the basal media comprises TRANSACT™ TIL expansion medium. In some aspects, the basal media comprises TIL rapid expansion medium. In some aspects, the basal medium further comprises immune cell serum replacement (ICSR). For example, in some aspects, the basal medium comprises OPTMIZER™ Complete supplemented with ICSR, AIM V™ supplemented with ICSR, IMMUNOCULT™ XF supplemented with ICSR, RPMI supplemented with ICSR, TEXMACS™ supplemented with ICSR, or any combination thereof. In some aspects, suitable basal media include Click's medium, OPTMIZER™ (CTS™) medium, STEMLINE® T cell expansion medium (Sigma-Aldrich), AIM V™ medium (CTS™), TEXMACS™ medium (Miltenyi Biotech), IMMUNOCULT® medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OPTMIZER™. In some aspects, suitable basal media include Click's medium, OPTIMIZER™ (CTS™) medium, STEMLINE® T cell expansion medium (Sigma-Aldrich), AIM V™ medium (CTS™), TEXMACS™ medium (Miltenyi Biotech), IMMUNOCULT® medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OPTIMIZER™. In some aspects, the basal media comprises one or more sodium salt in addition to the NaCl that is added to control the tonicity, e.g., NaCl added in combination with potassium ion.

As used herein, the term “cytokine” refers to small, secreted proteins released by cells that have a specific effect on the interactions and communications between cells. Non-limiting examples of cytokines include interleukins (e.g., interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23), interferons (IFN; e.g., IFN-α, IFN-β, and IFN-γ), tumor necrosis factor (TNF) family members, and transforming growth factor (TGF) family members. Certain aspects of the present disclosure are directed to methods of culturing cells, e.g., T cells and/or NK cells, in a medium comprising a cytokine. Some aspects of the present disclosure are directed to methods of expanding cells, e.g., T cells and/or NK cells, in a medium comprising a cytokine. In some aspects, the cytokine is an interleukin. In some aspects, the cytokine is selected from IL-2, IL-7, IL-15, IL-21 and any combination thereof. IL-2 (UniProtKB-P60568) is produced by T cells in response to antigenic or mitogenic stimulation. IL-2 is known to stimulate T cell proliferation and other activities crucial to regulation of the immune response. IL-7 (UniProtKB-P13232) is a hematopoietic growth factor capable of stimulating the proliferation of lymphoid progenitors. IL-7 is believed to play a role in proliferation during certain stages of B-cell maturation. IL-15 (UniProtKB-P40933), like IL-2, is a cytokine that stimulates the proliferation of T-lymphocytes. IL-21 (UniProtKB-Q9HBE4) is a cytokine with immunoregulatory activity. IL-21 is thought to promote the transition between innate and adaptive immunity and to induce the production of IgG1 and IgG3 in B-cells. IL-21 may also play a role in proliferation and maturation of natural killer (NK) cells in synergy with IL-15, and IL-21 may regulate proliferation of mature B- and T-cells in response to activating stimuli. In synergy with IL-15 and IL-18, IL-15 also stimulates interferon gamma production in T-cells and NK cells, and IL-21 may also inhibit dendritic cell activation and maturation during a T-cell-mediated immune response.

As used herein, the term “hyperkalemic,” e.g., “hyperkalemic medium,” refers to a medium that has an increased potassium concentration. In some aspects, the hyperkalemic medium comprises potassium ion at a concentration of at least 5 mM. In some aspects, the hyperkalemic medium comprises potassium ion at a concentration higher than 40 mM. In some aspects, the hyperkalemic medium a concentration of potassium ion of at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM. The term “metabolic reprogramming media,” “metabolic reprogramming medium,” or “MRM,” as used herein, refers to a hyperkalemic medium of the present disclosure. In certain aspects, the metabolic reprogramming media comprises about 40 mM to about 80 mM NaCl, about 40 mM to about 90 mM KCl, about 0.5 mM to about 2.8 mM calcium, and about 10 mM to about 24 mM glucose. In some aspects, the metabolic reprograming media further comprises an osmolality of about 250 to about 340 mOsmol.

As used herein, the term “higher than” means greater than but not equal to. For example, “higher than 4 mM” means any amount that is more than 4 mM, but which does not include 4 mM.

The term “preferentially,” as used herein, refers to the predominant outcome. For example, if the methods disclosed herein preferentially promote expansion of CD8⁺ TILs, it is to be understood that the predominant product of the expansion is CD8⁺ TILs. The term “preferentially” does not necessarily mean that 100% of, e.g., the resulting TILs are CD8⁺, rather the term suggests that CD8^{m} TILs are expanded to a greater extent than CD8− TILs.

As used herein, “administering” refers to the physical introduction of a therapeutic agent or a composition comprising a therapeutic agent to a subject, using any of the various methods and delivery systems. The different routes of administration for a therapeutic agent described herein (e.g., T cell, an NK cell, and/or a TIL cultured as described herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., a T cell, an NK cell, and/or a TIL cultured as described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell).

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The term “hematological malignancy” or “hematological cancer” refers to mammalian cancers and tumors of the hematopoietic and lymphoid tissues. Non-limiting examples of hematological malignancies include those affecting tissues of the blood, bone marrow, lymph nodes, and lymphatic system, including acute lymphoblastic leukemia (ALL), chronic lymphocytic lymphoma (CLL), small lymphocytic lymphoma (SLL), acute myelogenous leukemia (AML), chronic myelogenous leukemia (CIVIL), acute monocytic leukemia (AMoL), Hodgkin's lymphoma, and non-Hodgkin's lymphomas. Hematological malignancies are also referred to as “liquid tumors.” Liquid tumor cancers include, but are not limited to, leukemias, myelomas, and lymphomas, as well as other hematological malignancies. TILs obtained from liquid tumors may also be referred to herein as marrow infiltrating lymphocytes (MILs).

A “solid tumor,” as used herein, refers to an abnormal mass of tissue. Solid tumors may be benign or malignant. Non-limiting examples of solid tumors include sarcomas, carcinomas, and lymphomas, such as cancers of the lung, breast, prostate, colon, rectum, and bladder. The tissue structure of a solid tumor includes interdependent tissue compartments including the parenchyma (cancer cells) and the supporting stromal cells in which the cancer cells are dispersed, and which may provide a supporting microenvironment.

In some aspects, the cancer is selected from adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In some aspects, the cancer is selected from chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma. In some aspects, the cancer is selected from acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma. In some aspects, the cancer is selected from acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum. In some aspects, the cancer is selected from Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell (e.g. TIL), or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8⁺ T cell. In some aspects, a T cell is a NKT cell. In some aspects, an immune cell is a TIL. In some aspects, the TIL is a CD4⁺ TIL. In some aspects, the TIL is a CD8⁺ TIL.

As used herein, the term “anti-tumor immune response” refers to an immune response against a tumor antigen.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. As used herein, the phrase “subject in need thereof” includes subjects, such as mammalian subjects, that would benefit, e.g., from administration of immune cells (e.g., T cells and/or NK cells) cultured as described herein to control tumor growth.

The term “therapeutically effective amount” or “therapeutically effective dosage” refers to an amount of an agent (e.g., a T cell NK cell, and/or TIL cultured as described herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., cells cultured as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., T cells cultured as described herein), which is clinically proven to effect a significant decrease in cancer or slowing of progression (regression) of cancer, such as an advanced solid tumor. The ability of a therapeutic agent of the present disclosure (e.g., T cells cultured as described herein) to promote disease regression can be evaluated using a variety of methods known to the skilled practitioner, such as in human subjects during clinical trials, in animal model systems predictive of efficacy in humans, or by assaying the activity of the agent in in vitro assays.

The terms “effective” and “effectiveness” with regard to a treatment include both pharmacological effectiveness and physiological safety. Pharmacological effectiveness refers to the ability of a composition disclosed herein (e.g., cells cultured as described herein) to promote cancer regression in the patient. Physiological safety refers to the level of toxicity, or other adverse physiological effects at the cellular, organ, and/or organism level (adverse effects) resulting from administration of a composition disclosed herein (e.g., cells cultured as described herein).

The terms “chimeric antigen receptor” and “CAR,” as used herein, refer to a recombinant fusion protein that has an antigen-specific extracellular domain coupled to an intracellular domain that directs the cell to perform a specialized function upon binding of an antigen to the extracellular domain. In some aspects, a chimeric antigen receptor disclosed herein comprises a chimeric polypeptide of the present disclosure.

The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MHC-independent antigen and transduce activation signals via their intracellular domain.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 pM to about 10 pM, for example, about 0.1 pM to about 1 pM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, IgNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing VaVP) are also suitable for use in a TCR of the present disclosure.

As used herein, the term “T cell receptor” or “TCR” refers to a heterodimer composed of 2 different transmembrane polypeptide chains: an a chain and a β chain, each consisting of a constant region, which anchors the chain inside the T-cell surface membrane, and a variable region, which recognizes and binds to the antigen presented by MHCs. The TCR complex is associated with 6 polypeptides forming 2 heterodimers, CD3γε and CD3γε, and 1 homodimer CD3 ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

As used herein, an “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells and/or NK cells.

A “TCR mimic” or a “TCRm” refers to a type of antibody that recognize epitopes comprising both the peptide and the MHC-I molecule, similar to the recognition of such complexes by the TCR on T cells.

As used herein, the term “transduction efficiency” refers to: (i) the amount of material (e.g., exogenous polynucleotide) that can be physically introduced into a cell within a defined period of time; (ii) the amount of time it takes to physically introduce a given amount of material into a cell; (iii) the level to which a target material, e.g., an exogenous polynucleotide, i.e., a transgene, is taken up by a population of cells (e.g., the percentage of cells that express the transgene); or (iv) any combination of (i)-(iii). In some aspects, by increasing transduction efficiency, the culturing methods provided herein can allow for a greater amount of an exogenous nucleotide sequence to be introduced into a cell and/or decrease the amount of time required to introduce a given amount of an exogenous nucleotide sequence. Not to be bound by any one theory, in some aspects, such an effect can increase the expression of the encoded protein (e.g., c-Jun polypeptide) in the modified immune cell. As used herein, the terms “ug” and “uM“are used interchangeably with”μg“and” μM,” respectively.

Various aspects described herein are described in further detail in the following subsections.

II. Methods of the Disclosure

The present disclosure is directed to methods of culturing cells ex vivo or in vitro. In some aspects, the methods of the present disclosure comprise culturing or placing cells, e.g., a pluripotent cell, a multipotent cell, or an immune cell (e.g., a T cell and/or an NK cell) in a culture condition, wherein the culture (e.g., certain ion concentrations, tonicity of the medium, cytokines, and/or any combination thereof) is capable of reducing, limiting, or preventing the differentiation of the cells, thereby affecting or improving their use in a cell therapy.

In some aspects, the methods of the present disclosure comprise culturing or placing immune cells, e.g., TILs, in a culture condition, wherein the culture (e.g., certain ion concentrations, tonicity of the medium, cytokines, and or any combination thereof) is capable of enhancing the expansion of CD8⁺ TILs. In some aspects, the culture (e.g., certain ion concentrations, tonicity of the medium, cytokines, and or any combination thereof) is capable of limiting or preventing the differentiation of the TILs (e.g., CD8⁺ TILs and/or CD4⁺ TILs), thereby affecting or improving their use in a cell therapy. In some aspects, the present disclosure comprises culturing of TILs in a metabolic reprogramming media that is high in potassium concentration.

In some aspects, the cells, e.g., immune cells (e.g., T cells and/or NK cells), are cultured in a metabolic reprogramming media (MRM) disclosed herein. In some aspects, the immune cells, e.g., T cells and/or NK cells, cultured in MRM have a higher proportion of stem-like cells as compared to cells cultured using conventional methods, e.g., in a medium having less than 5 mM potassium ion. In some aspects, the immune cells, e.g., T cells and/or NK cells, cultured in MRM have a higher proportion of effector-like cells as compared to cells cultured using conventional methods, e.g., in a medium having less than 5 mM potassium ion. In some aspects, the immune cells, e.g., T cells and/or NK cells, cultured in MRM have a higher proportion of both stem-like and effector-like cells as compared to cells cultured using conventional methods, e.g., in a medium having less than 5 mM potassium ion. In some aspects, the immune cells, e.g., T cells and/or NK cells, cultured in MRM have a higher proliferative potential as compared to cells cultured using conventional methods, e.g., in a medium having less than 5 mM potassium ion.

Some aspects of the present disclosure are directed to methods of preparing a population of immune cells, e.g., T cells and/or NK cells, comprising culturing the cells in a medium comprising potassium ion at a concentration higher than 5 mM (e.g., a metabolic reprogramming medium disclosed herein). Some aspects of the present disclosure are directed to methods of preparing a population of T cells, comprising culturing the T cells in a medium comprising potassium ion at a concentration higher than 5 mM (e.g., a metabolic reprogramming medium disclosed herein). In some aspects, the present disclosure provides methods of preparing immune cells, e.g., T cells and/or NK cells, comprising culturing the cells in a medium comprising potassium ion at a concentration higher than 5 mM (e.g., higher than 40 mM, e.g., between 55 mM and 70 mM), are capable of preserving a stem-like phenotype (e.g., minimal differentiation) of the cultured cells. In some aspects, the present disclosure provides methods of preparing T cells, comprising culturing the T cells in a medium comprising potassium ion at a concentration higher than 5 mM (e.g., higher than 40 mM, e.g., between 55 mM and 70 mM), are capable of preserving a stem-like phenotype (e.g., minimal differentiation) of the cultured T cells. In some aspects, the cultured cells have more stem-like phenotypes (e.g., less differentiated) than cells grown in a medium having a lower potassium concentration.

Cell culture media are useful for the in vitro expansion of cell products used to expand and activate cells for the treatments of varieties of maladies. One of the most urgent unmet needs is for the expansion of cells that can be used in therapy, e.g., cells that can destroy cancer cells. Metastatic solid tumors are responsible for >500,000 deaths in the United States alone. It is known that T lymphocytes are capable of killing tumors in settings where other available treatments are ineffective. For example, surgery, chemotherapy, radiation therapy and ‘targeted’ or small molecule therapies are not effective for many common types of metastatic cancers such as those of the colon, breast, lung, pancreas, prostate, bile duct and other histologies. Cell-based cancer immunotherapies can represent curative therapy in these histologies.

The efficacy of cellular immunotherapy is dependent on the persistence, multipotency, and asymmetric cell division in vivo (phenotypic characteristics of T cell stemness). The media that are employed in the culturing and/or engineering of therapeutic cells can profoundly affect the metabolic, epigenetic, and phenotypic attributes of the cells. The formulations of media currently in use have not undergone significant change in approximately 40 years (see Moore et al., JAMA 199(8): 519-24 (1967), which is incorporated by reference herein in its entirety)). However, robust preclinical data disclosed herein demonstrate that the concentration of ions, especially potassium (K+), nutrients, and/or cytokines can significantly affect the multipotency of T cells during ex vivo manipulations. This effect is multifactorial and depends on the target of the therapeutic cells, cell function within the tumor microenvironment, and the quality of the therapeutic cells prior to transfer. Disclosed herein are strategies to promote antitumor cell stemness, e.g., prior to adoptive transfer.

In some aspects, the method of the present disclosure comprises placing, growing, or culturing the cells, e.g., immune cells, e.g., T cells and/or NK cells, in a medium comprising potassium ion at a concentration of at least about 5 mM, wherein the medium is hypotonic. In certain aspects, the concentration of potassium ion is at least about 50 mM. In some aspects, the method of the present disclosure comprises placing, growing, or culturing the cells, e.g., T cells, in a medium comprising potassium ion at a concentration of higher than about 40 mM, wherein the medium is hypotonic or isotonic. In certain aspects, the concentration of potassium ion is at least about 50 mM. In some aspects, the method of the present disclosure comprises placing, growing, or culturing the cells, e.g., T cells, in a medium comprising potassium ion at a concentration of at least about 50 mM, wherein the medium is hypotonic or isotonic. Some aspects of the present disclosure are directed to a method of preparing a population of chimeric antigen receptor (CAR) or TCR-expressing immune cells, e.g., T cells and/or NK cells, comprising placing the cells into a medium comprising potassium ion at a concentration of at least about 5 mM, wherein the medium is hypotonic. In certain aspects, the concentration of potassium ion is at least about 50 mM. Some aspects of the present disclosure are directed to a method of preparing a population of chimeric antigen receptor (CAR) or TCR-expressing immune cells, e.g., T cells, NK cells, and/or TIL, comprising placing the cells into a medium comprising potassium ion at a concentration of at least about 5 mM, wherein the medium is not hypertonic, e.g., wherein the medium is hypotonic or isotonic. Some aspects of the present disclosure are directed to a method of preparing a population of chimeric antigen receptor (CAR) or TCR-expressing immune cells, e.g., T cells, NK cells, and/or TIL, comprising placing the cells into a medium comprising potassium ion at a concentration of at least about 50 mM. In some aspects, the medium is hypotonic or isotonic. The medium can be prepared by adding a sufficient amount of a potassium salt. The tonicity of the medium can be modified by controlling the concentration of NaCl in the medium relative to the concentration of potassium ion. In some aspects, the total concentration of potassium ion and NaCl is between 110 mM and 140 mM.

In some aspects, the present disclosure also provides that cells, e.g., pluripotent, multipotent, and/or immune cells (e.g., T cells and/or NK cells), grown in a medium having a high concentration of potassium ion (e.g., higher than about 5 mM, e.g., higher than 40 mM, e.g., between 55 mM and 70 mM), but being isotonic or hypotonic, are capable of preserving a more stem-like phenotype of minimal differentiation than cells grown in a medium being hypertonic. In some aspects, therefore, the medium is not hypertonic after adding the potassium salt. In some aspects, the medium is hypotonic. In some aspects, the medium is isotonic. In certain aspects, the medium further comprises interleukin (IL)-2, IL-21, IL-7, IL-15, or any combination thereof. In some aspects, the medium further comprises sodium ion (e.g., NaCl), calcium ion, glucose, or any combination thereof.

In certain aspects, a population of cells, e.g., pluripotent, multipotent, and/or immune cells (e.g., T cells and/or NK cells), cultured using the methods disclosed herein exhibits an increased number of stem-like cells relative to a population of cells cultured using conventional methods, e.g., in a medium having less than 5 mM potassium ion or under hypertonic conditions. In some aspects, the cells, e.g., pluripotent, multipotent, and/or immune cells (e.g., T cells and/or NK cells), exhibit increased expression of markers characteristic of stem-like cells relative to the starting population of cells. In some aspects, the starting population of cells comprises cells obtained from a human subject. In some aspects, the starting population of cells comprises T cells obtained from a human subject. In some aspects, the starting population of T cells comprises T_{N} cells, T_{SCM} cells, T_{CM} cells, T_{EM} cells, or any combination thereof.

In some aspects, a population of immune cells, e.g., T cells and/or NK cells, cultured using the methods disclosed herein, exhibits an increased number of stem-like cells relative to a population of cells cultured using conventional methods, e.g., in a medium having less than 5 mM potassium ion. In some aspects, a population of T cells, cultured using the methods disclosed herein, exhibits an increased number of stem-like T cells relative to a population of T cells cultured using conventional methods, e.g., in a medium having less than 5 mM potassium ion. In some aspects, the immune cells, e.g., T cells and/or NK cells, exhibit increased expression of markers characteristic of stem-like cells relative to the starting population of immune cells (i.e., prior to the culturing). In some aspects, the T cells, exhibit increased expression of markers characteristic of stem-like cells relative to the starting population of T cells (i.e., prior to the culturing). In some aspects, the starting population of immune cells comprises immune cells (e.g., T cells and/or NK cells) obtained from a human subject. In some aspects, the starting population of immune cells comprises T cells obtained from a human subject. In some aspects, the starting population of immune T cells comprises T_{N} cells, T_{SCM} cells, T_{CM} cells, T_{EM} cells, or any combination thereof. In some aspects, the starting population of immune cells comprises T cells prior to transfection with a construct encoding a ligand binding protein as described herein.

Increased cell multipotency can be measured using any methods known in the art. In certain aspects, cell stemness is measured by antibody staining followed by gated flow cytometry. In some aspects, the cell stemness is measured by autophagy flux. In some aspects, the cell stemness is measured by glucose uptake. In some aspects, the cell stemness is measured by fatty acid uptake. In some aspects, the cell stemness is measured by mitochondrial biomass. In some aspects, the cell stemness is measured by RNA quantification/expression analysis (e.g., microarray, qPCR (taqman), RNA-Seq., single-cell RNA-Seq., or any combinations thereof). In some aspects, the cell stemness is measured by (e.g., transcripts that are linked to) a metabolism assay (e.g., a Seahorse metabolism assay, analysis of extracellular acidification rate (ECAR); analysis of oxygen consumption rate (OCR); analysis of spare respiratory capacity; and/or analysis of mitochondrial membrane potential). In some aspects, stemness is measured using one or more in vivo functional assays (e.g., assaying cell persistence, antitumor capacity, antitumor clearance, viral clearance, multipotency, cytokine release, cell killing, or any combination thereof).

In some aspects, the differentiation status of the cells, e.g., the pluripotent cells, the multipotent cells, and/or the immune cells (e.g., T cells and/or NK cells (e.g., CD8⁺ TILs, e.g., tumor reactive CD8⁺ TILs)), is characterized by increased numbers of cells expressing markers typical of less differentiated cells. In some aspects, the differentiation status of the cells, e.g., T cells, is characterized by increased numbers of cells expressing markers typical of less differentiated cells, e.g., T cells. In some aspects, an increase in the number of stem-like cells is characterized by increased numbers of immune cells (e.g., T cells and/or NK cells (e.g., CD8⁺ TILs, e.g., tumor reactive CD8⁺ TILs)) expressing markers typical of T_{N} and/or T_{SCM} cells. In certain aspects, an increase in the number of stem-like immune cells (e.g., T cells and/or NK cells (e.g., CD8⁺ TILs, e.g., tumor reactive CD8⁺ TILs)) is characterized by increased numbers of cells expressing markers typical of T_{SCM} cells. In some aspects, the population of immune cells (e.g., T cells and/or NK cells (e.g., CD8⁺ TILs, e.g., tumor reactive CD8⁺ TILs)) exhibits an increased number of cells that express CD45RA. In some aspects, the population of immune cells (e.g., T cells and/or NK cells (e.g., CD8⁺ TILs, e.g., tumor reactive CD8⁺ TILs)) exhibits an increased number of cells that express CCR7. In some aspects, the population of immune cells (e.g., T cells and/or NK cells (e.g., CD8⁺ TILs, e.g., tumor reactive CD8⁺ TILs)) exhibits an increased number of cells that express CD62L. In some aspects, the population of immune cells (e.g., T cells and/or NK cells (e.g., CD8⁺ TILs, e.g., tumor reactive CD8⁺ TILs)) exhibits an increased number of cells that express CD28. In some aspects, the immune cells (e.g., T cells and/or NK cells (e.g., CD8⁺ TILs, e.g., tumor reactive CD8⁺ TILs)) exhibit an increased number of cells that express CD95. In some aspects, the cells are CD45RO^{low}. In some aspects, the cells do not express CD45R0. In some aspects, the population of cells exhibits an increased number of cells that are D45RA⁺, CCR7T, and CD62L⁺. In some aspects, the population of cells exhibits an increased number of cells that are CD95⁺, CD45RA⁺, CCR7⁺, and CD62L⁺. In some aspects, the population of cells exhibits an increased number of cells that express TCF7. In some aspects, the population of cells exhibits an increased number of cells that are CD45RA⁺, CCR7⁺, CD62L⁺, and TCF7⁺. In some aspects, the population of immune cells (e.g., T cells and/or NK cells (e.g., CD8⁺ TILs, e.g., tumor reactive CD8⁺ TILs)) exhibits an increased number of cells that are CD95⁺, CD45RA⁺, CCR7⁺, CD62L⁺, and TCF7⁺. In some aspects, the cells express CD3. In some aspects, the population of immune cells (e.g., T cells and/or NK cells (e.g., CD8⁺ TILs, e.g., tumor reactive CD8⁺ TILs)) exhibits an increased number of cells that are CD3⁺, CD45RA⁺, CCR7⁺, CD62L⁺, and TCF7⁺. In some aspects, the population of immune cells (e.g., T cells and/or NK cells (e.g., CD8⁺ TILs, e.g., tumor reactive CD8⁺ TILs)) exhibits an increased number of cells that are CD3⁺, CD95⁺, CD45RA⁺, CCR7⁺, CD62L⁺, and TCF7⁺. In some aspects, the cells express CD27. In some aspects, the population of immune cells (e.g., T cells and/or NK cells (e.g., CD8⁺ TILs, e.g., tumor reactive CD8⁺ TILs)) exhibits an increased number of cells that are CD27⁺, CD3⁺, CD45RA⁺, CCR7⁺, CD62L⁺, and TCF7⁺. In some aspects, the population of immune cells (e.g., T cells and/or NK cells (e.g., CD8⁺ TILs, e.g., tumor reactive CD8⁺ TILs)) exhibits an increased number of cells that are CD27⁺, CD3⁺, CD95⁺, CD45RA⁺, CCR7⁺, CD62L⁺, and TCF7⁺. In some aspects, the population of cells exhibits an increased number of T_{SCM} cells. In some aspects, the population of cells exhibits an increased number of T_{N} cells. In some aspects, the population of cells exhibits an increased number of T_{SCM} and T_{N} cells. In some aspects, the population of cell exhibits an increased number of stem-like T cells. In some aspects the T cells are CD4⁺ cells. In some aspects, the T cells are CD8⁺ cells.

In some aspects, the number of stem-like cells in the culture is increased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%, relative to the number of stem-like cells prior to culture (e.g., with MRM). In some aspects, the number of stem-like cells in the culture is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, or at least about 20-fold, relative to the number of stem-like cells prior to culture (e.g., with MRM).

In some aspects, following culture of T cells according to the methods disclosed herein, stem-like T cells constitute at least about 1%, at least about 2%, at least about 3%, at least about 4%, at least about 5%, at least about 10%, or at least about 15% of the total number of CD8⁺ T cells in the culture.

In some aspects, following culture of T cells according to the methods disclosed herein, stem-like T constitute at least about 1%, at least about 2%, at least about 3%, at least about 4%, at least about 5%, at least about 10%, at least about 15%, of the total number of CD4+ T cells in the culture.

In some aspects, following culture of T cells according to the methods disclosed herein, stem-like T cells constitute at least about 10% to at least about 70% of the total number of T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD8⁺ T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD4⁺ T cells in the culture.

In some aspects, following culture of T cells according to the methods disclosed herein, at least about 10% to at least about 40% of the total number of T cells in the culture are CD39⁻/CD69⁻ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/CD69⁻ T cells.

In some aspects, following culture of T cells according to the methods disclosed herein, at least about 10% to at least about 70% of the total number of T cells in the culture are CD39⁻/TCF7⁺ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/TCF7⁺ T cells. In some aspects the T cells are CD4⁺ T cells. In some aspects the T cells are CD8⁺ T cells.

In some aspects, the cells, e.g., pluripotent, multipotent, and/or immune cells (e.g., T cells and/or NK cells), cultured according to the methods disclosed herein exhibit increased transduction efficiency. In some aspects, a greater percentage of cells express a target transgene, e.g., encoding a CAR, a TCR, or a TCRm, following transduction, wherein the cells are cultured according to the methods disclosed herein as compared to cells similarly transduced and cultured using conventional methods (e.g., in media containing less than 5 mM K+). In certain aspects, the cells, e.g., pluripotent, multipotent, and/or immune cells (e.g., T cells and/or NK cells), are transduced using a viral vector. In some aspects, the viral vector comprises an AAV. In some aspects, the viral vector comprises a retrovirus. In some aspects, the viral vector comprises a lentivirus. In certain aspects, a greater percentage of cells cultured according to the methods disclosed herein express a CAR or a TCR following lentiviral transduction of the cells, as compared to similarly transduced cells cultured using conventional methods. In some aspects, transduction efficiency is increased at least 1.5-fold relative to similarly transduced cells cultured using conventional methods. In some aspects, transduction efficiency is increase at least 2-fold relative to similarly transduced cells cultured using conventional methods.

In some aspects, the cells, e.g., pluripotent, multipotent, and/or immune cells (e.g., T cells and/or NK cells), are transduced before culturing according to the methods disclosed herein. In some aspects, the cells, e.g., pluripotent, multipotent, and/or immune cells (e.g., T cells and/or NK cells), are transduced after culturing according to the methods disclosed herein. In some aspects, the cells, e.g., pluripotent, multipotent, and/or immune cells (e.g., T cells and/or NK cells), are cultured according to the methods disclosed herein, e.g., in a hypotonic or isotonic medium comprising at least 50 mM potassium ion, prior to, during, and after transduction.

In certain aspects, the cells, e.g., immune cells, are transduced using a viral vector. In some aspects, the vector comprises a lentiviral vector, adenoviral vector, adeno-associated viral vector, vaccinia vector, herpes simplex viral vector, and Epstein-Barr viral vector. In some aspects, the viral vector comprises a retrovirus. In some aspects, the viral vector comprises a lentivirus. In some aspects, the viral vector comprises an AAV.

In some aspects, the cells, e.g., immune cells, are transduced using a non-viral method. In some aspects, the non-viral method includes the use of a transposon. In some aspects, use of a non-viral method of delivery permits reprogramming of cells, e.g., immune cells, e.g., T cells and/or NK cells, and direct infusion of the cells into the subject. In some aspects, the polynucleotide can be inserted into the genome of a target cell (e.g., a T cell) or a host cell (e.g., a cell for recombinant expression of the encoded proteins) by using CRISPR/Cas systems and genome edition alternatives such as zinc-finger nucleases (ZFNs), transcription activator-like effector nucleases (TALENs), and meganucleases (MNs).

In certain aspects, upon adoptive transfer of the cells, e.g., T cells and/or NK cells, cultured according to the methods disclosed herein, the transferred cells exhibit decreased cell exhaustion, as compared to cells cultured using conventional culture conditions, e.g., in media containing less than 5 mM K⁺. In some aspects, upon adoptive transfer of the T cells, optionally expressing a ligand binding protein, cultured according to the methods disclosed herein, the transferred T cells exhibit decreased cell exhaustion, as compared to T cells cultured using conventional methods, e.g., in media containing less than 5 mM K⁺. In certain aspects, upon adoptive transfer of the cultured cells, the transferred cells persist for a longer period of time in vivo, as compared to cells cultured using conventional culture conditions, e.g., in media containing less than 5 mM K⁺. In some aspects, the transferred cells, e.g., T cells and/or NK cells, have a greater in vivo efficacy, e.g., tumor-killing activity, as compared to cells cultured using conventional culture conditions, e.g., in media containing less than 5 mM K⁺. In some aspects, a lower dose of the cells cultured according to the methods disclosed herein is needed to elicit a response, e.g., decreased tumor volume, in a subject as compared to cells cultured using conventional culture conditions, e.g., in media containing less than 5 mM K⁺.

In some aspects, following culture of immune cells, e.g., TILs (e.g., CD8⁺ TILs (e.g., tumor reactive CD8+ TILs)), according to the methods disclosed herein, stem-like CD8⁺ TILs (e.g., stem-like tumor reactive CD8+ TILs) constitute at least about 1%, at least about 2%, at least about 3%, at least about 4%, at least about 5%, at least about 10%, at least about 15%, of the total number of CD8⁺ TILs in the culture.

In some aspects, upon adoptive transfer of the immune cells, e.g., TILs (e.g., CD8⁺ TILs (e.g., tumor reactive CD8⁺ TILs)), cultured according to the methods disclosed herein, the transferred cells exhibit decreased cell exhaustion, as compared to cells cultured using conventional culture conditions. In some aspects, upon adoptive transfer of the cultured TILs, the transferred CD8⁺-enriched TILs persist for a longer period of time in vivo, as compared to TILs cultured using conventional culture conditions. In some aspects, the transferred CD8⁺-enriched TILs, have a greater in vivo efficacy, e.g., tumor-killing activity, as compared to TILs cultured using conventional culture conditions. In some aspects, a lower dose of the CD8⁺-enriched TILs cultured according to the methods disclosed herein is needed to elicit a response, e.g., decreased tumor volume, in a subject as compared to cells cultured using conventional culture conditions.

In some aspects, the TILs are cultured in the metabolic reprogramming media, e.g., hyperkalemic medium disclosed herein for the entirety of ex vivo culture, e.g., from the time the tumor sample is first plated through the entire expansion process, and until administration. In some aspects, the TILs are cultured in the medium disclosed herein for the duration of expansion.

In some aspects, the immune cells (e.g., T cells and/or NK cells) are cultured according to the methods disclosed herein, e.g., in a medium comprising at least 5 mM potassium ion, immediately upon isolation from a subject. In some aspects, the immune cells, e.g., T cells and/or NK cells, are cultured according to the methods disclosed herein during expansion of the cells. In some aspects, the immune cells, e.g., T cells and/or NK cells, are cultured according to the methods disclosed herein during engineering of the cells, e.g., during transduction with a construct encoding a transgene, e.g., a ligand binding protein. In some aspects, the immune cells, e.g., T cells and/or NK cells, are cultured according to the methods disclosed herein following engineering of the cells, e.g., following transduction with a construct encoding a transgene., e.g., a ligand binding protein. In some aspects, the immune cells, e.g., T cells and/or NK cells, are cultured according to the methods disclosed herein throughout expansion and engineering. In some aspects, the immune cells, e.g., T cells and/or NK cells, are cultured according to the methods disclosed herein throughout viral genetic engineering. In some aspects, the immune cells, e.g., T cells and/or NK cells, are cultured according to the methods disclosed herein throughout non-viral genetic engineering. In some aspects, the immune cells, e.g., T cells and/or NK cells, are cultured according to the methods disclosed herein during introduction of ligand binding proteins to the immune cell (e.g., T cells and/or NK cells) to allow for tumor specific targeting (e.g., a CAR, TCR or a TCR mimic). In some aspects, the immune cells, e.g., T cells and/or NK cells, are cultured according to the methods disclosed herein throughout introduction of one or more endogenous genes that improve T cell function. In some aspects, the immune cells, e.g., T cells and/or NK cells, are cultured according to the methods disclosed herein throughout introduction of one or more synthetic genes that improve T cell function.

In some aspects, the immune cells, e.g., T cells and/or NK cells, are cultured according to the methods disclosed herein, e.g., in a medium comprising at least 5 mM potassium ion, for the entirety of ex vivo culture, e.g., from the time the immune cells, e.g., T cells and/or NK cells, are isolated from a subject, through growing, expansion, engineering, and until administration into a subject in need of adoptive cell therapy. In some aspects, the T cells are cultured according to the methods disclosed herein, e.g., in a medium comprising at least 5 mM potassium ion, for the entirety of ex vivo culture, e.g., from the time the T cells are isolated from a subject, through growing, expansion, engineering, and until administration into a subject in need of adoptive cell therapy. In some aspects, the immune cells, e.g., T cells and/or NK cells, are cultured according to the methods disclosed herein for the duration of expansion. In some aspects, the immune cells, e.g., T cells and/or NK cells, are cultured according to the methods disclosed herein until the total number of viable immune cells, e.g., T cells and/or NK cells, is at least about 10⁴, at least about 5×10⁴, at least about 10⁵, at least about 5×10⁵, at least about 10⁶, or at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total cells. In some aspects, the T cells are cultured according to the methods disclosed herein until the total number of viable T cells is at least about 10⁴, at least about 5×10⁴, at least about 10⁵, at least about 5×10⁵, at least about 10⁶, or at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total T cells.

In some aspects, the metabolic reprogramming media, e.g., hyperkalemic culture medium comprises a mitochondrial fuel. In some aspects, the metabolic reprogramming media, e.g., hyperkalemic culture medium, comprises O-Acetyl-L-carnitine hydrochloride. In some aspects, the metabolic reprogramming media, e.g., hyperkalemic culture medium, comprises at least about 0.1 mM, at least about 0.5 mM, at least about 1.0 mM, at least about 5 mM, or at least about 10 mM O-Acetyl-L-carnitine hydrochloride. In some aspects, the metabolic reprogramming media, e.g., hyperkalemic culture medium, comprises at least about 1.0 mM O-Acetyl-L-carnitine hydrochloride.

In some aspects, the metabolic reprogramming media, e.g., hyperkalemic culture medium, comprises inhibitor of glycolysis-mediated metabolism, e.g., a kinase inhibitor, e.g., a phosphoinositide 3-kinase inhibitor. In some aspects, the metabolic reprogramming media, e.g., hyperkalemic culture medium, comprises a phosphatidylinositol-3-kinase (PI3K) inhibitor, e.g., idelalisib (e.g., CAL-101; Selleckchem). In some aspects, the metabolic reprogramming media, e.g., hyperkalemic culture medium, comprises at least about 0.1 mM, at least about 0.5 mM, at least about 1.0 mM, at least about 5 mM, or at least about 10 mM idelalisib. In some aspects, the metabolic reprogramming media, e.g., hyperkalemic culture medium, comprises at least about 1.0 mM idelalisib.

In some aspects, the metabolic reprogramming media, e.g., hyperkalemic culture medium, further comprises one or more of (i) one or more cell expansion agents, (ii) sodium ion, (iii) one or more saccharides, (iv) calcium ion, and (v) one or more cytokines.

II.A. Potassium

Some aspects of the disclosure are directed to methods of culturing cells, e.g., pluripotent, multipotent, and/or immune cells (e.g., T cells and/or NK cells), in medium comprising an increased concentration of potassium ion (e.g., greater than about 5 mM, greater than about 40 mM, greater than about 45 mM, greater than about 50 mM, greater than about 55 mM, greater than about 60 mM, greater than about 65 mM, greater than about 70 mM), relative to conventional immune cell culture media, wherein the hyperkalemic media is hypotonic or isotonic. The present disclosure is based in part on the discovery that the tonicity of the medium combined with the level of potassium ion creates an ideal environment for promoting stemness while still retaining efficacy.

Some aspects of the present disclosure are directed to a method of increasing the yield of human immune cells and/or stem cells during ex vivo or in vitro culturing while increasing stemness of the human immune cells and/or stem cells comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of preparing a population of human immune cells and/or stem cells for immunotherapy comprising culturing human immune cells and/or stem cells in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of increasing sternness of human immune cells during ex vivo or in vitro culturing for immunotherapy comprising culturing human immune cells in a medium comprising potassium ion at a concentration between 40 mM and 80 mM and NaCl at a concentration between 30 mM and 100 mM, wherein the total concentration of potassium ion and NaCl is between 110 and 140 mM.

Some aspects of the disclosure are directed to methods of culturing cells, e.g., pluripotent, multipotent, and/or immune cells (e.g., T cells and/or NK cells), in medium comprising potassium ion at a concentration of at least about 5 mM, wherein the medium is hypotonic. Some aspects of the disclosure are directed to methods of culturing cells, e.g., pluripotent, multipotent, and/or immune cells (e.g., T cells and/or NK cells), in medium comprising potassium ion at a concentration higher than 40 mM. Some aspects of the disclosure are directed to methods of culturing cells, e.g., pluripotent, multipotent, and/or immune cells (e.g., T cells and/or NK cells), in medium comprising potassium ion at a concentration of at least about 50 mM. Some aspects of the disclosure are directed to methods of culturing cells, e.g., pluripotent, multipotent, and/or immune cells (e.g., T cells and/or NK cells), in medium comprising potassium ion at a concentration of at least about 50 mM, wherein the medium is hypotonic or isotonic. In some aspects, the cells, e.g., the T cells, NK cells, or TILs, are cultured in a medium disclosed herein, e.g., comprising potassium ion at a concentration of at least about 50 mM, immediately upon isolation from a subject. In some aspects, the cells, e.g., the T cells, NK cells, or TILs, are cultured in a medium disclosed herein, e.g., comprising potassium ion at a concentration of at least about 50 mM, during expansion of the cells. In some aspects, the cells, e.g., the T cells, NK cells, or TILs, are cultured in a medium disclosed herein, e.g., comprising potassium ion at a concentration of at least about 50 mM, during engineering of the cells, e.g., during transduction with a construct encoding a CAR or a TCR. In some aspects, the cells, e.g., the T cells, NK cells, or TILs, are cultured in a medium disclosed herein, e.g., comprising potassium ion at a concentration of at least about 50 mM, following engineering of the cells, e.g., following transduction with a construct encoding a CAR or a TCR. In some aspects, the cells, e.g., the T cells, NK cells, or TILs, are cultured in a medium disclosed herein, e.g., comprising potassium ion at a concentration of at least about 50 mM, throughout expansion and engineering. In certain aspects, the cells, e.g., the T cells, NK cells, or TILs, are cultured in a medium disclosed herein, e.g., comprising potassium ion at a concentration of at least about 50 mM, throughout viral genetic engineering. In certain aspects, the cells, e.g., the T cells, NK cells, or TILs, are cultured in a medium disclosed herein, e.g., comprising potassium ion at a concentration of at least about 50 mM, throughout non-viral genetic engineering. In certain aspects, the cells, e.g., the T cells, NK cells, or TILs, are cultured in a medium disclosed herein, e.g., comprising potassium ion at a concentration of at least about 50 mM, during introduction of receptors to allow for tumor specific targeting (e.g., a TCR, a TCRm, and/or a CAR). In certain aspects, the cells, e.g., the T cells, NK cells, or TILs, are cultured in a medium disclosed herein, e.g., comprising potassium ion at a concentration of at least about 50 mM, throughout introduction of one or more endogenous genes that improve T cell function. In certain aspects, the cells, e.g., the T cells, NK cells, or TILs, are cultured in a medium disclosed herein, e.g., comprising potassium ion at a concentration of at least about 50 mM, throughout introduction of one or more synthetic genes that improve T cell function.

Some aspects of the present disclosure are directed to methods of culturing TILs ex vivo or in vitro comprising placing a heterogeneous population of TILs in a metabolic reprogramming media, e.g., hyperkalemic medium. Some aspects of the present disclosure are directed to methods of increasing a number or percentage of CD8⁺ TILs ex vivo or in vitro comprising culturing a heterogeneous population of TILs in a metabolic reprogramming media, e.g., hyperkalemic medium. Other aspects of the present disclosure are directed to methods of preparing a CD8⁺-enriched population of tumor infiltrating lymphocytes (TILs), comprising culturing a heterogeneous population of TILs ex vivo or in vitro in a metabolic reprogramming media, e.g., hyperkalemic medium. In some aspects, the concentration of potassium ion is at least about 30 mM to at least about 100 mM. In some aspects, the concentration of potassium ion is at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 Mm. In some aspects the concentration of potassium ion is about 40 mM to about 90 mM.

Some aspects of the disclosure are directed to methods of culturing immune cells, e.g., T cells and/or NK cells, in a medium comprising an increased concentration of potassium ion (e.g., greater than about 5 mM, greater than about 40 mM, greater than about 45 mM, greater than about 50 mM, greater than about 55 mM, greater than about 60 mM, greater than about 65 mM, or greater than about 70 mM), i.e., a metabolic reprogramming medium disclosed herein, relative to a control medium. In some aspects, the metabolic reprogramming medium comprises at least about 5 mM to at least about 100 mM potassium ion, at least about 5 mM to at least about 90 mM potassium ion, at least about 5 mM to at least about 80 mM potassium ion, at least about 5 mM to at least about 75 mM potassium ion, at least about 5 mM to at least about 70 mM potassium ion, at least about 5 mM to at least about 65 mM potassium ion, at least about 5 mM to at least about 60 mM potassium ion, at least about 5 mM to at least about 55 mM potassium ion, at least about 5 mM to at least about 50 mM potassium ion, at least about 5 mM to at least about 45 mM potassium ion, at least about 5 mM to at least about 40 mM potassium ion, at least about 10 mM to at least about 80 mM potassium ion, at least about 10 mM to at least about 75 mM potassium ion, at least about 10 mM to at least about 70 mM potassium ion, at least about 10 mM to at least about 65 mM potassium ion, at least about 10 mM to at least about 60 mM potassium ion, at least about 10 mM to at least about 55 mM potassium ion, at least about 10 mM to at least about 50 mM potassium ion, at least about 10 mM to at least about 45 mM potassium ion, at least about 10 mM to at least about 40 mM potassium ion, at least about 20 mM to at least about 80 mM potassium ion, at least about 20 mM to at least about 75 mM potassium ion, at least about 20 mM to at least about 70 mM potassium ion, at least about 20 mM to at least about 65 mM potassium ion, at least about 20 mM to at least about 60 mM potassium ion, at least about 20 mM to at least about 55 mM potassium ion, at least about 20 mM to at least about 50 mM potassium ion, at least about 20 mM to at least about 45 mM potassium ion, at least about 20 mM to at least about 40 mM potassium ion, at least about 30 mM to at least about 80 mM potassium ion, at least about 30 mM to at least about 75 mM potassium ion, at least about 30 mM to at least about 70 mM potassium ion, at least about 30 mM to at least about 65 mM potassium ion, at least about 30 mM to at least about 60 mM potassium ion, at least about 30 mM to at least about 55 mM potassium ion, at least about 30 mM to at least about 50 mM potassium ion, at least about 30 mM to at least about 45 mM potassium ion, at least about 30 mM to at least about 40 mM potassium ion, at least about 40 mM to at least about 80 mM potassium ion, at least about 40 mM to at least about 75 mM potassium ion, at least about 40 mM to at least about 70 mM potassium ion, at least about 40 mM to at least about 65 mM potassium ion, at least about 40 mM to at least about 60 mM potassium ion, at least about 40 mM to at least about 55 mM potassium ion, at least about 40 mM to at least about 50 mM potassium ion, at least about 40 mM to at least about 45 mM potassium ion, at least about 45 mM to at least about 80 mM potassium ion, at least about 45 mM to at least about 75 mM potassium ion, at least about 45 mM to at least about 70 mM potassium ion, at least about 45 mM to at least about 65 mM potassium ion, at least about 45 mM to at least about 60 mM potassium ion, at least about 45 mM to at least about 55 mM potassium ion, at least about 45 mM to at least about 50 mM potassium ion, at least about 50 mM to at least about 80 mM potassium ion, at least about 50 mM to at least about 75 mM potassium ion, at least about 50 mM to at least about 70 mM potassium ion, at least about 50 mM to at least about 65 mM potassium ion, at least about 50 mM to at least about 60 mM potassium ion, or at least about 50 mM to at least about 55 mM potassium ion.

In some aspects, the concentration of potassium ion is at least about 5 mM, at least about 10 mM, at least about 15 mM, at least about 20 mM, at least about 25 mM, at least about 30 mM, at least about 35 mM, at least about 40 mM, or at least about 45 mM. In some aspects, the concentration of potassium ion is at least about 50 mM. In some aspects, the concentration of potassium ion is about 5 mM, about 10 mM, about 15 mM, about 20 mM, about 25 mM, about 30 mM, about 35 mM, about 40 mM, or about 45 mM. In some aspects, the concentration of potassium ion is about 40 mM. In some aspects, the concentration of potassium ion is about 45 mM. In some aspects, the concentration of potassium ion is about 50 mM.

In some aspects, the concentration of potassium ion is at least about 55 mM, at least about 60 mM, at least about 65 mM, at least about 70 mM, at least about 75 mM, at least about 80 mM, at least about 85 mM, at least about 90 mM, at least about 95 mM, at least about 100 mM, at least about 105 mM, at least about 110 mM, at least about 115 mM, at least about 120 mM. In some aspects, the concentration of potassium ion is about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM. In some aspects, the concentration of potassium ion is about 55 mM. In some aspects, the concentration of potassium ion is about 60 mM. In some aspects, the concentration of potassium ion is about 65 mM. In some aspects, the concentration of potassium ion is about 70 mM.

In some aspects, the concentration of potassium ion is about 5 mM to about 100 mM, wherein the medium is hypotonic. In some aspects, the concentration of potassium ion is about 5 mM to about 90 mM, about 5 mM to about 80 mM, about 5 mM to about 70 mM, about 5 mM to about 60 mM, or about 5 mM to about 50 mM, wherein the medium is hypotonic. In some aspects, the concentration of potassium ion is about 25 mM to about 100 mM, wherein the medium is hypotonic. In some aspects, the concentration of potassium ion is about 25 mM to about 90 mM, about 25 mM to about 80 mM, about 25 mM to about 70 mM, about 25 mM to about 60 mM, or about 25 mM to about 50 mM, wherein the medium is hypotonic. In some aspects, the concentration of potassium ion is about 40 mM to about 100 mM, wherein the medium is hypotonic. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM, wherein the medium is hypotonic. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the medium is hypotonic. In some aspects, the medium comprises at least about 50 mM potassium ion and less than about 90 mM NaCl. In some aspects, the total concentration of potassium ion and NaCl is between 110 mM and 140 mM.

In some aspects, the metabolic reprogramming medium comprises potassium ion at a concentration between about 40 mM and about 80 mM and NaCl at a concentration between about 30 mM and about 100 mM, wherein the total concentration of potassium ion and NaCl is between about 110 and about 140 mM. In some aspects, the concentration of potassium ion in a metabolic reprogramming medium of the present disclosure is about 5 mM to about 100 mM. In some aspects, the concentration of potassium ion in a metabolic reprogramming medium of the present disclosure is about 5 mM to about 100 mM, wherein the medium is hypotonic. In some aspects, the concentration of potassium ion in a metabolic reprogramming medium of the present disclosure is about 5 mM to about 90 mM, about 5 mM to about 80 mM, about 5 mM to about 70 mM, about 5 mM to about 60 mM, or about 5 mM to about 50 mM.

In some aspects, the concentration of potassium ion in a metabolic reprogramming medium of the present disclosure is about 25 mM to about 90 mM, about 25 mM to about 80 mM, about 25 mM to about 70 mM, about 25 mM to about 60 mM, or about 25 mM to about 50 mM, wherein the medium is hypotonic. In some aspects, the concentration of potassium ion in a metabolic reprogramming medium of the present disclosure is about 40 mM to about 100 mM. In some aspects, the concentration of potassium ion in a metabolic reprogramming medium of the present disclosure is about 40 mM to about 100 mM, wherein the medium is hypotonic. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM.

In some aspects, the concentration of potassium ion is about 50 mM to about 120 mM. In some aspects, the concentration of potassium ion is about 50 mM to about 115 mM, about 50 mM to about 110 mM, about 50 mM to about 105 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the medium is hypotonic. In some aspects, the medium comprises at least about 50 mM to about 120 mM potassium ion and less than about 90 mM to about 20 mM NaCl. In some aspects, the total concentration of potassium ion and NaCl is between 110 mM and 140 mM.

In some aspects, the concentration of potassium ion is about 55 mM to about 120 mM. In some aspects, the concentration of potassium ion is about 55 mM to about 115 mM, about 55 mM to about 110 mM, about 55 mM to about 105 mM, about 55 mM to about 100 mM, about 55 mM to about 95 mM, about 55 mM to about 90 mM, about 55 mM to about 85 mM, about 55 mM to about 80 mM, about 55 mM to about 75 mM, about 55 mM to about 70 mM, about 55 mM to about 65 mM, or about 55 mM to about 60 mM. In some aspects, the medium is hypotonic. In some aspects, the medium comprises at least about 55 mM to about 120 mM potassium ion and less than about 85 mM to about 20 mM NaCl. In some aspects, the total concentration of potassium ion and NaCl is between 110 mM and 140 mM.

In some aspects, the concentration of potassium ion is about 60 mM to about 120 mM. In some aspects, the concentration of potassium ion is about 60 mM to about 115 mM, about 60 mM to about 110 mM, about 60 mM to about 105 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, or about 60 mM to about 65 mM. In some aspects, the medium is hypotonic. In some aspects, the medium comprises at least about 60 mM to about 120 mM potassium ion and less than about 80 mM to about 20 mM NaCl. In some aspects, the total concentration of potassium ion and NaCl is between 110 mM and 140 mM.

In some aspects, the concentration of potassium ion is about 65 mM to about 120 mM. In some aspects, the concentration of potassium ion is about 65 mM to about 115 mM, about 65 mM to about 110 mM, about 65 mM to about 105 mM, about 65 mM to about 100 mM, about 65 mM to about 95 mM, about 65 mM to about 90 mM, about 65 mM to about 85 mM, about 65 mM to about 80 mM, about 65 mM to about 75 mM, or about 65 mM to about 70 mM. In some aspects, the medium is hypotonic. In some aspects, the medium comprises at least about 65 mM to about 120 mM potassium ion and less than about 75 mM to about 20 mM NaCl. In some aspects, the total concentration of potassium ion and NaCl is between 110 mM and 140 mM.

In some aspects, the concentration of potassium ion is about 70 mM to about 120 mM. In some aspects, the concentration of potassium ion is about 70 mM to about 115 mM, about 70 mM to about 110 mM, about 70 mM to about 105 mM, about 70 mM to about 100 mM, about 70 mM to about 95 mM, about 70 mM to about 90 mM, about 70 mM to about 85 mM, about 70 mM to about 80 mM, or about 70 mM to about 75 mM. In some aspects, the medium is hypotonic. In some aspects, the medium comprises at least about 70 mM to about 120 mM potassium ion and less than about 70 mM to about 20 mM NaCl. In some aspects, the total concentration of potassium ion and NaCl is between 110 mM and 140 mM.

In some aspects, the concentration of potassium ion is about 75 mM to about 120 mM. In some aspects, the concentration of potassium ion is about 75 mM to about 115 mM, about 75 mM to about 110 mM, about 75 mM to about 105 mM, about 75 mM to about 100 mM, about 75 mM to about 95 mM, about 75 mM to about 90 mM, about 75 mM to about 85 mM, or about 75 mM to about 80 mM. In some aspects, the medium is hypotonic. In some aspects, the medium comprises at least about 75 mM to about 120 mM potassium ion and less than about 65 mM to about 20 mM NaCl. In some aspects, the total concentration of potassium ion and NaCl is between 110 mM and 140 mM.

In some aspects, the concentration of potassium ion is about 80 mM to about 120 mM. In some aspects, the concentration of potassium ion is about 80 mM to about 115 mM, about 80 mM to about 110 mM, about 80 mM to about 105 mM, about 80 mM to about 100 mM, about 80 mM to about 95 mM, about 80 mM to about 90 mM, or about 80 mM to about 85 mM. In some aspects, the medium is hypotonic. In some aspects, the medium comprises at least about 80 mM to about 120 mM potassium ion and less than about 60 mM to about 20 mM NaCl. In some aspects, the total concentration of potassium ion and NaCl is between 110 mM and 140 mM.

In some aspects, the concentration of potassium ion is about 85 mM to about 120 mM. In some aspects, the concentration of potassium ion is about 85 mM to about 115 mM, about 85 mM to about 110 mM, about 85 mM to about 105 mM, about 85 mM to about 100 mM, about 85 mM to about 95 mM, or about 85 mM to about 90 mM. In some aspects, the medium is hypotonic. In some aspects, the medium comprises at least about 85 mM to about 120 mM potassium ion and less than about 65 mM to about 20 mM NaCl. In some aspects, the total concentration of potassium ion and NaCl is between 110 mM and 140 mM.

In some aspects, the concentration of potassium ion is about 90 mM to about 120 mM. In some aspects, the concentration of potassium ion is about 90 mM to about 115 mM, about 90 mM to about 110 mM, about 90 mM to about 105 mM, about 90 mM to about 100 mM, or about 90 mM to about 95 mM. In some aspects, the medium is hypotonic. In some aspects, the medium comprises at least about 90 mM to about 120 mM potassium ion and less than about 50 mM to about 20 mM NaCl. In some aspects, the total concentration of potassium ion and NaCl is between 110 mM and 140 mM.

In some aspects, the concentration of potassium ion is about 95 mM to about 120 mM. In some aspects, the concentration of potassium ion is about 95 mM to about 115 mM, about 95 mM to about 110 mM, about 95 mM to about 105 mM, or about 95 mM to about 100 mM. In some aspects, the medium is hypotonic. In some aspects, the medium comprises at least about 95 mM to about 120 mM potassium ion and less than about 55 mM to about 20 mM NaCl. In some aspects, the total concentration of potassium ion and NaCl is between 110 mM and 140 mM.

In some aspects, the concentration of potassium ion is about 100 mM to about 120 mM. In some aspects, the concentration of potassium ion is about 100 mM to about 115 mM, about 100 mM to about 110 mM, or about 100 mM to about 105 mM. In some aspects, the medium is hypotonic. In some aspects, the medium comprises at least about 100 mM to about 120 mM potassium ion and less than about 50 mM to about 20 mM NaCl. In some aspects, the total concentration of potassium ion and NaCl is between 110 mM and 140 mM.

In some aspects, the concentration of potassium ion is about 105 mM to about 120 mM. In some aspects, the concentration of potassium ion is about 105 mM to about 115 mM, or about 105 mM to about 110 mM. In some aspects, the medium is hypotonic. In some aspects, the medium comprises at least about 105 mM to about 120 mM potassium ion and less than about 35 mM to about 20 mM NaCl. In some aspects, the total concentration of potassium ion and NaCl is between 110 mM and 140 mM.

In some aspects, the concentration of potassium ion is about 110 mM to about 120 mM. In some aspects, the concentration of potassium ion is about 110 mM to about 115 mM. In some aspects, the medium is hypotonic. In some aspects, the medium comprises at least about 110 mM to about 120 mM potassium ion and less than about 30 mM to about 20 mM NaCl. In some aspects, the total concentration of potassium ion and NaCl is between 110 mM and 140 mM.

In some aspects, the concentration of potassium ion is about 50 mM to about 90 mM. In some aspects, the concentration of potassium ion is about 50 mM to about 80 mM. In some aspects, the concentration of potassium ion is about 60 mM to about 90 mM. In some aspects, the concentration of potassium ion is about 60 mM to about 80 mM. In some aspects, the concentration of potassium ion is about 70 mM to about 90 mM. In some aspects, the concentration of potassium ion is about 70 mM to about 80 mM. In some aspects, the concentration of potassium ion is about 80 mM to about 90 mM.

In some aspects, the concentration of potassium ion is about 50 mM to about 90 mM, and the concentration of NaCl is less than about 90 mM to about 50 mM. In some aspects, the concentration of potassium ion is about 50 mM to about 80 mM, and the concentration of NaCl is less than about 90 mM to about 60 mM. In some aspects, the concentration of potassium ion is about 60 mM to about 90 mM, and the concentration of NaCl is less than about 90 mM to about 60 mM. In some aspects, the concentration of potassium ion is about 60 mM to about 80 mM, and the concentration of NaCl is less than about 80 mM to about 60 mM. In some aspects, the concentration of potassium ion is about 70 mM to about 90 mM, and the concentration of NaCl is less than about 70 mM to about 50 mM. In some aspects, the concentration of potassium ion is about 70 mM to about 80 mM, and the concentration of NaCl is less than about 70 mM to about 60 mM. In some aspects, the concentration of potassium ion is about 80 mM to about 90 mM, and the concentration of NaCl is less than about 60 mM to about 50 mM. In some aspects, the total concentration of potassium ion and NaCl is between 110 mM and 140 mM.

In some aspects, the concentration of potassium ion is about 50 mM to about 55 mM. In some aspects, the concentration of potassium ion is about 50 mM to about 55 mM, and the concentration of NaCl is less than about 90 to about 85. In some aspects, the concentration of potassium ion is about 55 mM to about 60 mM. In some aspects, the concentration of potassium ion is about 55 mM to about 60 mM, and the concentration of NaCl is less than about 85 to about 80. In some aspects, the concentration of potassium ion is about 60 mM to about 65 mM. In some aspects, the concentration of potassium ion is about 60 mM to about 65 mM, and the concentration of NaCl is less than about 80 mM to about 75 mM. In some aspects, the concentration of potassium ion is about 65 mM to about 70 mM. In some aspects, the concentration of potassium ion is about 65 mM to about 70 mM, and the concentration of NaCl is less than about 75 mM to about 70 mM. In some aspects, the concentration of potassium ion is about 70 mM to about 75 mM. In some aspects, the concentration of potassium ion is about 70 mM to about 75 mM, and the concentration of NaCl is less than about 70 mM to about 65 mM. In some aspects, the concentration of potassium ion is about 75 mM to about 80 mM. In some aspects, the concentration of potassium ion is about 75 mM to about 80 mM, and the concentration of NaCl is less than about 65 mM to about 60 mM. In some aspects, the concentration of potassium ion is about 80 mM to about 85 mM. In some aspects, the concentration of potassium ion is about 80 mM to about 85 mM, and the concentration of NaCl is less than about 60 mM to about 55 mM. In some aspects, the concentration of potassium ion is about 85 mM to about 90 mM. In some aspects, the concentration of potassium ion is about 85 mM to about 90 mM, and the concentration of NaCl is less than about 55 mM to about 50 mM. In some aspects, the concentration of potassium ion is about 90 mM to about 95 mM. In some aspects, the concentration of potassium ion is about 90 mM to about 95 mM, and the concentration of NaCl is less than about 50 to about 45. In some aspects, the concentration of potassium ion is about 95 mM to about 100 mM. In some aspects, the concentration of potassium ion is about 95 mM to about 100 mM, and the concentration of NaCl is less than about 45 mM to about 40 mM. In some aspects, the concentration of potassium ion is about 100 mM to about 105 mM. In some aspects, the concentration of potassium ion is about 100 mM to about 105 mM, and the concentration of NaCl is less than about 40 mM to about 35 mM. In some aspects, the concentration of potassium ion is about 105 mM to about 110 mM. In some aspects, the concentration of potassium ion is about 105 mM to about 110 mM, and the concentration of NaCl is less than about 35 to about 30. In some aspects, the concentration of potassium ion is about 110 mM to about 115 mM. In some aspects, the concentration of potassium ion is about 110 mM to about 115 mM, and the concentration of NaCl is less than about 30 mM to about 25 mM. In some aspects, the concentration of potassium ion is about 115 mM to about 120 mM. In some aspects, the concentration of potassium ion is about 115 mM to about 120 mM, and the concentration of NaCl is less than about 25 mM to about 20 mM. In some aspects, the total concentration of potassium ion and NaCl is between 110 mM and 140 mM.

In certain aspects, the concentration of potassium ion is about 40 mM to about 90 mM, wherein the medium is hypotonic. In certain aspects, the concentration of potassium ion is about 40 mM to about 80 mM, wherein the medium is hypotonic. In certain aspects, the concentration of potassium ion is about 40 mM to about 70 mM, wherein the medium is hypotonic. In certain aspects, the concentration of potassium ion is about 50 mM to about 90 mM, wherein the medium is hypotonic. In certain aspects, the concentration of potassium ion is about 50 mM to about 80 mM, wherein the medium is hypotonic. In certain aspects, the concentration of potassium ion is about 50 mM to about 70 mM, wherein the medium is hypotonic. In certain aspects, the concentration of potassium ion is about 55 mM to about 90 mM, wherein the medium is hypotonic. In certain aspects, the concentration of potassium ion is about 55 mM to about 80 mM, wherein the medium is hypotonic. In certain aspects, the concentration of potassium ion is about 55 mM to about 70 mM, wherein the medium is hypotonic. In certain aspects, the concentration of potassium ion is about 60 mM to about 90 mM, wherein the medium is hypotonic. In certain aspects, the concentration of potassium ion is about 60 mM to about 80 mM, wherein the medium is hypotonic. In certain aspects, the concentration of potassium ion is about 60 mM to about 70 mM, wherein the medium is hypotonic. In certain aspects, the concentration of potassium ion is about 65 mM to about 90 mM, wherein the medium is hypotonic. In certain aspects, the concentration of potassium ion is about 65 mM to about 80 mM, wherein the medium is hypotonic. In certain aspects, the concentration of potassium ion is about 65 mM to about 70 mM, wherein the medium is hypotonic.

In certain aspects, the concentration of potassium ion is about 40 mM to about 90 mM, wherein the medium is isotonic. In certain aspects, the concentration of potassium ion is about 40 mM to about 80 mM, wherein the medium is isotonic. In certain aspects, the concentration of potassium ion is about 40 mM to about 70 mM, wherein the medium is isotonic. In certain aspects, the concentration of potassium ion is about 50 mM to about 90 mM, wherein the medium is isotonic. In certain aspects, the concentration of potassium ion is about 50 mM to about 80 mM, wherein the medium is isotonic. In certain aspects, the concentration of potassium ion is about 50 mM to about 70 mM, wherein the medium is isotonic. In certain aspects, the concentration of potassium ion is about 55 mM to about 90 mM, wherein the medium is isotonic. In certain aspects, the concentration of potassium ion is about 55 mM to about 80 mM, wherein the medium is isotonic. In certain aspects, the concentration of potassium ion is about 55 mM to about 70 mM, wherein the medium is isotonic. In certain aspects, the concentration of potassium ion is about 60 mM to about 90 mM, wherein the medium is isotonic. In certain aspects, the concentration of potassium ion is about 60 mM to about 80 mM, wherein the medium is isotonic. In certain aspects, the concentration of potassium ion is about 60 mM to about 70 mM, wherein the medium is isotonic. In certain aspects, the concentration of potassium ion is about 65 mM to about 90 mM, wherein the medium is isotonic. In certain aspects, the concentration of potassium ion is about 65 mM to about 80 mM, wherein the medium is isotonic. In certain aspects, the concentration of potassium ion is about 65 mM to about 70 mM, wherein the medium is isotonic.

In some aspects, the concentration of potassium ion is higher than about 4 mM, wherein the medium is hypotonic or isotonic. In some aspects, the concentration of potassium ion is about 4 mM, wherein the medium is hypotonic or isotonic. In some aspects, the concentration of potassium ion is higher than about 5 mM, wherein the medium is hypotonic or isotonic. In some aspects, the concentration of potassium ion is about 5 mM, wherein the medium is hypotonic or isotonic. In some aspects, the concentration of potassium ion is higher than about 6 mM, wherein the medium is hypotonic or isotonic. In some aspects, the concentration of potassium ion is about 6 mM, wherein the medium is hypotonic or isotonic. In some aspects, the concentration of potassium ion is higher than about 7 mM, wherein the medium is hypotonic or isotonic. In some aspects, the concentration of potassium ion is about 7 mM, wherein the medium is hypotonic or isotonic. In some aspects, the concentration of potassium ion is higher than about 8 mM, wherein the medium is hypotonic or isotonic. In some aspects, the concentration of potassium ion is about 8 mM, wherein the medium is hypotonic or isotonic. In some aspects, the concentration of potassium ion is higher than about 9 mM, wherein the medium is hypotonic or isotonic. In some aspects, the concentration of potassium ion is about 9 mM, wherein the medium is hypotonic or isotonic.

In some aspects, the concentration of potassium ion is higher than about 10 mM, wherein the medium is hypotonic or isotonic. In some aspects, the concentration of potassium ion is about 10 mM, wherein the medium is hypotonic or isotonic. In some aspects, the concentration of potassium ion is higher than about 11 mM, wherein the medium is hypotonic or isotonic. In some aspects, the concentration of potassium ion is about 11 mM, wherein the medium is hypotonic or isotonic. In some aspects, the concentration of potassium ion is higher than about 12 mM, wherein the medium is hypotonic or isotonic. In some aspects, the concentration of potassium ion is about 12 mM, wherein the medium is hypotonic or isotonic. In some aspects, the concentration of potassium ion is higher than about 13 mM, wherein the medium is hypotonic or isotonic. In some aspects, the concentration of potassium ion is about 13 mM, wherein the medium is hypotonic. In some aspects, the concentration of potassium ion is higher than about 14 mM, wherein the medium is hypotonic or isotonic. In some aspects, the concentration of potassium ion is about 14 mM, wherein the medium is hypotonic or isotonic. In some aspects, the concentration of potassium ion is higher than about 15 mM, wherein the medium is hypotonic or isotonic. In some aspects, the concentration of potassium ion is about 15 mM, wherein the medium is hypotonic or isotonic. In some aspects, the concentration of potassium ion is higher than about 16 mM, wherein the medium is hypotonic or isotonic. In some aspects, the concentration of potassium ion is about 16 mM, wherein the medium is hypotonic or isotonic. In some aspects, the concentration of potassium ion is higher than about 17 mM, wherein the medium is hypotonic or isotonic. In some aspects, the concentration of potassium ion is about 17 mM, wherein the medium is hypotonic or isotonic. In some aspects, the concentration of potassium ion is higher than about 18 mM, wherein the medium is hypotonic or isotonic. In some aspects, the concentration of potassium ion is about 18 mM, wherein the medium is hypotonic or isotonic. In some aspects, the concentration of potassium ion is higher than about 19 mM, wherein the medium is hypotonic or isotonic. In some aspects, the concentration of potassium ion is about 19 mM, wherein the medium is hypotonic or isotonic.

In some aspects, the concentration of potassium ion is higher than about 20 mM, wherein the medium is hypotonic or isotonic. In some aspects, the concentration of potassium ion is about 20 mM, wherein the medium is hypotonic or isotonic. In some aspects, the concentration of potassium ion is higher than about 21 mM, wherein the medium is hypotonic or isotonic. In some aspects, the concentration of potassium ion is about 21 mM, wherein the medium is hypotonic or isotonic. In some aspects, the concentration of potassium ion is higher than about 22 mM, wherein the medium is hypotonic or isotonic. In some aspects, the concentration of potassium ion is about 22 mM, wherein the medium is hypotonic or isotonic. In some aspects, the concentration of potassium ion is higher than about 23 mM, wherein the medium is hypotonic or isotonic. In some aspects, the concentration of potassium ion is about 23 mM, wherein the medium is hypotonic. In some aspects, the concentration of potassium ion is higher than about 24 mM, wherein the medium is hypotonic or isotonic. In some aspects, the concentration of potassium ion is about 24 mM, wherein the medium is hypotonic or isotonic. In some aspects, the concentration of potassium ion is higher than about 25 mM, wherein the medium is hypotonic or isotonic. In some aspects, the concentration of potassium ion is about 25 mM, wherein the medium is hypotonic or isotonic. In some aspects, the concentration of potassium ion is higher than about 26 mM, wherein the medium is hypotonic or isotonic. In some aspects, the concentration of potassium ion is about 26 mM, wherein the medium is hypotonic or isotonic. In some aspects, the concentration of potassium ion is higher than about 27 mM, wherein the medium is hypotonic or isotonic. In some aspects, the concentration of potassium ion is about 27 mM, wherein the medium is hypotonic or isotonic. In some aspects, the concentration of potassium ion is higher than about 28 mM, wherein the medium is hypotonic or isotonic. In some aspects, the concentration of potassium ion is about 28 mM, wherein the medium is hypotonic or isotonic. In some aspects, the concentration of potassium ion is higher than about 29 mM, wherein the medium is hypotonic or isotonic. In some aspects, the concentration of potassium ion is about 29 mM, wherein the medium is hypotonic or isotonic.

In some aspects, the concentration of potassium ion is higher than about 30 mM, wherein the medium is hypotonic or isotonic. In some aspects, the concentration of potassium ion is about 30 mM, wherein the medium is hypotonic or isotonic. In some aspects, the concentration of potassium ion is higher than about 31 mM, wherein the medium is hypotonic or isotonic. In some aspects, the concentration of potassium ion is about 31 mM, wherein the medium is hypotonic or isotonic. In some aspects, the concentration of potassium ion is higher than about 32 mM, wherein the medium is hypotonic or isotonic. In some aspects, the concentration of potassium ion is about 32 mM, wherein the medium is hypotonic or isotonic. In some aspects, the concentration of potassium ion is higher than about 33 mM, wherein the medium is hypotonic or isotonic. In some aspects, the concentration of potassium ion is about 33 mM, wherein the medium is hypotonic. In some aspects, the concentration of potassium ion is higher than about 34 mM, wherein the medium is hypotonic or isotonic. In some aspects, the concentration of potassium ion is about 34 mM, wherein the medium is hypotonic or isotonic. In some aspects, the concentration of potassium ion is higher than about 35 mM, wherein the medium is hypotonic or isotonic. In some aspects, the concentration of potassium ion is about 35 mM, wherein the medium is hypotonic or isotonic. In some aspects, the concentration of potassium ion is higher than about 36 mM, wherein the medium is hypotonic or isotonic. In some aspects, the concentration of potassium ion is about 36 mM, wherein the medium is hypotonic or isotonic. In some aspects, the concentration of potassium ion is higher than about 37 mM, wherein the medium is hypotonic or isotonic. In some aspects, the concentration of potassium ion is about 37 mM, wherein the medium is hypotonic or isotonic. In some aspects, the concentration of potassium ion is higher than about 38 mM, wherein the medium is hypotonic or isotonic. In some aspects, the concentration of potassium ion is about 38 mM, wherein the medium is hypotonic or isotonic. In some aspects, the concentration of potassium ion is higher than about 39 mM, wherein the medium is hypotonic or isotonic. In some aspects, the concentration of potassium ion is about 39 mM, wherein the medium is hypotonic or isotonic.

In some aspects, the concentration of potassium ion is higher than about 40 mM, wherein the medium is hypotonic. In some aspects, the concentration of potassium ion is about 40 mM, wherein the medium is hypotonic. In some aspects, the concentration of potassium ion is higher than about 41 mM, wherein the medium is hypotonic. In some aspects, the concentration of potassium ion is about 41 mM, wherein the medium is hypotonic. In some aspects, the concentration of potassium ion is higher than about 42 mM, wherein the medium is hypotonic. In some aspects, the concentration of potassium ion is about 42 mM, wherein the medium is hypotonic. In some aspects, the concentration of potassium ion is higher than about 43 mM, wherein the medium is hypotonic. In some aspects, the concentration of potassium ion is about 43 mM, wherein the medium is hypotonic. In some aspects, the concentration of potassium ion is higher than about 44 mM, wherein the medium is hypotonic. In some aspects, the concentration of potassium ion is about 44 mM, wherein the medium is hypotonic. In some aspects, the concentration of potassium ion is higher than about 45 mM, wherein the medium is hypotonic. In some aspects, the concentration of potassium ion is about 45 mM, wherein the medium is hypotonic. In some aspects, the concentration of potassium ion is higher than about 46 mM, wherein the medium is hypotonic. In some aspects, the concentration of potassium ion is about 46 mM, wherein the medium is hypotonic. In some aspects, the concentration of potassium ion is higher than about 47 mM, wherein the medium is hypotonic. In some aspects, the concentration of potassium ion is about 47 mM, wherein the medium is hypotonic. In some aspects, the concentration of potassium ion is higher than about 48 mM, wherein the medium is hypotonic. In some aspects, the concentration of potassium ion is about 48 mM, wherein the medium is hypotonic. In some aspects, the concentration of potassium ion is higher than about 49 mM, wherein the medium is hypotonic. In some aspects, the concentration of potassium ion is about 49 mM, wherein the medium is hypotonic.

In some aspects, the concentration of potassium ion is higher than about 40 mM, wherein the total concentration of potassium ion and NaCl in the medium is between 110 mM and 140 mM. In some aspects, the concentration of potassium ion is about 40 mM, wherein the total concentration of potassium ion and NaCl in the medium is between 110 mM and 140 mM. In some aspects, the concentration of potassium ion is higher than about 41 mM, wherein the total concentration of potassium ion and NaCl in the medium is between 110 mM and 140 mM. In some aspects, the concentration of potassium ion is about 41 mM, wherein the total concentration of potassium ion and NaCl in the medium is between 110 mM and 140 mM. In some aspects, the concentration of potassium ion is higher than about 42 mM, wherein the total concentration of potassium ion and NaCl in the medium is between 110 mM and 140 mM. In some aspects, the concentration of potassium ion is about 42 mM, wherein the total concentration of potassium ion and NaCl in the medium is between 110 mM and 140 mM. In some aspects, the concentration of potassium ion is higher than about 43 mM, wherein the total concentration of potassium ion and NaCl in the medium is between 110 mM and 140 mM. In some aspects, the concentration of potassium ion is about 43 mM, wherein the total concentration of potassium ion and NaCl in the medium is between 110 mM and 140 mM. In some aspects, the concentration of potassium ion is higher than about 44 mM, wherein the total concentration of potassium ion and NaCl in the medium is between 110 mM and 140 mM. In some aspects, the concentration of potassium ion is about 44 mM, wherein the total concentration of potassium ion and NaCl in the medium is between 110 mM and 140 mM. In some aspects, the concentration of potassium ion is higher than about 45 mM, wherein the total concentration of potassium ion and NaCl in the medium is between 110 mM and 140 mM. In some aspects, the concentration of potassium ion is about 45 mM, wherein the total concentration of potassium ion and NaCl in the medium is between 110 mM and 140 mM. In some aspects, the concentration of potassium ion is higher than about 46 mM, wherein the total concentration of potassium ion and NaCl in the medium is between 110 mM and 140 mM. In some aspects, the concentration of potassium ion is about 46 mM, wherein the total concentration of potassium ion and NaCl in the medium is between 110 mM and 140 mM. In some aspects, the concentration of potassium ion is higher than about 47 mM, wherein the total concentration of potassium ion and NaCl in the medium is between 110 mM and 140 mM. In some aspects, the concentration of potassium ion is about 47 mM, wherein the total concentration of potassium ion and NaCl in the medium is between 110 mM and 140 mM. In some aspects, the concentration of potassium ion is higher than about 48 mM, wherein the total concentration of potassium ion and NaCl in the medium is between 110 mM and 140 mM. In some aspects, the concentration of potassium ion is about 48 mM, wherein the total concentration of potassium ion and NaCl in the medium is between 110 mM and 140 mM. In some aspects, the concentration of potassium ion is higher than about 49 mM, wherein the total concentration of potassium ion and NaCl in the medium is between 110 mM and 140 mM. In some aspects, the concentration of potassium ion is about 49 mM, wherein the total concentration of potassium ion and NaCl in the medium is between 110 mM and 140 mM.

In some aspects, the concentration of potassium ion is higher than about 50 mM. In some aspects, the concentration of potassium ion is about 50 mM. In some aspects, the concentration of potassium ion is higher than about 51 mM. In some aspects, the concentration of potassium ion is about 51 mM. In some aspects, the concentration of potassium ion is higher than about 52 mM. In some aspects, the concentration of potassium ion is about 52 mM. In some aspects, the concentration of potassium ion is higher than about 53 mM. In some aspects, the concentration of potassium ion is about 53 mM. In some aspects, the concentration of potassium ion is higher than about 54 mM. In some aspects, the concentration of potassium ion is about 54 mM. In some aspects, the concentration of potassium ion is higher than about 55 mM. In some aspects, the concentration of potassium ion is about 55 mM. In some aspects, the concentration of potassium ion is higher than about 56 mM. In some aspects, the concentration of potassium ion is about 56 mM. In some aspects, the concentration of potassium ion is higher than about 57 mM. In some aspects, the concentration of potassium ion is about 57 mM. In some aspects, the concentration of potassium ion is higher than about 58 mM. In some aspects, the concentration of potassium ion is about 58 mM. In some aspects, the concentration of potassium ion is higher than about 59 mM. In some aspects, the concentration of potassium ion is about 59 mM. In certain aspects, the medium is hypotonic. In certain aspects, the medium is isotonic.

In some aspects, the concentration of potassium ion is higher than about 50 mM, wherein the total concentration of potassium ion and NaCl in the medium is between 110 mM and 140 mM. In some aspects, the concentration of potassium ion is about 50 mM, wherein the total concentration of potassium ion and NaCl in the medium is between 110 mM and 140 mM. In some aspects, the concentration of potassium ion is higher than about 51 mM, wherein the total concentration of potassium ion and NaCl in the medium is between 110 mM and 140 mM. In some aspects, the concentration of potassium ion is about 51 mM, wherein the total concentration of potassium ion and NaCl in the medium is between 110 mM and 140 mM. In some aspects, the concentration of potassium ion is higher than about 52 mM, wherein the total concentration of potassium ion and NaCl in the medium is between 110 mM and 140 mM. In some aspects, the concentration of potassium ion is about 52 mM, wherein the total concentration of potassium ion and NaCl in the medium is between 110 mM and 140 mM. In some aspects, the concentration of potassium ion is higher than about 53 mM, wherein the total concentration of potassium ion and NaCl in the medium is between 110 mM and 140 mM. In some aspects, the concentration of potassium ion is about 53 mM, wherein the total concentration of potassium ion and NaCl in the medium is between 110 mM and 140 mM. In some aspects, the concentration of potassium ion is higher than about 54 mM, wherein the total concentration of potassium ion and NaCl in the medium is between 110 mM and 140 mM. In some aspects, the concentration of potassium ion is about 54 mM, wherein the total concentration of potassium ion and NaCl in the medium is between 110 mM and 140 mM. In some aspects, the concentration of potassium ion is higher than about 55 mM, wherein the total concentration of potassium ion and NaCl in the medium is between 110 mM and 140 mM. In some aspects, the concentration of potassium ion is about 55 mM, wherein the total concentration of potassium ion and NaCl in the medium is between 110 mM and 140 mM. In some aspects, the concentration of potassium ion is higher than about 56 mM, wherein the total concentration of potassium ion and NaCl in the medium is between 110 mM and 140 mM. In some aspects, the concentration of potassium ion is about 56 mM, wherein the total concentration of potassium ion and NaCl in the medium is between 110 mM and 140 mM. In some aspects, the concentration of potassium ion is higher than about 57 mM, wherein the total concentration of potassium ion and NaCl in the medium is between 110 mM and 140 mM. In some aspects, the concentration of potassium ion is about 57 mM, wherein the total concentration of potassium ion and NaCl in the medium is between 110 mM and 140 mM. In some aspects, the concentration of potassium ion is higher than about 58 mM, wherein the total concentration of potassium ion and NaCl in the medium is between 110 mM and 140 mM. In some aspects, the concentration of potassium ion is about 58 mM, wherein the total concentration of potassium ion and NaCl in the medium is between 110 mM and 140 mM. In some aspects, the concentration of potassium ion is higher than about 59 mM, wherein the total concentration of potassium ion and NaCl in the medium is between 110 mM and 140 mM. In some aspects, the concentration of potassium ion is about 59 mM, wherein the total concentration of potassium ion and NaCl in the medium is between 110 mM and 140 mM.

In some aspects, the concentration of potassium ion is higher than about 60 mM. In some aspects, the concentration of potassium ion is about 60 mM. In some aspects, the concentration of potassium ion is higher than about 61 mM. In some aspects, the concentration of potassium ion is about 61 mM. In some aspects, the concentration of potassium ion is higher than about 62 mM. In some aspects, the concentration of potassium ion is about 62 mM. In some aspects, the concentration of potassium ion is higher than about 63 mM. In some aspects, the concentration of potassium ion is about 63 mM. In some aspects, the concentration of potassium ion is higher than about 64 mM. In some aspects, the concentration of potassium ion is about 64 mM. In some aspects, the concentration of potassium ion is higher than about 65 mM. In some aspects, the concentration of potassium ion is about 65 mM. In some aspects, the concentration of potassium ion is higher than about 66 mM. In some aspects, the concentration of potassium ion is about 66 mM. In some aspects, the concentration of potassium ion is higher than about 67 mM. In some aspects, the concentration of potassium ion is about 67 mM. In some aspects, the concentration of potassium ion is higher than about 68 mM. In some aspects, the concentration of potassium ion is about 68 mM. In some aspects, the concentration of potassium ion is higher than about 69 mM. In some aspects, the concentration of potassium ion is about 69 mM. In certain aspects, the medium is hypotonic. In certain aspects, the medium is isotonic.

In some aspects, the concentration of potassium ion is higher than about 60 mM, wherein the total concentration of potassium ion and NaCl in the medium is between 110 mM and 140 mM. In some aspects, the concentration of potassium ion is about 60 mM, wherein the total concentration of potassium ion and NaCl in the medium is between 110 mM and 140 mM. In some aspects, the concentration of potassium ion is higher than about 61 mM, wherein the total concentration of potassium ion and NaCl in the medium is between 110 mM and 140 mM. In some aspects, the concentration of potassium ion is about 61 mM, wherein the total concentration of potassium ion and NaCl in the medium is between 110 mM and 140 mM. In some aspects, the concentration of potassium ion is higher than about 62 mM, wherein the total concentration of potassium ion and NaCl in the medium is between 110 mM and 140 mM. In some aspects, the concentration of potassium ion is about 62 mM, wherein the total concentration of potassium ion and NaCl in the medium is between 110 mM and 140 mM. In some aspects, the concentration of potassium ion is higher than about 63 mM, wherein the total concentration of potassium ion and NaCl in the medium is between 110 mM and 140 mM. In some aspects, the concentration of potassium ion is about 63 mM, wherein the total concentration of potassium ion and NaCl in the medium is between 110 mM and 140 mM. In some aspects, the concentration of potassium ion is higher than about 64 mM, wherein the total concentration of potassium ion and NaCl in the medium is between 110 mM and 140 mM. In some aspects, the concentration of potassium ion is about 64 mM, wherein the total concentration of potassium ion and NaCl in the medium is between 110 mM and 140 mM. In some aspects, the concentration of potassium ion is higher than about 65 mM, wherein the total concentration of potassium ion and NaCl in the medium is between 110 mM and 140 mM. In some aspects, the concentration of potassium ion is about 65 mM, wherein the total concentration of potassium ion and NaCl in the medium is between 110 mM and 140 mM. In some aspects, the concentration of potassium ion is higher than about 66 mM, wherein the total concentration of potassium ion and NaCl in the medium is between 110 mM and 140 mM. In some aspects, the concentration of potassium ion is about 66 mM, wherein the total concentration of potassium ion and NaCl in the medium is between 110 mM and 140 mM. In some aspects, the concentration of potassium ion is higher than about 67 mM, wherein the total concentration of potassium ion and NaCl in the medium is between 110 mM and 140 mM. In some aspects, the concentration of potassium ion is about 67 mM, wherein the total concentration of potassium ion and NaCl in the medium is between 110 mM and 140 mM. In some aspects, the concentration of potassium ion is higher than about 68 mM, wherein the total concentration of potassium ion and NaCl in the medium is between 110 mM and 140 mM. In some aspects, the concentration of potassium ion is about 68 mM, wherein the total concentration of potassium ion and NaCl in the medium is between 110 mM and 140 mM. In some aspects, the concentration of potassium ion is higher than about 69 mM, wherein the total concentration of potassium ion and NaCl in the medium is between 110 mM and 140 mM. In some aspects, the concentration of potassium ion is about 69 mM, wherein the total concentration of potassium ion and NaCl in the medium is between 110 mM and 140 mM.

In some aspects, the concentration of potassium ion is higher than about 70 mM. In some aspects, the concentration of potassium ion is about 70 mM. In some aspects, the concentration of potassium ion is higher than about 71 mM. In some aspects, the concentration of potassium ion is about 71 mM. In some aspects, the concentration of potassium ion is higher than about 72 mM. In some aspects, the concentration of potassium ion is about 72 mM. In some aspects, the concentration of potassium ion is higher than about 73 mM. In some aspects, the concentration of potassium ion is about 73 mM. In some aspects, the concentration of potassium ion is higher than about 74 mM. In some aspects, the concentration of potassium ion is about 74 mM. In some aspects, the concentration of potassium ion is higher than about 75 mM. In some aspects, the concentration of potassium ion is about 75 mM. In some aspects, the concentration of potassium ion is higher than about 76 mM. In some aspects, the concentration of potassium ion is about 76 mM. In some aspects, the concentration of potassium ion is higher than about 77 mM. In some aspects, the concentration of potassium ion is about 77 mM. In some aspects, the concentration of potassium ion is higher than about 78 mM. In some aspects, the concentration of potassium ion is about 78 mM. In some aspects, the concentration of potassium ion is higher than about 79 mM. In some aspects, the concentration of potassium ion is about 79 mM. In certain aspects, the medium is hypotonic. In certain aspects, the medium is isotonic.

In some aspects, the concentration of potassium ion is higher than about 70 mM, wherein the total concentration of potassium ion and NaCl in the medium is between 110 mM and 140 mM. In some aspects, the concentration of potassium ion is about 70 mM, wherein the total concentration of potassium ion and NaCl in the medium is between 110 mM and 140 mM. In some aspects, the concentration of potassium ion is higher than about 71 mM, wherein the total concentration of potassium ion and NaCl in the medium is between 110 mM and 140 mM. In some aspects, the concentration of potassium ion is about 71 mM, wherein the total concentration of potassium ion and NaCl in the medium is between 110 mM and 140 mM. In some aspects, the concentration of potassium ion is higher than about 72 mM, wherein the total concentration of potassium ion and NaCl in the medium is between 110 mM and 140 mM. In some aspects, the concentration of potassium ion is about 72 mM, wherein the total concentration of potassium ion and NaCl in the medium is between 110 mM and 140 mM. In some aspects, the concentration of potassium ion is higher than about 73 mM, wherein the total concentration of potassium ion and NaCl in the medium is between 110 mM and 140 mM. In some aspects, the concentration of potassium ion is about 73 mM, wherein the total concentration of potassium ion and NaCl in the medium is between 110 mM and 140 mM. In some aspects, the concentration of potassium ion is higher than about 74 mM, wherein the total concentration of potassium ion and NaCl in the medium is between 110 mM and 140 mM. In some aspects, the concentration of potassium ion is about 74 mM, wherein the total concentration of potassium ion and NaCl in the medium is between 110 mM and 140 mM. In some aspects, the concentration of potassium ion is higher than about 75 mM, wherein the total concentration of potassium ion and NaCl in the medium is between 110 mM and 140 mM. In some aspects, the concentration of potassium ion is about 75 mM, wherein the total concentration of potassium ion and NaCl in the medium is between 110 mM and 140 mM. In some aspects, the concentration of potassium ion is higher than about 76 mM, wherein the total concentration of potassium ion and NaCl in the medium is between 110 mM and 140 mM. In some aspects, the concentration of potassium ion is about 76 mM, wherein the total concentration of potassium ion and NaCl in the medium is between 110 mM and 140 mM. In some aspects, the concentration of potassium ion is higher than about 77 mM, wherein the total concentration of potassium ion and NaCl in the medium is between 110 mM and 140 mM. In some aspects, the concentration of potassium ion is about 77 mM, wherein the total concentration of potassium ion and NaCl in the medium is between 110 mM and 140 mM. In some aspects, the concentration of potassium ion is higher than about 78 mM, wherein the total concentration of potassium ion and NaCl in the medium is between 110 mM and 140 mM. In some aspects, the concentration of potassium ion is about 78 mM, wherein the total concentration of potassium ion and NaCl in the medium is between 110 mM and 140 mM. In some aspects, the concentration of potassium ion is higher than about 79 mM, wherein the total concentration of potassium ion and NaCl in the medium is between 110 mM and 140 mM. In some aspects, the concentration of potassium ion is about 79 mM, wherein the total concentration of potassium ion and NaCl in the medium is between 110 mM and 140 mM.

In some aspects, the concentration of potassium ion is higher than about 80 mM. In some aspects, the concentration of potassium ion is about 80 mM. In some aspects, the concentration of potassium ion is higher than about 81 mM. In some aspects, the concentration of potassium ion is about 81 mM. In some aspects, the concentration of potassium ion is higher than about 82 mM. In some aspects, the concentration of potassium ion is about 82 mM. In some aspects, the concentration of potassium ion is higher than about 83 mM. In some aspects, the concentration of potassium ion is about 83 mM. In some aspects, the concentration of potassium ion is higher than about 84 mM. In some aspects, the concentration of potassium ion is about 84 mM. In some aspects, the concentration of potassium ion is higher than about 85 mM. In some aspects, the concentration of potassium ion is about 85 mM. In some aspects, the concentration of potassium ion is higher than about 86 mM. In some aspects, the concentration of potassium ion is about 86 mM. In some aspects, the concentration of potassium ion is higher than about 87 mM. In some aspects, the concentration of potassium ion is about 87 mM. In some aspects, the concentration of potassium ion is higher than about 88 mM. In some aspects, the concentration of potassium ion is about 88 mM. In some aspects, the concentration of potassium ion is higher than about 89 mM. In some aspects, the concentration of potassium ion is about 89 mM. In certain aspects, the medium is hypotonic. In certain aspects, the medium is isotonic.

In some aspects, the concentration of potassium ion is higher than about 80 mM, wherein the total concentration of potassium ion and NaCl in the medium is between 110 mM and 140 mM. In some aspects, the concentration of potassium ion is about 80 mM, wherein the total concentration of potassium ion and NaCl in the medium is between 110 mM and 140 mM. In some aspects, the concentration of potassium ion is higher than about 81 mM, wherein the total concentration of potassium ion and NaCl in the medium is between 110 mM and 140 mM. In some aspects, the concentration of potassium ion is about 81 mM, wherein the total concentration of potassium ion and NaCl in the medium is between 110 mM and 140 mM. In some aspects, the concentration of potassium ion is higher than about 82 mM, wherein the total concentration of potassium ion and NaCl in the medium is between 110 mM and 140 mM. In some aspects, the concentration of potassium ion is about 82 mM, wherein the total concentration of potassium ion and NaCl in the medium is between 110 mM and 140 mM. In some aspects, the concentration of potassium ion is higher than about 83 mM, wherein the total concentration of potassium ion and NaCl in the medium is between 110 mM and 140 mM. In some aspects, the concentration of potassium ion is about 83 mM, wherein the total concentration of potassium ion and NaCl in the medium is between 110 mM and 140 mM. In some aspects, the concentration of potassium ion is higher than about 84 mM, wherein the total concentration of potassium ion and NaCl in the medium is between 110 mM and 140 mM. In some aspects, the concentration of potassium ion is about 84 mM, wherein the total concentration of potassium ion and NaCl in the medium is between 110 mM and 140 mM. In some aspects, the concentration of potassium ion is higher than about 85 mM, wherein the total concentration of potassium ion and NaCl in the medium is between 110 mM and 140 mM. In some aspects, the concentration of potassium ion is about 85 mM, wherein the total concentration of potassium ion and NaCl in the medium is between 110 mM and 140 mM. In some aspects, the concentration of potassium ion is higher than about 86 mM, wherein the total concentration of potassium ion and NaCl in the medium is between 110 mM and 140 mM. In some aspects, the concentration of potassium ion is about 86 mM, wherein the total concentration of potassium ion and NaCl in the medium is between 110 mM and 140 mM. In some aspects, the concentration of potassium ion is higher than about 87 mM, wherein the total concentration of potassium ion and NaCl in the medium is between 110 mM and 140 mM. In some aspects, the concentration of potassium ion is about 87 mM, wherein the total concentration of potassium ion and NaCl in the medium is between 110 mM and 140 mM. In some aspects, the concentration of potassium ion is higher than about 88 mM, wherein the total concentration of potassium ion and NaCl in the medium is between 110 mM and 140 mM. In some aspects, the concentration of potassium ion is about 88 mM, wherein the total concentration of potassium ion and NaCl in the medium is between 110 mM and 140 mM. In some aspects, the concentration of potassium ion is higher than about 89 mM, wherein the total concentration of potassium ion and NaCl in the medium is between 110 mM and 140 mM. In some aspects, the concentration of potassium ion is about 89 mM, wherein the total concentration of potassium ion and NaCl in the medium is between 110 mM and 140 mM.

In some aspects, the concentration of potassium ion is higher than about 90 mM. In some aspects, the concentration of potassium ion is about 90 mM. In some aspects, the concentration of potassium ion is higher than about 91 mM. In some aspects, the concentration of potassium ion is about 91 mM. In some aspects, the concentration of potassium ion is higher than about 92 mM. In some aspects, the concentration of potassium ion is about 92 mM. In some aspects, the concentration of potassium ion is higher than about 93 mM. In some aspects, the concentration of potassium ion is about 93 mM. In some aspects, the concentration of potassium ion is higher than about 94 mM. In some aspects, the concentration of potassium ion is about 94 mM. In some aspects, the concentration of potassium ion is higher than about 95 mM. In some aspects, the concentration of potassium ion is about 95 mM. In some aspects, the concentration of potassium ion is higher than about 96 mM. In some aspects, the concentration of potassium ion is about 96 mM. In some aspects, the concentration of potassium ion is higher than about 97 mM. In some aspects, the concentration of potassium ion is about 97 mM. In some aspects, the concentration of potassium ion is higher than about 98 mM. In some aspects, the concentration of potassium ion is about 98 mM. In some aspects, the concentration of potassium ion is higher than about 99 mM. In some aspects, the concentration of potassium ion is about 99 mM. In certain aspects, the medium is hypotonic. In certain aspects, the medium is isotonic.

In some aspects, the concentration of potassium ion is higher than about 90 mM, wherein the total concentration of potassium ion and NaCl in the medium is between 110 mM and 140 mM. In some aspects, the concentration of potassium ion is about 90 mM, wherein the total concentration of potassium ion and NaCl in the medium is between 110 mM and 140 mM. In some aspects, the concentration of potassium ion is higher than about 91 mM, wherein the total concentration of potassium ion and NaCl in the medium is between 110 mM and 140 mM. In some aspects, the concentration of potassium ion is about 91 mM, wherein the total concentration of potassium ion and NaCl in the medium is between 110 mM and 140 mM. In some aspects, the concentration of potassium ion is higher than about 92 mM, wherein the total concentration of potassium ion and NaCl in the medium is between 110 mM and 140 mM. In some aspects, the concentration of potassium ion is about 92 mM, wherein the total concentration of potassium ion and NaCl in the medium is between 110 mM and 140 mM. In some aspects, the concentration of potassium ion is higher than about 93 mM, wherein the total concentration of potassium ion and NaCl in the medium is between 110 mM and 140 mM. In some aspects, the concentration of potassium ion is about 93 mM, wherein the total concentration of potassium ion and NaCl in the medium is between 110 mM and 140 mM. In some aspects, the concentration of potassium ion is higher than about 94 mM, wherein the total concentration of potassium ion and NaCl in the medium is between 110 mM and 140 mM. In some aspects, the concentration of potassium ion is about 94 mM, wherein the total concentration of potassium ion and NaCl in the medium is between 110 mM and 140 mM. In some aspects, the concentration of potassium ion is higher than about 95 mM, wherein the total concentration of potassium ion and NaCl in the medium is between 110 mM and 140 mM. In some aspects, the concentration of potassium ion is about 95 mM, wherein the total concentration of potassium ion and NaCl in the medium is between 110 mM and 140 mM. In some aspects, the concentration of potassium ion is higher than about 96 mM, wherein the total concentration of potassium ion and NaCl in the medium is between 110 mM and 140 mM. In some aspects, the concentration of potassium ion is about 96 mM, wherein the total concentration of potassium ion and NaCl in the medium is between 110 mM and 140 mM. In some aspects, the concentration of potassium ion is higher than about 97 mM, wherein the total concentration of potassium ion and NaCl in the medium is between 110 mM and 140 mM. In some aspects, the concentration of potassium ion is about 97 mM, wherein the total concentration of potassium ion and NaCl in the medium is between 110 mM and 140 mM. In some aspects, the concentration of potassium ion is higher than about 98 mM, wherein the total concentration of potassium ion and NaCl in the medium is between 110 mM and 140 mM. In some aspects, the concentration of potassium ion is about 98 mM, wherein the total concentration of potassium ion and NaCl in the medium is between 110 mM and 140 mM. In some aspects, the concentration of potassium ion is higher than about 99 mM, wherein the total concentration of potassium ion and NaCl in the medium is between 110 mM and 140 mM. In some aspects, the concentration of potassium ion is about 99 mM, wherein the total concentration of potassium ion and NaCl in the medium is between 110 mM and 140 mM.

In some aspects, the concentration of potassium ion is higher than about 100 mM. In some aspects, the concentration of potassium ion is about 100 mM. In some aspects, the concentration of potassium ion is higher than about 101 mM. In some aspects, the concentration of potassium ion is about 101 mM. In some aspects, the concentration of potassium ion is higher than about 102 mM. In some aspects, the concentration of potassium ion is about 102 mM. In some aspects, the concentration of potassium ion is higher than about 103 mM. In some aspects, the concentration of potassium ion is about 103 mM. In some aspects, the concentration of potassium ion is higher than about 104 mM. In some aspects, the concentration of potassium ion is about 104 mM. In some aspects, the concentration of potassium ion is higher than about 105 mM. In some aspects, the concentration of potassium ion is about 105 mM. In some aspects, the concentration of potassium ion is higher than about 106 mM. In some aspects, the concentration of potassium ion is about 106 mM. In some aspects, the concentration of potassium ion is higher than about 107 mM. In some aspects, the concentration of potassium ion is about 107 mM. In some aspects, the concentration of potassium ion is higher than about 108 mM. In some aspects, the concentration of potassium ion is about 108 mM. In some aspects, the concentration of potassium ion is higher than about 109 mM. In some aspects, the concentration of potassium ion is about 109 mM. In certain aspects, the medium is hypotonic. In certain aspects, the medium is isotonic.

In some aspects, the concentration of potassium ion is higher than about 100 mM, wherein the total concentration of potassium ion and NaCl in the medium is between 110 mM and 140 mM. In some aspects, the concentration of potassium ion is about 100 mM, wherein the total concentration of potassium ion and NaCl in the medium is between 110 mM and 140 mM. In some aspects, the concentration of potassium ion is higher than about 101 mM, wherein the total concentration of potassium ion and NaCl in the medium is between 110 mM and 140 mM. In some aspects, the concentration of potassium ion is about 101 mM, wherein the total concentration of potassium ion and NaCl in the medium is between 110 mM and 140 mM. In some aspects, the concentration of potassium ion is higher than about 102 mM, wherein the total concentration of potassium ion and NaCl in the medium is between 110 mM and 140 mM. In some aspects, the concentration of potassium ion is about 102 mM, wherein the total concentration of potassium ion and NaCl in the medium is between 110 mM and 140 mM. In some aspects, the concentration of potassium ion is higher than about 103 mM, wherein the total concentration of potassium ion and NaCl in the medium is between 110 mM and 140 mM. In some aspects, the concentration of potassium ion is about 103 mM, wherein the total concentration of potassium ion and NaCl in the medium is between 110 mM and 140 mM. In some aspects, the concentration of potassium ion is higher than about 104 mM, wherein the total concentration of potassium ion and NaCl in the medium is between 110 mM and 140 mM. In some aspects, the concentration of potassium ion is about 104 mM, wherein the total concentration of potassium ion and NaCl in the medium is between 110 mM and 140 mM. In some aspects, the concentration of potassium ion is higher than about 105 mM, wherein the total concentration of potassium ion and NaCl in the medium is between 110 mM and 140 mM. In some aspects, the concentration of potassium ion is about 105 mM, wherein the total concentration of potassium ion and NaCl in the medium is between 110 mM and 140 mM. In some aspects, the concentration of potassium ion is higher than about 106 mM, wherein the total concentration of potassium ion and NaCl in the medium is between 110 mM and 140 mM. In some aspects, the concentration of potassium ion is about 106 mM, wherein the total concentration of potassium ion and NaCl in the medium is between 110 mM and 140 mM. In some aspects, the concentration of potassium ion is higher than about 107 mM, wherein the total concentration of potassium ion and NaCl in the medium is between 110 mM and 140 mM. In some aspects, the concentration of potassium ion is about 107 mM, wherein the total concentration of potassium ion and NaCl in the medium is between 110 mM and 140 mM. In some aspects, the concentration of potassium ion is higher than about 108 mM, wherein the total concentration of potassium ion and NaCl in the medium is between 110 mM and 140 mM. In some aspects, the concentration of potassium ion is about 108 mM, wherein the total concentration of potassium ion and NaCl in the medium is between 110 mM and 140 mM. In some aspects, the concentration of potassium ion is higher than about 109 mM, wherein the total concentration of potassium ion and NaCl in the medium is between 110 mM and 140 mM. In some aspects, the concentration of potassium ion is about 109 mM, wherein the total concentration of potassium ion and NaCl in the medium is between 110 mM and 140 mM.

In some aspects, the concentration of potassium ion is higher than about 110 mM. In some aspects, the concentration of potassium ion is about 110 mM. In some aspects, the concentration of potassium ion is higher than about 111 mM. In some aspects, the concentration of potassium ion is about 111 mM. In some aspects, the concentration of potassium ion is higher than about 112 mM. In some aspects, the concentration of potassium ion is about 112 mM. In some aspects, the concentration of potassium ion is higher than about 113 mM. In some aspects, the concentration of potassium ion is about 113 mM. In some aspects, the concentration of potassium ion is higher than about 114 mM. In some aspects, the concentration of potassium ion is about 114 mM. In some aspects, the concentration of potassium ion is higher than about 115 mM. In some aspects, the concentration of potassium ion is about 115 mM. In some aspects, the concentration of potassium ion is higher than about 116 mM. In some aspects, the concentration of potassium ion is about 116 mM. In some aspects, the concentration of potassium ion is higher than about 117 mM. In some aspects, the concentration of potassium ion is about 117 mM. In some aspects, the concentration of potassium ion is higher than about 118 mM. In some aspects, the concentration of potassium ion is about 118 mM. In some aspects, the concentration of potassium ion is higher than about 119 mM. In some aspects, the concentration of potassium ion is about 119 mM. In certain aspects, the medium is hypotonic. In certain aspects, the medium is isotonic.

In some aspects, the concentration of potassium ion is higher than about 110 mM, wherein the total concentration of potassium ion and NaCl in the medium is between 110 mM and 140 mM. In some aspects, the concentration of potassium ion is about 110 mM, wherein the total concentration of potassium ion and NaCl in the medium is between 110 mM and 140 mM. In some aspects, the concentration of potassium ion is higher than about 111 mM, wherein the total concentration of potassium ion and NaCl in the medium is between 110 mM and 140 mM. In some aspects, the concentration of potassium ion is about 111 mM, wherein the total concentration of potassium ion and NaCl in the medium is between 110 mM and 140 mM. In some aspects, the concentration of potassium ion is higher than about 112 mM, wherein the total concentration of potassium ion and NaCl in the medium is between 110 mM and 140 mM. In some aspects, the concentration of potassium ion is about 112 mM, wherein the total concentration of potassium ion and NaCl in the medium is between 110 mM and 140 mM. In some aspects, the concentration of potassium ion is higher than about 113 mM, wherein the total concentration of potassium ion and NaCl in the medium is between 110 mM and 140 mM. In some aspects, the concentration of potassium ion is about 113 mM, wherein the total concentration of potassium ion and NaCl in the medium is between 110 mM and 140 mM. In some aspects, the concentration of potassium ion is higher than about 114 mM, wherein the total concentration of potassium ion and NaCl in the medium is between 110 mM and 140 mM. In some aspects, the concentration of potassium ion is about 114 mM, wherein the total concentration of potassium ion and NaCl in the medium is between 110 mM and 140 mM. In some aspects, the concentration of potassium ion is higher than about 115 mM, wherein the total concentration of potassium ion and NaCl in the medium is between 110 mM and 140 mM. In some aspects, the concentration of potassium ion is about 115 mM, wherein the total concentration of potassium ion and NaCl in the medium is between 110 mM and 140 mM. In some aspects, the concentration of potassium ion is higher than about 116 mM, wherein the total concentration of potassium ion and NaCl in the medium is between 110 mM and 140 mM. In some aspects, the concentration of potassium ion is about 116 mM, wherein the total concentration of potassium ion and NaCl in the medium is between 110 mM and 140 mM. In some aspects, the concentration of potassium ion is higher than about 117 mM, wherein the total concentration of potassium ion and NaCl in the medium is between 110 mM and 140 mM. In some aspects, the concentration of potassium ion is about 117 mM, wherein the total concentration of potassium ion and NaCl in the medium is between 110 mM and 140 mM. In some aspects, the concentration of potassium ion is higher than about 118 mM, wherein the total concentration of potassium ion and NaCl in the medium is between 110 mM and 140 mM. In some aspects, the concentration of potassium ion is about 118 mM, wherein the total concentration of potassium ion and NaCl in the medium is between 110 mM and 140 mM. In some aspects, the concentration of potassium ion is higher than about 119 mM, wherein the total concentration of potassium ion and NaCl in the medium is between 110 mM and 140 mM. In some aspects, the concentration of potassium ion is about 119 mM, wherein the total concentration of potassium ion and NaCl in the medium is between 110 mM and 140 mM.

In some aspects, the concentration of potassium ion is higher than about 120 mM. In some aspects, the concentration of potassium ion is about 120 mM. In some aspects, the concentration of potassium ion is higher than about 121 mM. In some aspects, the concentration of potassium ion is about 121 mM. In some aspects, the concentration of potassium ion is higher than about 122 mM. In some aspects, the concentration of potassium ion is about 122 mM. In some aspects, the concentration of potassium ion is higher than about 123 mM. In some aspects, the concentration of potassium ion is about 123 mM. In some aspects, the concentration of potassium ion is higher than about 124 mM. In some aspects, the concentration of potassium ion is about 124 mM. In some aspects, the concentration of potassium ion is higher than about 125 mM. In some aspects, the concentration of potassium ion is about 125 mM. In some aspects, the concentration of potassium ion is higher than about 126 mM. In some aspects, the concentration of potassium ion is about 126 mM. In some aspects, the concentration of potassium ion is higher than about 127 mM. In some aspects, the concentration of potassium ion is about 127 mM. In some aspects, the concentration of potassium ion is higher than about 128 mM. In some aspects, the concentration of potassium ion is about 128 mM. In some aspects, the concentration of potassium ion is higher than about 129 mM. In some aspects, the concentration of potassium ion is about 129 mM. In some aspects, the concentration of potassium ion is higher than about 130 mM. In some aspects, the concentration of potassium ion is about 130 mM. In certain aspects, the medium is hypotonic. In certain aspects, the medium is isotonic.

In some aspects, the concentration of potassium ion is higher than about 120 mM, wherein the total concentration of potassium ion and NaCl in the medium is between 110 mM and 140 mM. In some aspects, the concentration of potassium ion is about 120 mM, wherein the total concentration of potassium ion and NaCl in the medium is between 110 mM and 140 mM. In some aspects, the concentration of potassium ion is higher than about 121 mM, wherein the total concentration of potassium ion and NaCl in the medium is between 110 mM and 140 mM. In some aspects, the concentration of potassium ion is about 121 mM, wherein the total concentration of potassium ion and NaCl in the medium is between 110 mM and 140 mM. In some aspects, the concentration of potassium ion is higher than about 122 mM, wherein the total concentration of potassium ion and NaCl in the medium is between 110 mM and 140 mM. In some aspects, the concentration of potassium ion is about 122 mM, wherein the total concentration of potassium ion and NaCl in the medium is between 110 mM and 140 mM. In some aspects, the concentration of potassium ion is higher than about 123 mM, wherein the total concentration of potassium ion and NaCl in the medium is between 110 mM and 140 mM. In some aspects, the concentration of potassium ion is about 123 mM, wherein the total concentration of potassium ion and NaCl in the medium is between 110 mM and 140 mM. In some aspects, the concentration of potassium ion is higher than about 124 mM, wherein the total concentration of potassium ion and NaCl in the medium is between 110 mM and 140 mM. In some aspects, the concentration of potassium ion is about 124 mM, wherein the total concentration of potassium ion and NaCl in the medium is between 110 mM and 140 mM. In some aspects, the concentration of potassium ion is higher than about 125 mM, wherein the total concentration of potassium ion and NaCl in the medium is between 110 mM and 140 mM. In some aspects, the concentration of potassium ion is about 125 mM, wherein the total concentration of potassium ion and NaCl in the medium is between 110 mM and 140 mM. In some aspects, the concentration of potassium ion is higher than about 126 mM, wherein the total concentration of potassium ion and NaCl in the medium is between 110 mM and 140 mM. In some aspects, the concentration of potassium ion is about 126 mM, wherein the total concentration of potassium ion and NaCl in the medium is between 110 mM and 140 mM. In some aspects, the concentration of potassium ion is higher than about 127 mM, wherein the total concentration of potassium ion and NaCl in the medium is between 110 mM and 140 mM. In some aspects, the concentration of potassium ion is about 127 mM, wherein the total concentration of potassium ion and NaCl in the medium is between 110 mM and 140 mM. In some aspects, the concentration of potassium ion is higher than about 128 mM, wherein the total concentration of potassium ion and NaCl in the medium is between 110 mM and 140 mM. In some aspects, the concentration of potassium ion is about 128 mM, wherein the total concentration of potassium ion and NaCl in the medium is between 110 mM and 140 mM. In some aspects, the concentration of potassium ion is higher than about 129 mM, wherein the total concentration of potassium ion and NaCl in the medium is between 110 mM and 140 mM. In some aspects, the concentration of potassium ion is about 129 mM, wherein the total concentration of potassium ion and NaCl in the medium is between 110 mM and 140 mM. In some aspects, the concentration of potassium ion is higher than about 130 mM, wherein the total concentration of potassium ion and NaCl in the medium is between 110 mM and 140 mM. In some aspects, the concentration of potassium ion is about 130 mM, wherein the total concentration of potassium ion and NaCl in the medium is between 110 mM and 140 mM.

In some aspects, the medium comprising a high concentration of potassium ion can be prepared by adding a sufficient amount of a potassium salt in a medium. In some aspects, non-limiting examples of potassium salt include potassium aminetrichloroplatinate, potassium aquapentachlororuthenate, potassium bis(oxalato)platinate(II) dihydrate, potassium bisulfate, potassium borohydride, potassium bromide, potassium carbonate, potassium chloride, potassium chromate, potassium dichromate, potassium dicyanoargentate, potassium dicyanoaurate, potassium fluoride, potassium fluorosulfate, potassium hexachloroiridate, potassium hexachloroosmate, potassium hexachloropalladate, potassium hexachloroplatinate, potassium hexachlororhenate, potassium hexacyanochromate, potassium hexacyanoferrate, potassium hexacyanoruthenate(II) hydrate, potassium hexafluoroantimonate, potassium hexafluoronickelate, potassium hexafluorophosphate, potassium hexafluorotitanate, potassium hexafluorozirconate, potassium hexahydroxoantimonate, potassium hexaiodoplatinate, potassium hexaiodorhenate, potassium hydroxide, potassium iodate, potassium iodide, potassium manganate, potassium metavanadate, potassium molybdate, potassium nitrate, potassium nitrosodisulfonate, potassium osmate(VI) dihydrate, potassium pentachloronitrosylruthenate, potassium perchlorate, potassium perrhenate, potassium perruthenate, potassium persulfate, potassium phosphate dibasic, potassium phosphate monobasic, potassium pyrophosphate, potassium selenocyanate, potassium selenocyanate, potassium stannate trihydrate, potassium sulfate, potassium tellurate hydrate, potassium tellurite, potassium tetraborate tetrahydrate, potassium tetrabromoaurate, potassium tetrabromopalladate, potassium tetrachloropalladate, potassium tetrachloroplatinate, potassium tetracyanopalladate, potassium tetracyanoplatinate, potassium tetrafluoroborate, potassium tetranitroplatinate, potassium tetrathionate, potassium p-toluenethiosulfonate, and potassium hydroxycitrate tribasic monohydrate. In certain aspects, the potassium salt comprises potassium chloride (KCl). In certain aspects, the potassium salt comprises potassium gluconate. In certain aspects, the potassium salt comprises potassium citrate. In certain aspects, the potassium salt comprises potassium hydroxycitrate. In some aspects, the potassium salt comprises a combination of any of the potassium salts disclosed herein.

In some aspects, the medium comprises potassium ion at a concentration disclosed herein, and the medium has an osmolality of about 250 mOsmol. In some aspects, the medium comprises potassium ion at a concentration disclosed herein, and the medium has an osmolality of about 280 mOsmol.

In some aspects, the medium comprises about 50 mM potassium ion, and the medium has an osmolality of about 255 mOsmol. In some aspects, the medium comprises about 50 mM potassium ion, and the medium has an osmolality of about 250 mOsmol to about 260 mOsmol. In some aspects, the medium comprises about 50 mM potassium ion, and the medium has an osmolality of about 254 mOsmol to about 256 mOsmol. In some aspects, the medium comprises about 50 mM potassium ion, and the medium has an osmolality of about 254.7 mOsmol.

In some aspects, the medium comprises about 51 mM, about 52 mM, about 53 mM, or about 54 mM potassium ion, and the medium has an osmolality of about 255 mOsmol. In some aspects, the medium comprises about 51 mM, about 52 mM, about 53 mM, or about 54 mM potassium ion, and the medium has an osmolality of about 250 mOsmol to about 260 mOsmol. In some aspects, the medium comprises about 51 mM, about 52 mM, about 53 mM, or about 54 mM potassium ion, and the medium has an osmolality of about 254 mOsmol to about 256 mOsmol. In some aspects, the medium comprises about 51 mM, about 52 mM, about 53 mM, or about 54 mM potassium ion, and the medium has an osmolality of about 254.7 mOsmol. In some aspects, the medium comprises about 51 mM, about 52 mM, about 53 mM, or about 54 mM potassium ion, and the medium has an osmolality of about 255.2 mOsmol.

In some aspects, the medium comprises about 55 mM potassium ion, and the medium has an osmolality of about 255 mOsmol. In some aspects, the medium comprises about 55 mM potassium ion, and the medium has an osmolality of about 250 mOsmol to about 260 mOsmol. In some aspects, the medium comprises about 55 mM potassium ion, and the medium has an osmolality of about 254 mOsmol to about 256 mOsmol. In some aspects, the medium comprises about 55 mM potassium ion, and the medium has an osmolality of about 255.2 mOsmol.

In some aspects, the medium comprises about 56 mM, about 57 mM, about 58 mM, or about 59 mM potassium ion, and the medium has an osmolality of about 256 mOsmol. In some aspects, the medium comprises about 56 mM, about 57 mM, about 58 mM, or about 59 mM potassium ion, and the medium has an osmolality of about 250 mOsmol to about 260 mOsmol. In some aspects, the medium comprises about 56 mM, about 57 mM, about 58 mM, or about 59 mM potassium ion, and the medium has an osmolality of about 255 mOsmol to about 258 mOsmol. In some aspects, the medium comprises about 60 mM potassium ion, and the medium has an osmolality of about 257.2 mOsmol.

In some aspects, the medium comprises about 60 mM potassium ion, and the medium has an osmolality of about 257 mOsmol. In some aspects, the medium comprises about 60 mM potassium ion, and the medium has an osmolality of about 250 mOsmol to about 260 mOsmol. In some aspects, the medium comprises about 60 mM potassium ion, and the medium has an osmolality of about 256 mOsmol to about 258 mOsmol. In some aspects, the medium comprises about 60 mM potassium ion, and the medium has an osmolality of about 257.2 mOsmol.

In some aspects, the medium comprises about 61 mM, about 62 mM, about 63 mM, or about 64 mM potassium ion, and the medium has an osmolality of about 257 mOsmol. In some aspects, the medium comprises about 61 mM, about 62 mM, about 63 mM, or about 64 mM potassium ion, and the medium has an osmolality of about 250 mOsmol to about 260 mOsmol. In some aspects, the medium comprises about 61 mM, about 62 mM, about 63 mM, or about 64 mM potassium ion, and the medium has an osmolality of about 256 mOsmol to about 258 mOsmol. In some aspects, the medium comprises about 61 mM, about 62 mM, about 63 mM, or about 64 mM potassium ion, and the medium has an osmolality of about 257.2 mOsmol. In some aspects, the medium comprises about 61 mM, about 62 mM, about 63 mM, or about 64 mM potassium ion, and the medium has an osmolality of about 257.5 mOsmol.

In some aspects, the medium comprises about 65 mM potassium ion, and the medium has an osmolality of about 257 mOsmol. In some aspects, the medium comprises about 65 mM potassium ion, and the medium has an osmolality of about 250 mOsmol to about 260 mOsmol. In some aspects, the medium comprises about 65 mM potassium ion, and the medium has an osmolality of about 257 mOsmol to about 258 mOsmol. In some aspects, the medium comprises about 65 mM potassium ion, and the medium has an osmolality of about 257.5 mOsmol.

In some aspects, the medium comprises about 66 mM, about 67 mM, about 68 mM, or about 69 mM potassium ion, and the medium has an osmolality of about 257 mOsmol. In some aspects, the medium comprises about 66 mM, about 67 mM, about 68 mM, or about 69 mM potassium ion, and the medium has an osmolality of about 255 mOsmol to about 265 mOsmol. In some aspects, the medium comprises about 66 mM, about 67 mM, about 68 mM, or about 69 mM potassium ion, and the medium has an osmolality of about 257 mOsmol to about 260 mOsmol. In some aspects, the medium comprises about 66 mM, about 67 mM, about 68 mM, or about 69 mM potassium ion, and the medium has an osmolality of about 257.5 mOsmol. In some aspects, the medium comprises about 66 mM, about 67 mM, about 68 mM, or about 69 mM potassium ion, and the medium has an osmolality of about 259.7 mOsmol.

In some aspects, the medium comprises about 70 mM potassium ion, and the medium has an osmolality of about 259 mOsmol. In some aspects, the medium comprises about 70 mM potassium ion, and the medium has an osmolality of about 255 mOsmol to about 265 mOsmol. In some aspects, the medium comprises about 70 mM potassium ion, and the medium has an osmolality of about 259 mOsmol to about 261 mOsmol. In some aspects, the medium comprises about 70 mM potassium ion, and the medium has an osmolality of about 259.7 mOsmol.

In some aspects, the medium comprises about 71 mM, about 72 mM, about 73 mM, or about 74 mM potassium ion, and the medium has an osmolality of about 259 mOsmol. In some aspects, the medium comprises about 71 mM, about 72 mM, about 73 mM, or about 74 mM potassium ion, and the medium has an osmolality of about 255 mOsmol to about 265 mOsmol. In some aspects, the medium comprises about 71 mM, about 72 mM, about 73 mM, or about 74 mM potassium ion, and the medium has an osmolality of about 259 mOsmol to about 261 mOsmol. In some aspects, the medium comprises about 71 mM, about 72 mM, about 73 mM, or about 74 mM potassium ion, and the medium has an osmolality of about 259.7 mOsmol. In some aspects, the medium comprises about 71 mM, about 72 mM, about 73 mM, or about 74 mM potassium ion, and the medium has an osmolality of about 260 mOsmol.

In some aspects, the medium comprises about 75 mM potassium ion, and the medium has an osmolality of about 260 mOsmol. In some aspects, the medium comprises about 75 mM potassium ion, and the medium has an osmolality of about 255 mOsmol to about 265 mOsmol. In some aspects, the medium comprises about 75 mM potassium ion, and the medium has an osmolality of about 259 mOsmol to about 261 mOsmol. In some aspects, the medium comprises about 75 mM potassium ion, and the medium has an osmolality of about 260 mOsmol.

In some aspects, the medium comprises about 81 mM, about 82 mM, about 83 mM, or about 84 mM potassium ion, and the medium has an osmolality of about 260 mOsmol. In some aspects, the medium comprises about 81 mM, about 82 mM, about 83 mM, or about 84 mM potassium ion, and the medium has an osmolality of about 261 mOsmol. In some aspects, the medium comprises about 81 mM, about 82 mM, about 83 mM, or about 84 mM potassium ion, and the medium has an osmolality of about 262 mOsmol. In some aspects, the medium comprises about 81 mM, about 82 mM, about 83 mM, or about 84 mM potassium ion, and the medium has an osmolality of about 263 mOsmol. In some aspects, the medium comprises about 81 mM, about 82 mM, about 83 mM, or about 84 mM potassium ion, and the medium has an osmolality of about 255 mOsmol to about 265 mOsmol. In some aspects, the medium comprises about 81 mM, about 82 mM, about 83 mM, or about 84 mM potassium ion, and the medium has an osmolality of about 261 mOsmol to about 263 mOsmol. In some aspects, the medium comprises about 81 mM, about 82 mM, about 83 mM, or about 84 mM potassium ion, and the medium has an osmolality of about 262.26 mOsmol.

In some aspects, the medium comprises about 80 mM potassium ion, and the medium has an osmolality of about 262 mOsmol. In some aspects, the medium comprises about 80 mM potassium ion, and the medium has an osmolality of about 255 mOsmol to about 265 mOsmol. In some aspects, the medium comprises about 80 mM potassium ion, and the medium has an osmolality of about 261 mOsmol to about 263 mOsmol. In some aspects, the medium comprises about 80 mM potassium ion, and the medium has an osmolality of about 262.26 mOsmol.

In some aspects, the medium comprises about 81 mM, about 82 mM, about 83 mM, about 84 mM, or about 85 mM potassium ion, and the medium has an osmolality of about 262 mOsmol. In some aspects, the medium comprises about 81 mM, about 82 mM, about 83 mM, about 84 mM, or about 85 mM potassium ion, and the medium has an osmolality of about 263 mOsmol. In some aspects, the medium comprises about 81 mM, about 82 mM, about 83 mM, about 84 mM, or about 85 mM potassium ion, and the medium has an osmolality of about 264 mOsmol. In some aspects, the medium comprises about 81 mM, about 82 mM, about 83 mM, about 84 mM, or about 85 mM potassium ion, and the medium has an osmolality of about 265 mOsmol. In some aspects, the medium comprises about 81 mM, about 82 mM, about 83 mM, about 84 mM, or about 85 mM potassium ion, and the medium has an osmolality of about 255 mOsmol to about 265 mOsmol. In some aspects, the medium comprises about 81 mM, about 82 mM, about 83 mM, about 84 mM, or about 85 mM potassium ion, and the medium has an osmolality of about 260 mOsmol to about 270 mOsmol. In some aspects, the medium comprises about 81 mM, about 82 mM, about 83 mM, about 84 mM, or about 85 mM potassium ion, and the medium has an osmolality of about 261 mOsmol to about 263 mOsmol. In some aspects, the medium comprises about 81 mM, about 82 mM, about 83 mM, about 84 mM, or about 85 mM potassium ion, and the medium has an osmolality of about 263 mOsmol to about 265 mOsmol.

In some aspects, the medium further comprises a cell expansion agent. As used herein, a “cell expansion agent” refers to an agent, e.g., small molecule, polypeptide, or any combination thereof, that promotes the in vitro and/or ex vivo growth and proliferation of cultured cells, e.g., pluripotent, multipotent, and/or immune cells (e.g., T cells and/or NK cells). In some aspects, the cell expansion agent comprises a PI3K inhibitor. In some aspects, the medium further comprises an AKT inhibitor. In some aspects, the medium further comprises a PI3K inhibitor and an AKT inhibitor. In some aspects, the PI3K inhibitor comprises LY294002. In some aspects, the PI3K inhibitor comprises IC87114. In some aspects, the PI3K inhibitor comprises idelalisib (see, e.g., Peterson et al., Blood Adv. 2(3):210-23 (2018)). In some aspects, the medium further comprises a GSK3B inhibitor. In some aspects, the GSK3B inhibitor comprises TWS119. In some aspects, the medium further comprises an ACLY inhibitor. In some aspects, the ACLY inhibitor comprises potassium hydroxycitrate tribasic monohydrate. In some aspects, the PI3K inhibitor comprises hydroxyl citrate. In some aspects, the PI3K inhibitor comprises pictilisib. In some aspects, the PI3K inhibitor comprises CAL-101. In some aspects, the AKT inhibitor comprises MK2206, A443654, or AKTi-VIII (CAS 612847-09-3).

In some aspects, the cells, e.g., pluripotent, multipotent, and/or immune cells (e.g., T cells and/or NK cells), are cultured in the medium disclosed herein for the entirety of ex vivo culture, e.g., from the time the T cells are isolated form a subject, through growing, expansion, engineering, and until administration. In some aspects, the cells, e.g., pluripotent, multipotent, and/or immune cells (e.g., T cells and/or NK cells), are cultured in the medium disclosed herein for the duration of expansion. In some aspects, the cells, e.g., pluripotent, multipotent, and/or immune cells (e.g., T cells and/or NK cells), are cultured in the medium disclosed herein for the duration of expansion. In some aspects, the cells, e.g., pluripotent, multipotent, and/or immune cells (e.g., T cells and/or NK cells), are cultured in the medium disclosed herein until the total number of viable cells, e.g., pluripotent, multipotent, and/or immune cells (e.g., T cells and/or NK cells), is at least about 10⁴, 5×10⁴, 10⁵, 5×10⁵, 10⁶, or 5×10⁶ total cells.

II.B. Sodium

Certain aspects of the present disclosure are directed to methods of culturing cells, e.g., pluripotent, multipotent, or immune cells (e.g., T cells and/or NK cells), comprising placing the cells in a medium comprising (i) potassium ion at a concentration of at least about 5 mM and (ii) sodium ion (e.g., NaCl) at a concentration of less than about 115 mM, wherein the medium is hypotonic or isotonic. Some aspects of the present disclosure are directed to methods of culturing cells, e.g., pluripotent, multipotent, and/or immune cells (e.g., T cells and/or NK cells), comprising placing the cells in a medium comprising (i) potassium ion at a concentration higher than 40 mM and (ii) sodium ion (e.g., NaCl) at a concentration of less than about 115 mM. Certain aspects of the present disclosure are directed to methods of culturing T cells, comprising placing the T cells in a medium comprising (i) potassium ion at a concentration of at least about 50 mM and (ii) sodium ion (e.g., NaCl) at a concentration of less than about 115 mM. In some aspects, the medium is hypotonic. In some aspects, the medium is isotonic.

In some aspects, the target concentration of sodium is reached by starting with a basal medium comprising a higher concentration of sodium ion (e.g., NaCl), and diluting the solution to reach the target concentration of sodium ion (e.g., NaCl). In some aspects, the target concentration of sodium is reached by raising the concentration of sodium ion (e.g., NaCl) by adding one or more sodium salts (e.g., more NaCl). Non-limiting examples of sodium salts include sodium (meta)periodate, sodium arsenyl tartrate hydrate, sodium azide, sodium benzyloxide, sodium bromide, sodium carbonate, sodium chloride, sodium chromate, sodium cyclohexanebutyrate, sodium ethanethiolate, sodium fluoride, sodium fluorophosphate, sodium formate, sodium hexachloroiridate(III) hydrate, sodium hexachloroiridate(IV) hexahydrate, sodium hexachloroplatinate(IV) hexahydrate, sodium hexachlororhodate(III), sodium hexafluoroaluminate, sodium hexafluoroantimonate(V), sodium hexafluoroarsenate(V), sodium hexafluoroferrate(III), sodium hexafluorophosphate, sodium hexafluorosilicate, sodium hexahydroxyplatinate(IV), sodium hexametaphosphate, sodium hydrogen difluoride, sodium hydrogen sulfate, sodium hydrogencyanamide, sodium hydroxide, sodium iodide, sodium metaborate tetrahydrate, sodium metasilicate nonahydrate, sodium metavanadate, sodium molybdate, sodium nitrate, sodium nitrite, sodium oxalate, sodium perborate monohydrate, sodium percarbonate, sodium perchlorate, sodium periodate, sodium permanganate, sodium perrhenate, sodium phosphate, sodium pyrophosphate, sodium selenate, sodium selenite, sodium stannate, sodium sulfate, sodium tellurite, sodium tetraborate, sodium tetrachloroaluminate, sodium tetrachloroaurate(III), sodium tetrachloropalladate(II), sodium tetrachloroplatinate(II), sodium thiophosphate tribasic, sodium thiosulfate, sodium thiosulfate pentahydrate, sodium yttrium oxyfluoride, Trisodium trimetaphosphate, and any combination thereof. In certain aspects, the sodium salt comprises sodium chloride (NaCl). In certain aspects, the sodium salt comprises sodium gluconate. In certain aspects, the sodium salt comprises sodium bicarbonate. In certain aspects, the sodium salt comprises sodium hydroxycitrate. In certain aspects, the sodium salt comprises sodium phosphate.

In some aspects, the concentration of the sodium ion (e.g., NaCl) is less than that of the basal medium. In some aspects, the concentration of the sodium ion (e.g., NaCl) is reduced as the concentration of potassium ion is increased. In some aspects, the concentration of the sodium ion (e.g., NaCl) is from about 25 mM to about 115 mM. In some aspects, the concentration of the sodium ion (e.g., NaCl) is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In certain aspects, the concentration of the sodium ion (e.g., NaCl) is from about 40 mM to about 80 mM. In certain aspects, the concentration of the sodium ion (e.g., NaCl) is from about 50 mM to about 85 mM. In certain aspects, the concentration of the sodium ion (e.g., NaCl) is from about 55 mM to about 80 mM. In certain aspects, the concentration of the sodium ion (e.g., NaCl) is from about 30 mM to about 35 mM. In certain aspects, the concentration of the sodium ion (e.g., NaCl) is from about 35 mM to about 40 mM. In certain aspects, the concentration of the sodium ion (e.g., NaCl) is from about 40 mM to about 45 mM. In certain aspects, the concentration of the sodium ion (e.g., NaCl) is from about 45 mM to about 50 mM. In certain aspects, the concentration of the sodium ion (e.g., NaCl) is from about 50 mM to about 55 mM. In certain aspects, the concentration of the sodium ion (e.g., NaCl) is from about 55 mM to about 60 mM. In certain aspects, the concentration of the sodium ion (e.g., NaCl) is from about 60 mM to about 65 mM. In certain aspects, the concentration of the sodium ion (e.g., NaCl) is from about 65 mM to about 70 mM. In certain aspects, the concentration of the sodium ion (e.g., NaCl) is from about 70 mM to about 75 mM. In certain aspects, the concentration of the sodium ion (e.g., NaCl) is from about 75 mM to about 80 mM. In certain aspects, the concentration of the sodium ion (e.g., NaCl) is from about 80 mM to about 85 mM.

In some aspects, the concentration of the sodium ion (e.g., NaCl) is about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In certain aspects, the concentration of sodium ion (e.g., NaCl) is about 40 mM. In certain aspects, the concentration of sodium ion (e.g., NaCl) is about 45 mM. In certain aspects, the concentration of sodium ion (e.g., NaCl) is about 50 mM. In certain aspects, the concentration of sodium ion (e.g., NaCl) is about 55 mM. In certain aspects, the concentration of sodium ion (e.g., NaCl) is about 55.6 mM. In certain aspects, the concentration of sodium ion (e.g., NaCl) is about 59.3 mM. In certain aspects, the concentration of sodium ion (e.g., NaCl) is about 60 mM. In certain aspects, the concentration of sodium ion (e.g., NaCl) is about 63.9 mM. In certain aspects, the concentration of sodium ion (e.g., NaCl) is about 65 mM. In certain aspects, the concentration of sodium ion (e.g., NaCl) is about 67.6 mM. In certain aspects, the concentration of sodium ion (e.g., NaCl) is about 70 mM. In certain aspects, the concentration of sodium ion (e.g., NaCl) is about 72.2 mM. In certain aspects, the concentration of sodium ion (e.g., NaCl) is about 75 mM. In certain aspects, the concentration of sodium ion (e.g., NaCl) is about 76 mM. In certain aspects, the concentration of sodium ion (e.g., NaCl) is about 80 mM. In certain aspects, the concentration of sodium ion (e.g., NaCl) is about 80.5 mM. In some aspects, the medium comprises about 40 mM to about 90 mM potassium ion and about 40 mM to about 80 mM sodium ion (e.g., NaCl).

In some aspects, the medium comprises about 50 mM to about 75 mM potassium ion and about 80 mM to about 90 mM sodium ion (e.g., NaCl). In some aspects, the medium comprises about 55 mM to about 75 mM potassium ion and about 80 mM to about 90 mM sodium ion (e.g., NaCl). In some aspects, the medium comprises about 60 mM to about 75 mM potassium ion and about 80 mM to about 90 mM sodium ion (e.g., NaCl). In some aspects, the medium comprises about 65 mM to about 75 mM potassium ion and about 80 mM to about 85 mM sodium ion (e.g., NaCl). In some aspects, the medium comprises about 65 mM potassium ion and about 80 mM to about 85 mM sodium ion (e.g., NaCl). In some aspects, the medium comprises about 66 mM potassium ion and about 80 mM to about 85 mM sodium ion (e.g., NaCl). In some aspects, the medium comprises about 67 mM potassium ion and about 80 mM to about 85 mM sodium ion (e.g., NaCl). In some aspects, the medium comprises about 68 mM potassium ion and about 80 mM to about 85 mM sodium ion (e.g., NaCl). In some aspects, the medium comprises about 69 mM potassium ion and about 80 mM to about 85 mM sodium ion (e.g., NaCl). In some aspects, the medium comprises about 70 mM potassium ion and about 80 mM to about 85 mM sodium ion (e.g., NaCl). In some aspects, the medium comprises about 71 mM potassium ion and about 80 mM to about 85 mM sodium ion (e.g., NaCl). In some aspects, the medium comprises about 72 mM potassium ion and about 80 mM to about 85 mM sodium ion (e.g., NaCl). In some aspects, the medium comprises about 73 mM potassium ion and about 80 mM to about 85 mM sodium ion (e.g., NaCl). In some aspects, the medium comprises about 74 mM potassium ion and about 80 mM to about 85 mM sodium ion (e.g., NaCl). In some aspects, the medium comprises about 75 mM potassium ion and about 80 mM to about 85 mM sodium ion (e.g., NaCl). In some aspects, the medium comprises about 65 mM potassium ion and about 80 mM sodium ion (e.g., NaCl). In some aspects, the medium comprises about 65 mM potassium ion and about 85 mM sodium ion (e.g., NaCl). In some aspects, the medium comprises about 65 mM potassium ion and about 90 mM sodium ion (e.g., NaCl). In some aspects, the medium comprises about 70 mM potassium ion and about 80 mM sodium ion (e.g., NaCl). In some aspects, the medium comprises about 70 mM potassium ion and about 85 mM sodium ion (e.g., NaCl). In some aspects, the medium comprises about 70 mM potassium ion and about 90 mM sodium ion (e.g., NaCl). In some aspects, the medium comprises about 75 mM potassium ion and about 80 mM sodium ion (e.g., NaCl). In some aspects, the medium comprises about 75 mM potassium ion and about 85 mM sodium ion (e.g., NaCl). In some aspects, the medium comprises about 75 mM potassium ion and about 90 mM sodium ion (e.g., NaCl).

In some aspects, the medium comprises about 40 mM to about 90 mM potassium ion and about 30 mM to about 109 mM NaCl, wherein the concentration of NaCl (mM) is equal to or lower than (135—potassium ion concentration). In some aspects, the medium comprises about 40 mM potassium ion and less than or equal to about 95 mM NaCl (e.g., about 95 mM, about 94 mM, about 93 mM, about 92 mM, about 91 mM, about 90 mM, about 85 mM, about 80 mM, about 75 mM, about 70 mM, about 65 mM, about 60 mM, about 55 mM, or about 50 mM NaCl). In some aspects, the medium comprises about 45 mM potassium ion and less than or equal to about 90 mM NaCl (e.g., about 90 mM, about 89 mM, about 88 mM, about 87 mM, about 86 mM, about 85 mM, about 80 mM, about 75 mM, about 70 mM, about 65 mM, about 60 mM, about 55 mM, or about 50 mM NaCl). In some aspects, the medium comprises about 50 mM potassium ion and less than or equal to about 85 mM NaCl (e.g., about 85 mM, about 84 mM, about 83 mM, about 82 mM, about 81 mM, about 80 mM, about 75 mM, about 70 mM, about 65 mM, about 60 mM, about 55 mM, or about 50 mM NaCl). In some aspects, the medium comprises about 55 mM potassium ion and less than or equal to about 80 mM NaCl (e.g., about 80 mM, about 79 mM, about 78 mM, about 77 mM, about 76 mM, about 75 mM, about 70 mM, about 65 mM, about 60 mM, about 55 mM, or about 50 mM NaCl). In some aspects, the medium comprises about 60 mM potassium ion and less than or equal to about 75 mM NaCl (e.g., about 75 mM, about 74 mM, about 73 mM, about 72 mM, about 71 mM, about 70 mM, about 65 mM, about 60 mM, about 55 mM, or about 50 mM NaCl). In some aspects, the medium comprises about 65 mM potassium ion and less than or equal to about 70 mM NaCl (e.g., about 70 mM, about 69 mM, about 68 mM, about 67 mM, about 66 mM, about 65 mM, about 60 mM, about 55 mM, or about 50 mM NaCl). In some aspects, the medium comprises about 70 mM potassium ion and less than or equal to about 70 mM NaCl (e.g., about 65 mM, about 64 mM, about 63 mM, about 62 mM, about 61 mM, about 60 mM, about 55 mM, or about 50 mM NaCl). In some aspects, the medium comprises about 75 mM potassium ion and less than or equal to about 60 mM NaCl (e.g., about 60 mM, about 59 mM, about 58 mM, about 57 mM, about 56 mM, about 55 mM, about 50 mM, about 45 mM, or about 40 mM NaCl). In some aspects, the medium comprises about 80 mM potassium ion and less than or equal to about 55 mM NaCl (e.g., about 55 mM, about 54 mM, about 53 mM, about 52 mM, about 51 mM, about 50 mM, about 45 mM, about 40 mM, or about 35 mM NaCl). In some aspects, the medium comprises about 85 mM potassium ion and less than or equal to about 50 mM NaCl (e.g., about 50 mM, about 49 mM, about 48 mM, about 47 mM, about 46 mM, about 45 mM, about 40 mM, about 35 mM, or about 30 mM NaCl). In some aspects, the medium comprises about 90 mM potassium ion and less than or equal to about 45 mM NaCl (e.g., about 45 mM, about 44 mM, about 43 mM, about 42 mM, about 41 mM, about 40 mM, about 35 mM, about 30 mM, or about 25 mM NaCl). In some aspects, the medium comprises about 70 mM potassium ion and about 60 mM NaCl. In some aspects, the medium comprises about 70 mM potassium ion and about 61 mM NaCl. In some aspects, the medium comprises about 70 mM potassium ion and about 62 mM NaCl.

In some aspects, the medium comprises about 50 mM potassium ion and about 80.5 mM sodium ion (e.g., NaCl). In some aspects, the medium is hypotonic. In some aspects, the medium is isotonic. In some aspects, the medium comprises about 50 mM potassium ion and about 80.5 mM sodium ion, and the medium has an osmolality of about 254.7 mOsmol.

In some aspects, the medium comprises about 55 mM potassium ion and about 76 mM sodium ion (e.g., NaCl). In some aspects, the medium is hypotonic. In some aspects, the medium is isotonic. In some aspects, the medium comprises about 55 mM potassium ion and about 76 mM sodium ion, and the medium has an osmolality of about 255.2 mOsmol.

In some aspects, the medium comprises about 60 mM potassium ion and about 72.2 mM sodium ion (e.g., NaCl). In some aspects, the medium is hypotonic. In some aspects, the medium is isotonic. In some aspects, the medium comprises about 60 mM potassium ion and about 72.2 mM sodium ion, and the medium has an osmolality of about 257.2 mOsmol.

In some aspects, the medium comprises about 65 mM potassium ion and about 67.6 mM sodium ion (e.g., NaCl). In some aspects, the medium is hypotonic. In some aspects, the medium is isotonic. In some aspects, the medium comprises about 65 mM potassium ion and about 67.6 mM sodium ion, and the medium has an osmolality of about 257.5 mOsmol.

In some aspects, the medium comprises about 70 mM potassium ion and about 63.9 mM sodium ion (e.g., NaCl). In some aspects, the medium is hypotonic. In some aspects, the medium is isotonic. In some aspects, the medium comprises about 70 mM potassium ion and about 63.9 mM sodium ion, and the medium has an osmolality of about 259.7 mOsmol.

In some aspects, the medium comprises about 75 mM potassium ion and about 59.3 mM sodium ion (e.g., NaCl). In some aspects, the medium is hypotonic. In some aspects, the medium is isotonic. In some aspects, the medium comprises about 75 mM potassium ion and about 59.3 mM sodium ion, and the medium has an osmolality of about 260 mOsmol.

In some aspects, the medium comprises about 80 mM potassium ion and about 55.6 mM sodium ion (e.g., NaCl). In some aspects, the medium is hypotonic. In some aspects, the medium is isotonic. In some aspects, the medium comprises about 80 mM potassium ion and about 55.6 mM sodium ion, and the medium has an osmolality of about 262.26 mOsmol.

In some aspects, the medium comprises about 50 mM potassium ion and about 75 mM NaCl. In some aspects, the medium is hypotonic. In some aspects, the medium is isotonic.

Some aspects of the present disclosure are directed to methods of culturing cells, e.g., pluripotent, multipotent, and/or immune cells (e.g., T cells and/or NK cells), comprising placing the cells in a medium comprising (i) potassium ion at a concentration higher than 40 mM and (ii) NaCl at a concentration of less than about 100 mM. Certain aspects of the present disclosure are directed to methods of culturing T cells, comprising placing the T cells in a medium comprising (i) potassium ion at a concentration of at least about 50 mM and (ii) NaCl at a concentration of less than about 90 mM.

II.C. Saccharides

Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 5 mM and (ii) a saccharide, wherein the medium is hypotonic or isotonic. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration higher than 40 mM and (ii) a saccharide. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 50 mM and (ii) a saccharide. In some aspects, the medium is hypotonic. In some aspects, the medium is isotonic. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 50 mM and (ii) a saccharide; wherein the total concentration of potassium ion and NaCl is between 110 mM and 140 mM.

In some aspects, the target concentration of the saccharide is reached by starting with a basal medium comprising a higher concentration of the saccharide, and diluting the solution to reach the target concentration of the saccharide. In some aspects, the target concentration of the saccharide is reached by raising the concentration of the saccharide by adding the saccharide until the desired concentration is reached.

In some aspects, the saccharide is a monosaccharide, a disaccharide, or a polysaccharide. In some aspects, the saccharide is selected from glucose, fructose, galactose, mannose, maltose, sucrose, lactose, trehalose, or any combination thereof. In certain aspects, the saccharide is glucose. In some aspects, the medium comprises (i) potassium ion at a concentration of at least about 5 mM and (ii) glucose. In some aspects, the medium comprises (i) potassium ion at a concentration higher than 40 mM and (ii) glucose. In some aspects, the medium comprises (i) potassium ion at a concentration of at least about 5 mM and (ii) mannose. In some aspects, the medium comprises (i) potassium ion at a concentration of at least about 50 mM and (ii) mannose. In some aspects, the medium is hypotonic. In some aspects, the medium is isotonic. In some aspects, the medium comprises (i) potassium ion at a concentration higher than 40 mM and (ii) glucose; wherein the total concentration of potassium ion and NaCl is between 110 mM and 140 mM. In some aspects, the medium comprises (i) potassium ion at a concentration higher than 50 mM and (ii) glucose; wherein the total concentration of potassium ion and NaCl is between 110 mM and 140 mM. In some aspects, the medium comprises (i) potassium ion at a concentration of at least about 40 mM and (ii) mannose; wherein the total concentration of potassium ion and NaCl is between 110 mM and 140 mM. In some aspects, the medium comprises (i) potassium ion at a concentration of at least about 50 mM and (ii) mannose; wherein the total concentration of potassium ion and NaCl is between 110 mM and 140 mM.

In some aspects, the media, e.g., the MRM, comprises (i) potassium ion at a concentration higher than 5 mM and (ii) glucose. In some aspects, the media, e.g., the MRM, comprises (i) potassium ion at a concentration of at least about 30 mM to at least about 100 mM and (ii) glucose. In some aspects, the MRM comprises (i) potassium ion at a concentration higher than 40 mM and (ii) glucose. In some aspects, the media, e.g., the MRM, comprises (i) potassium ion at a concentration higher than 5 mM and (ii) mannose. In some aspects, the MRM comprises (i) potassium ion at a concentration of at least about 30 mM to at least about 100 mM and (ii) mannose. In some aspects, the MRM comprises (i) potassium ion at a concentration of higher than 40 mM and (ii) mannose. In some aspects, the MRM comprises (i) potassium ion at a concentration of at least about 50 mM and (ii) mannose. In some aspects, the MRM is hypotonic. In some aspects, the MRM is isotonic. In some aspects, the MRM comprises (i) potassium ion at a concentration higher than 40 mM and (ii) glucose; wherein the total concentration of potassium ion and NaCl is between 110 mM and 140 mM. In some aspects, the MRM comprises (i) potassium ion at a concentration higher than 50 mM and (ii) glucose; wherein the total concentration of potassium ion and NaCl is between 110 mM and 140 mM. In some aspects, the MRM comprises (i) potassium ion at a concentration of at least about 40 mM and (ii) mannose; wherein the total concentration of potassium ion and NaCl is between 110 mM and 140 mM. In some aspects, the MRM comprises (i) potassium ion at a concentration of at least about 50 mM and (ii) mannose; wherein the total concentration of potassium ion and NaCl is between 110 mM and 140 mM.

In some aspects, the concentration of the saccharide, e.g., glucose, is about 10 mM to about 24 mM. In some aspects, the concentration of the saccharide, e.g., glucose, is less than about 4.29 g/L. In some aspects, the concentration of the saccharide, e.g., glucose, is less than about 24 mM. In some aspects, the concentration of the saccharide, e.g., glucose, is more than about 5 mM. In some aspects, the concentration of the saccharide, e.g., glucose, is about 5 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 25 mM, about 10 mM to about 20 mM, about 10 mM to about 5 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 15 mM. In some aspects, the concentration of the saccharide, e.g., glucose, is from about 14 mM to about 14.5 mM. In some aspects, the concentration of the saccharide, e.g., glucose, is from about 14.5 mM to about 15 mM. In some aspects, the concentration of the saccharide, e.g., glucose, is from about 15 mM to about 15.5 mM. In some aspects, the concentration of the saccharide, e.g., glucose, is from about 15.5 mM to about 16 mM. In some aspects, the concentration of the saccharide, e.g., glucose, is from about 16 mM to about 16.5 mM. In some aspects, the concentration of the saccharide, e.g., glucose, is from about 16.5 mM to about 17 mM. In some aspects, the concentration of the saccharide, e.g., glucose, is from about 17 mM to about 17.5 mM. In some aspects, the concentration of the saccharide, e.g., glucose, is from about 17.5 mM to about 18 mM.

In some aspects, the concentration of the saccharide, e.g., glucose, is about 5 mM, about 6 mM, about 7 mM, about 8 mM, about 9 mM, about 10 mM, is about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 20.5 mM, about 21 mM, about 22 mM, about 23 mM, about 24 mM, or about 25 mM.

In some aspects, the concentration of the saccharide, e.g., glucose, is about 5 mM. In some aspects, the concentration of the saccharide, e.g., glucose, is about 6 mM. In some aspects, the concentration of the saccharide, e.g., glucose, is about 7 mM. In some aspects, the concentration of the saccharide, e.g., glucose, is about 8 mM. In some aspects, the concentration of the saccharide, e.g., glucose, is about 9 mM. In some aspects, the concentration of the saccharide, e.g., glucose, is about 10 mM. In some aspects, the concentration of the saccharide, e.g., glucose, is about 10.5 mM. In some aspects, the concentration of the saccharide, e.g., glucose, is about 11 mM. In some aspects, the concentration of the saccharide, e.g., glucose, is about 11.5 mM. In some aspects, the concentration of the saccharide, e.g., glucose, is about 12 mM. In some aspects, the concentration of the saccharide, e.g., glucose, is about 12.5 mM. In some aspects, the concentration of the saccharide, e.g., glucose, is about 13 mM. In some aspects, the concentration of the saccharide, e.g., glucose, is about 13.5 mM. In some aspects, the concentration of the saccharide, e.g., glucose, is about 14 mM. In some aspects, the concentration of the saccharide, e.g., glucose, is about 14.5 mM. In some aspects, the concentration of the saccharide, e.g., glucose, is about 15 mM. In some aspects, the concentration of the saccharide, e.g., glucose, is about 15.4 mM. In some aspects, the concentration of the saccharide, e.g., glucose, is about 15.9 mM. In some aspects, the concentration of the saccharide, e.g., glucose, is about 16.3 mM. In some aspects, the concentration of the saccharide, e.g., glucose, is about 16.8 mM. In some aspects, the concentration of the saccharide, e.g., glucose, is about 17.2 mM. In some aspects, the concentration of the saccharide, e.g., glucose, is about 17.7 mM.

In some aspects, the medium comprises about 50 mM potassium ion and about 17.7 mM glucose. In some aspects, the medium is hypotonic. In some aspects, the medium is isotonic. In some aspects, the medium comprises about 50 mM potassium ion and about 17.7 mM glucose, and the medium has an osmolality of about 254.7 mOsmol.

In some aspects, the medium comprises about 55 mM potassium ion and about 17.2 mM glucose. In some aspects, the medium is hypotonic. In some aspects, the medium is isotonic. In some aspects, the medium comprises about 55 mM potassium ion and about 17.2 mM glucose, and the medium has an osmolality of about 255.2 mOsmol.

In some aspects, the medium comprises about 60 mM potassium ion and about 16.8 mM glucose. In some aspects, the medium comprises about 60 mM potassium ion and about 16.8 mM glucose, and the medium is hypotonic. In some aspects, the medium is isotonic. In some aspects, the medium has an osmolality of about 257.2 mOsmol.

In some aspects, the medium comprises about 65 mM potassium ion and about 16.3 mM glucose. In some aspects, the medium is hypotonic. In some aspects, the medium is isotonic. In some aspects, the medium comprises about 65 mM potassium ion and about 16.3 mM glucose, and the medium has an osmolality of about 257.5 mOsmol.

In some aspects, the medium comprises about 70 mM potassium ion and about 15.9 mM glucose. In some aspects, the medium is hypotonic. In some aspects, the medium is isotonic. In some aspects, the medium comprises about 70 mM potassium ion and about 15.9 mM glucose, and the medium has an osmolality of about 259.7 mOsmol.

In some aspects, the medium comprises about 75 mM potassium ion and about 15.4 mM glucose. In some aspects, the medium is hypotonic. In some aspects, the medium is isotonic. In some aspects, the medium comprises about 75 mM potassium ion and about 15.4 mM glucose, and the medium has an osmolality of about 260 mOsmol.

In some aspects, the medium comprises about 80 mM potassium ion and about 15 mM glucose. In some aspects, the medium is hypotonic. In some aspects, the medium is isotonic. In some aspects, the medium comprises about 80 mM potassium ion and about 15 mM glucose, and the medium has an osmolality of about 262.26 mOsmol.

II.D. Calcium

Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 5 mM and (ii) sodium ion, wherein the medium is hypotonic or isotonic. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration higher than 40 mM and (ii) sodium ion. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 50 mM and (ii) sodium ion. In some aspects, the medium is hypotonic. In some aspects, the medium is isotonic. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 40 mM and (ii) NaCl; wherein the total concentration of potassium ion and NaCl is between 110 mM and 140 mM. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 50 mM and (ii) NaCl; wherein the total concentration of potassium ion and NaCl is between 110 mM and 140 mM.

In some aspects, the target concentration of calcium is reached by starting with a basal medium comprising a higher concentration of calcium ion, and diluting the solution to reach the target concentration of calcium ion. In some aspects, the target concentration of calcium is reached by raising the concentration of calcium ion by adding one or more calcium salts. Non-limiting examples of calcium salts include calcium bromide, calcium carbonate, calcium chloride, calcium cyanamide, calcium fluoride, calcium hydride, calcium hydroxide, calcium iodate, calcium iodide, calcium nitrate, calcium nitrite, calcium oxalate, calcium perchlorate tetrahydrate, calcium phosphate monobasic, calcium phosphate tribasic, calcium sulfate, calcium thiocyanate tetrahydrate, hydroxyapatite, or any combination thereof. In some aspects, the calcium salt comprises calcium chloride (CaCl₂)). In some aspects, the calcium salt comprises calcium gluconate.

In some aspects, the concentration of the calcium ion is less than that of the basal medium. In some aspects, the concentration of the calcium ion is greater than that of the basal medium. In some aspects, the concentration of calcium ion is more than about 0.4 mM. In some aspects, the concentration of calcium ion is less than about 2.8 mM. In some aspects, the concentration of calcium ion is less than about 2.5 mM. In some aspects, the concentration of calcium ion is less than about 2.0 mM. In some aspects, the concentration of calcium ion is less than about 1.9 mM. In some aspects, the concentration of calcium ion is less than about 1.8 mM. In some aspects, the concentration of calcium ion is less than about 1.7 mM. In some aspects, the concentration of calcium ion is less than about 1.6 mM. In some aspects, the concentration of calcium ion is less than about 1.5 mM. In some aspects, the concentration of calcium ion is less than about 1.4 mM. In some aspects, the concentration of calcium ion is less than about 1.3 mM. In some aspects, the concentration of calcium ion is less than about 1.2 mM. In some aspects, the concentration of calcium ion is less than about 1.1 mM. In some aspects, the concentration of calcium ion is less than about 1.0 mM.

In some aspects, the concentration of calcium ion is from about 0.4 mM to about 2.8 mM, about 0.4 mM to about 2.7 mM, about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 0.8 to about 0.9 mM, about 0.8 to about 1.0 mM, about 0.8 to about 1.1 mM, about 0.8 to about 1.2 mM, about 0.8 to about 1.3 mM, about 0.8 to about 1.4 mM, about 0.8 to about 1.5 mM, about 0.8 to about 1.6 mM, about 0.8 to about 1.7 mM, about 0.8 to about 1.8 mM, about 0.9 to about 1.0 mM, about 0.9 to about 1.1 mM, about 0.9 to about 1.2 mM, about 0.9 to about 1.3 mM, about 0.9 to about 1.4 mM, about 0.9 to about 1.5 mM, about 0.9 to about 1.6 mM, about 0.9 to about 1.7 mM, about 0.9 to about 1.8 mM, about 1.0 to about 1.1 mM, about 1.0 to about 1.2 mM, about 1.0 to about 1.3 mM, about 1.0 to about 1.4 mM, about 1.0 to about 1.5 mM, about 1.0 to about 1.6 mM, about 1.0 to about 1.7 mM, about 1.0 to about 1.8 mM, about 1.1 to about 1.2 mM, about 1.1 to about 1.3 mM, about 1.1 to about 1.4 mM, about 1.1 to about 1.5 mM, about 1.1 to about 1.6 mM, about 1.1 to about 1.7 mM, about 1.1 to about 1.8 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

In some aspects, the concentration of calcium ion is from about 0.8 mM to about 1.8 mM. In some aspects, the concentration of calcium ion is from about 0.9 mM to about 1.8 mM. In some aspects, the concentration of calcium ion is from about 1.0 mM to about 1.8 mM. In some aspects, the concentration of calcium ion is from about 1.1 mM to about 1.8 mM. In some aspects, the concentration of calcium ion is from about 1.2 mM to about 1.8 mM. In some aspects, the concentration of calcium ion is from about 0.8 mM to about 1.8 mM. In some aspects, the concentration of calcium ion is from about 0.8 mM to about 0.9 mM. In some aspects, the concentration of calcium ion is from about 0.9 mM to about 1.0 mM. In some aspects, the concentration of calcium ion is from about 1.0 mM to about 1.1 mM. In some aspects, the concentration of calcium ion is from about 1.1 mM to about 1.2 mM. In some aspects, the concentration of calcium ion is from about 1.2 mM to about 1.3 mM. In some aspects, the concentration of calcium ion is from about 1.3 mM to about 1.4 mM. In some aspects, the concentration of calcium ion is from about 1.4 mM to about 1.5 mM. In some aspects, the concentration of calcium ion is from about 1.5 mM to about 1.6 mM. In some aspects, the concentration of calcium ion is from about 1.7 mM to about 1.8 mM.

In some aspects, the concentration of calcium ion is about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some aspects, the concentration of calcium ion is about 0.6 mM. In some aspects, the concentration of calcium ion is about 0.7 mM. In some aspects, the concentration of calcium ion is about 0.8 mM. In some aspects, the concentration of calcium ion is about 0.9 mM. In some aspects, the concentration of calcium ion is about 1.0 mM. In some aspects, the concentration of calcium ion is about 1.1 mM. In some aspects, the concentration of calcium ion is about 1.2 mM. In some aspects, the concentration of calcium ion is about 1.3 mM. In some aspects, the concentration of calcium ion is about 1.4 mM. In some aspects, the concentration of calcium ion is about 1.5 mM. In some aspects, the concentration of calcium ion is about 1.6 mM. In some aspects, the concentration of calcium ion is about 1.7 mM. In some aspects, the concentration of calcium ion is about 1.8 mM.

In some aspects, the medium comprises about 50 mM potassium ion and about 1.8 mM calcium ion. In some aspects, the medium is hypotonic. In some aspects, the medium is isotonic. In some aspects, the medium comprises about 50 mM potassium ion and about 1.8 mM calcium ion, and the medium has an osmolality of about 254.7 mOsmol. In some aspects, the medium comprises about 50 mM potassium ion, about 1.8 mM calcium ion, and NaCl; wherein the total concentration of potassium ion and NaCl is between 110 mM and 140 mM.

In some aspects, the medium comprises about 55 mM potassium ion and about 1.7 mM calcium ion. In some aspects, the medium is hypotonic. In some aspects, the medium is isotonic. In some aspects, the medium comprises about 55 mM potassium ion and about 1.7 mM calcium ion, and the medium has an osmolality of about 255.2 mOsmol. In some aspects, the medium is isotonic. In some aspects, the medium comprises about 55 mM potassium ion, about 1.7 mM calcium ion, and NaCl; wherein the total concentration of potassium ion and NaCl is between 110 mM and 140 mM.

In some aspects, the medium comprises about 60 mM potassium ion and about 1.6 mM calcium ion. In some aspects, the medium is hypotonic. In some aspects, the medium is isotonic. In some aspects, the medium comprises about 60 mM potassium ion and about 1.6 mM calcium ion, and the medium has an osmolality of about 257.2 mOsmol. In some aspects, the medium comprises about 60 mM potassium ion, about 1.6 mM calcium ion, and NaCl; wherein the total concentration of potassium ion and NaCl is between 110 mM and 140 mM.

In some aspects, the medium comprises about 65 mM potassium ion and about 1.5 mM calcium ion. In some aspects, the medium is hypotonic. In some aspects, the medium is isotonic. In some aspects, the medium comprises about 65 mM potassium ion and about 1.5 mM calcium ion, and the medium has an osmolality of about 257.5 mOsmol. In some aspects, the medium comprises about 65 mM potassium ion, about 1.5 mM calcium ion, and NaCl; wherein the total concentration of potassium ion and NaCl is between 110 mM and 140 mM.

In some aspects, the medium comprises about 70 mM potassium ion and about 1.4 mM calcium ion. In some aspects, the medium is hypotonic. In some aspects, the medium is isotonic. In some aspects, the medium comprises about 70 mM potassium ion and about 1.4 mM calcium ion, and the medium has an osmolality of about 259.7 mOsmol. In some aspects, the medium comprises about 70 mM potassium ion, about 1.4 mM calcium ion, and NaCl; wherein the total concentration of potassium ion and NaCl is between 110 mM and 140 mM.

In some aspects, the medium comprises about 75 mM potassium ion and about 1.3 mM calcium ion. In some aspects, the medium is hypotonic. In some aspects, the medium is isotonic. In some aspects, the medium comprises about 75 mM potassium ion and about 1.3 mM calcium ion, and the medium has an osmolality of about 260 mOsmol. In some aspects, the medium comprises about 75 mM potassium ion, about 1.3 mM calcium ion, and NaCl; wherein the total concentration of potassium ion and NaCl is between 110 mM and 140 mM.

In some aspects, the medium comprises about 80 mM potassium ion and about 1.2 mM calcium ion. In some aspects, the medium is hypotonic. In some aspects, the medium is isotonic. In some aspects, the medium comprises about 80 mM potassium ion and about 1.2 mM calcium ion, and the medium has an osmolality of about 262.26 mOsmol. In some aspects, the medium comprises about 80 mM potassium ion, about 1.2 mM calcium ion, and NaCl; wherein the total concentration of potassium ion and NaCl is between 110 mM and 140 mM.

Some aspects of the present disclosure are directed to methods of culturing immune cells, e.g., T cells and/or NK cells, in a medium comprising (i) potassium ion at a concentration of at least about 5 mM and (ii) calcium ion. In some aspects, the medium is hypotonic or isotonic.

II.E. Tonicity

Certain aspects of the present disclosure are directed to methods of culturing a cell, e.g., a pluripotent cell, a multipotent cell, or an immune cell (e.g., a T cell and/or an NK cell), in a medium comprising a higher concentration of potassium ion than conventional medium (e.g., greater than about 5 mM potassium ion), wherein the medium is not hypertonic. It was surprisingly found that culturing pluripotent/multipotent/immune cells in a non-hypertonic medium having a high potassium ion concentration (e.g., greater than 5 mM potassium ion) results in an increase in the number of stem-like (e.g., less differentiated or undifferentiated) cells in the culture. Cells cultured under such conditions also retain effector function and the ability to proliferate. This effect is most pronounced if (i) the medium has a high concentration of potassium (e.g., greater than 5 mM, e.g., greater than 50 mM) and the medium is hypotonic or isotonic. Thus, though increasing potassium ion in a culture medium might otherwise increase tonicity, the data disclosed herein suggest that an increase in potassium ion alone is not as effective at promoting stem-like properties in a population of cells as increasing potassium ion under hypotonic or isotonic conditions.

In some aspects of the present disclosure, the tonicity of the medium is adjusted. In some aspects of the present disclosure, the tonicity of the metabolic reprogramming medium (e.g., (concentration of potassium ion and concentration of NaCl)×2) is adjusted based on the concentration of potassium ion and/or NaCl. In some aspects, the tonicity of the medium is lower than that of the basal medium. In some aspects, the tonicity of the medium is higher than that of the basal medium. In some aspect, the tonicity of the medium is the same as that of the basal medium.

Medium used in the present disclosure can be hypotonic or isotonic. The tonicity of the medium can be affected by a number of factors, including the concentration of potassium ion in the media. In some aspects, increased potassium ion concentration is paired with an increase or a decrease in the concentration of one or more other factors. In some aspects, this pairing affects the tonicity of the medium. In some aspects, the concentration of potassium ion is increased while the concentration of sodium ion, e.g., NaCl, is decreased.

In some aspects, the media useful for the present media can be prepared based on the function of potassium ion and tonicity. See Table 2. For example, in some aspects, if the media useful for the present disclosure is hypotonic (e.g., less than 280 mOsm) and comprises at least about 50 mM of potassium ion, a concentration of NaCl that is sufficient to maintain the media as hypotonic can be determined based on the following formula: NaCl concentration=(desired tonicity (280)/2)−potassium ion concentration. (i.e., the concentration of NaCl (mM) is equal to or lower than (140−potassium ion concentration)). In some aspects, the hypotonic media disclosed herein comprises a total concentration of potassium ion and NaCl between 110 mM and 140 mM. Therefore, for hypotonic media, the concentration of potassium ion can be set at a concentration between 50 mM and 90 mM, and the sodium ion concentration can be between 90 mM and 50 mM, or lower, so long as the total concentration of potassium ion and NaCl is between 110 mM and 140 mM. In some aspects, the hypotonic media disclosed herein comprises a total concentration of potassium ion and NaCl between 115 mM and 140 mM. In some aspects, the hypotonic media disclosed herein comprises a total concentration of potassium ion and NaCl between 120 mM and 140 mM.

In some aspects, the media is isotonic (between 280 mOsm and 300 mOsm) and comprises a concentration of potassium ion between about 50 mM and 70 mM. The corresponding concentration of sodium ion can be again calculated based on the formula: NaCl concentration=(desired tonicity/2)−potassium ion concentration. For example, if the concentration of potassium is 50 mM and the desired tonicity is 300 mOsm, the sodium concentration can be 100 mM.

In some aspects, the medium is isotonic. In some aspects, the medium has a tonicity of about 280 mOsm/L. In some aspects, the medium has a tonicity of 280 mOsm/L. In some aspects, the medium has a tonicity of 280 mOsm/L±1 mOsm/L. In some aspects, the medium has a tonicity of 280 mOsm/L 2 mOsm/L. In some aspects, the medium has a tonicity of 280 mOsm/L±3 mOsm/L. In some aspects, the medium has a tonicity of 280 mOsm/L±4 mOsm/L. In some aspects, the medium has a tonicity of 280 mOsm/L±5 mOsm/L. In some aspects, the medium has a tonicity of 280 mOsm/L±6 mOsm/L. In some aspects, the medium has a tonicity of 280 mOsm/L±7 mOsm/L. In some aspects, the medium has a tonicity of 280 mOsm/L±8 mOsm/L. In some aspects, the medium has a tonicity of 280 mOsm/L±9 mOsm/L. In some aspects, the medium has a tonicity of 280 mOsm/L±10 mOsm/L. In some aspects, the medium has a tonicity of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L. In some aspects, the medium has a tonicity of about 285 mOsm/L, about 290 mOsm/L, about 295 mOsm/L, about 300 mOsm/L, about 305 mOsm/L, about 310 mOsm/L, or about 315 mOsm/L.

In some aspects, the medium is hypotonic. In some aspects, the medium has a tonicity lower than about 280 mOsm/L. In some aspects, the medium has a tonicity lower than about 280 mOsm/L; as measured by adding the potassium ion concentration and the NaCl concentration, and multiplying by two (see Table 2). In some aspects, the medium has a tonicity lower than 280 mOsm/L. In some aspects, the medium has a tonicity lower than 280 mOsm/L; as measured by adding the potassium ion concentration and the NaCl concentration, and multiplying by two. In some aspects, the medium has a tonicity lower than 275 mOsm/L. In some aspects, the medium has a tonicity lower than 275 mOsm/L; as measured by adding the potassium ion concentration and the NaCl concentration, and multiplying by two; as measured by adding the potassium ion concentration and the NaCl concentration, and multiplying by two. In some aspects, the medium has a tonicity lower than 270 mOsm/L. In some aspects, the medium has a tonicity lower than 270 mOsm/L; as measured by adding the potassium ion concentration and the NaCl concentration, and multiplying by two. In some aspects, the medium has a tonicity lower than 265 mOsm/L. In some aspects, the medium has a tonicity lower than 265 mOsm/L; as measured by adding the potassium ion concentration and the NaCl concentration, and multiplying by two. In some aspects, the medium has a tonicity lower than 260 mOsm/L. In some aspects, the medium has a tonicity lower than 260 mOsm/L; as measured by adding the potassium ion concentration and the NaCl concentration, and multiplying by two. In some aspects, the medium has a tonicity lower than 265 mOsm/L. In some aspects, the medium has a tonicity lower than 265 mOsm/L; as measured by adding the potassium ion concentration and the NaCl concentration, and multiplying by two. In some aspects, the medium has a tonicity lower than 260 mOsm/L. In some aspects, the medium has a tonicity lower than 260 mOsm/L; as measured by adding the potassium ion concentration and the NaCl concentration, and multiplying by two. In some aspects, the medium has a tonicity lower than 255 mOsm/L. In some aspects, the medium has a tonicity lower than 255 mOsm/L; as measured by adding the potassium ion concentration and the NaCl concentration, and multiplying by two. In some aspects, the medium has a tonicity lower than about 250 mOsm/L. In some aspects, the medium has a tonicity lower than about 250 mOsm/L; as measured by adding the potassium ion concentration and the NaCl concentration, and multiplying by two. In some aspects, the medium has a tonicity lower than about 245 mOsm/L. In some aspects, the medium has a tonicity lower than about 245 mOsm/L; as measured by adding the potassium ion concentration and the NaCl concentration, and multiplying by two. In some aspects, the medium has a tonicity lower than about 240 mOsm/L. In some aspects, the medium has a tonicity lower than about 240 mOsm/L; as measured by adding the potassium ion concentration and the NaCl concentration, and multiplying by two. In some aspects, the medium has a tonicity lower than about 235 mOsm/L. In some aspects, the medium has a tonicity lower than about 235 mOsm/L; as measured by adding the potassium ion concentration and the NaCl concentration, and multiplying by two. In some aspects, the medium has a tonicity lower than about 230 mOsm/L. In some aspects, the medium has a tonicity lower than about 230 mOsm/L; as measured by adding the potassium ion concentration and the NaCl concentration, and multiplying by two. In some aspects, the medium has a tonicity lower than about 225 mOsm/L. In some aspects, the medium has a tonicity lower than about 225 mOsm/L. In some aspects, the tonicity is higher than about 220 mOsm/L; as measured by adding the potassium ion concentration and the NaCl concentration, and multiplying by two. In some aspects, the medium has a tonicity from about 230 mOsm/L to about 280 mOsm/L. In some aspects, the medium has a tonicity from about 240 mOsm/L to about 280 mOsm/L.

In some aspects, the medium has an osmolality lower than about 220 mOsm/L. In some aspects, the medium has an osmolality lower than about 215 mOsm/L. In some aspects, the medium has an osmolality lower than about 210 mOsm/L. In some aspects, the medium has an osmolality lower than about 205 mOsm/L. In some aspects, the medium has an osmolality lower than about 200 mOsm/L.

In some aspects, the medium has a tonicity from about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L. In some aspects, the medium has a tonicity from about 250 mOsm/L to about 270 mOsm/L. In some aspects, the medium has a tonicity from about 250 mOsm/L to about 255 mOsm/L, about 250 mOsm/L to about 260 mOsm/L, about 250 mOsm/L to about 265 mOsm/L, about 255 mOsm/L to about 260 mOsm/L, about 255 mOsm/L to about 265 mOsm/L, about 255 mOsm/L to about 265 mOsm/L, about 260 mOsm/L to about 265 mOsm/L, or about 254 mOsm/L to about 263 mOsm/L. In some aspects, the medium has a tonicity from about 254 mOsm/L to about 255 mOsm/L. In some aspects, the medium has a tonicity from about 255 mOsm/L to about 256 mOsm/L. In some aspects, the medium has a tonicity from about 256 mOsm/L to about 257 mOsm/L. In some aspects, the medium has a tonicity from about 257 mOsm/L to about 258 mOsm/L. In some aspects, the medium has a tonicity from about 258 mOsm/L to about 259 mOsm/L. In some aspects, the medium has a tonicity from about 260 mOsm/L to about 261 mOsm/L. In some aspects, the medium has a tonicity from about 261 mOsm/L to about 262 mOsm/L. In some aspects, the medium has a tonicity from about 262 mOsm/L to about 263 mOsm/L. In some aspects, the medium has a tonicity from about 263 mOsm/L to about 264 mOsm/L. In some aspects, the medium has a tonicity from about 264 mOsm/L to about 265 mOsm/L. In some aspects, the medium has a tonicity from about 220 mOsm/L to about 280 mOsm/L; as measured by adding the potassium ion concentration and the NaCl concentration, and multiplying by two.

In some aspects, the medium has a tonicity of about 100 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L. In some aspects, the osmolality is measured by adding the potassium ion concentration and the NaCl concentration, and multiplying by two.

In some aspects, the medium has a tonicity of about 250 mOsm/L. In some aspects, the medium has a tonicity of about 262.26 mOsm/L. In some aspects, the medium has a tonicity of about 260 mOsm/L. In some aspects, the medium has a tonicity of about 259.7 mOsm/L. In some aspects, the medium has a tonicity of about 257.5 mOsm/L. In some aspects, the medium has a tonicity of about 257.2 mOsm/L. In some aspects, the medium has a tonicity of about 255.2 mOsm/L. In some aspects, the medium has a tonicity of about 254.7. In some aspects, the medium has a tonicity of about 255 mOsm/L. In some aspects, the medium has a tonicity of about 260 mOsm/L. In some aspects, the osmolality is measured by adding the potassium ion concentration and the NaCl concentration, and multiplying by two.

In some aspects, the medium comprises about 50 mM potassium ion and (i) about 80.5 mM sodium ion; (ii) about 17.7 mM glucose; (iii) about 1.8 mM calcium ion; or (iv) any combination of (i)-(iii). In some aspects, the medium is hypotonic. In some aspects, the medium is isotonic. In some aspects, the medium comprises about 50 mM potassium ion and (i) about 80.5 mM sodium ion; (ii) about 17.7 mM glucose; (iii) about 1.8 mM calcium ion; or (iv) any combination of (i)-(iii); and the medium has an osmolality of about 254.7 mOsmol. In some aspects, the medium comprises about 50 mM potassium ion and (i) about 80.5 mM NaCl; (ii) about 17.7 mM glucose; (iii) about 1.8 mM calcium ion; or (iv) any combination of (i)-(iii). In some aspects, the medium comprises about 50 mM potassium ion and (i) about 80.5 mM NaCl; (ii) about 17.7 mM glucose; and (iii) about 1.8 mM calcium ion.

In some aspects, the medium comprises about 55 mM potassium ion and (i) about 76 mM sodium ion; (ii) about 17.2 mM glucose; (iii) about 1.7 mM calcium ion; or (iv) any combination of (i)-(iii). In some aspects, the medium is hypotonic. In some aspects, the medium is isotonic. In some aspects, the medium comprises about 55 mM potassium ion and (i) about 76 mM sodium ion; (ii) about 17.2 mM glucose; (iii) about 1.7 mM calcium ion; or (iv) any combination of (i)-(iii); and the medium has an osmolality of about 255.2 mOsmol. In some aspects, the medium comprises about 55 mM potassium ion and (i) about 76 mM NaCl; (ii) about 17.2 mM glucose; (iii) about 1.7 mM calcium ion; or (iv) any combination of (i)-(iii). In some aspects, the medium comprises about 55 mM potassium ion and (i) about 76 mM NaCl; (ii) about 17.2 mM glucose; and (iii) about 1.7 mM calcium ion.

In some aspects, the medium comprises about 60 mM potassium ion and (i) about 72.2 mM sodium ion; (ii) about 16.8 mM glucose; (iii) about 1.6 mM calcium ion; or (iv) any combination of (i)-(iii). In some aspects, the medium is hypotonic. In some aspects, the medium is isotonic. In some aspects, the medium comprises about 60 mM potassium ion and (i) about 72.2 mM sodium ion; (ii) about 16.8 mM glucose; (iii) about 1.6 mM calcium ion; or (iv) any combination of (i)-(iii); and the medium has an osmolality of about 257.2 mOsmol. In some aspects, the medium comprises about 60 mM potassium ion and (i) about 72.2 mM NaCl; (ii) about 16.8 mM glucose; (iii) about 1.6 mM calcium ion; or (iv) any combination of (i)-(iii). In some aspects, the medium comprises about 60 mM potassium ion and (i) about 72.2 mM NaCl; (ii) about 16.8 mM glucose; and (iii) about 1.6 mM calcium ion.

In some aspects, the medium comprises about 65 mM potassium ion and (i) about 67.6 mM sodium ion; (ii) about 16.3 mM glucose; (iii) about 1.5 mM calcium ion; or (iv) any combination of (i)-(iii). In some aspects, the medium is hypotonic. In some aspects, the medium is isotonic. In some aspects, the medium comprises about 65 mM potassium ion and (i) about 67.6 mM sodium ion; (ii) about 16.3 mM glucose; (iii) about 1.5 mM calcium ion; or (iv) any combination of (i)-(iii); and the medium has an osmolality of about 257.5 mOsmol. In some aspects, the medium comprises about 65 mM potassium ion and (i) about 67.6 mM NaCl; (ii) about 16.3 mM glucose; (iii) about 1.5 mM calcium ion; or (iv) any combination of (i)-(iii). In some aspects, the medium comprises about 65 mM potassium ion and (i) about 67.6 mM NaCl; (ii) about 16.3 mM glucose; and (iii) about 1.5 mM calcium ion.

In some aspects, the medium comprises about 70 mM potassium ion and (i) about 63.9 mM sodium ion; (ii) about 15.9 mM glucose; (iii) about 1.4 mM calcium ion; or (iv) any combination of (i)-(iii). In some aspects, the medium is hypotonic. In some aspects, the medium is isotonic. In some aspects, the medium comprises about 70 mM potassium ion and (i) about 63.9 mM sodium ion; (ii) about 15.9 mM glucose; (iii) about 1.4 mM calcium ion; or (iv) any combination of (i)-(iii); and the medium has an osmolality of about 259.7 mOsmol. In some aspects, the medium comprises about 70 mM potassium ion and (i) about 63.9 mM NaCl; (ii) about 15.9 mM glucose; (iii) about 1.4 mM calcium ion; or (iv) any combination of (i)-(iii). In some aspects, the medium comprises about 70 mM potassium ion and (i) about 63.9 mM NaCl; (ii) about 15.9 mM glucose; and (iii) about 1.4 mM calcium ion.

In some aspects, the medium comprises about 75 mM potassium ion and (i) about 59.3 mM sodium ion; (ii) about 15.4 mM glucose; (iii) about 1.3 mM calcium ion; or (iv) any combination of (i)-(iii). In some aspects, the medium is hypotonic. In some aspects, the medium is isotonic. In some aspects, the medium comprises about 75 mM potassium ion and (i) about 59.3 mM sodium ion; (ii) about 15.4 mM glucose; (iii) about 1.3 mM calcium ion; or (iv) any combination of (i)-(iii); and the medium has an osmolality of about 260 mOsmol. In some aspects, the medium comprises about 75 mM potassium ion and (i) about 59.3 mM NaCl; (ii) about 15.4 mM glucose; (iii) about 1.3 mM calcium ion; or (iv) any combination of (i)-(iii). In some aspects, the medium comprises about 75 mM potassium ion and (i) about 59.3 mM NaCl; (ii) about 15.4 mM glucose; and (iii) about 1.3 mM calcium ion.

In some aspects, the medium comprises about 80 mM potassium ion and (i) about 55.6 mM sodium ion; (ii) about 15 mM glucose; (iii) about 1.2 mM calcium ion; or (iv) any combination of (i)-(iii). In some aspects, the medium is hypotonic. In some aspects, the medium is isotonic. In some aspects, the medium comprises about 80 mM potassium ion and (i) about 55.6 mM sodium ion; (ii) about 15 mM glucose; (iii) about 1.2 mM calcium ion; or (iv) any combination of (i)-(iii); and the medium has an osmolality of about 262.26 mOsmol. In some aspects, the medium comprises about 80 mM potassium ion and (i) about 55.6 mM NaCl; (ii) about 15 mM glucose; (iii) about 1.2 mM calcium ion; or (iv) any combination of (i)-(iii). In some aspects, the medium comprises about 80 mM potassium ion and (i) about 55.6 mM NaCl; (ii) about 15 mM glucose; and (iii) about 1.2 mM calcium ion.

The tonicity of the medium can be adjusted, e.g., to an isotonic or hypotonic state disclosed herein, at any point. In some aspects, the tonicity of the medium can be adjusted, e.g., to an isotonic or hypotonic state disclosed herein, before the cells are added to the medium. In some aspects, the cells are cultured in the hypotonic or isotonic medium prior to cell engineering, e.g., prior to transduction with a construct expressing a CAR, a TCR, or a TCR mimic. In some aspects, the cells are cultured in the hypotonic or isotonic medium during cell engineering, e.g., during transduction with a construct expressing a CAR, a TCR, or a TCR mimic. In some aspects the cells are cultured in the hypotonic or isotonic medium after cell engineering, e.g., after transduction with a construct expressing a CAR, a TCR, or a TCR mimic. In some aspects, the cells are cultured in the hypotonic or isotonic medium throughout cell expansion.

II.F. Cytokines

Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 5 mM, as disclosed herein, and (ii) a cytokine, wherein the medium is hypotonic or isotonic. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration higher than 40 mM, as disclosed herein, and (ii) a cytokine. Certain aspects of the present disclosure are directed to methods of culturing cells, e.g., pluripotent, multipotent, and/or immune cells (e.g., T cells and/or NK cells), comprising placing the cells in a medium comprising (i) potassium ion at a concentration of at least about 50 mM, as disclosed herein, and (ii) a cytokine. In some aspects, the medium is hypotonic. In some aspects, the medium is isotonic.

Some aspects of the present disclosure are directed to methods of increasing a number of less-differentiated immune cells, e.g., stem-like T cells, NK cells, or TILs, e.g., cells having one or more T_{SCM}-like phenotype and/or function, in immune cells ex vivo or in vitro, comprising placing the cells in a medium comprising (i) potassium at a concentration of at least about 5 mM, as disclosed herein, and (ii) a cytokine, wherein the medium is hypotonic or isotonic. Some aspects of the present disclosure are directed to methods of increasing a number of less-differentiated immune cells, e.g., stem-like T cells, NK cells, or TILs, e.g., cells having one or more T_{SCM}-like phenotype and/or function, in immune cells ex vivo or in vitro, comprising placing the cells in a medium comprising (i) potassium at a concentration of at least about 50 mM, as disclosed herein, and (ii) a cytokine. In some aspects, the medium is hypotonic. In some aspects, the medium is isotonic. In some aspects, the cytokine is selected from IL-2, IL-7, IL-15, IL-21, and any combination thereof.

In some aspects, a media, e.g., an MRM, disclosed herein comprises a cytokine. In some aspects, the MRM is hypotonic. In some aspects, the MRM is isotonic. In some aspects, the MRM is hypertonic. In some aspects, the cytokine is selected from IL-2, IL-7, IL-15, IL-21, and any combination thereof. In some aspects, the MRM does not comprise IL-2. In some aspects, the MRM comprises IL2 and IL21. In some aspects, the MRM comprises IL2, IL21, and IL15. In some aspects, the MRM comprises IL-2 during an initial culture of TIL cells. In some aspects, the MRM comprises IL-2 and IL-21 during an initial culture of TIL cells. In some aspects, the MRM comprises IL-2, IL-15, and IL-21 during a secondary expansion, e.g., a rapid expansion, of TIL cells. In some aspects, the MRM comprises IL-2, IL-15, and IL-21 during a final expansion of TIL cells. In some aspects, the concentrations of IL-2 and/or IL-21 in the secondary expansion and/or the final expansion are lower than the concentrations of IL-2 and/or IL-21 in the initial culture.

The cytokine can be added to the medium at any point. In some aspects, the cytokine is added to the medium before the cells are added to the medium. In some aspects, the cells are cultured in the medium comprising (i) potassium at a concentration disclosed herein, and (ii) a cytokine prior to cell engineering, e.g., prior to transduction with a construct expressing a CAR or a TCR. In some aspects, the cells are cultured in the medium comprising (i) potassium at a concentration disclosed herein, and (ii) a cytokine during cell engineering, e.g., during transduction with a construct expressing a CAR or a TCR. In some aspects, the cells are cultured in the medium comprising (i) potassium at a concentration disclosed herein, and (ii) a cytokine after cell engineering, e.g., after transduction with a construct expressing a CAR or a TCR. In some aspects, the cells are cultured in the medium comprising (i) potassium at a concentration disclosed herein, and (ii) a cytokine throughout cell expansion.

The cytokine can be added to the MRM at any point. In some aspects, the cytokine is added to the MRM before the TILs (e.g., the tumor sample), are added to the medium. In some aspects, the TILs (e.g., the tumor sample), are cultured in the MRM comprising (i) potassium at a concentration disclosed herein, and (ii) a cytokine throughout TIL culture including expansion. In some aspects, the TILs (e.g., the tumor sample), are cultured in the MRM comprising (i) potassium at a concentration disclosed herein, and (ii) a cytokine throughout TIL expansion.

In some aspects, the medium comprises (i) at least about 5 mM potassium ion and (ii) IL-2. In some aspects, the medium comprises (i) more than 40 mM potassium ion and (ii) IL-2. In some aspects, the medium comprises (i) at least about 50 mM potassium ion and (ii) IL-2. In some aspects, the medium comprises (i) at least about 5 mM potassium ion and (ii) IL-7. In some aspects, the medium comprises (i) more than 40 mM potassium ion and (ii) IL-7. In some aspects, the medium comprises (i) at least about 50 mM potassium ion and (ii) IL-7. In some aspects, the medium comprises (i) at least about 5 mM potassium ion and (ii) IL-15. In some aspects, the medium comprises (i) more than 40 mM potassium ion and (ii) IL-15. In some aspects, the medium comprises (i) at least about 50 mM potassium ion and (ii) IL-15. In some aspects, the medium comprises (i) at least about 5 mM potassium ion and (ii) IL-21. In some aspects, the medium comprises (i) more than 40 mM potassium ion and (ii) IL-21. In some aspects, the medium comprises (i) at least about 50 mM potassium ion and (ii) IL-21. In some aspects, the medium comprises (i) at least about 5 mM potassium ion and (ii) IL-2, and the medium does not comprise IL-7. In some aspects, the medium comprises (i) more than 40 mM potassium ion and (ii) IL-2, and the medium does not comprise IL-7. In some aspects, the medium comprises (i) at least about 50 mM potassium ion and (ii) IL-2, and the medium does not comprise IL-7. In some aspects, the medium comprises (i) at least about 5 mM potassium ion and (ii) IL-2, and the medium does not comprise IL-15. In some aspects, the medium comprises (i) more than 40 mM potassium ion and (ii) IL-2, and the medium does not comprise IL-15. In some aspects, the medium comprises (i) at least about 50 mM potassium ion and (ii) IL-2, and the medium does not comprise IL-15. In some aspects, the medium comprises (i) at least about 5 mM potassium ion and (ii) IL-2, and the medium does not comprise IL-7 and IL-15. In some aspects, the medium comprises (i) more than 40 mM potassium ion and (ii) IL-2, and the medium does not comprise IL-7 and IL-15. In some aspects, the medium comprises (i) at least about 50 mM potassium ion and (ii) IL-2, and the medium does not comprise IL-7 and IL-15. In some aspects, the medium comprises (i) at least about 5 mM potassium ion and (ii) IL-2 and IL-21. In some aspects, the medium comprises (i) more than 40 mM potassium ion and (ii) IL-2 and IL-21. In some aspects, the medium comprises (i) at least about 50 mM potassium ion and (ii) IL-2 and IL-21. In some aspects, the medium comprises (i) at least about 5 mM potassium ion and (ii) IL-7 and IL-21. In some aspects, the medium comprises (i) more than 40 mM potassium ion and (ii) IL-7 and IL-21. In some aspects, the medium comprises (i) at least about 50 mM potassium ion and (ii) IL-7 and IL-21. In some aspects, the medium comprises (i) at least about 5 mM potassium ion and (ii) IL-15 and IL-21. In some aspects, the medium comprises (i) more than 40 mM potassium ion and (ii) IL-15 and IL-21. In some aspects, the medium comprises (i) at least about 50 mM potassium ion and (ii) IL-15 and IL-21. In some aspects, the medium is hypotonic. In some aspects, the medium is isotonic. In some aspects, the medium further comprises NaCl, wherein the total concentration of potassium ion and NaCl is from 110 mM to 140 mM.

In some aspects, the MRM comprises (i) at least about 30 mM to at least about 100 mM potassium ion and (ii) IL-2. In some aspects, the MRM comprises (i) more than 40 mM potassium ion and (ii) IL-2. In some aspects, the MRM comprises (i) at least about 50 mM potassium ion and (ii) IL-2. In some aspects, the MRM comprises (i) at least about 30 mM to at least about 100 mM potassium ion and (ii) IL-7. In some aspects, the MRM comprises (i) more than 40 mM potassium ion and (ii) IL-7. In some aspects, the MRM comprises (i) at least about 50 mM potassium ion and (ii) IL-7. In some aspects, the MRM comprises (i) at least about 30 mM to at least about 100 mM potassium ion and (ii) IL-15. In some aspects, the MRM comprises (i) more than 40 mM potassium ion and (ii) IL-15. In some aspects, the MRM comprises (i) at least about 50 mM potassium ion and (ii) IL-15. In some aspects, the MRM comprises (i) at least about 30 mM to at least about 100 mM potassium ion and (ii) IL-21. In some aspects, the MRM comprises (i) more than 40 mM potassium ion and (ii) IL-21. In some aspects, the MRM comprises (i) at least about 50 mM potassium ion and (ii) IL-21. In some aspects, the MRM does not comprise IL-7 and/or IL-15.

Certain aspects of the present disclosure are directed to a method of increasing a number of less-differentiated immune cells, e.g., stem-like T cells, NK cells, or TILs, e.g., cells having a T_{SCM}-like phenotype, in immune cells ex vivo or in vitro comprising culturing the cells in a medium comprising potassium ion at a concentration of at least about 50 mM, wherein the medium comprises IL-2. In some aspects, the medium does not comprise IL-7 and/or IL-15. In some aspects, the number of less-differentiated-cells, e.g., stem-like T cells, NK cells, or TILs, e.g., cells having a T_{SCM}-like phenotype, following culture in the medium is higher than the number of less-differentiated cells cultured in a medium comprising IL-2, IL-7, and IL-15.

In some aspects, the medium, e.g., MRM, described herein (e.g., comprising potassium ion at a concentration greater than 5 mM) comprises between about 50 IU/mL to about 500 IU/mL of IL-2. In some aspects, the medium, e.g., MRM, comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-2.

Therefore, in some aspects, the medium, e.g., MRM, comprises (i) potassium ion at a concentration higher than 5 mM and (ii) about 50 IU/mL of IL-2. In some aspects, the medium, e.g., MRM, comprises (i) potassium ion at a concentration higher than 5 mM and (ii) about 60 IU/mL of IL-2. In some aspects, the medium, e.g., MRM, comprises (i) potassium ion at a concentration higher than 5 mM and (ii) about 70 IU/mL of IL-2. In some aspects, the medium, e.g., MRM, comprises (i) potassium ion at a concentration higher than 5 mM and (ii) about 80 IU/mL of IL-2. In some aspects, the medium, e.g., MRM, comprises (i) potassium ion at a concentration higher than 5 mM and (ii) about 90 IU/mL of IL-2. In some aspects, the medium, e.g., MRM, comprises (i) potassium ion at a concentration higher than 5 mM and (ii) about 100 IU/mL of IL-2. In some aspects, the medium, e.g., MRM, comprises (i) potassium ion at a concentration higher than 5 mM and (ii) about 125 IU/mL of IL-2. In some aspects, the medium, e.g., MRM, comprises (i) potassium ion at a concentration higher than 5 mM and (ii) about 150 IU/mL of IL-2. In some aspects, the medium, e.g., MRM, comprises (i) potassium ion at a concentration higher than 5 mM and (ii) about 175 IU/mL of IL-2. In some aspects, the medium, e.g., MRM, comprises (i) potassium ion at a concentration higher than 5 mM and (ii) about 200 IU/mL of IL-2. In some aspects, the medium, e.g., MRM, comprises (i) potassium ion at a concentration higher than 5 mM and (ii) about 225 IU/mL of IL-2. In some aspects, the medium, e.g., MRM, comprises (i) potassium ion at a concentration higher than 5 mM and (ii) about 250 IU/mL of IL-2. In some aspects, the medium, e.g., MRM, comprises (i) potassium ion at a concentration higher than 5 mM and (ii) about 275 IU/mL of IL-2. In some aspects, the medium, e.g., MRM, comprises (i) potassium ion at a concentration higher than 5 mM and (ii) about 300 IU/mL of IL-2. In some aspects, the medium, e.g., MRM, comprises (i) potassium ion at a concentration higher than 5 mM and (ii) about 350 IU/mL of IL-2. In some aspects, the medium, e.g., MRM, comprises (i) potassium ion at a concentration higher than 5 mM and (ii) about 400 IU/mL of IL-2. In some aspects, the medium, e.g., MRM, comprises (i) potassium ion at a concentration higher than 5 mM and (ii) about 450 IU/mL of IL-2. In some aspects, the medium, e.g., MRM, comprises (i) potassium ion at a concentration higher than 5 mM and (ii) about 500 IU/mL of IL-2. In some aspects, the medium, e.g., MRM, comprising potassium ion and IL-2 further comprises NaCl at a concentration less than about 115 nM.

In some aspects, the medium comprises at least about 0.1 ng/mL IL-2. In some aspects, the medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-2.

In some aspects, the medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-2. In some aspects, the medium comprises at least about 1.0 ng/mL IL-2. In some aspects, the medium comprises at least about 2.0 ng/mL IL-2. In some aspects, the medium comprises at least about 3.0 ng/mL IL-2. In some aspects, the medium comprises at least about 4.0 ng/mL IL-2. In some aspects, the medium comprises at least about 5.0 ng/mL IL-2. In some aspects, the medium comprises at least about 6.0 ng/mL IL-2. In some aspects, the medium comprises at least about 7.0 ng/mL IL-2. In some aspects, the medium comprises at least about 8.0 ng/mL IL-2. In some aspects, the medium comprises at least about 9.0 ng/mL IL-2. In some aspects, the medium comprises at least about 10 ng/mL IL-2.

In some aspects, the media, e.g., the MRM, comprises at least about 0.1 ng/mL IL-2. In some aspects, the media, e.g., the MRM, comprises from about 50 ng/mL to about 600 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 100 ng/mL to about 600 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 250 ng/mL to about 350 ng/mL, about 300 ng/mL to about 600 ng/mL, about 300 ng/mL to about 500 ng/mL, about 300 ng/mL to about 450 ng/mL, about 300 ng/mL to about 400 ng/mL, about 300 ng/mL to about 350 ng/mL, about 250 ng/mL to about 300 ng/mL, or about 275 ng/mL to about 325 ng/mL IL-2.

In some aspects, the media, e.g., the MRM, comprises at least about 50 ng/mL, at least about 60 ng/mL, at least about 70 ng/mL, at least about 80 ng/mL, at least about 90 ng/mL, at least about 100 ng/mL, at least about 110 ng/mL, at least about 120 ng/mL, at least about 130 ng/mL, at least about 140 ng/mL, at least about 150 ng/mL, at least about 160 ng/mL, at least about 170 ng/mL, at least about 180 ng/mL, at least about 190 ng/mL, at least about 200 ng/mL, at least about 210 ng/mL, at least about 220 ng/mL, at least about 230 ng/mL, at least about 240 ng/mL, at least about 250 ng/mL, at least about 260 ng/mL, at least about 270 ng/mL, at least about 280 ng/mL, at least about 290 ng/mL, at least about 300 ng/mL, at least about 310 ng/mL, at least about 320 ng/mL, at least about 330 ng/mL, at least about 340 ng/mL, at least about 350 ng/mL, at least about 360 ng/mL, at least about 370 ng/mL, at least about 380 ng/mL, at least about 390 ng/mL, at least about 400 ng/mL, at least about 410 ng/mL, at least about 420 ng/mL, at least about 430 ng/mL, at least about 440 ng/mL, at least about 450 ng/mL, at least about 460 ng/mL, at least about 470 ng/mL, at least about 480 ng/mL, at least about 490 ng/mL, at least about 500 ng/mL, at least about 510 ng/mL, at least about 520 ng/mL, at least about 530 ng/mL, at least about 540 ng/mL, at least about 550 ng/mL, at least about 560 ng/mL, at least about 570 ng/mL, at least about 580 ng/mL, at least about 590 ng/mL, or at least about 600 ng/mL IL-2. In some aspects, the MRM comprises at least about 50 ng/mL IL-2. In some aspects, the media, e.g., the MRM, comprises at least about 60 ng/mL IL-2. In some aspects, the media, e.g., the MRM, comprises at least about 70 ng/mL IL-2. In some aspects, the media, e.g., the MRM, comprises at least about 73.6 ng/mL IL-2. In some aspects, the media, e.g., the MRM, comprises at least about 75 ng/mL IL-2. In some aspects, the media, e.g., the MRM, comprises at least about 80 ng/mL IL-2. In some aspects, the media, e.g., the MRM, comprises at least about 90 ng/mL IL-2. In some aspects, the media, e.g., the MRM, comprises at least about 100 ng/mL IL-2. In some aspects, the media, e.g., the MRM, comprises at least about 200 ng/mL IL-2. In some aspects, the media, e.g., the MRM, comprises at least about 300 ng/mL IL-2. In some aspects, the media, e.g., the MRM, comprises at least about 400 ng/mL IL-2. In some aspects, the media, e.g., the MRM, comprises at least about 500 ng/mL IL-2. In some aspects, the media, e.g., the MRM, comprises at least about 600 ng/mL IL-2.

In some aspects, the media, e.g., the MRM, comprises at least about 30 mM to at least about 100 mM potassium ion and about 300 ng/mL IL-2. In some aspects, the media, e.g., the MRM, comprises more than 40 mM potassium ion and about 300 ng/mL IL-2. In some aspects, the media, e.g., the MRM, comprises at least about 45 mM potassium ion and about 300 ng/mL IL-2. In some aspects, the media, e.g., the MRM, comprises at least about 50 mM potassium ion and about 300 ng/mL IL-2. In some aspects, the media, e.g., the MRM, comprises at least about 55 mM potassium ion and about 300 ng/mL IL-2. In some aspects, the media, e.g., the MRM, comprises at least about 60 mM potassium ion and about 300 ng/mL IL-2. In some aspects, the media, e.g., the MRM, comprises at least about 65 mM potassium ion and about 300 ng/mL IL-2. In some aspects, the media, e.g., the MRM, comprises at least about 70 mM potassium ion and about 300 ng/mL IL-2. In some aspects, the media, e.g., the MRM, comprises at least about 75 mM potassium ion and about 300 ng/mL IL-2. In some aspects, the media, e.g., the MRM, comprises at least about 80 mM potassium ion and about 300 ng/mL IL-2. In some aspects, the media, e.g., the MRM, comprises at least about 85 mM potassium ion and about 300 ng/mL IL-2. In some aspects, the media, e.g., the MRM, comprises at least about 90 mM potassium ion and about 300 ng/mL IL-2. In some aspects, the media, e.g., the MRM, comprises (i) at least about 70 mM potassium ion, (ii) about 60 mM sodium, (iii) about 1.4 mM calcium, (iv) about 16 mM glucose, and (v) about 10 ng/mL IL-2.

In some aspects, the medium comprises at least about 0.1 ng/mL IL-21. In some aspects, the medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-21.

In some aspects, the medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-21. In some aspects, the medium comprises at least about 1.0 ng/mL IL-21. In some aspects, the medium comprises at least about 2.0 ng/mL IL-21. In some aspects, the medium comprises at least about 3.0 ng/mL IL-21. In some aspects, the medium comprises at least about 4.0 ng/mL IL-21. In some aspects, the medium comprises at least about 5.0 ng/mL IL-21. In some aspects, the medium comprises at least about 6.0 ng/mL IL-21. In some aspects, the medium comprises at least about 7.0 ng/mL IL-21. In some aspects, the medium comprises at least about 8.0 ng/mL IL-21. In some aspects, the medium comprises at least about 9.0 ng/mL IL-21. In some aspects, the medium comprises at least about 10 ng/mL IL-21. In some aspects, the media, e.g., the MRM, comprises at least about 10 ng/mL IL-21. In some aspects, the media, e.g., the MRM, comprises at least about 15 ng/mL IL-21. In some aspects, the media, e.g., the MRM, comprises at least about 20 ng/mL IL-21. In some aspects, the MRM comprises at least about 25 ng/mL IL-21. In some aspects, the MRM comprises at least about 30 ng/mL IL-21. In some aspects, the MRM comprises at least about 35 ng/mL IL-21.

In some aspects, the medium, e.g., MRM, described herein (e.g., comprising potassium ion at a concentration greater than 5 mM) comprises between about 50 IU/mL to about 500 IU/mL of IL-21. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-21.

In some aspects, the medium, e.g., MRM, comprises (i) potassium ion at a concentration higher than 5 mM and (ii) about 50 IU/mL of IL-21. In some aspects, the medium, e.g., MRM, comprises (i) potassium ion at a concentration higher than 5 mM and (ii) about 60 IU/mL of IL-21. In some aspects, the medium, e.g., MRM, comprises (i) potassium ion at a concentration higher than 5 mM and (ii) about 70 IU/mL of IL-21. In some aspects, the medium, e.g., MRM, comprises (i) potassium ion at a concentration higher than 5 mM and (ii) about 80 IU/mL of IL-21. In some aspects, the medium, e.g., MRM, comprises (i) potassium ion at a concentration higher than 5 mM and (ii) about 90 IU/mL of IL-21. In some aspects, the medium, e.g., MRM, comprises (i) potassium ion at a concentration higher than 5 mM and (ii) about 100 IU/mL of IL-21. In some aspects, the medium, e.g., MRM, comprises (i) potassium ion at a concentration higher than 5 mM and (ii) about 125 IU/mL of IL-21. In some aspects, the medium, e.g., MRM, comprises (i) potassium ion at a concentration higher than 5 mM and (ii) about 150 IU/mL of IL-21. In some aspects, the medium, e.g., MRM, comprises (i) potassium ion at a concentration higher than 5 mM and (ii) about 175 IU/mL of IL-21. In some aspects, the medium, e.g., MRM, comprises (i) potassium ion at a concentration higher than 5 mM and (ii) about 200 IU/mL of IL-21. In some aspects, the medium, e.g., MRM, comprises (i) potassium ion at a concentration higher than 5 mM and (ii) about 225 IU/mL of IL-21. In some aspects, the medium, e.g., MRM, comprises (i) potassium ion at a concentration higher than 5 mM and (ii) about 250 IU/mL of IL-21. In some aspects, the medium, e.g., MRM, comprises (i) potassium ion at a concentration higher than 5 mM and (ii) about 275 IU/mL of IL-21. In some aspects, the medium, e.g., MRM, comprises (i) potassium ion at a concentration higher than 5 mM and (ii) about 300 IU/mL of IL-21. In some aspects, the medium, e.g., MRM, comprises (i) potassium ion at a concentration higher than 5 mM and (ii) about 350 IU/mL of IL-21. In some aspects, the medium, e.g., MRM, comprises (i) potassium ion at a concentration higher than 5 mM and (ii) about 400 IU/mL of IL-21. In some aspects, the medium, e.g., MRM, comprises (i) potassium ion at a concentration higher than 5 mM and (ii) about 450 IU/mL of IL-21. In some aspects, the medium, e.g., MRM, comprises (i) potassium ion at a concentration higher than 5 mM and (ii) about 500 IU/mL of IL-21. In some aspects, the medium, e.g., MRM, comprising potassium ion and IL-21 further comprises NaCl at a concentration less than about 115 nM.

In some aspects, the medium, e.g., MRM, described herein (e.g., comprising potassium ion at a concentration greater than 5 mM) comprises between about 500 IU/mL to about 1,500 IU/mL of IL-7. In some aspects, the culture medium comprises about 500 IU/mL, about 550 IU/mL, about 600 IU/mL, about 650 IU/mL, about 700 IU/mL, about 750 IU/mL, about 800 IU/mL, about 850 IU/mL, about 900 IU/mL, about 950 IU/mL, about 1,000 IU/mL, about 1,050 IU/mL, about 1,100 IU/mL, about 1,150 IU/mL, about 1,200 IU/mL, about 1,250 IU/mL, about 1,300 IU/mL, about 1,350 IU/mL, about 1,400 IU/mL, about 1,450 IU/mL, or about 1,500 IU/mL of IL-7.

In some aspects, the medium, e.g., MRM, useful for the present disclosure comprises (i) potassium ion at a concentration higher than 5 mM and (ii) about 500 IU/mL of IL-7. In some aspects, the medium, e.g., MRM, comprises (i) potassium ion at a concentration higher than 5 mM and (ii) about 550 IU/mL of IL-7. In some aspects, the medium, e.g., MRM, comprises (i) potassium ion at a concentration higher than 5 mM and (ii) about 600 IU/mL of IL-7. In some aspects, the medium, e.g., MRM, comprises (i) potassium ion at a concentration higher than 5 mM and (ii) about 650 IU/mL of IL-7. In some aspects, the medium, e.g., MRM, comprises (i) potassium ion at a concentration higher than 5 mM and (ii) about 700 IU/mL of IL-7. In some aspects, the medium, e.g., MRM, comprises (i) potassium ion at a concentration higher than 5 mM and (ii) about 750 IU/mL of IL-7. In some aspects, the medium, e.g., MRM, comprises (i) potassium ion at a concentration higher than 5 mM and (ii) about 800 IU/mL of IL-7. In some aspects, the medium, e.g., MRM, comprises (i) potassium ion at a concentration higher than 5 mM and (ii) about 850 IU/mL of IL-7. In some aspects, the medium, e.g., MRM, comprises (i) potassium ion at a concentration higher than 5 mM and (ii) about 900 IU/mL of IL-7. In some aspects, the medium, e.g., MRM, comprises (i) potassium ion at a concentration higher than 5 mM and (ii) about 950 IU/mL of IL-7. In some aspects, the medium, e.g., MRM, comprises (i) potassium ion at a concentration higher than 5 mM and (ii) about 1,000 IU/mL of IL-7. In some aspects, the medium, e.g., MRM, comprises (i) potassium ion at a concentration higher than 5 mM and (ii) about 1,050 IU/mL of IL-7. In some aspects, the medium, e.g., MRM, comprises (i) potassium ion at a concentration higher than 5 mM and (ii) about 1,100 IU/mL of IL-7. In some aspects, the medium, e.g., MRM, comprises (i) potassium ion at a concentration higher than 5 mM and (ii) about 1,150 IU/mL of IL-7. In some aspects, the medium, e.g., MRM, comprises (i) potassium ion at a concentration higher than 5 mM and (ii) about 1,200 IU/mL of IL-7. In some aspects, the medium, e.g., MRM, comprises (i) potassium ion at a concentration higher than 5 mM and (ii) about 1,250 IU/mL of IL-7. In some aspects, the medium, e.g., MRM, comprises (i) potassium ion at a concentration higher than 5 mM and (ii) about 1,300 IU/mL of IL-7. In some aspects, the medium, e.g., MRM, comprises (i) potassium ion at a concentration higher than 5 mM and (ii) about 1,350 IU/mL of IL-7. In some aspects, the medium, e.g., MRM, comprises (i) potassium ion at a concentration higher than 5 mM and (ii) about 1,400 IU/mL of IL-7. In some aspects, the medium, e.g., MRM, comprises (i) potassium ion at a concentration higher than 5 mM and (ii) about 1,450 IU/mL of IL-7. In some aspects, the medium, e.g., MRM, comprises (i) potassium ion at a concentration higher than 5 mM and (ii) about 1,500 IU/mL of IL-7. In some aspects, the medium, e.g., MRM, comprising potassium ion and IL-7 further comprises NaCl at a concentration less than about 115 nM.

In some aspects, the medium comprises at least about 0.1 ng/mL IL-7. In some aspects, the medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-7.

In some aspects, the medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-7. In some aspects, the medium comprises at least about 1.0 ng/mL IL-7. In some aspects, the medium comprises at least about 2.0 ng/mL IL-7. In some aspects, the medium comprises at least about 3.0 ng/mL IL-7. In some aspects, the medium comprises at least about 4.0 ng/mL IL-7. In some aspects, the medium comprises at least about 5.0 ng/mL IL-7. In some aspects, the medium comprises at least about 6.0 ng/mL IL-7. In some aspects, the medium comprises at least about 7.0 ng/mL IL-7. In some aspects, the medium comprises at least about 8.0 ng/mL IL-7. In some aspects, the medium comprises at least about 9.0 ng/mL IL-7. In some aspects, the medium comprises at least about 10 ng/mL IL-7.

In some aspects, the medium, e.g., MRM, described herein (e.g., comprising potassium ion at a concentration greater than 5 mM) comprises between about 50 IU/mL to about 500 IU/mL of IL-15. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-15.

Therefore, in some aspects, the medium, e.g., MRM, comprises (i) potassium ion at a concentration higher than 5 mM and (ii) about 50 IU/mL of IL-15. In some aspects, the medium, e.g., MRM, comprises (i) potassium ion at a concentration higher than 5 mM and (ii) about 60 IU/mL of IL-15. In some aspects, the medium, e.g., MRM, comprises (i) potassium ion at a concentration higher than 5 mM and (ii) about 70 IU/mL of IL-15. In some aspects, the medium, e.g., MRM, comprises (i) potassium ion at a concentration higher than 5 mM and (ii) about 80 IU/mL of IL-15. In some aspects, the medium, e.g., MRM, comprises (i) potassium ion at a concentration higher than 5 mM and (ii) about 90 IU/mL of IL-15. In some aspects, the medium, e.g., MRM, comprises (i) potassium ion at a concentration higher than 5 mM and (ii) about 100 IU/mL of IL-15. In some aspects, the medium, e.g., MRM, comprises (i) potassium ion at a concentration higher than 5 mM and (ii) about 125 IU/mL of IL-15. In some aspects, the medium, e.g., MRM, comprises (i) potassium ion at a concentration higher than 5 mM and (ii) about 150 IU/mL of IL-15. In some aspects, the medium, e.g., MRM, comprises (i) potassium ion at a concentration higher than 5 mM and (ii) about 175 IU/mL of IL-15. In some aspects, the medium, e.g., MRM, comprises (i) potassium ion at a concentration higher than 5 mM and (ii) about 200 IU/mL of IL-15. In some aspects, the medium, e.g., MRM, comprises (i) potassium ion at a concentration higher than 5 mM and (ii) about 225 IU/mL of IL-15. In some aspects, the medium, e.g., MRM, comprises (i) potassium ion at a concentration higher than 5 mM and (ii) about 250 IU/mL of IL-15. In some aspects, the medium, e.g., MRM, comprises (i) potassium ion at a concentration higher than 5 mM and (ii) about 275 IU/mL of IL-15. In some aspects, the medium, e.g., MRM, comprises (i) potassium ion at a concentration higher than 5 mM and (ii) about 300 IU/mL of IL-15. In some aspects, the medium, e.g., MRM, comprises (i) potassium ion at a concentration higher than 5 mM and (ii) about 350 IU/mL of IL-15. In some aspects, the medium, e.g., MRM, comprises (i) potassium ion at a concentration higher than 5 mM and (ii) about 400 IU/mL of IL-15. In some aspects, the medium, e.g., MRM, comprises (i) potassium ion at a concentration higher than 5 mM and (ii) about 450 IU/mL of IL-15. In some aspects, the medium, e.g., MRM, comprises (i) potassium ion at a concentration higher than 5 mM and (ii) about 500 IU/mL of IL-15. In some aspects, the medium, e.g., MRM, comprising potassium ion and IL-15 further comprises NaCl at a concentration less than about 115 nM.

In some aspects, the medium comprises at least about 0.1 ng/mL IL-15. In some aspects, the medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-15.

In some aspects, the medium comprises at least about 0.1 ng/mL, at least about 0.2 ng/mL, at least about 0.3 ng/mL, at least about 0.4 ng/mL, at least about 0.5 ng/mL, at least about 0.6 ng/mL, at least about 0.7 ng/mL, at least about 0.8 ng/mL, at least about 0.9 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-15. In some aspects, the medium comprises at least about 1.0 ng/mL IL-15. In some aspects, the medium comprises at least about 2.0 ng/mL IL-15. In some aspects, the medium comprises at least about 3.0 ng/mL IL-15. In some aspects, the medium comprises at least about 4.0 ng/mL IL-15. In some aspects, the medium comprises at least about 5.0 ng/mL IL-15. In some aspects, the medium comprises at least about 6.0 ng/mL IL-15. In some aspects, the medium comprises at least about 7.0 ng/mL IL-15. In some aspects, the medium comprises at least about 8.0 ng/mL IL-15. In some aspects, the medium comprises at least about 9.0 ng/mL IL-15. In some aspects, the medium comprises at least about 10 ng/mL IL-15. In some aspects, the medium further comprises NaCl, wherein the total concentration of potassium ion and NaCl is from 110 mM to 140 mM.

In some aspects, the media, e.g., the MRM, comprises at least about 30 mM to at least about 100 mM potassium ion, about 300 ng/mL IL-2, and about 0.4 ng/mL IL-15. In some aspects, the media, e.g., the MRM, comprises more than 40 mM potassium ion, about 300 ng/mL IL-2, and about 0.4 ng/mL IL-15. In some aspects, the media, e.g., the MRM, comprises at least about 45 mM potassium ion, about 300 ng/mL IL-2, and about 0.4 ng/mL IL-15. In some aspects, the media, e.g., the MRM, comprises at least about 50 mM potassium ion, about 300 ng/mL IL-2, and about 0.4 ng/mL IL-15. In some aspects, the media, e.g., the MRM, comprises at least about 55 mM potassium ion, about 300 ng/mL IL-2, and about 0.4 ng/mL IL-15. In some aspects, the media, e.g., the MRM, comprises at least about 60 mM potassium ion, about 300 ng/mL IL-2, and about 0.4 ng/mL IL-15. In some aspects, the media, e.g., the MRM, comprises at least about 65 mM potassium ion, about 300 ng/mL IL-2, and about 0.4 ng/mL IL-15. In some aspects, the media, e.g., the MRM, comprises at least about 70 mM potassium ion, about 300 ng/mL IL-2, and about 0.4 ng/mL IL-15. In some aspects, the media, e.g., the MRM, comprises at least about 75 mM potassium ion, about 300 ng/mL IL-2, and about 0.4 ng/mL IL-15. In some aspects, the media, e.g., the MRM, comprises at least about 80 mM potassium ion, about 300 ng/mL IL-2, and about 0.4 ng/mL IL-15. In some aspects, the media, e.g., the MRM, comprises at least about 85 mM potassium ion, about 300 ng/mL IL-2, and about 0.4 ng/mL IL-15. In some aspects, the media, e.g., the MRM, comprises at least about 90 mM potassium ion, about 300 ng/mL IL-2, and about 0.4 ng/mL IL-15. In some aspects, the media, e.g., the MRM, comprises (i) at least about 70 mM potassium ion, (ii) about 60 mM sodium, (iii) about 1.4 mM calcium, (iv) about 16 mM glucose, (v) about 300 ng/mL IL-2, and (vi) about 0.4 ng/mL IL-15.

In certain aspects, the medium comprises more than 40 mM potassium ion and about 10 ng/mL IL-2, wherein the medium is hypotonic. In certain aspects, the medium comprise at least about 45 mM potassium ion and about 10 ng/mL IL-2, wherein the medium is hypotonic. In certain aspects, the medium comprise at least about 50 mM potassium ion and about 10 ng/mL IL-2, wherein the medium is hypotonic. In certain aspects, the medium comprise at least about 55 mM potassium ion and about 10 ng/mL IL-2, wherein the medium is hypotonic. In certain aspects, the medium comprise at least about 60 mM potassium ion and about 10 ng/mL IL-2, wherein the medium is hypotonic. In certain aspects, the medium comprise at least about 65 mM potassium ion and about 10 ng/mL IL-2, wherein the medium is hypotonic. In certain aspects, the medium comprise at least about 70 mM potassium ion and about 10 ng/mL IL-2, wherein the medium is hypotonic. In certain aspects, the medium comprise at least about 75 mM potassium ion and about 10 ng/mL IL-2, wherein the medium is hypotonic. In certain aspects, the medium comprise at least about 80 mM potassium ion and about 10 ng/mL IL-2, wherein the medium is hypotonic. In certain aspects, the medium comprise at least about 85 mM potassium ion and about 10 ng/mL IL-2, wherein the medium is hypotonic. In certain aspects, the medium comprise at least about 90 mM potassium ion and about 10 ng/mL IL-2, wherein the medium is hypotonic. In certain aspects, the medium comprises (i) at least about 70 mM potassium ion, (ii) about 60 mM sodium, (iii) about 1.4 mM calcium, (iv) about 16 mM glucose, and (v) about 10 ng/mL IL-2, wherein the medium is hypotonic.

In certain aspects, the medium comprises more than 40 mM potassium ion and about 10 ng/mL IL-2 and about 1 ng/mL IL-7, wherein the medium is hypotonic. In certain aspects, the medium comprise at least about 45 mM potassium ion and about 10 ng/mL IL-2 and about 1 ng/mL IL-7, wherein the medium is hypotonic. In certain aspects, the medium comprise at least about 40 mM potassium ion and about 50 ng/mL IL-2 and about 1 ng/mL IL-7, wherein the medium is hypotonic. In certain aspects, the medium comprise at least about 55 mM potassium ion and about 10 ng/mL IL-2 and about 1 ng/mL IL-7, wherein the medium is hypotonic. In certain aspects, the medium comprise at least about 60 mM potassium ion and about 10 ng/mL IL-2 and about 1 ng/mL IL-7, wherein the medium is hypotonic. In certain aspects, the medium comprise at least about 65 mM potassium ion and about 10 ng/mL IL-2 and about 1 ng/mL IL-7, wherein the medium is hypotonic. In certain aspects, the medium comprise at least about 70 mM potassium ion and about 10 ng/mL IL-2 and about 1 ng/mL IL-7, wherein the medium is hypotonic. In certain aspects, the medium comprise at least about 75 mM potassium ion and about 10 ng/mL IL-2 and about 1 ng/mL IL-7, wherein the medium is hypotonic. In certain aspects, the medium comprise at least about 80 mM potassium ion and about 10 ng/mL IL-2 and about 1 ng/mL IL-7, wherein the medium is hypotonic. In certain aspects, the medium comprise at least about 85 mM potassium ion and about 10 ng/mL IL-2 and about 1 ng/mL IL-7, wherein the medium is hypotonic. In certain aspects, the medium comprise at least about 90 mM potassium ion and about 10 ng/mL IL-2 and about 1 ng/mL IL-7, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 10 ng/mL IL-2, and (vi) about 1 ng/mL IL-7, wherein the medium is hypotonic.

In certain aspects, the medium comprises more than 40 mM potassium ion and about 10 ng/mL IL-2 and about 1 ng/mL IL-15, wherein the medium is hypotonic. In certain aspects, the medium comprise at least about 45 mM potassium ion and about 10 ng/mL IL-2 and about 1 ng/mL IL-15, wherein the medium is hypotonic. In certain aspects, the medium comprise at least about 50 mM potassium ion and about 10 ng/mL IL-2 and about 1 ng/mL IL-15, wherein the medium is hypotonic. In certain aspects, the medium comprise at least about 55 mM potassium ion and about 10 ng/mL IL-2 and about 1 ng/mL IL-15, wherein the medium is hypotonic. In certain aspects, the medium comprise at least about 60 mM potassium ion and about 10 ng/mL IL-2 and about 1 ng/mL IL-15, wherein the medium is hypotonic. In certain aspects, the medium comprise at least about 65 mM potassium ion and about 10 ng/mL IL-2 and about 1 ng/mL IL-15, wherein the medium is hypotonic. In certain aspects, the medium comprise at least about 70 mM potassium ion and about 10 ng/mL IL-2 and about 1 ng/mL IL-15, wherein the medium is hypotonic. In certain aspects, the medium comprise at least about 75 mM potassium ion and about 10 ng/mL IL-2 and about 1 ng/mL IL-15, wherein the medium is hypotonic. In certain aspects, the medium comprise at least about 80 mM potassium ion and about 10 ng/mL IL-2 and about 1 ng/mL IL-15, wherein the medium is hypotonic. In certain aspects, the medium comprise at least about 85 mM potassium ion and about 10 ng/mL IL-2 and about 1 ng/mL IL-15, wherein the medium is hypotonic. In certain aspects, the medium comprise at least about 90 mM potassium ion and about 10 ng/mL IL-2 and about 1 ng/mL IL-15, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 10 ng/mL IL-2, and (vi) about 1 ng/mL IL-15, wherein the medium is hypotonic.

In certain aspects, the medium comprises more than 40 mM potassium ion and about 10 ng/mL IL-2, about 1 ng/mL IL-7, and about 1 ng/mL IL-15, wherein the medium is hypotonic. In certain aspects, the medium comprise at least about 45 mM potassium ion and about 10 ng/mL IL-2, about 1 ng/mL IL-7, and about 1 ng/mL IL-15, wherein the medium is hypotonic. In certain aspects, the medium comprise at least about 50 mM potassium ion and about 10 ng/mL IL-2, about 1 ng/mL IL-7, and about 1 ng/mL IL-15, wherein the medium is hypotonic. In certain aspects, the medium comprise at least about 55 mM potassium ion and about 10 ng/mL IL-2, about 1 ng/mL IL-7, and about 1 ng/mL IL-15, wherein the medium is hypotonic. In certain aspects, the medium comprise at least about 60 mM potassium ion and about 10 ng/mL IL-2, about 1 ng/mL IL-7, and about 1 ng/mL IL-15, wherein the medium is hypotonic. In certain aspects, the medium comprise at least about 65 mM potassium ion and about 10 ng/mL IL-2, about 1 ng/mL IL-7, and about 1 ng/mL IL-15, wherein the medium is hypotonic. In certain aspects, the medium comprise at least about 70 mM potassium ion and about 10 ng/mL IL-2, about 1 ng/mL IL-7, and about 1 ng/mL IL-15, wherein the medium is hypotonic. In certain aspects, the medium comprise at least about 75 mM potassium ion and about 10 ng/mL IL-2, about 1 ng/mL IL-7, and about 1 ng/mL IL-15, wherein the medium is hypotonic. In certain aspects, the medium comprise at least about 80 mM potassium ion and about 10 ng/mL IL-2, about 1 ng/mL IL-7, and about 1 ng/mL IL-15, wherein the medium is hypotonic. In certain aspects, the medium comprise at least about 85 mM potassium ion and about 10 ng/mL IL-2, about 1 ng/mL IL-7, and about 1 ng/mL IL-15, wherein the medium is hypotonic. In certain aspects, the medium comprise at least about 90 mM potassium ion and about 10 ng/mL IL-2, about 1 ng/mL IL-7, and about 1 ng/mL IL-15, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 10 ng/mL IL-2, (vi) about 1 ng/mL IL-7, and (vii) about 1 ng/mL IL-15, wherein the medium is hypotonic.

In certain aspects, the medium comprises more than 40 mM potassium ion and about 10 ng/mL IL-2 and about 1 ng/mL IL-21, wherein the medium is hypotonic. In certain aspects, the medium comprise at least about 45 mM potassium ion and about 10 ng/mL IL-2 and about 1 ng/mL IL-21, wherein the medium is hypotonic. In certain aspects, the medium comprise at least about 50 mM potassium ion and about 10 ng/mL IL-2 and about 1 ng/mL IL-21, wherein the medium is hypotonic. In certain aspects, the medium comprise at least about 55 mM potassium ion and about 10 ng/mL IL-2 and about 1 ng/mL IL-21, wherein the medium is hypotonic. In certain aspects, the medium comprise at least about 60 mM potassium ion and about 10 ng/mL IL-2 and about 1 ng/mL IL-21, wherein the medium is hypotonic. In certain aspects, the medium comprise at least about 65 mM potassium ion and about 10 ng/mL IL-2 and about 1 ng/mL IL-21, wherein the medium is hypotonic. In certain aspects, the medium comprise at least about 70 mM potassium ion and about 10 ng/mL IL-2 and about 1 ng/mL IL-21, wherein the medium is hypotonic. In certain aspects, the medium comprise at least about 75 mM potassium ion and about 10 ng/mL IL-2 and about 1 ng/mL IL-21, wherein the medium is hypotonic. In certain aspects, the medium comprise at least about 80 mM potassium ion and about 10 ng/mL IL-2 and about 1 ng/mL IL-21, wherein the medium is hypotonic. In certain aspects, the medium comprise at least about 85 mM potassium ion and about 10 ng/mL IL-2 and about 1 ng/mL IL-21, wherein the medium is hypotonic. In certain aspects, the medium comprise at least about 90 mM potassium ion and about 10 ng/mL IL-2 and about 1 ng/mL IL-21, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 10 ng/mL IL-2, and (vi) about 1 ng/mL IL-21, wherein the medium is hypotonic.

In certain aspects, the medium comprises more than 40 mM potassium ion and about 1 ng/mL IL-7 and about 1 ng/mL IL-21, wherein the medium is hypotonic. In certain aspects, the medium comprise at least about 45 mM potassium ion and about 1 ng/mL IL-7 and about 1 ng/mL IL-21, wherein the medium is hypotonic. In certain aspects, the medium comprise at least about 50 mM potassium ion and about 1 ng/mL IL-7 and about 1 ng/mL IL-21, wherein the medium is hypotonic. In certain aspects, the medium comprise at least about 55 mM potassium ion and about 1 ng/mL IL-7 and about 1 ng/mL IL-21, wherein the medium is hypotonic. In certain aspects, the medium comprise at least about 60 mM potassium ion and about 1 ng/mL IL-7 and about 1 ng/mL IL-21, wherein the medium is hypotonic. In certain aspects, the medium comprise at least about 65 mM potassium ion and about 1 ng/mL IL-7 and about 1 ng/mL IL-21, wherein the medium is hypotonic. In certain aspects, the medium comprise at least about 70 mM potassium ion and about 1 ng/mL IL-7 and about 1 ng/mL IL-21, wherein the medium is hypotonic. In certain aspects, the medium comprise at least about 75 mM potassium ion and about 1 ng/mL IL-7 and about 1 ng/mL IL-21, wherein the medium is hypotonic. In certain aspects, the medium comprise at least about 80 mM potassium ion and about 1 ng/mL IL-7 and about 1 ng/mL IL-21, wherein the medium is hypotonic. In certain aspects, the medium comprise at least about 85 mM potassium ion and about 1 ng/mL IL-7 and about 1 ng/mL IL-21, wherein the medium is hypotonic. In certain aspects, the medium comprise at least about 90 mM potassium ion and about 1 ng/mL IL-7 and about 1 ng/mL IL-21, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 1 ng/mL IL-7, and (vi) about 1 ng/mL IL-21, wherein the medium is hypotonic.

In certain aspects, the medium comprises more than 40 mM potassium ion and about 1 ng/mL IL-15 and about 1 ng/mL IL-21, wherein the medium is hypotonic. In certain aspects, the medium comprise at least about 45 mM potassium ion and about 1 ng/mL IL-15 and about 1 ng/mL IL-21, wherein the medium is hypotonic. In certain aspects, the medium comprise at least about 50 mM potassium ion and about 1 ng/mL IL-15 and about 1 ng/mL IL-21, wherein the medium is hypotonic. In certain aspects, the medium comprise at least about 55 mM potassium ion and about 1 ng/mL IL-15 and about 1 ng/mL IL-21, wherein the medium is hypotonic. In certain aspects, the medium comprise at least about 60 mM potassium ion and about 1 ng/mL IL-15 and about 1 ng/mL IL-21, wherein the medium is hypotonic. In certain aspects, the medium comprise at least about 65 mM potassium ion and about 1 ng/mL IL-15 and about 1 ng/mL IL-21, wherein the medium is hypotonic. In certain aspects, the medium comprise at least about 70 mM potassium ion and about 1 ng/mL IL-15 and about 1 ng/mL IL-21, wherein the medium is hypotonic. In certain aspects, the medium comprise at least about 75 mM potassium ion and about 1 ng/mL IL-15 and about 1 ng/mL IL-21, wherein the medium is hypotonic. In certain aspects, the medium comprise at least about 80 mM potassium ion and about 1 ng/mL IL-15 and about 1 ng/mL IL-21, wherein the medium is hypotonic. In certain aspects, the medium comprise at least about 85 mM potassium ion and about 1 ng/mL IL-15 and about 1 ng/mL IL-21, wherein the medium is hypotonic. In certain aspects, the medium comprise at least about 90 mM potassium ion and about 1 ng/mL IL-15 and about 1 ng/mL IL-21, wherein the medium is hypotonic. In certain aspects, the medium comprises (i) at least about 70 mM potassium ion, (ii) about 60 mM sodium, (iii) about 1.4 mM calcium, (iv) about 16 mM glucose, (v) about 1 ng/mL IL-15, and (vi) about 1 ng/mL IL-2111, wherein the medium is hypotonic.

In certain aspects, the medium comprises more than 40 mM potassium ion, NaCl, and about 10 ng/mL IL-2, wherein the total concentration of potassium ion and NaCl in the medium is between 110 mM and 140 mM. In certain aspects, the medium comprise at least about 45 mM potassium ion, NaCl, and about 10 ng/mL IL-2, wherein the total concentration of potassium ion and NaCl in the medium is between 110 mM and 140 mM. In certain aspects, the medium comprise at least about 50 mM potassium ion, NaCl, and about 10 ng/mL IL-2, wherein the total concentration of potassium ion and NaCl in the medium is between 110 mM and 140 mM. In certain aspects, the medium comprise at least about 55 mM potassium ion, NaCl, and about 10 ng/mL IL-2, wherein the total concentration of potassium ion and NaCl in the medium is between 110 mM and 140 mM. In certain aspects, the medium comprise at least about 60 mM potassium ion, NaCl, and about 10 ng/mL IL-2, wherein the total concentration of potassium ion and NaCl in the medium is between 110 mM and 140 mM. In certain aspects, the medium comprise at least about 65 mM potassium ion, NaCl, and about 10 ng/mL IL-2, wherein the total concentration of potassium ion and NaCl in the medium is between 110 mM and 140 mM. In certain aspects, the medium comprise at least about 70 mM potassium ion, NaCl, and about 10 ng/mL IL-2, wherein the total concentration of potassium ion and NaCl in the medium is between 110 mM and 140 mM. In certain aspects, the medium comprise at least about 75 mM potassium ion, NaCl, and about 10 ng/mL IL-2, wherein the total concentration of potassium ion and NaCl in the medium is between 110 mM and 140 mM. In certain aspects, the medium comprise at least about 80 mM potassium ion, NaCl, and about 10 ng/mL IL-2, wherein the total concentration of potassium ion and NaCl in the medium is between 110 mM and 140 mM. In certain aspects, the medium comprise at least about 85 mM potassium ion, NaCl, and about 10 ng/mL IL-2, wherein the total concentration of potassium ion and NaCl in the medium is between 110 mM and 140 mM. In certain aspects, the medium comprise at least about 90 mM potassium ion, NaCl, and about 10 ng/mL IL-2, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, and (v) about 10 ng/mL IL-2.

In certain aspects, the medium comprises more than 40 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 45 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 40 mM potassium ion, NaCl, and about 50 ng/mL IL-2 and about 1 ng/mL IL-7, wherein the total concentration of potassium ion and NaCl in the medium is between 110 mM and 140 mM. In certain aspects, the medium comprise at least about 55 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 60 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 65 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 70 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 75 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 80 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 85 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprise at least about 90 mM potassium ion, NaCl, and about 10 ng/mL IL-2 and about 1 ng/mL IL-7, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 10 ng/mL IL-2, and (vi) about 1 ng/mL IL-7.

In certain aspects, the medium comprises more than 40 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 45 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 50 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 55 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 60 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 65 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 70 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 75 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 80 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 85 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprise at least about 90 mM potassium ion, NaCl, and about 10 ng/mL IL-2 and about 1 ng/mL IL-15, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 10 ng/mL IL-2, and (vi) about 1 ng/mL IL-15.

In certain aspects, the medium comprises more than 40 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 45 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 50 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 55 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 60 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 65 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 70 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 75 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 80 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 85 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprise at least about 90 mM potassium ion, NaCl, and about 10 ng/mL IL-2, about 1 ng/mL IL-7, and about 1 ng/mL IL-15, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 10 ng/mL IL-2, (vi) about 1 ng/mL IL-7, and (vii) about 1 ng/mL IL-15.

In certain aspects, the medium comprises more than 40 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 45 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 50 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 55 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 60 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 65 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 70 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 75 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 80 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 85 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprise at least about 90 mM potassium ion, NaCl, and about 10 ng/mL IL-2 and about 1 ng/mL IL-21, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 10 ng/mL IL-2, and (vi) about 1 ng/mL IL-21.

In certain aspects, the medium comprises more than 40 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 45 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 50 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 55 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 60 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 65 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 70 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 75 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 80 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 85 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprise at least about 90 mM potassium ion, NaCl, and about 1 ng/mL IL-7 and about 1 ng/mL IL-21, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 1 ng/mL IL-7, and (vi) about 1 ng/mL IL-21.

In certain aspects, the medium comprises more than 40 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 45 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 50 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 55 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 60 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 65 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 70 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 75 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 80 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 85 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprise at least about 90 mM potassium ion, NaCl, and about 1 ng/mL IL-15 and about 1 ng/mL IL-21, wherein the total concentration of potassium ion and NaCl in the medium is between 110 mM and 140 mM. In certain aspects, the medium comprises (i) at least about 70 mM potassium ion, (ii) about 60 mM NaCl, (iii) about 1.4 mM calcium, (iv) about 16 mM glucose, (v) about 1 ng/mL IL-15, and (vi) about 1 ng/mL IL-21, wherein the total concentration of potassium ion and NaCl in the medium is between 110 mM and 140 mM.

In some aspects, the media comprises more than 40 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 50 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 55 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 60 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 65 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 70 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 75 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM. In some aspects, the media comprises about 80 mM potassium ion, NaCl, about 200 IU/mL IL-2, about 1200 IU/mL IL-7, about 200 IU/mL IL-15, wherein the total concentration of potassium ion and NaCl in the medium is between 110 mM and 140 mM.

II.G. Basal Media

Certain aspects of the present disclosure are directed to methods of culturing cells, e.g., pluripotent, multipotent, and/or immune cells (e.g., T cells and/or NK cells), comprising placing the cells in a medium comprising potassium ion at a concentration of at least about 5 mM, wherein the media is hypotonic or isotonic. Some aspects of the present disclosure are directed to methods of culturing cells, e.g., pluripotent, multipotent, and/or immune cells (e.g., T cells and/or NK cells), comprising placing the cells in a medium comprising potassium ion at a concentration higher than 40 mM. Some aspects of the present disclosure are directed to methods of culturing cells, e.g., pluripotent, multipotent, and/or immune cells (e.g., T cells and/or NK cells), comprising placing the cells in a medium comprising potassium ion at a concentration higher than 40 mM and NaCl at a concentration less than 100 mM. Some aspects of the present disclosure are directed to methods of culturing cells, e.g., pluripotent, multipotent, and/or immune cells (e.g., T cells and/or NK cells), comprising placing the cells in a medium comprising potassium ion at a concentration of at least about 50 mM. Some aspects of the present disclosure are directed to methods of culturing cells, e.g., pluripotent, multipotent, and/or immune cells (e.g., T cells and/or NK cells), comprising placing the cells in a medium comprising potassium ion at a concentration of at least about 50 mM and NaCl at a concentration of less than 90 mM. In some aspects, the medium is prepared by adding potassium ion to a basal medium. Any basal medium known in the art that is used to culture cells, e.g., pluripotent, multipotent, and/or immune cells (e.g., T cells and/or NK cells), can be used.

In some aspects, the basal medium further comprises one or more essential amino acids. In some aspects, the basal media comprises one or more essential amino acids selected form L-arginine, L-cysteine, L-isoleucine, L-leucine, L-lysine, L-methionine, L-phenylalanine, L-threonine, L-tryptophan, L-histidine, L-tyrosine, L-valine, and L-glutamine, or any combination thereof. In some aspects, the basal media comprises L-glutamine.

In some aspects, the basal media comprises at least about 0.01 mM of one or more essential amino acids. In some aspects, the basal media comprises about 0.01 mM to about 10 mM of one or more essential amino acids. In some aspects, the basal media comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM of one or more essential amino acids.

In some aspects, the basal media comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM of one or more essential amino acids.

In certain aspects, the basal medium comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM of one or more essential amino acids.

In some aspects, the basal medium comprises L-glutamine. In some aspects, the basal media comprises at least about 0.01 mM L-glutamine. In some aspects, the basal media comprises about 0.01 mM to about 10 mM L-glutamine. In some aspects, the basal media comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM L-glutamine.

In some aspects, the basal media comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM L-glutamine.

In certain aspects, the basal medium comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM L-glutamine. In some aspects, the basal medium comprises about 1.7 mM L-glutamine. In certain aspects, the basal medium comprises about 1.68 mM L-glutamine.

In some aspects, the basal media comprises about 0.14 mM L-glutamine. In some aspects, the basal media comprises about 0.15 mM L-glutamine. In some aspects, the basal media comprises about 1.76 mM L-glutamine. In some aspects, the basal media comprises about 1.83 mM L-glutamine. In some aspects, the basal media comprises about 1.84 mM L-glutamine. In some aspects, the basal media comprises about 1.97 mM L-glutamine. In some aspects, the basal media comprises about 2.05 mM L-glutamine. In some aspects, the basal media comprises about 2.11 mM L-glutamine. In some aspects, the basal media comprises about 2.18 mM L-glutamine. In some aspects, the basal media comprises about 5.41 mM L-glutamine. In some aspects, the basal media comprises about 5.47 mM L-glutamine. In some aspects, the basal media comprises about <0.10 mM L-glutamine.

In some aspects, the basal medium comprises L-glutamic acid. In some aspects, the basal media comprises at least about 0.01 mM L-glutamic acid. In some aspects, the basal media comprises about 0.01 mM to about 10 mM L-glutamic acid. In some aspects, the basal media comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM L-glutamic acid.

In some aspects, the basal media comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM L-glutamic acid.

In certain aspects, the basal medium comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM L-glutamic acid.

In some aspects, the basal media comprises about 0.15 mM L-glutamic acid. In some aspects, the basal media comprises about 0.17 mM L-glutamic acid. In some aspects, the basal media comprises about 0.18 mM L-glutamic acid. In some aspects, the basal media comprises about 0.19 mM L-glutamic acid. In some aspects, the basal media comprises about 0.85 mM L-glutamic acid. In some aspects, the basal media comprises about 0.86 mM L-glutamic acid. In some aspects, the basal media comprises about 0.9 mM L-glutamic acid. In some aspects, the basal media comprises about 0.95 mM L-glutamic acid. In some aspects, the basal media comprises about 1.06 mM L-glutamic acid. In some aspects, the basal media comprises about 1.09 mM L-glutamic acid. In some aspects, the basal media comprises about <0.10 mM L-glutamic acid.

In some aspects, the basal medium comprises a dipeptide. In some aspects, the basal medium comprises glutamine-glutamine (Gln-Gln). In some aspects, the basal medium comprises alanyl-glutamine (Ala-Gln).

In some aspects, the basal media comprises at least about 0.1 mM dipeptide (e.g., Ala-Gln). In some aspects, the basal media comprises about 0.1 mM to about 50 mM dipeptide (e.g., Ala-Gln). In some aspects, the basal media comprises about 0.1 mM to about 40 mM, about 0.1 mM to about 35 mM, about 0.1 mM to about 30 mM, about 0.1 mM to about 25 mM, about 0.1 mM to about 20 mM, about 1 mM to about 20 mM, about 2 mM to about 20 mM, about 3 mM to about 20 mM, about 4 mM to about 20 mM, about 5 mM to about 20 mM, about 6 mM to about 20 mM, about 7 mM to about 20 mM, about 8 mM to about 20 mM, about 9 mM to about 20 mM, about 10 mM to about 20 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, or about 9 mM to about 10 mM dipeptide (e.g., Ala-Gln).

In some aspects, the basal media comprises at least about 0.1 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 25 mM, at least about 30 mM, or at least about 50 mM dipeptide (e.g., Ala-Gln).

In certain aspects, the basal medium comprises about 1 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM dipeptide (e.g., Ala-Gln). In some aspects, the basal medium comprises about 1.7 mM dipeptide (e.g., Ala-Gln). In certain aspects, the basal medium comprises about 1.68 mM dipeptide (e.g., Ala-Gln).

In certain aspects, the basal medium comprises about 6 mM, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, or about 7.2 mM dipeptide (e.g., Ala-Gln). In some aspects, the basal medium comprises about 6.8 mM dipeptide (e.g., Ala-Gln). In some aspects, the basal medium comprises about 6.81 mM dipeptide (e.g., Ala-Gln). In some aspects, the basal medium comprises about 6.9 mM dipeptide (e.g., Ala-Gln). In some aspects, the basal medium comprises about 6.96 mM dipeptide (e.g., Ala-Gln). In certain aspects, the basal medium comprises about 7.0 mM dipeptide (e.g., Ala-Gln).

In certain aspects, the basal media comprises less than about 5 mM ammonia (NH₃). In some aspects, the basal media comprises less than about 4 mM, less than about 3.5 mM, less than about 3 mM, less than about 2.5 mM, less than about 2 mM, less than about 1.5 mM, less than about 1 mM, less than about 0.5 mM, less than about 0.4 mM, less than about 0.3 mM, less than about 0.2 mM, or less than about 0.1 mM ammonia. In certain aspects, the basal media comprises about 0.01 mM ammonia to less than about 2 mM ammonia, about 0.01 mM ammonia to less than about 1.9 mM ammonia, about 0.01 mM ammonia to less than about 1.8 mM ammonia, about 0.01 mM ammonia to less than about 1.7 mM ammonia, about 0.01 mM ammonia to less than about 1.6 mM ammonia, about 0.01 mM ammonia to less than about 1.5 mM ammonia, about 0.01 mM ammonia to less than about 1.4 mM ammonia, about 0.01 mM ammonia to less than about 1.3 mM ammonia, about 0.01 mM ammonia to less than about 1.2 mM ammonia, about 0.01 mM ammonia to less than about 1.1 mM ammonia, about 0.01 mM ammonia to less than about 1 mM ammonia, about 0.01 mM ammonia to less than about 0.9 mM ammonia, about 0.01 mM ammonia to less than about 0.8 mM ammonia, about 0.01 mM ammonia to less than about 0.7 mM ammonia, about 0.01 mM ammonia to less than about 0.6 mM ammonia, about 0.01 mM ammonia to less than about 0.5 mM ammonia, about 0.01 mM ammonia to less than about 0.4 mM ammonia, about 0.01 mM ammonia to less than about 0.3 mM ammonia, about 0.01 mM ammonia to less than about 0.2 mM ammonia, or about 0.01 mM ammonia to less than about 0.1 mM ammonia. In some aspects, the basal media comprises about 1.2 mM ammonia. In some aspects, the basal media comprises about 1.25 mM ammonia. In some aspects, the basal media comprises about 1.259 mM ammonia. In some aspects, the basal media comprises about 1.28 mM ammonia. In some aspects, the basal media comprises about 1.3 mM ammonia. In some aspects, the basal media comprises about 0.3 mM ammonia. In some aspects, the basal media comprises about 0.34 mM ammonia. In some aspects, the basal media comprises about 0.35 mM ammonia. In some aspects, the basal media comprises about 0.36 mM ammonia. In some aspects, the basal media comprises about 0.37 mM ammonia. In some aspects, the basal media comprises less than about 0.3 mM ammonia. In some aspects, the basal media comprises less than about 0.29 mM ammonia. In some aspects, the basal media comprises less than about 0.28 mM ammonia. In some aspects, the basal media comprises less than about 0.278 mM ammonia. In some aspects, the basal media does not comprise ammonia.

In some aspects, the basal media comprises lactate. In certain aspects, the basal media does not comprise lactate.

In some aspects, the basal media comprises potassium, e.g., the basal media comprises potassium prior to addition of potassium, as described herein. In some aspects, the basal media comprises less than about 10 mM potassium ion. In some aspects, the basal media comprises about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, or about 6.0 mM potassium ion. In certain aspects, the basal media comprises about 4 mM potassium ion.

In some aspects, the basal medium is selected from a balanced salt solution (e.g., PBS, DPBS, HBSS, EBSS), Dulbecco's Modified Eagle's Medium (DMEM), Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™ XF, AIM V™, TEXMACS™ medium, and any combination thereof. In some aspects, the basal media comprises PRIME-XV T cell CDM. In some aspects, the basal media comprises OPTMIZER™. In some aspects, the basal media comprises OPTMIZER™ Pro. In some aspects, the basal media comprises X-VIVO™ 15 (LONZA). In some aspects, the basal media comprises IMMUNOCULT™. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal media comprises OPTMIZER™ complete.

In some aspects, the media, e.g., the MRM, e.g., secondary TIL expansion medium and/or third (or final) TIL expansion medium, further comprises a CD3 agonist and/or a CD28 agonist. The CD3 agonist and/or the CD28 agonist can stimulate TILs that are being cultured in the media. In some aspects, a CD3 agonist can be any molecule that is capable of binding to CD3 complex and activating CD3. In some aspects, a CD3 agonist is a small molecule. In some aspects, a CD3 agonist is a protein. In some aspects, a CD3 agonist is an anti-CD3 antibody. The term “anti-CD3 antibody” as used herein refers to an antibody or variant thereof, e.g., a monoclonal antibody and including human, humanized, chimeric or murine antibodies which are directed against the CD3 complex in T cells. In some aspects, an anti-CD3 antibody comprises OKT-3, also known as muromonab, and UCHT-1. Other anti-CD3 antibodies include, for example, visilizumab, otelixizumab, and teplizumab.

The term “OKT-3” or “OKT3” refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof A hybridoma capable of producing OKT-3 is deposited with European Collection of Authenticated Cell Cultures (ECACC) and assigned Catalogue No. 86022706. A hybridoma capable of producing OKT-3 is also deposited with the American Type Culture Collection and assigned the ATCC accession number CRL 8001.

In some aspects, a CD28 agonist can be any molecule that is capable of activating CD28 or its downstream pathway. In some aspects, a CD28 agonist is a small molecule. In some aspects, a CD28 agonist is a protein. In some aspects, a CD28 agonist is an anti-CD28 antibody. The term “anti-CD28 antibody” as used herein refers to an antibody or variant thereof, e.g., a monoclonal antibody and including human, humanized, chimeric or murine antibodies which are directed against CD28 and activate T cells. In some aspects, an anti-CD28 antibody comprises Catalog #100182-1 (BPS Bioscience), Catalog #100186-1 (BPS Bioscience).

In some aspects, the CD3 agonist and the CD28 agonist are added in the media, e.g., the MRM, together. In some aspects, the CD3 agonist and the CD28 agonist are added in the media, e.g., the MRM, concurrently in one composition. In some aspects, the CD3 agonist and the CD28 agonist are added in sequence. In some aspects, the media, e.g., the MRM, e.g., secondary TIL expansion media and/or third (or final) TIL expansion media, comprises and/or is supplemented with a substituent comprising both a CD3 agonist and a CD28 agonist, e.g., TRANSACT™. In some aspects, the media, e.g., the MRM, comprises at least about 1:100 TRANSACT™. In some aspects, the media, e.g., the MRM, comprises at least about 1:150 TRANSACT™. In some aspects, the media, e.g., the MRM, comprises at least about 1:200 TRANSACT™. In some aspects, the media, e.g., the MRM, comprises at least about 1:250 TRANSACT™. In some aspects, the media, e.g., the MRM, comprises at least about 1:300 TRANSACT™. In some aspects, the media, e.g., the MRM, comprises at least about 1:350 TRANSACT™. In some aspects, the media, e.g., the MRM, comprises at least about 1:400 TRANSACT™. In some aspects, the media, e.g., the MRM, comprises at least about 1:450 TRANSACT™. In some aspects, the media, e.g., the MRM, comprises at least about 1:500 TRANSACT™.

In some aspects, the media, e.g., the MRM, e.g., secondary TIL expansion media and/or third (or final) TIL expansion media, comprises and/or is supplemented with a TRANSACT™ alternative. Artificial antigen presenting cells (aAPCs) such as genetically engineered human K562 aAPCs can be used for rapid expansion of TILs. In some aspects, the aAPC is generated by transducing K562 cells with a polycistronic lentiviral vector comprising genes encoding CD70, CD80, CD86, 41BB ligand, and OX40 ligand. K562 cells do not express HLA-A, HLA-B, or HLA-DR molecules, which makes them a powerful tool for T cell expansion when transduced with the above mentioned co-stimulatory ligands (See, e.g., Suhoski et al., Molecular therapy, 2007). In some aspects, secondary TIL expansion and/or third TIL expansion comprises co-culturing the TILs with aAPCs+OKT3. In some aspects, secondary TIL expansion and/or third TIL expansion comprises co-culturing the TILs with irradiated APCs (e.g., PBMC) in the presence of OKT3 (e.g., at least about 30 ng/mL OKT3) instead of TRANSACT™. In some aspects, the ratio of immune cells (e.g., TILs) to feeder cells (e.g., aAPCs) is at least about 1:50, at least about 1:100, at least about 1:150, at least about 1:200, at least about 1:250, at least about 1:300, at least about 1:350, at least about 1:400, at least about 1:450, or at least about 1:500. In some aspects, the ratio of immune cells (e.g., TILs) to feeder cells (e.g., aAPCs) is at least about 1:100. In some aspects, the ratio of immune cells (e.g., TILs) to feeder cells (e.g., aAPCs) is at least about 1:200.

In some aspects, the media, e.g., the MRM, e.g., secondary TIL expansion media and/or third (or final) TIL expansion media, comprise and/or are supplemented with a CD27 ligand CD27L). CD27 ligand CD70) is capable of binding to its receptor, and then upon binding, the receptor is capable of generating and long-term maintenance of T cell immunity. CD27 is a member of the TNF-receptor superfamily. CD27, a transmembrane homodimeric phosphoglycoprotein of 120 kDa, also appears to have a costimulatory role. CD27L, CD70, is a transmembrane glycoprotein expressed on T and B cells in response to antigen stimulation; it is thus considered a marker of the early stages of activation. In vitro, the interaction of CD27 on a T cell and CD70 on a B cell enhances T cell activation in terms of proliferation but only relatively low amounts of IL-2 are secreted. Studies of knockout mice have shown that CD27 plays a minor part in naive T cell activation but is crucial for the generation of T cell memory.

In some aspects, the media, e.g., the MRM, e.g., secondary TIL expansion media and/or third (or final) TIL expansion media, comprises about 0.1 μg/ml to about 50 μg/ml CD27L. In some aspects, the media, e.g., the MRM, comprises and/or is supplemented with about 0.1 μg/ml to about 40 μg/ml, about 0.1 μg/ml to about 30 μg/ml, about 0.1 μg/ml to about 20 μg/ml, about 0.1 μg/ml to about 10 μg/ml, about 0.1 μg/ml to about 5 μg/ml, about 1 μg/ml to about 10 μg/ml, about 2 μg/ml to about 10 μg/ml, about 3 μg/ml to about 10 μg/ml, about 4 μg/ml to about 10 μg/ml, about 5 μg/ml to about 10 μg/ml, about 1 μg/ml to about 9 μg/ml, about 1 μg/ml to about 8 μg/ml, about 1 μg/ml to about 7 μg/ml, about 1 μg/ml to about 6 μg/ml, about 1 μg/ml to about 5 μg/ml, about 3 μg/ml to about 7 μg/ml, about 4 μg/ml to about 6 μg/ml, about 3 μg/ml to about 6 μg/ml, or about 4 μg/ml to about 7 μg/ml CD27L.

In some aspects, the media, e.g., the MRM, e.g., secondary TIL expansion media and/or third (or final) TIL expansion media, comprises and/or is supplemented with at least about 0.1 μg/ml, at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 11 μg/ml, at least about 12 μg/ml, at least about 13 μg/ml, at least about 14 μg/ml, at least about 15 μg/ml, at least about 16 μg/ml, at least about 17 μg/ml, at least about 18 μg/ml, at least about 19 μg/ml, at least about 20 μg/ml, at least about 25 μg/ml, at least about 30 μg/ml, or at least about 50 μg/ml CD27L. In some aspects, the media, e.g., the MRM, comprises and/or is supplemented with at least about 5 μg/ml CD27L.

In some aspects, the media, e.g., the MRM, e.g., secondary TIL expansion medium and/or third (or final) TIL expansion medium, comprises and/or is supplemented with 4-1BB ligand (4-1BBL). 4-1BBL (4-1BB ligand, CD137L) is found on APCs (antigen presenting cells) and binds to 4-1BB (also known as CD137), a type 2 transmembrane glycoprotein receptor belonging to the TNF superfamily, which is expressed on activated T Lymphocytes. 4-1BB ligand can be used to activate T cells in vitro. In some aspects, the media, e.g., the MRM, e.g., secondary TIL expansion media and/or third (or final) TIL expansion media, comprise about 0.1 μg/ml to about 50 μg/ml CD27L. In some aspects, the media, e.g., the MRM, comprises and/or is supplemented with about 0.1 μg/ml to about 10 μg/ml, about 0.1 μg/ml to about 9 μg/ml, about 0.1 μg/ml to about 8 μg/ml, about 0.1 μg/ml to about 7 μg/ml, about 0.1 μg/ml to about 6 μg/ml, about 0.1 μg/ml to about 5 μg/ml, about 0.1 μg/ml to about 4 μg/ml, about 0.1 μg/ml to about 3 μg/ml, about 0.1 μg/ml to about 2 μg/ml, about 0.1 μg/ml to about 1 μg/ml, 1 μg/ml to about 10 μg/ml, about 1 μg/ml to about 5 μg/ml, about 1 μg/ml to about 4 μg/ml, about 1 μg/ml to about 3 μg/ml, or about 1 μg/ml to about 2 μg/ml 4-1BBL.

In some aspects, the media, e.g., the MRM, e.g., secondary TIL expansion media and/or third (or final) TIL expansion media, comprise and/or are supplemented with at least about 0.1 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1 μg/ml, at least about 1.1 μg/ml, at least about 1.2 μg/ml, at least about 1.3 μg/ml, at least about 1.4 μg/ml, at least about 1.5 μg/ml, at least about 1.6 μg/ml, at least about 1.7 μg/ml, at least about 1.8 μg/ml, at least about 1.9 μg/ml, at least about μg/ml, at least about 2 μg/ml, at least about 3 μg/ml, at least about 4 μg/ml, at least about 5 μg/ml, or at least about 10 μg/ml 4-1BBL. In some aspects, the media, e.g., the MRM, comprises and/or is supplemented with at least about 1 μg/ml 4-1BBL.

In some aspects, a 4-1BBL is added in the media, e.g., the MRM, together with a CD27L. In some aspects, a 4-1BBL is added in the media, e.g., the MRM, concurrently with a CD27L. In some aspects, a 4-1BBL is added in the media, e.g., the MRM, with a CD27L in sequence. In some aspects, the media, e.g., the MRM, used during an expansion process (e.g., a secondary expansion and/or a final expansion) comprises TRANSACT™, 4-1BBL, and CD27L. In some aspects, the media, e.g., the MRM, comprises at least about 1:100 TRANSACT™, at least about 1 μg/ml 4-1BBL, and at least about 5 μg/ml CD27L. In some aspects, the media, e.g., the MRM, used during an expansion process (e.g., a secondary expansion and/or a final expansion) comprises at least about 1:100 TRANSACT™, at least about 1 μg/ml 4-1BBL, and at least about 5 μg/ml CD27L.

In some aspects, the media, e.g., the MRM, e.g., initial TIL culture medium, secondary TIL expansion medium and/or third (or final) TIL expansion medium, is modified from a basal medium selected from a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™ XF, AIM V™, TEXMACS™ medium, and any combination thereof. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal media comprises OPTMIZER™ complete. In some aspects, suitable basal medium includes Click's medium, OPTIMIZER™ (CTS®) medium, STEMLINE® T cell expansion medium (Sigma-Aldrich), AIM V™ medium (CTS®), TEXMACS® medium (Miltenyi Biotech), IMMUNOCULT™ medium (Stem Cell Technologies), PRIME-XV® T-Cell Expansion XSFM (Irvine Scientific), Iscoves medium, and/or RPMI-1640 medium.

The present disclosure comprises a media, e.g., the MRM, comprising basal media, NaCl, KCl, calcium, and glucose, wherein the concentration of NaCl is between about 40 mM and about 80 mM, the concentration of KCl is between 40 and 90 mM, the concentration of calcium is between about 0.5 mM and about 2.8 mM, and the concentration of glucose between about 10 mM and about 24 mM.

In some aspects, the media, e.g., the MRM, further comprises immune cells. In some aspects, the immune cells comprises TILs.

In some aspects, the media, e.g., the MRM, further comprises IL-2, IL-7, IL-15, IL-21, or any combination thereof. In some aspects, the media, e.g., the MRM, further comprises IL-2 and IL-21. In some aspects, the concentration of IL-2 is about 200 ng/ml to about 400 ng/ml (e.g., about 200 ng/ml, about 300 ng/ml, or about 400 ng/ml). In some aspects, the concentration of IL-21 is about 20 ng/ml to about 40 ng/ml, (e.g., about 20 ng/ml, about 30 ng/ml, or about 40 ng/ml).

In some aspects, the media, e.g., the MRM, further comprises about 2.5% serum supplement (CTS™ Immune Cell SR, Thermo Fisher), 2 mM L-glutamine, 2 mM L-glutamax, MEM Non-Essential Amino Acids Solution, Pen-strep, 20 μg/ml FUNGIN™, sodium pyruvate, or any combination thereof. In some aspects, the media, e.g., the MRM, further comprises O-Acetyl-L-carnitine hydrochloride. In some aspects, the media, e.g., the MRM, further comprises a kinase inhibitor.

In some aspects, the media, e.g., the MRM, further comprises a CD3 agonist. In some aspects, the CD3 agonist is an anti-CD3 antibody. In some aspects, the anti-CD3 antibody comprises OKT-3.

In some aspects, the media, e.g., the MRM, further comprises a CD28 agonist. In some aspects, the CD28 agonist is an anti-CD28 antibody. In some aspects, the media, e.g., the MRM, further comprises a CD27 ligand CD27L). In some aspects, the media, e.g., the MRM, further comprises a 4-1BB ligand (4-1BBL).

In some aspects, the present disclosure includes a cell culture comprising the media, e.g., the MRM, disclosed herein, a cell bag comprising the media, e.g., the MRM, disclosed herein, or a bioreactor comprising the media, e.g., the MRM, disclosed herein.

II.H. Cells

Some aspects of the present disclosure are directed to methods of culturing cells, e.g., immune cells (e.g., T cells and/or NK cells), comprising placing the cells in a hypotonic or isotonic medium comprising potassium ion at a concentration of at least about 5 mM, as disclosed herein. Certain aspects of the present disclosure are directed to methods of culturing cells, e.g., immune cells (e.g., T cells and/or NK cells), comprising placing the cells in a medium comprising potassium ion at a concentration higher than 40 mM, as disclosed herein. Certain aspects of the present disclosure are directed to methods of culturing cells, e.g., immune cells (e.g., T cells and/or NK cells), comprising placing the cells in a medium comprising potassium ion at a concentration of at least about 50 mM, as disclosed herein. Some aspects of the present disclosure are directed to methods of culturing cells, e.g., immune cells (e.g., T cells and/or NK cells), comprising placing the cells in a medium comprising potassium ion at a concentration of at least about 40 mM to at least about 90 mM, as disclosed herein.

Some aspects of the present disclosure are directed to methods of culturing cells (e.g., human immune cells and/or stem cells disclosed herein), comprising placing the cells in a medium comprising potassium ion at a concentration of at least about 40 mM and NaCl at a concentration of less than 100 mM, as disclosed herein. Certain aspects of the present disclosure are directed to methods of culturing cells (e.g., human immune cells and/or stem cells disclosed herein), comprising placing the cells in a medium comprising potassium ion at a concentration of at least about 50 mM and NaCl at a concentration of less than 90 mM, as disclosed herein.

The cells that are placed in the medium can be cells that are collected and/or isolated from a subject in need of a therapy. In some aspects, the cells that are placed in the medium have been engineered prior to the culturing. In some aspects, the cells that are placed in the medium have been expanded. In some aspects, the TILs that are placed in the medium have been expanded prior to being placed in a media, e.g., an MRM, disclosed herein. The cells that are placed in the medium can be referred to as starting (initial, i.e., patient sample, apheresis sample, buffy coat) cells. The cells that result from culturing them in the media disclosed herein can be referred to as resulting (cultured) cells.

The methods disclosed herein provide culture conditions that promote a less-differentiated phenotype for cultured pluripotent or multipotent cells. As such, the methods and media disclosed herein are useful for the culture of multipotent (e.g., pluripotent) cell type. In some aspects, the cell culturable using the methods disclosed herein is a multipotent cell. In some aspects, the cells are pluripotent cells. In certain aspects, the pluripotent cells comprise embryonic stem cells (ESCs). In certain aspects, the pluripotent cells comprise induced pluripotent stem cells (iPSCs). In some aspects, the cells are hematopoietic stem cells.

In certain aspects, the cells are immune cells. In some aspects, the starting immune cells are isolated from a human subject. In some aspects, the starting immune cells are isolated from a human subject for allogeneic cell therapy. In some aspects, the starting immune cells are isolated from a human subject for autologous cell therapy. In some aspects, the cells are T cells. In some aspects, the cells are NK cells. In some aspects, the cells are TILs. In some aspects, the cells are Tregs. In some aspects, the cells, e.g., T cells and/or NK cells, are isolated from a human subject. In some aspects, the immune cells are tumor-infiltrating T cells or tumor-infiltrating NK cells. In certain aspects, the cells, e.g., T cells and/or NK cells, are engineered. In some aspects, the immune cells, e.g., T cells and/or NK cells, are engineered to comprise a chimeric antigen receptor (CAR). In some aspects, the immune cells, e.g., T cells and/or NK cells, are engineered to comprise an engineered T cell receptor (TCR).

In some aspects, the cells, e.g., T cells and/or NK cells, are engineered before culturing according to the methods disclosed herein. In some aspects, the cells, e.g., T cells and/or NK cells, are engineered after culturing according to the methods disclosed herein. In some aspects, the cells, e.g., T cells and/or NK cells, are cultured according to the methods disclosed herein, e.g., in a hypotonic or isotonic medium comprising at least 50 mM potassium ion, prior to, during, and after cell engineering. In some aspects, the cells, e.g., T cells and/or NK cells, are engineered to express a chimeric antigen receptor (CAR). In some aspects, the cells, e.g., T cells and/or NK cells, are engineered to express an engineered T cell receptor (TCR). In certain aspects, culturing the cells, e.g., T cells and/or NK cells, under the conditions disclosed herein, e.g., in a hypotonic or isotonic medium comprising at least about 50 mM potassium ion, results in higher transduction efficiency. In some aspects, transduction efficiency is at least about 2-fold greater in cells, e.g., T cells and/or NK cells, cultured in hypotonic or isotonic medium comprising at least about 60 mM potassium ion, according to the methods disclosed herein, as compared to cells, e.g., T cells and/or NK cells, cultured in medium comprising 4 mM potassium ion or less. In some aspects, transduction efficiency is at least about 2.5-fold greater in cells, e.g., T cells and/or NK cells, cultured in hypotonic or isotonic medium comprising at least about 65 mM potassium ion, according to the methods disclosed herein, as compared to cells, e.g., T cells and/or NK cells, cultured in medium comprising 4 mM potassium ion or less.

In some aspects, the cell comprises a construct expressing an antigen receptor and/or another additional polypeptide. In some aspects, the antigen receptor comprises an antibody, an engineered antibody such as scFv, a CAR, an engineered TCR, a TCR mimic (e.g., an antibody-T cell receptor (abTCR) or a chimeric antibody-T cell receptor (caTCR)), or a chimeric signaling receptor (CSR). By way of example, a TCR can comprise an engineered TCR in which the antigen-binding domain of a TCR (e.g., an alpha/beta TCR or a gamma/delta TCR) has been replaced by that of an antibody (with or without the antibody's constant domains); the engineered TCR then becomes specific for the antibody's antigen while retaining the TCR's signaling functions. A chimeric signaling receptor can comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ). See, e.g., EP340793B1, WO 2017/070608, WO 2018/200582, WO 2018/200583, WO 2018/200585, and Xu et al., Cell Discovery (2018) 4:62.

In some aspects, the antigen receptor targets an antigen of interest (e.g., a tumor antigen or an antigen of a pathogen). The antigens can include, without limitation, AFP (alpha-fetoprotein), avβ6 or another integrin, BCMA, B7-H3, B7-H6, Braf, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein a), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In certain aspects, the antigen receptor targets hTERT. In some aspects, the antigen receptor targets KRAS. In some aspects, the antigen receptor targets Braf. In some aspects, the antigen receptor targets TGFβRII. In some aspects, the antigen receptor targets MAGE A10/A4. In some aspects, the antigen receptor targets AFP. In some aspects, the antigen receptor targets PRAME. In some aspects, the antigen receptor targets MAGE A1. In some aspects, the antigen receptor targets WT-1. In some aspects, the antigen receptor targets NY-ESO. In some aspects, the antigen receptor targets PRAME. In some aspects, the antigen receptor targets NY-ESO. In some aspects, the antigen receptor targets CD19.

In some aspects, the antigen receptor targets BCMA. In some aspects, the antigen receptor targets CD147. In some aspects, the antigen receptor targets CD19. In some aspects, the antigen receptor targets CD19 and CD22. In some aspects, the antigen receptor targets CD19 and CD28. In some aspects, the antigen receptor targets CD20. In some aspects, the antigen receptor targets CD20 and CD19. In some aspects, the antigen receptor targets CD22. In some aspects, the antigen receptor targets CD30. In some aspects, the antigen receptor targets CEA. In some aspects, the antigen receptor targets DLL3. In some aspects, the antigen receptor targets EGFRvIII. In some aspects, the antigen receptor targets GD2. In some aspects, the antigen receptor targets HER2. In some aspects, the antigen receptor targets IL-IRAP. In some aspects, the antigen receptor targets mesothelin. In some aspects, the antigen receptor targets methothelin. In some aspects, the antigen receptor targets NKG2D. In some aspects, the antigen receptor targets PSMA. In some aspects, the antigen receptor targets TnMUC1.

In some aspects, the immune cells are TILs. In some aspects, the TILs are present in a tumor sample obtained from a subject. Accordingly, in some aspects, the method comprises placing a tumor sample into an MRM disclosed herein. During standard TIL culture, tumor samples, e.g., a tumor biopsy or a fragment thereof, is plated in an initial TIL culture medium, and cultured for at least about 14-19 days. In some aspects, the tumor sample, e.g., the tumor biopsy, is cultured in an MRM in an initial TIL culture for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 day. In some aspects, the initial TIL culture lasts about 14 days. In some aspects the initial TIL culture lasts sufficient number of days until a cell yield of about 2×10⁶ to about 10×10⁶ cells are produced.

In some aspects, the proportion of CD8⁺ TILs to non-CD8⁺ TILs (e.g., the proportion of CD8⁺ TILs to CD4⁺ TILs) is increased following the initial TIL culture, as compared to the proportion of CD8⁺ TILs to non-CD8⁺ TILs prior to the initial TIL culture. In some aspects, the proportion of CD8⁺ TILs to non-CD8⁺ TILs (e.g., the proportion of CD8⁺TILs to CD4⁺ TILs) is increased following the initial TIL culture, as compared to the proportion of CD8⁺ TILs to non-CD8⁺ TILs following an initial TIL culture in a basal medium or a medium that does not comprise an increased concentration of potassium ion (control medium). In some aspects, the proportion of CD8⁺ TILs is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 45-fold, or at least about 50-fold. In some aspects, the proportion of CD8⁺ TILs is increased by at least about 40-fold. In some aspects, the proportion of CD8⁺ TILs is increased by at least about 50-fold.

In some aspects, the proportion of CD8⁺ TILs is increased by at least about 20%, at least about 25%, at least about 30%, at least about 35%, at least about 40%, at least about 45%, at least about 50%, at least about 60%, at least about 70%, at least about 75%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500%. In some aspects, the proportion of CD8⁺ TILs is increased by at least about 20%. In some aspects, the proportion of CD8⁺ TILs is increased by at least about 40%. In some aspects, the proportion of CD8⁺ TILs is increased by at least about 60%. In some aspects, the proportion of CD8⁺ TILs is increased by at least about 80%. In some aspects, the proportion of CD8⁺ TILs is increased by at least about 100%.

In some aspects, the proportion of CD8⁺ TILs is increased to at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total number of TILs in the culture. In some aspects, the proportion of CD8⁺ TILs is increased to at least about 20% of the total number of TILs in the culture. In some aspects, the proportion of CD8⁺ TILs is increased to at least about 30% of the total number of TILs in the culture. In some aspects, the proportion of CD8⁺ TILs is increased to at least about 40% of the total number of TTLs in the culture. In some aspects, the proportion of CD8⁺ TTLs is increased to at least about 50% of the total number of TILs in the culture. In some aspects, the proportion of CD8⁺ TTLs is increased to at least about 60% of the total number of TILs in the culture. In some aspects, the proportion of CD8⁺ TILs is increased to at least about 70% of the total number of TILs in the culture. In some aspects, the proportion of CD8⁺ TILs is increased to at least about 75% of the total number of TILs in the culture. In some aspects, the proportion of CD8⁺ TTLs is increased to at least about 80% of the total number of TILs in the culture. In some aspects, the proportion of CD8⁺ TTLs is increased to at least about 90% of the total number of TTLs in the culture.

In some aspects, the tumor sample is isolated from a human subject. In some aspects, the starting tumor sample isolated from a human subject, and the TILs therein are expanded for an allogeneic cell therapy. In some aspects, the tumor sample is isolated from a human subject, and the TILs therein are expanded for an autologous cell therapy.

II.H.1. Chimeric Antigen Receptor (CAR)

In some aspects, the cell, e.g., T cell and/or NK cell, comprises a CAR. In some aspects, the cell that can be prepared to express a CAR (e.g., a CAR T cell) is, e.g., a CD8⁺ T cell or CD4+ T cell. In some aspects, a CAR-expressing cell disclosed herein is a CAR T cell, e.g., a mono CAR T cell, a genome-edited CAR T cell, a dual CAR T cell, or a tandem CAR T cell. Examples of such CAR T cells are provided in International Application No. PCT/US2019/044195.

In some aspects, the CAR specifically binds (i.e., target) one or more antigens expressed on a tumor cell, such as a malignant B cell, a malignant T cell, or a malignant plasma cell.

In some aspects, the CAR specifically binds to (i.e., targets) an antigen selected from the group consisting of CD19, TRAC, TCR(3, BCMA, CLL-1, CS1, CD38, CD19, TSHR, CD123, CD22, CD30, CD 70, CD171, CD33, EGFRvIII, GD2, GD3, Tn Ag, PSMA, ROR1, ROR2, GPC1, GPC2, FLT3, FAP, TAG72, CD44v6, CEA, EPCAM, B37113, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, ECFR, NCAM, prostase, PAP, ELF2M, Ephrin B2, IGF-1 receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysilica acid, PLAC1, GloboH, NY-R-1, UPK2, HAVCR1, ADRB3, PANX3, GPR20, LY6K, OR51E2, TARP, WTI, NY-ESO-1, LAGE-la, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant hERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, or any combinations thereof.

In some aspects, the CAR targets BCMA. In some aspects, the CAR targets CD147. In some aspects, the CAR targets CD19. In some aspects, the CAR targets ROR1. In some aspects, the CAR targets GPC3. In some aspects, the CAR targets GPC2. In some aspects, the CAR targets CD19 and CD22. In some aspects, the CAR targets CD19 and CD28. In some aspects, the CAR targets CD20. In some aspects, the CAR targets CD20 and CD19. In some aspects, the CAR targets CD22. In some aspects, the CAR targets CD30. In some aspects, the CAR targets CEA. In some aspects, the CAR targets DLL3. In some aspects, the CAR targets EGFRvIII. In some aspects, the CAR targets GD2. In some aspects, the CAR targets HER2. In some aspects, the CAR targets IL-1RAP. In some aspects, the CAR targets mesothelin. In some aspects, the CAR targets methothelin. In some aspects, the CAR targets NKG2D. In some aspects, the CAR targets PSMA. In some aspects, the CAR targets TnMUC1.

In certain aspects, the CAR comprises an antigen-binding domain that specifically binds an antigen in complex with an MHC. In some aspects, the CAR comprises an antigen-binding domain from a TCRm, e.g., any TCRm disclosed herein.

II.H.2. T Cell Receptor-Engineered (TCR) Cells

In some aspects, an immune cell, e.g., a T cell and/or an NK cell, disclosed herein comprises a T cell receptor (TCR), e.g., an engineered TCR. In some aspects, the TCR specifically binds to a tumor antigen. As used herein, the term “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells and/or NK cells. In some aspects, the TCR specifically binds a neoantigen identified from a cancer patient.

In some aspects, the TCR specifically binds (i.e., target) one or more antigens expressed on a tumor cell, such as a malignant B cell, a malignant T cell, or a malignant plasma cell.

In certain aspects, an engineered cell of the present disclosure can express a T cell receptor (TCR) targeting an antigen. In some aspects, the TCR engineered cells can target main types: shared tumor-associated antigens (shared TAAs) and unique tumor-associated antigens (unique TAAs), or tumor-specific antigens. The former can include, without any limitation, cancer-testis (CT) antigens, overexpressed antigens, and differentiation antigens, while the latter can include, without any limitation, neoantigens and oncoviral antigens. Human papillomavirus (HPV) E6 protein and HPV E7 protein belong to the category of oncoviral antigens.

In some aspects, the TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the TCR engineered cells can target glycoprotein (gp100), melanoma antigen recognized by T cells (MART-1), and/or tyrosinase, which are mainly found in melanomas and normal melanocytes. In some aspects, the TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the TCR engineered cells can target mesothelin, another kind of overexpressed antigen that is highly expressed in mesothelioma but is also present on mesothelial cells of several tissues, including trachea.

In some aspects, the TCR engineered cells can target any neoantigen, which can be formed by random somatic mutations specific to individual tumors. In some aspects, the TCR specifically binds to (i.e., targets) a cancer antigen selected from the group consisting of AFP, Braf, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2 1FLT3, FAP, TAG72, CD44v6, CEA, EPCAM, B71D, KIT, IL-13Ra2, mesothelin, IL-1 IRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MACE A1, ETV6-AML, sperm protein 17, XAGE1, Tie 2, MAD-CT-1, MAD-CT 2, Fos-related anigen 1, p53, p53 mutant, prostein, surviving, telomerase, PCTA-1/Galectin 8, MelanA/MNARTI, Ras mutant, hTERT, sarcoma translocation breakpoints, ML_IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AK AP-4, SSX2, RAG-1, human telomerase reverse transcriptase, RU1, RU2, intestinal carboxyl esterase, mut hsp70-2, CD79a, CD79b, CD72, LAIR1, FCAR, LILRA2, CD300L-F, CLEC12A, BST2, EMR-2, LY75, GPC3, FCRL5, IGLL1, CD2, CD3ε, CD4, CD5, CD7, the extracellular portion of the APRIL protein, or any combinations thereof,

In certain aspects, the TCR specifically binds (i.e., targets) hTERT. In some aspects, the TCR specifically binds (i.e., targets) KRAS. In some aspects, the TCR specifically binds (i.e., targets) Braf In some aspects, the TCR specifically binds (i.e., targets) TGFβRII. In some aspects, the TCR specifically binds (i.e., targets) MAGE A10/A4. In some aspects, the TCR specifically binds (i.e., targets) AFP. In some aspects, the TCR specifically binds (i.e., targets) PRAME. In some aspects, the TCR specifically binds (i.e., targets) MAGE A1. In some aspects, the TCR specifically binds (i.e., targets) WT-1. In some aspects, the TCR specifically binds (i.e., targets) NY-ESO. In some aspects, the TCR specifically binds (i.e., targets) PRAME. In some aspects, the TCR specifically binds (i.e., targets) NY-ESO. In some aspects, the TCR specifically binds (i.e., targets) CD19. In certain aspects, the TCR specifically binds a neoantigen identified from a cancer patient.

In some aspects, the TCR comprises an intracellular gamma/delta domain. In some aspects, the TCR is an antibody-T-cell receptor (AbTCR) (see, e.g., Xu et al., Cell Discovery 4:62 (2018), which is incorporated by reference herein in its entirety.

II.H.3. T Cell Receptor Mimics (TCRm)

In some aspects, an immune cell, e.g., a T cell and/or an NK cell, disclosed herein comprises a T cell receptor mimic (TCRm), also known as a TCR-like antibody. TCRm are a type of antibody that recognize epitopes comprising both the peptide and the MHC-I molecule, similar to the recognition of such complexes by the TCR on T cells (see, e.g., Traneska et al., Front. Immunol. 8(1001):1-12 (2017), which is incorporated by reference herein in its entirety). In some aspects, the TCRm specifically binds to a tumor antigen. In certain aspects, the TCRm specifically binds a neoantigen identified from a cancer patient.

In some aspects, the TCRm specifically binds (i.e., target) one or more antigens expressed on a tumor cell, such as a malignant B cell, a malignant T cell, or a malignant plasma cell. In some aspects, the TCRm is a monoclonal antibody. In some aspects, the TCRm specifically binds to WT1. In some aspects, the TCRm specifically binds to a fragment of WT1. In some aspects, the TCRm comprises ESK1 (see, e.g., Ataie et al., J. Mol. Biol. 428(1):194-205 (2016), which is incorporated by reference herein in its entirety). In some aspects, the TCRm specifically binds to MAGE-A1. In some aspects, the TCRm specifically binds to p68 RNA helicase/HLA-A*02:01. In some aspects, the TCRm specifically binds to hCG-b/HLA-A*02:01. In some aspects, the TCRm specifically binds to Her2-E75/HLA-A*02:01. In some aspects, the TCRm specifically binds to PR-1 in context of HLA-A*02:01 (see, e.g., Oncoimmunology 5(1):e1049803 (June 2015), which is incorporated by reference herein in its entirety). In some aspects, the TCRm specifically binds to the survivin-2B-derived nonamer peptide, AYACNTSTL (SV2B80-88), presented on HLA-A*24 (SV2B80-88/HLA-A*24) (see, e.g., Kurosawa et al., Nature Scientific Reports 9(9827):1-11 (2019), which is incorporated by reference herein in its entirety). In some aspects, the TCRm specifically binds one or more tumor-associated PRAME peptide/HLA-I antigens (see, e.g., J Clin Invest. 127(7):2705-18 (2017), which is incorporated by reference herein in its entirety). In some aspects, the TCRm specifically binds to tyrosinase. In some aspects, the TCRm specifically binds telomerase catalytic subunit. In some aspects, the TCRm specifically binds to glycoprotein 100 (gp100). In some aspects, the TCRm specifically binds to mucin 1 (MUC1). In some aspects, the TCRm specifically binds to human telomerase reverse transcriptase (hTERT). In some aspects, the TCRm specifically binds to NYESO-1. In some aspects, the TCRm specifically binds to MART-1. In some aspects, the TCRm specifically binds to PRAME.

In some aspects, the TCRm specifically binds to a viral antigen. In some aspects, the TCRm specifically binds to Env183/A2 (Hep B/HLA-A*02:01). In some aspects, the TCRm specifically binds to KP14/1 and KP15/11 (HIV envelope gp160/HLAA*02:01). In some aspects, the TCRm specifically binds to RL36A (West Nile Virus/mouse H-2db). In some aspects, the TCRm specifically binds to a viral epitope derived from HTLV. In some aspects, the TCRm specifically binds to a viral epitope derived from influenza. In some aspects, the TCRm specifically binds to a viral epitope derived from CMV. In some aspects, the TCRm specifically binds to a viral epitope derived from HIV.

II.I. TIL Isolation, Expansion, and Harvest

Any method of TIL isolation, culture, and/or expansion can be modified according to the methods disclosed herein, e.g., by culturing and/or expanding the TILs in a culture medium described herein.

In general, TILs are obtained from a tumor sample obtained from a human subject. Any methods for obtaining a tumor biopsy from a subject can be used in the methods disclosed herein, so long as the tumor sample contains a mixture of tumor and TILs. In some aspects, the tumor sample is isolated through a tumor resection. In some aspects, the tumor sample is isolated by a needle biopsy. In some aspects, the tumor sample comprises a solid tumor, including a primary tumor, invasive tumor or metastatic tumor. In other aspects, the tumor sample comprises a liquid tumor, such as a tumor obtained from a hematological malignancy. The tumor may be of any cancer type, including, but not limited to, breast, pancreatic, prostate, colorectal, cervical, lung, brain, renal, stomach, liver (including but not limited to hepatocellular carcinoma) and skin (including but not limited to squamous cell carcinoma, basal cell carcinoma, and melanoma). In some aspects, the tumor sample is cryopreserved prior to TIL isolation/expansion. In some aspects the tumor sample is fresh, e.g., not cryopreserved. In some aspects, the tumor sample is placed directly into MRM media.

In some aspects, the donor patient (e.g., the subject from which the tumor is obtained) is treatment naive (i.e., the patient has not received a prior therapy for the treatment of the tumor). In some aspects, the donor patient has received one or more prior therapy for the treatment of the tumor. In some aspects, the subject has received at least one prior therapy, at least two prior therapies, at least three prior therapies, or at least four prior therapies. In some aspects, the subject is relapsed or refractory to one or more prior therapy.

In some aspects, the subject has received one or more prior anticancer therapy. In some aspects, the prior anticancer therapy comprises a standard of care therapy. In some aspects, the prior anticancer therapy comprises an immunotherapy. In some aspects, the prior therapy comprises an immunotherapy comprising a checkpoint inhibitor. In some aspects, the prior therapy comprises an immunotherapy comprising an anti-PD-1 antibody, an anti-CTLA-4 antibody, an anti-LAG-3 antibody, or any combination thereof.

In some aspects, the subject is administered one or more therapy that enhances the isolation and/or expansion of TILs prior to resection of the tumor sample. In some aspects, the subject is administered a kinase inhibitor or an ITK inhibitor. Examples of kinase inhibitors and/or ITK inhibitors can be found, for example, in Int'l Publication No. WO2019217753, which is incorporated by reference herein in its entirety. In some aspects, the kinase inhibitor and/or the ITK inhibitor is added to the culture medium during the initial expansion and/or the second expansion. In some aspects, the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket. In some aspects, the ITK inhibitor is selected from the group consisting of ibrutinib, dasatinib, bosutinib, nilotinib, erlotinib, BMS509744, CTA056, GSK2250665A, PF06465469, and any combination thereof.

In some aspects, the tumor sample is cut into smaller fragments. In some aspects, the one or more of the smaller fragments is at least about 1 mm², at least about 1.5 mm², at least about 2 mm², at least about 2.5 mm², at least about 3 mm², at least about 3.5 mm², at least about 4 mm², at least about 4.5 mm², at least about 5 mm², at least about 5.5 mm², at least about 6 mm², or at least about 6.5 mm². In some aspects, the one or more of the smaller fragments is at least about 1 mm³, at least about 1.5 mm³, at least about 2 mm³, at least about 2.5 mm³, at least about 3 mm³, at least about 3.5 mm³, at least about 4 mm³, at least about 4.5 mm³, at least about 5 mm³, at least about 5.5 mm³, at least about 6 mm³, at least about 6.5 mm³, at least about 7 mm³, at least about 7.5 mm³, at least about 8 mm³, at least about 8.5 mm³, at least about 9 mm³, at least about 9.5 mm³, or at least about 10 mm³. In some aspects, the tumor samples are subjected to an enzymatic digest, by culturing the tumor samples in an enzymatic media (e.g., RPMI 1640 buffer supplemented with glutamate (e.g., about 2 mM), gentamicine (e.g., about 10 mcg/mL), DNase (e.g., about 30 units/mL), and collagenase (e.g., about 1.0 mg/mL)). In some aspects, the tumor digests are produced by placing the tumor in the enzymatic media and mechanically dissociating (i.e., disaggregating) the tumor (e.g., for about 1 minute), followed by incubation at 370 C. in 5% CO₂ (e.g., for 30 minutes), followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide can be performed to remove these cells.

The mechanical and/or enzymatic dissociation can be performed in any medium. In some aspects, the mechanical and/or enzymatic dissociation is performed in an MRM medium disclosed herein.

In some aspects, the mechanical dissociation comprises applying a physical pressure to the resected tumor. In some aspects, the mechanical dissociation comprises repeated physical pressure. In some aspects, the repeated physical pressure is applied at least about 50 times, at least about 60 times, at least about 70 times, at least about 80 times, at least about 90 times, at least about 100 times, at least about 110 times, at least about 120 times, at least about 130 times, at least about 140 times, at least about 150 times, at least about 160 times, at least about 170 times, at least about 180 times, at least about 190 times, at least about 200 times, at least about 210 times, at least about 220 times, at least about 230 times, at least about 240 times, at least about 250 times, at least about 260 times, at least about 270 times, at least about 280 times, at least about 290 times, at least about 300 times, at least about 310 times, at least about 320 times, at least about 330 times, at least about 340 times, at least about 350 times, or at least about 360 times per minute. In some aspects, the repeated physical pressure is applied at least about 120 to 260 times per minute. In some aspects, the repeated physical pressure is applied up to about 6 N/cm², up to about 5.5 N/cm², up to about 5.0 N/cm², up to about 4.5 N/cm², up to about 4.0 N/cm², up to about 3.5 N/cm², up to about 3.0 N/cm². In some aspects, the mechanical dissociation proceeds for about 90 minutes or less, about 85 minutes or less, about 80 minutes or less, about 75 minutes or less, about 70 minutes or less, about 65 minutes or less, about 60 minutes or less, about 55 minutes or less, or about 50 minutes or less. In some aspects, the mechanical dissociation is applied at room temperature. In some aspects, the mechanical dissociation is applied at less than room temperature. In some aspects, the mechanical dissociation is applied according to the methods disclosed in and/or using a device disclosed in Int'l Publication No. WO 2021/123832, which is incorporated by reference herein in its entirety.

In some aspects, the tumor sample (i.e., the resected tumor tissue sample or the dissociated tumor sample) or the fragments thereof is placed into a culture medium, e.g., a culture medium disclosed herein, wherein the culture medium further comprises IL-2. In some aspects, the culture medium comprises at least about 4000 IU/ml IL-2, at least about 4500 IU/ml IL-2, at least about 5500 IU/ml IL-2, at least about 6000 IU/ml IL-2, or at least about 6500 IU/ml IL-2. In some aspects, the culture medium comprises at least about 600 IU/ml IL-2. In some aspects, the culture medium comprises at least about 100 ng/mL IL-2. In some aspects, the culture medium comprises at least about 200 ng/mL IL-2. In some aspects, the culture medium comprises at least about 300 ng/mL IL-2. In some aspects, the culture medium comprises at least about 400 ng/mL IL-2. In some aspects, the culture medium comprises at least about 500 ng/mL IL-2. In some aspects, the culture medium comprises at least about 600 ng/mL IL-2.

In some aspects, the culture medium comprises at least about 1500 IU/mL IL-2. In some aspects, the culture medium comprises from about 1500 IU/mL to about 12,000 IU/mL IL-2. In some aspects, the culture medium comprises at least about 1500 IU/mL, at least about 1600 IU/mL, at least about 1700 IU/mL, at least about 1800 IU/mL, at least about 1900 IU/mL, at least about 2000 IU/mL, at least about 2100 IU/mL, at least about 2200 IU/mL, at least about 2300 IU/mL, at least about 2400 IU/mL, at least about 2500 IU/mL, at least about 2600 IU/mL, at least about 2700 IU/mL, at least about 2800 IU/mL, at least about 2900 IU/mL, at least about 3000 IU/mL, at least about 3100 IU/mL, at least about 3200 IU/mL, at least about 3300 IU/mL, at least about 3400 IU/mL, at least about 3500 IU/mL, at least about 3600 IU/mL, at least about 3700 IU/mL, at least about 3800 IU/mL, at least about 3900 IU/mL, at least about 4000 IU/mL, at least about 4100 IU/mL, at least about 4200 IU/mL, at least about 4300 IU/mL, at least about 4400 IU/mL, at least about 4500 IU/mL, at least about 4600 IU/mL, at least about 4700 IU/mL, at least about 4800 IU/mL, at least about 4900 IU/mL, at least about 5000 IU/mL, at least about 5100 IU/mL, at least about 5200 IU/mL, at least about 5300 IU/mL, at least about 5400 IU/mL, at least about 5500 IU/mL, at least about 5600 IU/mL, at least about 5700 IU/mL, at least about 5800 IU/mL, at least about 5900 IU/mL, at least about 6000 IU/mL, at least about 6100 IU/mL, at least about 6200 IU/mL, at least about 6300 IU/mL, at least about 6400 IU/mL, at least about 6500 IU/mL, at least about 6600 IU/mL, at least about 6700 IU/mL, at least about 6800 IU/mL, at least about 6900 IU/mL, at least about 7000 IU/mL IL-2, at least about 7100 IU/mL, at least about 7200 IU/mL, at least about 7300 IU/mL, at least about 7400 IU/mL, at least about 7500 IU/mL, at least about 7600 IU/mL, at least about 7700 IU/mL, at least about 7800 IU/mL, at least about 7900 IU/mL, or at least about 8000 IU/mL IL-2. In some aspects, the culture medium comprises at least about 3000 IU/mL IL-2. In some aspects, TILs are cultured in culture medium during a second culture (e.g., REP culture), as described herein, wherein the culture medium comprises about 3000 IU/mL. In some aspects, the culture medium comprises at least about 6000 IU/mL IL-2. In some aspects, TILs are cultured in culture medium during an initial culture, as described herein, wherein the culture medium comprises about 6000 IU/mL.

In other aspects, the tumor sample or the fragments thereof is placed into a culture medium, e.g., a culture medium disclosed herein, wherein the culture medium further comprises IL-21. In some aspects, the culture medium comprises at least about 1.0 ng/mL IL-21. In some aspects, the culture medium comprises at least about 2.0 ng/mL IL-21. In some aspects, the culture medium comprises at least about 3.0 ng/mL IL-21. In some aspects, the culture medium comprises at least about 4.0 ng/mL IL-21. In some aspects, the culture medium comprises at least about 5.0 ng/mL IL-21. In some aspects, the culture medium comprises at least about 6.0 ng/mL IL-21. In some aspects, the culture medium comprises at least about 7.0 ng/mL IL-21. In some aspects, the culture medium comprises at least about 8.0 ng/mL IL-21. In some aspects, the culture medium comprises at least about 9.0 ng/mL IL-21. In some aspects, the culture medium comprises at least about 10 ng/mL IL-21. In some aspects, the culture medium comprises at least about 15 ng/mL IL-21. In some aspects, the culture medium comprises at least about 20 ng/mL IL-21. In some aspects, the culture medium comprises at least about 30 ng/mL IL-21.

Individual tumor fragments can be cultured together in a single culture chamber, e.g., well, or individual tumor fragments can be cultured in separate culture chambers, e.g., wells. A standard culture medium for promoting TIL evasion from cultured tumor samples comprises RPMI 1640 supplemented with Glutamax (Gibco/Invitrogen; Carlsbad, Calif), 1×Pen-Strep (Gibco/Invitrogen; Carlsbad, Calif.), 50 μM 2-mercaptoethanol (Gibco/Invitrogen; Carlsbad, Calif.), 20 μg/ml Gentamicin (Gibco/Invitrogen; Carlsbad, Calif.), and 1 mM pyruvate (Gibco/Invitrogen; Carlsbad, Calif.). In some aspects, a standard culture medium is modified according to the present disclosure. In some aspects, a standard culture medium comprises CTS™ OPTMIZER™ supplemented with serum supplement (CTS™ Immune Cell SR, Thermo Fisher), L-glutamine (Gibco), L-glutamax (Gibco), MEM Non-Essential Amino Acids Solution (Gibco), Pen-strep (Gibco), FUNGIN™ (InvivoGen), Sodium pyruvate (Gibco), IL-2, IL-21, of O-Acetyl-L-carnitine hydrochloride (Sigma), and any combination thereof. In some aspects, a standard culture medium comprises CTS™ OPTMIZER™ supplemented with about 2.5% serum supplement (CTS™ Immune Cell SR, Thermo Fisher), about 2 mM L-glutamine (Gibco), about 2 mM L-glutamax (Gibco), MEM Non-Essential Amino Acids Solution (Gibco), Pen-strep (Gibco), about 20 μg/ml FUNGIN™ (InvivoGen), Sodium pyruvate (Gibco), about IL-2 (300 ng/mL), about IL-21 (30 ng/ml), and about 1 mM of O-Acetyl-L-carnitine hydrochloride (Sigma).

In some aspects, tumor samples or fragments thereof are cultured in an initial culture for at least about 1 week, at least about 2 weeks, or at least about 3 weeks. In some aspects, tumor samples or fragments thereof are cultured for at least about 2 weeks. As used herein, “tumor samples” refers to tumor tissue and/or disaggregated tumor tissue (i.e., a cell suspension resulting from mechanical and/or chemical disaggregation of tumor tissue). In some aspects, the tumor samples or fragments are cultured in an initial culture for about 7 days, about 8 days, about 9 days, about 10 days, about 11 days, about 12 days, about 13 days, or about 14 days.

In some aspects, the initial culture further comprises contacting the tumor samples or fragments with a tumor necrosis factor receptor superfamily (TNFRSF) agonist. In some aspects, the TNFRSF agonist comprises a 4-1BB agonist, an OX40 agonist, a CD27 agonist, a GITR agonist, a HVEM agonist, a CD95 agonist, or any combination thereof. In some aspects, the TNFRSF agonist is any TNFRSF agonist disclosed in U.S. Publication No. US 2020/0121719 A1, which is incorporated by reference herein in its entirety. In some aspects, the initial culture further comprises contacting the tumor samples or fragments thereof with about 10-500 ng/ml 4-1BB ligand. In some aspects, initial culture further comprises contacting the tumor samples or fragments thereof with about 50 ng/ml, about 60 ng/ml, about 70 ng/ml, about 75 ng/ml, about 80 ng/ml, about 90 ng/ml, about 100 ng/ml, about 125 ng/ml, about 150 ng/ml, about 175 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, about 1000 ng/ml, or about 1100 ng/ml 4-1BB ligand. In some aspects, initial culture further comprises contacting the tumor samples or fragments thereof with about 100 ng/ml 4-1BB ligand. In some aspects, the tumor samples or fragments thereof are contacted with the 4-1BB ligand on about day 3 of the initial culture, on about day 4 of the initial culture, on about day 5 of the initial culture, on about day 6 of the initial culture, or on about day 7 of the initial culture. In some aspects, the tumor samples or fragments thereof are contacted with the 4-1BB ligand on about day 5 of the initial culture.

In some aspects, the initial culture further comprises contacting the tumor samples or fragments thereof with TRANSACT™. In some aspects, initial culture further comprises contacting the tumor samples or fragments thereof with TRANSACT™ (e.g., about 1:50, about 1:100, about 1:150, about 1:200, about 1:250, about 1:300, about 1:350, or about 1:400). In some aspects, the tumor samples or fragments thereof are contacted with the TRANSACT™ on about day 4 of the initial culture, on about day 5 of the initial culture, on about day 6 of the initial culture, or on about day 7 of the initial culture. In some aspects, the tumor samples or fragments thereof are contacted with the TRANSACT™ on about day 5 of the initial culture. In some aspects, the initial culture further comprises contacting the tumor samples or fragments thereof with both 4-1BB ligand and TRANSACT™. In some aspects, the tumor samples or fragments thereof are contacted with both 4-1BB ligand and TRANSACT™ on about day 3 of the initial culture. In some aspects, the tumor samples or fragments thereof are contacted with both 4-1BB ligand and TRANSACT™ on about day 4 of the initial culture. In some aspects, the tumor samples or fragments thereof are contacted with both 4-1BB ligand and TRANSACT™ on about day 5 of the initial culture. In some aspects, the tumor samples or fragments thereof are contacted with both 4-1BB ligand and TRANSACT™ on about day 6 of the initial culture. In some aspects, the tumor samples or fragments thereof are contacted with both 4-1BB ligand and TRANSACT™ on about day 7 of the initial culture. In some aspects, the tumor samples or fragments thereof are contacted with both 4-1BB ligand and TRANSACT™ on about day 8 of the initial culture.

In some aspects, tumor samples or fragments thereof are cultured in an initial culture until cell yield in the initial culture reaches at least about 1×10⁵ to at least about 1×10⁸, at least about 5×10⁵ to at least about 1×10⁸, at least about 1×10⁶ to at least about 1×10⁸, at least about 2×10⁶ to at least about 1×10⁸, at least about 3×10⁶ to at least about 1×10⁸, at least about 4×10⁶ to at least about 1×10⁸, at least about 5×10⁶ to at least about 1×10⁸, at least about 1×10⁵ to at least about 5×10⁷, at least about 5×10⁵ to at least about 10×10⁶, at least about 1×10⁶ to at least about 10×10⁶, at least about 2×10⁶ to at least about 10×10⁶, at least about 3×10⁶ to at least about 10×10⁶, at least about 4×10⁶ to at least about 10×10⁶, or at least about 5×10⁶ to at least about 10×10⁶ cells per cultured fragment. In some aspects, tumor samples or fragments thereof are cultured in an initial culture until cell yield in the initial culture reaches at least about 2×10⁶-10×10⁶ cells per fragment.

In some aspects, tumor samples or fragments thereof are cultured in an initial culture until cell yield in the initial culture reaches at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶ cells per fragment. In some aspects, tumor samples or fragments thereof are cultured in an initial culture until cell yield in the initial culture reaches at least about 2×10⁶ cells per fragment. In some aspects, tumor samples or fragments thereof are cultured in an initial culture until cell yield in the initial culture reaches at least about 3×10⁶ cells per fragment. In some aspects, tumor samples or fragments thereof are cultured in an initial culture until cell yield in the initial culture reaches at least about 4×10⁶ cells per fragment. In some aspects, tumor samples or fragments thereof are cultured in an initial culture until cell yield in the initial culture reaches at least about 5×10⁶ cells per fragment. In some aspects, tumor samples or fragments thereof are cultured in an initial culture until cell yield in the initial culture reaches at least about 6×10⁶ cells per fragment. In some aspects, tumor samples or fragments thereof are cultured in an initial culture until cell yield in the initial culture reaches at least about 7×10⁶ cells per fragment. In some aspects, tumor samples or fragments thereof are cultured in an initial culture until cell yield in the initial culture reaches at least about 8×10⁶ cells per fragment. In some aspects, tumor samples or fragments thereof are cultured in an initial culture until cell yield in the initial culture reaches at least about 9×10⁶ cells per fragment. In some aspects, tumor samples or fragments thereof are cultured in an initial culture until cell yield in the initial culture reaches at least about 10×10⁶ cells per fragment. In some aspects, the cells (e.g., TILs) are passed through a strainer following the initial culture. In some aspects, the cells (e.g., TILs) are passed through an at least about 10 μm, an at least about 15 μm, an at least about 20 μm, an at least about 25 μm, an at least about 30 μm, an at least about 35 μm, an at least about 40 μm, an at least about 45 μm, an at least about 50 μm strainer following the initial culture. In some aspects, the cells (e.g., TILs) are passed through an about 40 μm strainer following the initial culture.

In some aspects, the TILs are subjected to a secondary expansion. In some aspects, the secondary expansion step is carried out in one or more gas permeable flasks (e.g., GREX flasks). In some aspects, the TILs are transitioned to the secondary expansion without opening the closed system. In some aspects, the TILs from the first expansion are screened for tumor-specific cytolytic activity prior to advancing the TILs to the secondary expansion. In some aspects, the TILs are screened for expression of one or more biomarkers prior to advancing to secondary expansion. In some aspects, the biomarker comprises expression of one or more gene typically expressed by more naive TILs, e.g., CD8⁺, CD27⁺, CD3⁺, CD95⁺, CD45RA⁺, CCR7⁺, CD62L⁺, TCF7⁺, or any combination thereof. In some aspects, the TILs are screened for expression of PD-1 prior to advancing to secondary expansion. In some aspects, the TILs from the first expansion are not screened prior to advancing the TILs to the secondary expansion. In some aspects, all TILs obtained in the initial expansion are subjected to the secondary expansion. In some aspects, the TILs from the first expansion are pooled prior to advancement to secondary expansion.

In some aspects, the TILs are subjected to a secondary expansion using a Rapid Expansion Protocol (REP). See, e.g., Dudley, et al., Science 298:850-54 (2002); Dudley, et al., J. Clin. Oncol. 23:2346-57 (2005); Dudley, et al., J. Clin. Oncol. 26:5233-39 (2008); Riddell, et al., Science 257:238-41 (1992); and Dudley, et al., J. Immunother. 26:332-42 (2003), each of which is incorporated by reference herein in its entirety. In some aspects, TILs are rapidly expanded using non-specific T-cell receptor stimulation in the presence of feeder lymphocytes and interleukin-2 (IL-2), IL-7, IL-15, IL-21, or combinations thereof. In certain aspects, the TTLs are rapidly expanded in the presence of IL-2, IL-15, and IL-21. In some aspects, the concentration of IL-2 in the media during rapid expansion is lower than the concentration of IL-2 in the media during the initial culture. In some aspects, the concentration of IL-2 during rapid expansion is less than 300 ng/ml. In some aspects, the concentration of IL-2 during rapid expansion is about 50 ng/ml, about 55 ng/ml, about 60 ng/ml, about 65 ng/ml, about 70 ng/ml, about 73.6 ng/ml, about 75 ng/ml, about 80 ng/ml, about 85 ng/ml, about 90 ng/ml, about 95 ng/ml, about 100 ng/ml, about 10⁵ ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 175 ng/ml, about 200 ng/ml, about 225 ng/ml, about 250 ng/ml, or about 275 ng/ml. In some aspects, the concentration of IL-2 during rapid expansion is about 50 ng/ml. In some aspects, the concentration of IL-2 during rapid expansion is about 55 ng/ml. In some aspects, the concentration of IL-2 during rapid expansion is about 60 ng/ml. In some aspects, the concentration of IL-2 during rapid expansion is about 65 ng/ml. In some aspects, the concentration of IL-2 during rapid expansion is about 70 ng/ml. In some aspects, the concentration of IL-2 during rapid expansion is about 73.6 ng/ml. In some aspects, the concentration of IL-2 during rapid expansion is about 75 ng/ml. In some aspects, the concentration of IL-2 during rapid expansion is about 80 ng/ml. In some aspects, the concentration of IL-2 during rapid expansion is about 85 ng/ml. In some aspects, the concentration of IL-2 during rapid expansion is about 90 ng/ml. In some aspects, the concentration of IL-2 during rapid expansion is about 95 ng/ml. In some aspects, the concentration of IL-2 during rapid expansion is about 100 ng/ml.

In some aspects, the concentration of IL-21 in the media during rapid expansion is lower than the concentration of IL-21 in the media during the initial culture. In some aspects, the concentration of IL-21 during rapid expansion is less than 30 ng/ml. In some aspects, the concentration of IL-21 during rapid expansion is about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, or about 29 ng/ml. In some aspects, the concentration of IL-21 during rapid expansion is about 5 ng/ml. In some aspects, the concentration of IL-21 during rapid expansion is about 6 ng/ml. In some aspects, the concentration of IL-21 during rapid expansion is about 7 ng/ml. In some aspects, the concentration of IL-21 during rapid expansion is about 8 ng/ml. In some aspects, the concentration of IL-21 during rapid expansion is about 9 ng/ml. In some aspects, the concentration of IL-21 during rapid expansion is about 10 ng/ml. In some aspects, the concentration of IL-21 during rapid expansion is about 11 ng/ml. In some aspects, the concentration of IL-21 during rapid expansion is about 12 ng/ml. In some aspects, the concentration of IL-21 during rapid expansion is about 13 ng/ml. In some aspects, the concentration of IL-21 during rapid expansion is about 14 ng/ml. In some aspects, the concentration of IL-21 during rapid expansion is about 15 ng/ml.

In some aspects, the concentration of IL-15 in the media during rapid expansion is about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml, about 1.5 ng/ml, about 1.6 ng/ml, about 1.7 ng/ml, about 1.8 ng/ml, about 1.9 ng/ml, about 2.0 ng/ml, about 2.25 ng/ml, about 2.5 ng/ml, about 2.75 ng/ml, about 3.0 ng/ml, about 3.5 ng/ml, about 4.0 ng/ml, about 4.5 ng/ml, or about 5.0 ng/ml. In some aspects, the concentration of IL-15 during rapid expansion is about 0.1 ng/ml. In some aspects, the concentration of IL-15 during rapid expansion is about 0.2 ng/ml. In some aspects, the concentration of IL-15 during rapid expansion is about 0.3 ng/ml. In some aspects, the concentration of IL-15 during rapid expansion is about 0.4 ng/ml. In some aspects, the concentration of IL-15 during rapid expansion is about 0.5 ng/ml. In some aspects, the concentration of IL-15 during rapid expansion is about 0.6 ng/ml. In some aspects, the concentration of IL-15 during rapid expansion is about 0.7 ng/ml. In some aspects, the concentration of IL-15 during rapid expansion is about 0.8 ng/ml. In some aspects, the concentration of IL-15 during rapid expansion is about 0.9 ng/ml. In some aspects, the concentration of IL-15 during rapid expansion is about 1.0 ng/ml.

The non-specific T-cell receptor stimulus can include, e.g., OKT3 (e.g., about 30 ng/ml), a mouse monoclonal anti-CD3 antibody (available from Ortho-McNeil®, Raritan, N.J. or Miltenyi Biotec, Bergisch Gladbach, Germany). In some aspects, TILs are rapidly expanded by stimulation of peripheral blood mononuclear cells (PBMC) in vitro with one or more antigens (including antigenic portions thereof, such as epitope(s), or a cell of the cancer, which can be optionally expressed from a vector, such as an human leukocyte antigen A2 (HLA-A2) binding peptide, e.g., approximately 0.3 μM MART-1:26-35 (27 L) or gp100:209-217 (210M)), in the presence of a T-cell growth factor, such as around 200-400 IU/ml of a T-cell growth factor, such as 300 IU/ml IL-2 or IL-15. In some aspects, TILs are expanded by stimulation using TRANSACT™. In some aspects, the in vitro-induced TILs are rapidly expanded by stimulation with the same antigen(s) of the cancer pulsed onto HLA-A2-expressing antigen-presenting cells. In some aspects, the TILs can be stimulated with irradiated, autologous lymphocytes or with irradiated HLA-A2⁺ allogeneic lymphocytes and IL-2.

In some aspects, the TILs are stimulated during the second expansion by culturing the cells in a medium comprising TRANSACT™ and optionally 4-1BBL and/or CD27L. In some aspects, the TILs are stimulated during the second expansion by culturing the cells in a medium comprising TRANSACT™, 4-1BBL, and CD27L. In some aspects, the TILs are stimulated during the second expansion by culturing the cells in a medium comprising at least about 1:100 TRANSACT™, at least about 1 μg/ml 4-1BBL, and at least about 5 μg/ml CD27L.

In some aspects, one or more TILs are genetically modified before, during, or after TIL expansion. Genetic modification of the TILs can be achieved using any methods known in the art. In some aspects, one or more TILs are modified using a Cas9 endonuclease (CRISPR; see, e.g., US2017067021A1, which is incorporated by reference herein in its entirety), TALEN, a zing-finger endonuclease, site directed mutagenesis, or any combination thereof. In some aspects, one or more TILs are genetically modified to disrupt or ablate expression of human cytokine inducible SH2-containing protein (CISH; see, e.g., U.S. Ser. No. 10/406,177B2, which is incorporated by reference herein in its entirety). In some aspects, one or more TILs is modified using an AAV, e.g., one or more of the TILs comprise an AAV. In some aspects one or more TILs is modified using a lentivirus or a retrovirus. In some aspects, one or more TILs are genetically modified to express an exogenous modified or engineered T cell receptor (TCR). In some aspects, one or more TILs are genetically modified to express chimeric antigen receptor (CAR). In some aspects, one or more TILs are genetically modified to express CD86. In some aspects, one or more TILs are genetically modified to express OX40L. In some aspects, one or more TILs are genetically modified to express 4-1BBL. In some aspects, one or more TILs are genetically modified to express an anti-PD1 antibody.

In some aspects, the TILs are expanded in a culture medium that further comprises a tumor necrosis factor receptor superfamily (TNFRSF) agonist. Any TNFRSF agonist can be used in the methods disclosed herein. Non-limiting examples of TNFRSF agonists can be found, for example, in US20200121719A1, which is incorporated by reference herein in its entirety. In some aspects, the TNFRSF agonist is added after the initial culture. In some aspects, the TNFRSF agonist is added during the second and/or or final expansion.

In some aspects, the TILs are expanded in a culture medium that further comprises a 4-1BB agonist. Any 4-1BB agonist can be used in the methods disclosed herein. In some aspects, the 4-1BB agonist comprises a 4-1BB antibody. Non-limiting examples of 4-1BB agonists can be found, for example, in US20200032209A1, which is incorporated by reference herein in its entirety. In some aspects, the 4-1BB agonist is added after the initial culture. In some aspects, the 4-1BB agonist is added during the second or final expansion.

In some aspects, the TILs are stimulated during the second expansion by culturing the cells in a medium comprising TRANSACT™ and optionally 4-1BBL and/or CD27L. In some aspects, the TILs are stimulated during the second expansion by culturing the cells in a medium comprising TRANSACT™, 4-1BBL, and CD27L. In some aspects, the TILs are stimulated during the second expansion by culturing the cells in a medium comprising at least about 1:100 TRANSACT™, at least about 1 μg/ml 4-1BBL, and at least about 5 μg/ml CD27L.

In some aspects, the TILs are expanded in a culture medium that further comprises an adenosine a2a receptor antagonist. Any adenosine a2a receptor antagonist can be used in the methods disclosed herein. Non-limiting examples of adenosine a2a receptor antagonist can be found, for example, in US20210137930A1, which is incorporated by reference herein in its entirety. In some aspects, the adenosine a2a receptor antagonist is selected from the group consisting of vipadenant, CPI-444 (ciforadenant), SCH58261, ZM241385, SCH420814, SYN115, 8-CSC, KW-6002, A2A receptor antagonist 1, ADZ4635, ST4206, KF21213, SCH412348, and 7MMG-49, or pharmaceutically acceptable salts, solvates, hydrates, cocrystals, or prodrugs thereof, and combinations thereof. In some aspects, the adenosine a2a receptor antagonist is added during the initial culture. In some aspects, the adenosine a2a receptor antagonist is added during the second and/or or final expansion.

In some aspects, the TILs are expanded in a culture medium that further comprises an AKT pathway inhibitor (AKTi). Any AKTi can be used in the methods disclosed herein. Non-limiting examples of AKTi that can be used in the present disclosure can be found, for example, in WO2020096927, which is incorporated by reference herein in its entirety. In some aspects, the AKTi is selected from the group consisting of afuresertib, uprosertib, ipatasertib, AT7867, AT13148, and pharmaceutically acceptable salts, solvates, hydrates, cocrystals, or prodrugs thereof. In some aspects, the AKTi is an mTOR inhibitor, e.g., AZD8055 or pharmaceutically acceptable salts, solvates, hydrates, cocrystals, or prodrugs thereof. In some aspects, the AKTi is an PI3K inhibitor, e.g., LY294002 or pharmaceutically acceptable salts, solvates, hydrates, cocrystals, or prodrugs thereof. In some aspects, the AKTi is added during the initial culture. In some aspects, the AKTi is added during the second and/or or final expansion.

In some aspects, the expanded cells are reactivated or stimulated by contacting the expanded TILs with one or more antigen presenting cell. Any antigen presenting cell can be used in the methods disclosed herein. In some aspects, the antigen presenting cell is a genetically modified cell. In some aspects, the antigen presenting cell comprises a tumor antigen or a fragment thereof on the cell surface. In some aspects, the expanded TILs are contacted with antigen presenting cells which comprises more than one tumor antigen or a fragment thereof on the cell surface.

In some aspects, the antigen presenting cell (APC) is genetically engineered. In some aspects, the APC is genetically engineered for tunable expression of one or more transgene, e.g., an antigen or a stimulatory signal. In some aspects, the APC is genetically engineered according to a method disclosed in WO2020/086742, which is incorporated by reference herein in its entirety. In some aspects, the APC is genetically engineered to express one or more stimulatory molecule. In some aspects, the APC is genetically engineered to express CD86, OC40L, 4-1BBL, or any combination thereof. In some aspects, the APC is an APC disclosed in U.S. Pat. No. 10,415,015, which is incorporated by reference herein in its entirety.

In some aspects, the TILs are cultured in a secondary TIL media until cell yield in the secondary expansion reaches at least about 1×10⁷ to at least about 50×10⁷, at least about 2×10⁷ to at least about 40×10⁷, at least about 3×10⁷ to at least about 30×10⁷, at least about 4×10⁷ to at least about 25×10⁷, at least about 5×10⁷ to at least about 20×10⁷, at least about 1×10⁷ to at least about 20×10⁷, at least about 2×10⁷ to at least about 20×10⁷, at least about 3×10⁷ to at least about 20×10⁷, or at least about 4×10⁷ to at least about 20×10⁷ cells. In some aspects, the TILs are cultured in a secondary TIL media until cell yield in the secondary expansion reaches at least about 5×10⁷ to at least about 20×10⁷ cells. In some aspects, the TILs are cultured in a secondary TIL media until cell yield in the secondary expansion reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells. In some aspects, the TILs are cultured in a secondary TIL media until cell yield in the secondary expansion reaches at least about 5×10⁷ cells. In some aspects, the TILs are cultured in a secondary TIL media until cell yield in the secondary expansion reaches at least about 6×10⁷ cells. In some aspects, the TILs are cultured in a secondary TIL media until cell yield in the secondary expansion reaches at least about 7×10⁷ cells. In some aspects, the TILs are cultured in a secondary expansion until cell yield in the secondary TIL media reaches at least about 8×10⁷ cells. In some aspects, the TILs are cultured in a secondary expansion until cell yield in the secondary TIL media reaches at least about 9×10⁷ cells. In some aspects, the TILs are cultured in a secondary TIL media until cell yield in the secondary expansion reaches at least about 10×10⁷ cells. In some aspects, the TILs are cultured in a secondary TIL media until cell yield in the secondary expansion reaches at least about 15×10⁷ cells. In some aspects, the TILs are cultured in a secondary TIL media until cell yield in the secondary expansion reaches at least about 20×10⁷ cells.

In some aspects, TILs are subjected to a final expansion. In some aspects, the media during final expansion comprises IL-2, IL-7, IL-15, IL-21, or combinations thereof. In certain aspects, the media during final expansion comprises IL-2, IL-15, and IL-21. In some aspects, the concentration of IL-2 in the media during final expansion is lower than the concentration of IL-2 in the media during the initial culture. In some aspects, the concentration of IL-2 during final expansion is less than 300 ng/ml. In some aspects, the concentration of IL-2 during final expansion is about 50 ng/ml, about 55 ng/ml, about 60 ng/ml, about 65 ng/ml, about 70 ng/ml, about 73.6 ng/ml, about 75 ng/ml, about 80 ng/ml, about 85 ng/ml, about 90 ng/ml, about 95 ng/ml, about 100 ng/ml, about 10⁵ ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 175 ng/ml, about 200 ng/ml, about 225 ng/ml, about 250 ng/ml, or about 275 ng/ml. In some aspects, the concentration of IL-2 during final expansion is about 50 ng/ml. In some aspects, the concentration of IL-2 during final expansion is about 55 ng/ml. In some aspects, the concentration of IL-2 during final expansion is about 60 ng/ml. In some aspects, the concentration of IL-2 during final expansion is about 65 ng/ml. In some aspects, the concentration of IL-2 during final expansion is about 70 ng/ml. In some aspects, the concentration of IL-2 during final expansion is about 73.6 ng/ml. In some aspects, the concentration of IL-2 during final expansion is about 75 ng/ml. In some aspects, the concentration of IL-2 during final expansion is about 80 ng/ml. In some aspects, the concentration of IL-2 during final expansion is about 85 ng/ml. In some aspects, the concentration of IL-2 during final expansion is about 90 ng/ml. In some aspects, the concentration of IL-2 during final expansion is about 95 ng/ml. In some aspects, the concentration of IL-2 during final expansion is about 100 ng/ml.

In some aspects, the concentration of IL-21 in the media during final expansion is lower than the concentration of IL-21 in the media during the initial culture. In some aspects, the concentration of IL-21 during final expansion is less than 30 ng/ml. In some aspects, the concentration of IL-21 during final expansion is about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, or about 29 ng/ml. In some aspects, the concentration of IL-21 during final expansion is about 5 ng/ml. In some aspects, the concentration of IL-21 during final expansion is about 6 ng/ml. In some aspects, the concentration of IL-21 during final expansion is about 7 ng/ml. In some aspects, the concentration of IL-21 during final expansion is about 8 ng/ml. In some aspects, the concentration of IL-21 during final expansion is about 9 ng/ml. In some aspects, the concentration of IL-21 during final expansion is about 10 ng/ml. In some aspects, the concentration of IL-21 during final expansion is about 11 ng/ml. In some aspects, the concentration of IL-21 during final expansion is about 12 ng/ml. In some aspects, the concentration of IL-21 during final expansion is about 13 ng/ml. In some aspects, the concentration of IL-21 during final expansion is about 14 ng/ml. In some aspects, the concentration of IL-21 during final expansion is about 15 ng/ml.

In some aspects, the concentration of IL-15 in the media during final expansion is about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml, about 1.5 ng/ml, about 1.6 ng/ml, about 1.7 ng/ml, about 1.8 ng/ml, about 1.9 ng/ml, about 2.0 ng/ml, about 2.25 ng/ml, about 2.5 ng/ml, about 2.75 ng/ml, about 3.0 ng/ml, about 3.5 ng/ml, about 4.0 ng/ml, about 4.5 ng/ml, or about 5.0 ng/ml. In some aspects, the concentration of IL-15 during final expansion is about 0.1 ng/ml. In some aspects, the concentration of IL-15 during final expansion is about 0.2 ng/ml. In some aspects, the concentration of IL-15 during final expansion is about 0.3 ng/ml. In some aspects, the concentration of IL-15 during final expansion is about 0.4 ng/ml. In some aspects, the concentration of IL-15 during final expansion is about 0.5 ng/ml. In some aspects, the concentration of IL-15 during final expansion is about 0.6 ng/ml. In some aspects, the concentration of IL-15 during final expansion is about 0.7 ng/ml. In some aspects, the concentration of IL-15 during final expansion is about 0.8 ng/ml. In some aspects, the concentration of IL-15 during final expansion is about 0.9 ng/ml. In some aspects, the concentration of IL-15 during final expansion is about 1.0 ng/ml.

In some aspects, the final expansion comprises a stimulation. In some aspects the stimulation is the same as the stimulation used during the secondary expansion. In some aspects, the TILs are stimulated during the final expansion by culturing the cells in an MRM comprising TRANSACT™, 4-1BBL, CD27L, or any combination thereof. In some aspects, the TILs are stimulated during the final expansion by culturing the cells in an MRM comprising TRANSACT™ and optionally 4-1BBL and/or CD27L. In some aspects, the TILs are stimulated during the final expansion by culturing the cells in an MRM comprising at least about 1:100 TRANSACT™, at least about 1 μg/ml 4-1BBL, and at least about 5 μg/ml CD27L.

In some aspects, the final expansion step is carried out in static GREX. In some aspects, the final expansion is carried out in a stirred tank. In some aspects the final expansion step is carried out in a bioreactor. In some aspects, the final expansion is continued until the cell yield in the final TIL media reaches at least about 40×10⁹ to at least about 100×10⁹, at least about 40×10⁹ to at least about 90×10⁹, at least about 40×10⁹ to at least about 80×10⁹, at least about 40×10⁹ to at least about 70×10⁹, at least about 40×10⁹ to at least about 60×10⁹, at least about 40×10⁹ to at least about 50×10⁹, at least about 10×10⁹ to at least about 100×10⁹, at least about 20×10⁹ to at least about 100×10⁹, at least about 30×10⁹ to at least about 100×10⁹, at least about 30×10⁹ to at least about 50×10⁹, or at least about 35×10⁹ to at least about 45×10⁹ cells. In some aspects, the final expansion is continued until the cell yield in the final TIL media reaches at least about 40×10⁹ to at least about 100×10⁹ cells. In some aspects, the final expansion is continued until the cell yield in the final TIL media reaches at least about 40×10⁹, at least about 45×10⁹, at least about 50×10⁹, at least about 55×10⁹, at least about 60×10⁹, at least about 65×10⁹, at least about 70×10⁹, at least about 75×10⁹, at least about 80×10⁹, at least about 85×10⁹, at least about 90×10⁹, at least about 95×10⁹, or at least about 100×10⁹ cells. In some aspects, the final expansion is continued until the cell yield in the final TIL media reaches at least about 40×10⁹ cells. In some aspects, the final expansion is continued until the cell yield in the final TIL media reaches at least about 50×10⁹ cells. In some aspects, the final expansion is continued until the cell yield in the final TIL media reaches at least about 60×10⁹ cells. In some aspects, the final expansion is continued until the cell yield in the final TIL media reaches at least about 70×10⁹ cells. In some aspects, the final expansion is continued until the cell yield in the final TIL media reaches at least about 80×10⁹ cells. In some aspects, the final expansion is continued until the cell yield in the final TIL media reaches at least about 90×10⁹ cells. In some aspects, the final expansion is continued until the cell yield in the final TIL media reaches at least about 100×10⁹ cells.

In some aspects, the final expansion is continued until the cell yield in the final TIL media for at least about 7 to at least about 21 days. In some aspects, the final expansion is continued until the cell yield in the final TIL media for at least about 7 days. In some aspects, the final expansion is continued until the cell yield in the final TIL media for at least about 8 days. In some aspects, the final expansion is continued until the cell yield in the final TIL media for at least about 9 days. In some aspects, the final expansion is continued until the cell yield in the final TIL media for at least about 10 days. In some aspects, the final expansion is continued until the cell yield in the final TIL media for at least about 11 days. In some aspects, the final expansion is continued until the cell yield in the final TIL media for at least about 12 days. In some aspects, the final expansion is continued until the cell yield in the final TIL media for at least about 13 days. In some aspects, the final expansion is continued until the cell yield in the final TIL media for at least about 14 days. In some aspects, the final expansion is continued until the cell yield in the final TIL media for at least about 15 days. In some aspects, the final expansion is continued until the cell yield in the final TIL media for at least about 16 days. In some aspects, the final expansion is continued until the cell yield in the final TIL media for at least about 17 days. In some aspects, the final expansion is continued until the cell yield in the final TIL media for at least about 18 days. In some aspects, the final expansion is continued until the cell yield in the final TIL media for at least about 19 days. In some aspects, the final expansion is continued until the cell yield in the final TIL media for at least about 20 days. In some aspects, the final expansion is continued until the cell yield in the final TIL media for at least about 21 days.

In some aspects, the expanded TILs are harvested. TILs can be harvested using any method, including by centrifugation. In some aspects, TILs are harvest using an automated system. Cell harvesters and/or cell processing systems are commercially available from a variety of sources, and any cell-based harvester can be used in the methods disclosed herein. In some aspects, the cell harvester and/or cell processing systems is a membrane-based cell harvester. In some aspects, the cell harvesting is conducted using a cell processing system, e.g., the LOVO system (Fresenius Kabi). In some aspects, the cell harvester and/or cell processing system can perform cell separation, washing, fluid-exchange, concentration, and/or other cell processing steps in a closed, sterile system.

In some aspects, the harvest is performed from a closed system bioreactor. In some aspects, a closed system is employed for the TIL expansion. In some aspects, a single bioreactor is employed. In some aspects, the closed system bioreactor is a single bioreactor. Examples of methods of expanding TILs ex vivo in open and closed systems can be found, for example, in U.S. Pat. No. 10,166,257, which is incorporated by reference herein in its entirety.

In some aspects, the expanded TILs are cryopreserved. The TILs can be cryopreserved using any methods. Various methods of cryopreserving mammalian cells, including TILs, have been described, e.g., by (i) General Protocol for the Cryopreservation of Mammalian Cells, UNC (2007), available at unclineberger.org/tissueculture/protocols/general-protocol-for-the-cryopreservation-of-mammalian-cells/; and (ii) Clarke et al., Improved post-thaw recovery of peripheral blood stem/progenitor cells using a novel intracellular-like cryopreservation solution, Cytotherapy 2009-6-6, available at sigmaaldrich.com/catalog/papers/19499402; each of which is incorporated by reference herein in its entirety.

In some aspects, the TILs are cultured according to the following:

(1) Tumor samples are isolated from a subject, and tumors are cut into fragments and/or mechanically or chemically disaggregated.
(2) The resulting tumor samples or fragments thereof are then cultured in an initial culture comprising a metabolic reprogramming media disclosed herein further supplemented with 300 ng/mL or 6000 IU/ml IL-2 and 30 ng/ml IL-21.
(3) Optionally, on day 5 following the start of the initial culture, the TILs are contacted with TRANSACT™ (1:200) and 100 ng/mL 4-1BB ligand, and the TILs are then cultured for an additional 5-9 days or until about 10×10⁶ to about 200×10⁶ cells are reached. TILs are then pooled.
(4) At least 0.5×10⁶ TILs from step 3 are then mixed with 100-200 times excess of irradiated PBMC feeder cells and cultured in media (e.g., a metabolic reprogramming media disclosed herein) supplemented with 30 ng/ml anti-CD3 antibody (e.g., OKT3), 75 ng/mL IL-2, 10 ng/mL IL-21, and 0.4 ng/mL IL-15. This secondary (REP) culture is continued until a therapeutically effective amount of TILs is obtained, as described herein.

III. Compositions of the Disclosure

III.A. Cell Culture Medium

Some aspects of the present disclosure are directed to a hypotonic or isotonic cell culture medium comprising at least about 5 mM potassium ion, as disclosed herein. Certain aspects of the present disclosure are directed to a cell culture medium comprising higher than 40 mM potassium ion, as disclosed herein. Certain aspects of the present disclosure are directed to a cell culture medium comprising at least about 50 mM potassium ion, as disclosed herein. In some aspects, the medium is hypotonic. In some aspects, the medium is isotonic. In some aspects, the medium is a metabolic reprograming medium.

Some aspects of the present disclosure are directed to a cell culture medium comprising at least about 50 mM potassium ion and less than 90 mM NaCl, as disclosed herein. In some aspects, the total concentration of potassium ion and NaCl is at least 110 mM. In some aspects, the medium is a metabolic reprograming medium.

In some aspects, the medium comprises about 40 mM potassium ion, and the medium has a tonicity of about 250 mOsmol. In some aspects, the medium comprises about 41 mM potassium ion, and the medium has a tonicity of about 250 mOsmol. In some aspects, the medium comprises about 42 mM potassium ion, and the medium has a tonicity of about 250 mOsmol. In some aspects, the medium comprises about 43 mM potassium ion, and the medium has a tonicity of about 250 mOsmol. In some aspects, the medium comprises about 44 mM potassium ion, and the medium has a tonicity of about 250 mOsmol. In some aspects, the medium comprises about 45 mM potassium ion, and the medium has a tonicity of about 250 mOsmol. In some aspects, the medium comprises about 46 mM potassium ion, and the medium has a tonicity of about 250 mOsmol. In some aspects, the medium comprises about 47 mM potassium ion, and the medium has a tonicity of about 250 mOsmol. In some aspects, the medium comprises about 48 mM potassium ion, and the medium has a tonicity of about 250 mOsmol. In some aspects, the medium comprises about 49 mM potassium ion, and the medium has a tonicity of about 250 mOsmol.

In some aspects, the medium comprises about 50 mM potassium ion, and the medium has a tonicity of about 255 mOsmol. In some aspects, the medium comprises about 50 mM potassium ion, and the medium has a tonicity of about 250 mOsmol to about 260 mOsmol. In some aspects, the medium comprises about 50 mM potassium ion, and the medium has a tonicity of about 254 mOsmol to about 256 mOsmol. In some aspects, the medium comprises about 50 mM potassium ion, and the medium has a tonicity of about 254.7 mOsmol.

In some aspects, the medium comprises about 51 mM, about 52 mM, about 53 mM, or about 54 mM potassium ion, and the medium has a tonicity of about 255 mOsmol. In some aspects, the medium comprises about 51 mM, about 52 mM, about 53 mM, or about 54 mM potassium ion, and the medium has a tonicity of about 250 mOsmol to about 260 mOsmol. In some aspects, the medium comprises about 51 mM, about 52 mM, about 53 mM, or about 54 mM potassium ion, and the medium has a tonicity of about 254 mOsmol to about 256 mOsmol. In some aspects, the medium comprises about 51 mM, about 52 mM, about 53 mM, or about 54 mM potassium ion, and the medium has a tonicity of about 254.7 mOsmol. In some aspects, the medium comprises about 51 mM, about 52 mM, about 53 mM, or about 54 mM potassium ion, and the medium has a tonicity of about 255.2 mOsmol.

In some aspects, the medium comprises about 55 mM potassium ion, and the medium has a tonicity of about 255 mOsmol. In some aspects, the medium comprises about 55 mM potassium ion, and the medium has a tonicity of about 250 mOsmol to about 260 mOsmol. In some aspects, the medium comprises about 55 mM potassium ion, and the medium has a tonicity of about 254 mOsmol to about 256 mOsmol. In some aspects, the medium comprises about 55 mM potassium ion, and the medium has a tonicity of about 255.2 mOsmol.

In some aspects, the medium comprises about 56 mM, about 57 mM, about 58 mM, or about 59 mM potassium ion, and the medium has a tonicity of about 256 mOsmol. In some aspects, the medium comprises about 56 mM, about 57 mM, about 58 mM, or about 59 mM potassium ion, and the medium has a tonicity of about 250 mOsmol to about 260 mOsmol. In some aspects, the medium comprises about 56 mM, about 57 mM, about 58 mM, or about 59 mM potassium ion, and the medium has a tonicity of about 255 mOsmol to about 258 mOsmol. In some aspects, the medium comprises about 60 mM potassium ion, and the medium has a tonicity of about 257.2 mOsmol.

In some aspects, the medium comprises about 60 mM potassium ion, and the medium has a tonicity of about 257 mOsmol. In some aspects, the medium comprises about 60 mM potassium ion, and the medium has a tonicity of about 250 mOsmol to about 260 mOsmol. In some aspects, the medium comprises about 60 mM potassium ion, and the medium has a tonicity of about 256 mOsmol to about 258 mOsmol. In some aspects, the medium comprises about 60 mM potassium ion, and the medium has a tonicity of about 257.2 mOsmol.

In some aspects, the medium comprises about 61 mM, about 62 mM, about 63 mM, or about 64 mM potassium ion, and the medium has a tonicity of about 257 mOsmol. In some aspects, the medium comprises about 61 mM, about 62 mM, about 63 mM, or about 64 mM potassium ion, and the medium has a tonicity of about 250 mOsmol to about 260 mOsmol. In some aspects, the medium comprises about 61 mM, about 62 mM, about 63 mM, or about 64 mM potassium ion, and the medium has a tonicity of about 256 mOsmol to about 258 mOsmol. In some aspects, the medium comprises about 61 mM, about 62 mM, about 63 mM, or about 64 mM potassium ion, and the medium has a tonicity of about 257.2 mOsmol. In some aspects, the medium comprises about 61 mM, about 62 mM, about 63 mM, or about 64 mM potassium ion, and the medium has a tonicity of about 257.5 mOsmol.

In some aspects, the medium comprises about 65 mM potassium ion, and the medium has a tonicity of about 257 mOsmol. In some aspects, the medium comprises about 65 mM potassium ion, and the medium has a tonicity of about 250 mOsmol to about 260 mOsmol. In some aspects, the medium comprises about 65 mM potassium ion, and the medium has a tonicity of about 257 mOsmol to about 258 mOsmol. In some aspects, the medium comprises about 65 mM potassium ion, and the medium has a tonicity of about 257.5 mOsmol.

In some aspects, the medium comprises about 66 mM, about 67 mM, about 68 mM, or about 69 mM potassium ion, and the medium has a tonicity of about 257 mOsmol. In some aspects, the medium comprises about 66 mM, about 67 mM, about 68 mM, or about 69 mM potassium ion, and the medium has a tonicity of about 255 mOsmol to about 265 mOsmol. In some aspects, the medium comprises about 66 mM, about 67 mM, about 68 mM, or about 69 mM potassium ion, and the medium has a tonicity of about 257 mOsmol to about 260 mOsmol. In some aspects, the medium comprises about 66 mM, about 67 mM, about 68 mM, or about 69 mM potassium ion, and the medium has a tonicity of about 257.5 mOsmol. In some aspects, the medium comprises about 66 mM, about 67 mM, about 68 mM, or about 69 mM potassium ion, and the medium has a tonicity of about 259.7 mOsmol.

In some aspects, the medium comprises about 70 mM potassium ion, and the medium has a tonicity of about 259 mOsmol. In some aspects, the medium comprises about 70 mM potassium ion, and the medium has a tonicity of about 255 mOsmol to about 265 mOsmol. In some aspects, the medium comprises about 70 mM potassium ion, and the medium has a tonicity of about 259 mOsmol to about 261 mOsmol. In some aspects, the medium comprises about 70 mM potassium ion, and the medium has a tonicity of about 259.7 mOsmol.

In some aspects, the medium comprises about 71 mM, about 72 mM, about 73 mM, or about 74 mM potassium ion, and the medium has a tonicity of about 259 mOsmol. In some aspects, the medium comprises about 71 mM, about 72 mM, about 73 mM, or about 74 mM potassium ion, and the medium has a tonicity of about 255 mOsmol to about 265 mOsmol. In some aspects, the medium comprises about 71 mM, about 72 mM, about 73 mM, or about 74 mM potassium ion, and the medium has a tonicity of about 259 mOsmol to about 261 mOsmol. In some aspects, the medium comprises about 71 mM, about 72 mM, about 73 mM, or about 74 mM potassium ion, and the medium has a tonicity of about 259.7 mOsmol. In some aspects, the medium comprises about 71 mM, about 72 mM, about 73 mM, or about 74 mM potassium ion, and the medium has a tonicity of about 260 mOsmol.

In some aspects, the medium comprises about 75 mM potassium ion, and the medium has a tonicity of about 260 mOsmol. In some aspects, the medium comprises about 75 mM potassium ion, and the medium has a tonicity of about 255 mOsmol to about 265 mOsmol. In some aspects, the medium comprises about 75 mM potassium ion, and the medium has a tonicity of about 259 mOsmol to about 261 mOsmol. In some aspects, the medium comprises about 75 mM potassium ion, and the medium has a tonicity of about 260 mOsmol.

In some aspects, the medium comprises about 81 mM, about 82 mM, about 83 mM, or about 84 mM potassium ion, and the medium has a tonicity of about 260 mOsmol. In some aspects, the medium comprises about 81 mM, about 82 mM, about 83 mM, or about 84 mM potassium ion, and the medium has a tonicity of about 261 mOsmol. In some aspects, the medium comprises about 81 mM, about 82 mM, about 83 mM, or about 84 mM potassium ion, and the medium has a tonicity of about 262 mOsmol. In some aspects, the medium comprises about 81 mM, about 82 mM, about 83 mM, or about 84 mM potassium ion, and the medium has a tonicity of about 263 mOsmol. In some aspects, the medium comprises about 81 mM, about 82 mM, about 83 mM, or about 84 mM potassium ion, and the medium has a tonicity of about 255 mOsmol to about 265 mOsmol. In some aspects, the medium comprises about 81 mM, about 82 mM, about 83 mM, or about 84 mM potassium ion, and the medium has a tonicity of about 261 mOsmol to about 263 mOsmol. In some aspects, the medium comprises about 81 mM, about 82 mM, about 83 mM, or about 84 mM potassium ion, and the medium has a tonicity of about 262.26 mOsmol.

In some aspects, the medium comprises about 80 mM potassium ion, and the medium has a tonicity of about 262 mOsmol. In some aspects, the medium comprises about 80 mM potassium ion, and the medium has a tonicity of about 255 mOsmol to about 265 mOsmol. In some aspects, the medium comprises about 80 mM potassium ion, and the medium has a tonicity of about 261 mOsmol to about 263 mOsmol. In some aspects, the medium comprises about 80 mM potassium ion, and the medium has a tonicity of about 262.26 mOsmol.

In some aspects, the medium comprises about 81 mM, about 82 mM, about 83 mM, about 84 mM, or about 85 mM potassium ion, and the medium has a tonicity of about 262 mOsmol. In some aspects, the medium comprises about 81 mM, about 82 mM, about 83 mM, about 84 mM, or about 85 mM potassium ion, and the medium has a tonicity of about 263 mOsmol. In some aspects, the medium comprises about 81 mM, about 82 mM, about 83 mM, about 84 mM, or about 85 mM potassium ion, and the medium has a tonicity of about 264 mOsmol. In some aspects, the medium comprises about 81 mM, about 82 mM, about 83 mM, about 84 mM, or about 85 mM potassium ion, and the medium has a tonicity of about 265 mOsmol. In some aspects, the medium comprises about 81 mM, about 82 mM, about 83 mM, about 84 mM, or about 85 mM potassium ion, and the medium has a tonicity of about 255 mOsmol to about 265 mOsmol. In some aspects, the medium comprises about 81 mM, about 82 mM, about 83 mM, about 84 mM, or about 85 mM potassium ion, and the medium has a tonicity of about 260 mOsmol to about 270 mOsmol. In some aspects, the medium comprises about 81 mM, about 82 mM, about 83 mM, about 84 mM, or about 85 mM potassium ion, and the medium has a tonicity of about 261 mOsmol to about 263 mOsmol. In some aspects, the medium comprises about 81 mM, about 82 mM, about 83 mM, about 84 mM, or about 85 mM potassium ion, and the medium has a tonicity of about 263 mOsmol to about 265 mOsmol.

In some aspects, the medium comprises sodium ion. In some aspects, the concentration of the sodium ion is less than that of the basal medium. In some aspects, the concentration of the sodium ion is reduced as the concentration of potassium ion is increased. In some aspects, the concentration of the sodium ion is from about 25 mM to about 115 mM. In some aspects, the concentration of the sodium ion is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In certain aspects, the concentration of the sodium ion is from about 50 mM to about 85 mM. In certain aspects, the concentration of the sodium ion is from about 55 mM to about 80 mM. In certain aspects, the concentration of the sodium ion is from about 30 mM to about 35 mM. In certain aspects, the concentration of the sodium ion is from about 35 mM to about 40 mM. In certain aspects, the concentration of the sodium ion is from about 40 mM to about 45 mM. In certain aspects, the concentration of the sodium ion is from about 45 mM to about 50 mM. In certain aspects, the concentration of the sodium ion is from about 50 mM to about 55 mM. In certain aspects, the concentration of the sodium ion is from about 55 mM to about 60 mM. In certain aspects, the concentration of the sodium ion is from about 60 mM to about 65 mM. In certain aspects, the concentration of the sodium ion is from about 65 mM to about 70 mM. In certain aspects, the concentration of the sodium ion is from about 70 mM to about 75 mM. In certain aspects, the concentration of the sodium ion is from about 75 mM to about 80 mM. In certain aspects, the concentration of the sodium ion is from about 80 mM to about 85 mM.

In some aspects, the concentration of the sodium ion is about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In certain aspects, the concentration of sodium ion is about 55 mM. In certain aspects, the concentration of sodium ion is about 55.6 mM. In certain aspects, the concentration of sodium ion is about 59.3 mM. In certain aspects, the concentration of sodium ion is about 60 mM. In certain aspects, the concentration of sodium ion is about 63.9 mM. In certain aspects, the concentration of sodium ion is about 65 mM. In certain aspects, the concentration of sodium ion is about 67.6 mM. In certain aspects, the concentration of sodium ion is about 70 mM. In certain aspects, the concentration of sodium ion is about 72.2 mM. In certain aspects, the concentration of sodium ion is about 75 mM. In certain aspects, the concentration of sodium ion is about 76 mM. In certain aspects, the concentration of sodium ion is about 80 mM. In certain aspects, the concentration of sodium ion is about 80.5 mM.

In some aspects, the medium comprises about 50 mM potassium ion and about 80.5 mM sodium ion. In some aspects, the medium has a tonicity of about 254.7 mOsmol. In some aspects, the medium comprises about 55 mM potassium ion and about 76 mM sodium ion. In some aspects, the medium has a tonicity of about 255.2 mOsmol. In some aspects, the medium comprises about 60 mM potassium ion and about 72.2 mM sodium ion. In some aspects, the medium has a tonicity of about 257.2 mOsmol. In some aspects, the medium comprises about 65 mM potassium ion and about 67.6 mM sodium ion. In some aspects, the medium has a tonicity of about 257.5 mOsmol. In some aspects, the medium comprises about 70 mM potassium ion and about 63.9 mM sodium ion. In some aspects, the medium has a tonicity of about 259.7 mOsmol. In some aspects, the medium comprises about 75 mM potassium ion and about 59.3 mM sodium ion. In some aspects, the medium has a tonicity of about 260 mOsmol. In some aspects, the medium comprises about 80 mM potassium ion and about 55.6 mM sodium ion. In some aspects, the medium has a tonicity of about 262.26 mOsmol.

In some aspects, the medium comprises about 40 mM potassium ion and a NaCl concentration of 100 mM. In some aspects, the medium comprises about 41 mM potassium ion and a NaCl concentration of 99 mM. In some aspects, the medium comprises about 42 mM potassium ion and a NaCl concentration of 98 mM. In some aspects, the medium comprises about 43 mM potassium ion and a NaCl concentration of 97 mM. In some aspects, the medium comprises about 44 mM potassium ion and a NaCl concentration of 96 mM. In some aspects, the medium comprises about 45 mM potassium ion and a NaCl concentration of 95 mM. In some aspects, the medium comprises about 46 mM potassium ion and a NaCl concentration of 94 mM. In some aspects, the medium comprises about 47 mM potassium ion and a NaCl concentration of 93 mM. In some aspects, the medium comprises about 48 mM potassium ion and a NaCl concentration of 92 mM. In some aspects, the medium comprises about 49 mM potassium ion and a NaCl concentration of 91 mM.

In some aspects, the medium comprises about 50 mM potassium ion and a NaCl concentration of 90 mM. In some aspects, the medium comprises about 51 mM potassium ion and a NaCl concentration of 89 mM. In some aspects, the medium comprises about 52 mM potassium ion and a NaCl concentration of 88 mM. In some aspects, the medium comprises about 53 mM potassium ion and a NaCl concentration of 87 mM. In some aspects, the medium comprises about 54 mM potassium ion and a NaCl concentration of 86 mM. In some aspects, the medium comprises about 55 mM potassium ion and a NaCl concentration of 85 mM. In some aspects, the medium comprises about 56 mM potassium ion and a NaCl concentration of 84 mM. In some aspects, the medium comprises about 57 mM potassium ion and a NaCl concentration of 83 mM. In some aspects, the medium comprises about 58 mM potassium ion and a NaCl concentration of 82 mM. In some aspects, the medium comprises about 59 mM potassium ion and a NaCl concentration of 81 mM.

In some aspects, the medium comprises about 60 mM potassium ion and a NaCl concentration of 80 mM. In some aspects, the medium comprises about 61 mM potassium ion and a NaCl concentration of 79 mM. In some aspects, the medium comprises about 62 mM potassium ion and a NaCl concentration of 78 mM. In some aspects, the medium comprises about 63 mM potassium ion and a NaCl concentration of 77 mM. In some aspects, the medium comprises about 64 mM potassium ion and a NaCl concentration of 76 mM. In some aspects, the medium comprises about 65 mM potassium ion and a NaCl concentration of 75 mM. In some aspects, the medium comprises about 66 mM potassium ion and a NaCl concentration of 74 mM. In some aspects, the medium comprises about 67 mM potassium ion and a NaCl concentration of 73 mM. In some aspects, the medium comprises about 68 mM potassium ion and a NaCl concentration of 72 mM. In some aspects, the medium comprises about 69 mM potassium ion and a NaCl concentration of 71 mM.

In some aspects, the medium comprises about 70 mM potassium ion and a NaCl concentration of 70 mM. In some aspects, the medium comprises about 71 mM potassium ion and a NaCl concentration of 69 mM. In some aspects, the medium comprises about 72 mM potassium ion and a NaCl concentration of 68 mM. In some aspects, the medium comprises about 73 mM potassium ion and a NaCl concentration of 67 mM. In some aspects, the medium comprises about 74 mM potassium ion and a NaCl concentration of 66 mM. In some aspects, the medium comprises about 75 mM potassium ion and a NaCl concentration of 65 mM. In some aspects, the medium comprises about 76 mM potassium ion and a NaCl concentration of 64 mM. In some aspects, the medium comprises about 77 mM potassium ion and a NaCl concentration of 63 mM. In some aspects, the medium comprises about 78 mM potassium ion and a NaCl concentration of 62 mM. In some aspects, the medium comprises about 79 mM potassium ion and a NaCl concentration of 61 mM.

In some aspects, the medium comprises about 80 mM potassium ion and a NaCl concentration of 60 mM. In some aspects, the medium comprises about 81 mM potassium ion and a NaCl concentration of 59 mM. In some aspects, the medium comprises about 82 mM potassium ion and a NaCl concentration of 58 mM. In some aspects, the medium comprises about 83 mM potassium ion and a NaCl concentration of 57 mM. In some aspects, the medium comprises about 84 mM potassium ion and a NaCl concentration of 56 mM. In some aspects, the medium comprises about 85 mM potassium ion and a NaCl concentration of 55 mM. In some aspects, the medium comprises about 86 mM potassium ion and a NaCl concentration of 54 mM. In some aspects, the medium comprises about 87 mM potassium ion and a NaCl concentration of 53 mM. In some aspects, the medium comprises about 88 mM potassium ion and a NaCl concentration of 52 mM. In some aspects, the medium comprises about 89 mM potassium ion and a NaCl concentration of 51 mM.

In some aspects, the medium comprises about 890 mM potassium ion and a NaCl concentration of 50 mM. In some aspects, the medium comprises about 91 mM potassium ion and a NaCl concentration of 49 mM. In some aspects, the medium comprises about 92 mM potassium ion and a NaCl concentration of 48 mM. In some aspects, the medium comprises about 93 mM potassium ion and a NaCl concentration of 47 mM. In some aspects, the medium comprises about 94 mM potassium ion and a NaCl concentration of 46 mM. In some aspects, the medium comprises about 95 mM potassium ion and a NaCl concentration of 45 mM. In some aspects, the medium comprises about 96 mM potassium ion and a NaCl concentration of 44 mM. In some aspects, the medium comprises about 97 mM potassium ion and a NaCl concentration of 43 mM. In some aspects, the medium comprises about 98 mM potassium ion and a NaCl concentration of 42 mM. In some aspects, the medium comprises about 99 mM potassium ion and a NaCl concentration of 41 mM.

In some aspects, the medium comprises NaCl. In some aspects, the concentration of the NaCl is less than that of the basal medium. In some aspects, the concentration of the NaCl is reduced as the concentration of potassium ion is increased. In some aspects, the concentration of the NaCl is from about 25 mM to about 115 mM. In some aspects, the concentration of the NaCl is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In certain aspects, the concentration of the NaCl is from about 50 mM to about 85 mM. In certain aspects, the concentration of the NaCl is from about 55 mM to about 80 mM. In certain aspects, the concentration of the NaCl is from about 30 mM to about 35 mM. In certain aspects, the concentration of the NaCl is from about 35 mM to about 40 mM. In certain aspects, the concentration of the NaCl is from about 40 mM to about 45 mM. In certain aspects, the concentration of the NaCl is from about 45 mM to about 50 mM. In certain aspects, the concentration of the NaCl is from about 50 mM to about 55 mM. In certain aspects, the concentration of the NaCl is from about 55 mM to about 60 mM. In certain aspects, the concentration of the NaCl is from about 60 mM to about 65 mM. In certain aspects, the concentration of the NaCl is from about 65 mM to about 70 mM. In certain aspects, the concentration of the NaCl is from about 70 mM to about 75 mM. In certain aspects, the concentration of the NaCl is from about 75 mM to about 80 mM. In certain aspects, the concentration of the NaCl is from about 80 mM to about 85 mM.

In some aspects, the medium comprises calcium ion. In some aspects, the concentration of calcium ion is more than about 0.4 mM. In some aspects, the concentration of calcium ion is less than about 2.8 mM. In some aspects, the concentration of calcium ion is less than about 2.5 mM. In some aspects, the concentration of calcium ion is less than about 2.0 mM. In some aspects, the concentration of calcium ion is less than about 1.9 mM. In some aspects, the concentration of calcium ion is less than about 1.8 mM. In some aspects, the concentration of calcium ion is less than about 1.7 mM. In some aspects, the concentration of calcium ion is less than about 1.6 mM. In some aspects, the concentration of calcium ion is less than about 1.5 mM. In some aspects, the concentration of calcium ion is less than about 1.4 mM. In some aspects, the concentration of calcium ion is less than about 1.3 mM. In some aspects, the concentration of calcium ion is less than about 1.2 mM. In some aspects, the concentration of calcium ion is less than about 1.1 mM. In some aspects, the concentration of calcium ion is less than about 1.0 mM.

In some aspects, the concentration of calcium ion is from about 0.4 mM to about 2.8 mM, about 0.4 mM to about 2.7 mM, about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 0.8 to about 0.9 mM, about 0.8 to about 1.0 mM, about 0.8 to about 1.1 mM, about 0.8 to about 1.2 mM, about 0.8 to about 1.3 mM, about 0.8 to about 1.4 mM, about 0.8 to about 1.5 mM, about 0.8 to about 1.6 mM, about 0.8 to about 1.7 mM, about 0.8 to about 1.8 mM, about 0.9 to about 1.0 mM, about 0.9 to about 1.1 mM, about 0.9 to about 1.2 mM, about 0.9 to about 1.3 mM, about 0.9 to about 1.4 mM, about 0.9 to about 1.5 mM, about 0.9 to about 1.6 mM, about 0.9 to about 1.7 mM, about 0.9 to about 1.8 mM, about 1.0 to about 1.1 mM, about 1.0 to about 1.2 mM, about 1.0 to about 1.3 mM, about 1.0 to about 1.4 mM, about 1.0 to about 1.5 mM, about 1.0 to about 1.6 mM, about 1.0 to about 1.7 mM, about 1.0 to about 1.8 mM, about 1.1 to about 1.2 mM, about 1.1 to about 1.3 mM, about 1.1 to about 1.4 mM, about 1.1 to about 1.5 mM, about 1.1 to about 1.6 mM, about 1.1 to about 1.7 mM, about 1.1 to about 1.8 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

In some aspects, the concentration of calcium ion is from about 0.8 mM to about 1.8 mM. In some aspects, the concentration of calcium ion is from about 0.9 mM to about 1.8 mM. In some aspects, the concentration of calcium ion is from about 1.0 mM to about 1.8 mM. In some aspects, the concentration of calcium ion is from about 1.1 mM to about 1.8 mM. In some aspects, the concentration of calcium ion is from about 1.2 mM to about 1.8 mM. In some aspects, the concentration of calcium ion is from about 0.8 mM to about 1.8 mM. In some aspects, the concentration of calcium ion is from about 0.8 mM to about 0.9 mM. In some aspects, the concentration of calcium ion is from about 0.9 mM to about 1.0 mM. In some aspects, the concentration of calcium ion is from about 1.0 mM to about 1.1 mM. In some aspects, the concentration of calcium ion is from about 1.1 mM to about 1.2 mM. In some aspects, the concentration of calcium ion is from about 1.2 mM to about 1.3 mM. In some aspects, the concentration of calcium ion is from about 1.3 mM to about 1.4 mM. In some aspects, the concentration of calcium ion is from about 1.4 mM to about 1.5 mM. In some aspects, the concentration of calcium ion is from about 1.5 mM to about 1.6 mM. In some aspects, the concentration of calcium ion is from about 1.7 mM to about 1.8 mM.

In some aspects, the concentration of calcium ion is about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some aspects, the concentration of calcium ion is about 0.6 mM. In some aspects, the concentration of calcium ion is about 0.7 mM. In some aspects, the concentration of calcium ion is about 0.8 mM. In some aspects, the concentration of calcium ion is about 0.9 mM. In some aspects, the concentration of calcium ion is about 1.0 mM. In some aspects, the concentration of calcium ion is about 1.1 mM. In some aspects, the concentration of calcium ion is about 1.2 mM. In some aspects, the concentration of calcium ion is about 1.3 mM. In some aspects, the concentration of calcium ion is about 1.4 mM. In some aspects, the concentration of calcium ion is about 1.5 mM. In some aspects, the concentration of calcium ion is about 1.6 mM. In some aspects, the concentration of calcium ion is about 1.7 mM. In some aspects, the concentration of calcium ion is about 1.8 mM.

In some aspects, the medium comprises about 50 mM potassium ion and about 1.8 mM calcium ion. In some aspects, the medium further comprises less than 90 mM NaCl.

In some aspects, the medium comprises about 55 mM potassium ion and about 1.7 mM calcium ion. In some aspects, the medium further comprises less than 85 mM NaCl.

In some aspects, the medium comprises about 60 mM potassium ion and about 1.6 mM calcium ion. In some aspects, the medium has an osmolality of about 257.2 mOsmol. In some aspects, the medium further comprises less than 80 mM NaCl.

In some aspects, the medium comprises about 65 mM potassium ion and about 1.5 mM calcium ion. In some aspects, the medium further comprises less than 75 mM NaCl.

In some aspects, the medium comprises about 70 mM potassium ion and about 1.4 mM calcium ion. In some aspects, the medium further comprises less than 70 mM NaCl.

In some aspects, the medium comprises about 75 mM potassium ion and about 1.3 mM calcium ion. In some aspects, the medium further comprises less than 65 mM NaCl.

In some aspects, the medium comprises about 80 mM potassium ion and about 1.2 mM calcium ion. In some aspects, the medium further comprises less than 60 mM NaCl.

In some aspects, the medium comprises a saccharide. In some aspects, the saccharide is a monosaccharide, a disaccharide, or a polysaccharide. In some aspects, the saccharide is selected from glucose, fructose, galactose, mannose, maltose, sucrose, lactose, trehalose, and any combination thereof. In certain aspects, the saccharide is glucose. In some aspects, the medium comprises (i) potassium ion at a concentration of at least about 50 mM and (ii) glucose. In some aspects, the medium comprises (i) potassium ion at a concentration of at least about 50 mM and (ii) mannose. In some aspects, the medium comprises more than one saccharide. In some aspects, the medium comprises glucose and galactose. In some aspects, the medium comprises glucose and fructose. In some aspects, the medium comprises glucose and mannose. In some aspects, the medium comprises glucose and maltose. In some aspects, the medium comprises glucose and sucrose. In some aspects, the medium comprises glucose and lactose. In some aspects, the medium comprises glucose and trehalose.

In some aspects, the concentration of the saccharide, e.g., glucose, is less than about 4.29 g/L. In some aspects, the concentration of the saccharide, e.g., glucose, is less than about 24 mM. In some aspects, the concentration of the saccharide, e.g., glucose, is more than about 5 mM. In some aspects, the concentration of the saccharide, e.g., glucose, is more than about 5 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 25 mM, about 10 mM to about 20 mM, about 10 mM to about 5 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 15 mM. In some aspects, the concentration of the saccharide, e.g., glucose, is from about 14 mM to about 14.5 mM. In some aspects, the concentration of the saccharide, e.g., glucose, is from about 14.5 mM to about 15 mM. In some aspects, the concentration of the saccharide, e.g., glucose, is from about 15 mM to about 15.5 mM. In some aspects, the concentration of the saccharide, e.g., glucose, is from about 15.5 mM to about 16 mM. In some aspects, the concentration of the saccharide, e.g., glucose, is from about 16 mM to about 16.5 mM. In some aspects, the concentration of the saccharide, e.g., glucose, is from about 16.5 mM to about 17 mM. In some aspects, the concentration of the saccharide, e.g., glucose, is from about 17 mM to about 17.5 mM. In some aspects, the concentration of the saccharide, e.g., glucose, is from about 17.5 mM to about 18 mM.

In some aspects, the concentration of the saccharide, e.g., glucose, is about 5 mM, about 6 mM, about 7 mM, about 8 mM, about 9 mM, about 10 mM, is about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 20.5 mM, about 21 mM, about 22 mM, about 23 mM, about 24 mM, or about 25 mM.

In some aspects, the concentration of the saccharide, e.g., glucose, is about 13 mM. In some aspects, the concentration of the saccharide, e.g., glucose, is about 13.5 mM. In some aspects, the concentration of the saccharide, e.g., glucose, is about 14 mM. In some aspects, the concentration of the saccharide, e.g., glucose, is about 14.5 mM. In some aspects, the concentration of the saccharide, e.g., glucose, is about 15 mM. In some aspects, the concentration of the saccharide, e.g., glucose, is about 15.4 mM. In some aspects, the concentration of the saccharide, e.g., glucose, is about 15.9 mM. In some aspects, the concentration of the saccharide, e.g., glucose, is about 16.3 mM. In some aspects, the concentration of the saccharide, e.g., glucose, is about 16.8 mM. In some aspects, the concentration of the saccharide, e.g., glucose, is about 17.2 mM. In some aspects, the concentration of the saccharide, e.g., glucose, is about 17.7 mM.

In some aspects, the medium comprises about 50 mM potassium ion and about 17.7 mM glucose. In some aspects, the medium further comprises less than 90 mM NaCl.

In some aspects, the medium comprises about 55 mM potassium ion and about 17.2 mM glucose. In some aspects, the medium further comprises less than 85 mM NaCl.

In some aspects, the medium comprises about 60 mM potassium ion and about 16.8 mM glucose. In some aspects, the medium further comprises less than 80 mM NaCl.

In some aspects, the medium comprises about 65 mM potassium ion and about 16.3 mM glucose. In some aspects, the medium further comprises less than 75 mM NaCl.

In some aspects, the medium comprises about 70 mM potassium ion and about 15.9 mM glucose. In some aspects, the medium further comprises less than 70 mM NaCl.

In some aspects, the medium comprises about 75 mM potassium ion and about 15.4 mM glucose. In some aspects, the medium further comprises less than 65 mM NaCl.

In some aspects, the medium comprises about 80 mM potassium ion and about 15 mM glucose. In some aspects, the medium further comprises less than 60 mM NaCl.

In some aspects, the medium is isotonic. In some aspects, the medium has a tonicity of about 280 mOsm/L. In some aspects, the medium has a tonicity of 280 mOsm/L. In some aspects, the medium has a tonicity of 280 mOsm/L±1 mOsm/L. In some aspects, the medium has a tonicity of 280 mOsm/L 2 mOsm/L. In some aspects, the medium has a tonicity of 280 mOsm/L±3 mOsm/L. In some aspects, the medium has a tonicity of 280 mOsm/L±4 mOsm/L. In some aspects, the medium has a tonicity of 280 mOsm/L±5 mOsm/L. In some aspects, the medium has a tonicity of 280 mOsm/L±6 mOsm/L. In some aspects, the medium has a tonicity of 280 mOsm/L±7 mOsm/L. In some aspects, the medium has a tonicity of 280 mOsm/L±8 mOsm/L. In some aspects, the medium has a tonicity of 280 mOsm/L±9 mOsm/L. In some aspects, the medium has a tonicity of 280 mOsm/L±10 mOsm/L. In some aspects, the medium has a tonicity of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L. In some aspects, the medium has a tonicity of about 285 mOsm/L, about 290 mOsm/L, about 295 mOsm/L, about 300 mOsm/L, about 305 mOsm/L, about 310 mOsm/L, or about 315 mOsm/L. In some aspects, tonicity is measured by adding the concentrations of potassium ion and NaCl, and multiplying by two.

In some aspects, the medium is hypotonic. In some aspects, the medium has a tonicity lower than about 280 mOsm/L. In some aspects, the medium has a tonicity lower than 280 mOsm/L. In some aspects, the medium has a tonicity lower than 275 mOsm/L. In some aspects, the medium has a tonicity lower than 270 mOsm/L. In some aspects, the medium has a tonicity lower than 265 mOsm/L. In some aspects, the medium has a tonicity lower than 260 mOsm/L. In some aspects, the medium has a tonicity lower than 265 mOsm/L. In some aspects, the medium has a tonicity lower than 260 mOsm/L. In some aspects, the medium has a tonicity lower than 255 mOsm/L. In some aspects, the medium has a tonicity lower than about 250 mOsm/L. In some aspects, the medium has a tonicity lower than about 245 mOsm/L. In some aspects, the medium has a tonicity lower than about 240 mOsm/L. In some aspects, the medium has a tonicity lower than about 235 mOsm/L. In some aspects, the medium has a tonicity lower than about 230 mOsm/L. In some aspects, the medium has a tonicity lower than about 225 mOsm/L. In some aspects, the medium has a tonicity lower than about 220 mOsm/L. In some aspects, the medium has a tonicity lower than about 215 mOsm/L. In some aspects, the medium has a tonicity lower than about 210 mOsm/L. In some aspects, the medium has a tonicity lower than about 205 mOsm/L. In some aspects, the medium has a tonicity lower than about 200 mOsm/L. In some aspects, tonicity is measured by adding the concentrations of potassium ion and NaCl, and multiplying by two.

In some aspects, the medium has a tonicity from about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L. In some aspects, the medium has a tonicity from about 250 mOsm/L to about 270 mOsm/L. In some aspects, the medium has a tonicity from about 250 mOsm/L to about 255 mOsm/L, about 250 mOsm/L to about 260 mOsm/L, about 250 mOsm/L to about 265 mOsm/L, about 255 mOsm/L to about 260 mOsm/L, about 255 mOsm/L to about 265 mOsm/L, about 255 mOsm/L to about 265 mOsm/L, about 260 mOsm/L to about 265 mOsm/L, or about 254 mOsm/L to about 263 mOsm/L. In some aspects, the medium has a tonicity from about 254 mOsm/L to about 255 mOsm/L. In some aspects, the medium has a tonicity from about 255 mOsm/L to about 256 mOsm/L. In some aspects, the medium has a tonicity from about 256 mOsm/L to about 257 mOsm/L. In some aspects, the medium has a tonicity from about 257 mOsm/L to about 258 mOsm/L. In some aspects, the medium has a tonicity from about 258 mOsm/L to about 259 mOsm/L. In some aspects, the medium has a tonicity from about 260 mOsm/L to about 261 mOsm/L. In some aspects, the medium has a tonicity from about 261 mOsm/L to about 262 mOsm/L. In some aspects, the medium has a tonicity from about 262 mOsm/L to about 263 mOsm/L. In some aspects, the medium has a tonicity from about 263 mOsm/L to about 264 mOsm/L. In some aspects, the medium has a tonicity from about 264 mOsm/L to about 265 mOsm/L. In some aspects, tonicity is measured by adding the concentrations of potassium ion and NaCl, and multiplying by two.

In some aspects, the medium has a tonicity of about 100 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L. In some aspects, tonicity is measured by adding the concentrations of potassium ion and NaCl, and multiplying by two.

In some aspects, the medium has a tonicity of about 250 mOsm/L. In some aspects, the medium has a tonicity of about 262.26 mOsm/L. In some aspects, the medium has a tonicity of about 260 mOsm/L. In some aspects, the medium has a tonicity of about 259.7 mOsm/L. In some aspects, the medium has a tonicity of about 257.5 mOsm/L. In some aspects, the medium has a tonicity of about 257.2 mOsm/L. In some aspects, the medium has a tonicity of about 255.2 mOsm/L. In some aspects, the medium has a tonicity of about 254.7. In some aspects, the medium has a tonicity of about 255 mOsm/L. In some aspects, the medium has a tonicity of about 260 mOsm/L. In some aspects, tonicity is measured by adding the concentrations of potassium ion and NaCl, and multiplying by two.

In some aspects, the medium comprises about 50 mM potassium ion and (i) about 80.5 mM sodium ion; (ii) about 17.7 mM glucose; (iii) about 1.8 mM calcium ion; or (iv) any combination of (i)-(iii). In some aspects, the medium has a tonicity of about 254.7 mOsmol. In some aspects, the medium comprises about 50 mM potassium ion and (i) about 80.5 mM NaCl; (ii) about 17.7 mM glucose; (iii) about 1.8 mM calcium ion; or (iv) any combination of (i)-(iii).

In some aspects, the medium comprises about 55 mM potassium ion and (i) about 76 mM sodium ion; (ii) about 17.2 mM glucose; (iii) about 1.7 mM calcium ion; or (iv) any combination of (i)-(iii). In some aspects, the medium has a tonicity of about 255.2 mOsmol. In some aspects, the medium comprises about 55 mM potassium ion and (i) about 76 mM sodium ion; (ii) about 17.2 mM glucose; (iii) and about 1.7 mM calcium ion; wherein the medium has a tonicity of about 255.2 mOsmol. In some aspects, the medium comprises about 55 mM potassium ion and (i) about 76 mM NaCl; (ii) about 17.2 mM glucose; (iii) about 1.7 mM calcium ion; or (iv) any combination of (i)-(iii).

In some aspects, the medium comprises about 60 mM potassium ion and (i) about 72.2 mM sodium ion; (ii) about 16.8 mM glucose; (iii) about 1.6 mM calcium ion; or (iv) any combination of (i)-(iii). In some aspects, the medium has a tonicity of about 257.2 mOsmol. In some aspects, the medium comprises about 60 mM potassium ion and (i) about 72.2 mM sodium ion; (ii) about 16.8 mM glucose; and (iii) about 1.6 mM calcium ion; wherein the medium has a tonicity of about 257.2 mOsmol. In some aspects, the medium comprises about 60 mM potassium ion and (i) about 72.2 mM NaCl; (ii) about 16.8 mM glucose; (iii) about 1.6 mM calcium ion; or (iv) any combination of (i)-(iii).

In some aspects, the medium comprises about 65 mM potassium ion and (i) about 67.6 mM sodium ion; (ii) about 16.3 mM glucose; (iii) about 1.5 mM calcium ion; or (iv) any combination of (i)-(iii). In some aspects, the medium has a tonicity of about 257.5 mOsmol. In some aspects, the medium comprises about 65 mM potassium ion and (i) about 67.6 mM sodium ion; (ii) about 16.3 mM glucose; and (iii) about 1.5 mM calcium ion; wherein the medium has a tonicity of about 257.5 mOsmol. In some aspects, the medium comprises about 65 mM potassium ion and (i) about 67.6 mM NaCl; (ii) about 16.3 mM glucose; (iii) about 1.5 mM calcium ion; or (iv) any combination of (i)-(iii).

In some aspects, the medium comprises about 70 mM potassium ion and (i) about 63.9 mM sodium ion; (ii) about 15.9 mM glucose; (iii) about 1.4 mM calcium ion; or (iv) any combination of (i)-(iii). In some aspects, the medium has a tonicity of about 259.7 mOsmol. In some aspects, the medium comprises about 70 mM potassium ion and (i) about 63.9 mM sodium ion; (ii) about 15.9 mM glucose; and (iii) about 1.4 mM calcium ion; wherein the medium has a tonicity of about 259.7 mOsmol. In some aspects, the medium comprises about 70 mM potassium ion and (i) about 63.9 mM NaCl; (ii) about 15.9 mM glucose; (iii) about 1.4 mM calcium ion; or (iv) any combination of (i)-(iii).

In some aspects, the medium comprises about 75 mM potassium ion and (i) about 59.3 mM sodium ion; (ii) about 15.4 mM glucose; (iii) about 1.3 mM calcium ion; or (iv) any combination of (i)-(iii). In some aspects, the medium has a tonicity of about 260 mOsmol. In some aspects, the medium comprises about 75 mM potassium ion and (i) about 59.3 mM sodium ion; (ii) about 15.4 mM glucose; and (iii) about 1.3 mM calcium ion; wherein the medium has a tonicity of about 260 mOsmol. In some aspects, the medium comprises about 75 mM potassium ion and (i) about 59.3 mM NaCl; (ii) about 15.4 mM glucose; (iii) about 1.3 mM calcium ion; or (iv) any combination of (i)-(iii).

In some aspects, the medium comprises about 80 mM potassium ion and (i) about 55.6 mM sodium ion; (ii) about 15 mM glucose; (iii) about 1.2 mM calcium ion; or (iv) any combination of (i)-(iii). In some aspects, the medium has a tonicity of about 262.26 mOsmol. In some aspects, the medium comprises about 80 mM potassium ion and (i) about 55.6 mM sodium ion; (ii) about 15 mM glucose; and (iii) about 1.2 mM calcium ion; wherein the medium has a tonicity of about 262.26 mOsmol. In some aspects, the medium comprises about 80 mM potassium ion and (i) about 55.6 mM NaCl; (ii) about 15 mM glucose; (iii) about 1.2 mM calcium ion; or (iv) any combination of (i)-(iii).

III.B. Cells Prepared According to the Methods

Certain aspects of the present disclosure are directed to a cell composition comprising one or more pluripotent, multipotent, and/or immune cell (e.g., T cell and/or NK cell) cultured according to the methods disclosed herein. Some aspects of the present disclosure are directed to a cell composition produced by a method of increasing the yield of human immune cells and/or stem cells during ex vivo or in vitro culturing while increasing stemness of the human immune cells and/or stem cells comprising culturing human immune cells and/or stem cells in a medium comprising potassium ion at a concentration between 40 mM and 80 mM and NaCl at a concentration between 100 mM and 30 mM, wherein the total concentration of potassium ion and NaCl is between 110 and 140 mM. Some aspects of the present disclosure are directed to a cell composition produced by a method of preparing a population of human immune cells and/or stem cells for immunotherapy comprising culturing human immune cells and/or stem cells in a medium comprising potassium ion at a concentration between 40 mM and 80 mM and NaCl at a concentration between 100 mM and 30 mM, wherein the total concentration of potassium ion and NaCl is between 110 and 140 mM. Some aspects of the present disclosure are directed to a cell composition produced by a method of increasing stemness of human immune cells during ex vivo or in vitro culturing for immunotherapy comprising culturing human immune cells in a medium comprising potassium ion at a concentration between 40 mM and 80 mM and NaCl at a concentration between 100 mM and 30 mM, wherein the total concentration of potassium ion and NaCl is between 110 and 140 mM.

Cells cultured according to the methods and/or in the medium disclosed herein have an increased number of less-differentiated cells as compared to comparable cells cultured according to conventional methods. In some aspects, the cells cultured according to the methods disclosed herein exhibit increased expression of one or more marker typical of a stem-like phenotype. In some aspects, cell populations cultured according to the methods and/or in a metabolic reprogramming medium disclosed herein have an increased number of effector-like cells as compared to comparable cells cultured according to conventional methods, e.g., in media containing less than 5 mM K⁺. In some aspects, cell populations cultured according to the methods and/or in a metabolic reprogramming medium disclosed herein have both an increased number of stem-like and effector-like cells as compared to comparable cells cultured according to conventional methods, e.g., in media containing less than 5 mM K⁺. In some aspects, the cells cultured according to the methods disclosed herein exhibit greater proliferative potential compared to cells cultured according to conventional methods. In some aspects, the cells cultured according to the methods disclosed herein exhibit increased transduction efficiency. In some aspects, the cells cultured according to the methods disclosed herein exhibit increased in vivo viability upon transplantation in a subject. In some aspects, the cells cultured according to the methods disclosed herein exhibit increased cell potency. In some aspects, the cells cultured according to the methods disclosed herein exhibit decreased cell exhaustion. In some aspects, the cells cultured according to the methods disclosed herein exhibit increased in vivo persistence upon transplantation in a subject. In some aspects, the cells cultured according to the methods disclosed herein exhibit increased in vivo activity upon transplantation in a subject. In some aspects, the cells cultured according to the methods disclosed herein exhibit a more durable in vivo response upon transplantation in a subject. In some aspects, the subject is a human.

In some aspects, at least about 5% of the cells in the cell composition have a stem-like phenotype. In some aspects, at least about 10% of the cells in the cell composition have a stem-like phenotype. In some aspects, at least about 15% of the cells in the cell composition have a stem-like phenotype. In some aspects, at least about 20% of the cells in the cell composition have a stem-like phenotype. In some aspects, at least about 25% of the cells in the cell composition have a stem-like phenotype. In some aspects, at least about 30% of the cells in the cell composition have a stem-like phenotype. In some aspects, at least about 35% of the cells in the cell composition have a stem-like phenotype. In some aspects, at least about 40% of the cells in the cell composition have a stem-like phenotype. In some aspects, at least about 45% of the cells in the cell composition have a stem-like phenotype. In some aspects, at least about 50% of the cells in the cell composition have a stem-like phenotype. In some aspects, at least about 55% of the cells in the cell composition have a stem-like phenotype. In some aspects, at least about 60% of the cells in the cell composition have a stem-like phenotype. In some aspects, at least about 65% of the cells in the cell composition have a stem-like phenotype. In some aspects, at least about 70% of the cells in the cell composition have a stem-like phenotype.

In some aspects, following culture of T cells according to the methods disclosed herein, stem-like T cells constitute at least about 10% to at least about 70% of the total number of T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD8⁺ T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD4+ T cells in the culture.

In some aspects, the number of cells having a stem-like phenotype in the cell composition is increased at least about 1.5-fold as compared to the number of cells in the cell composition prior to the culture. In some aspects, the number of cells having a stem-like phenotype in the cell composition is increased at least about 2.0-fold as compared to the number of cells in the cell composition prior to the culture. In some aspects, the number of cells having a stem-like phenotype in the cell composition is increased at least about 2.5-fold as compared to the number of cells in the cell composition prior to the culture. In some aspects, the number of cells having a stem-like phenotype in the cell composition is increased at least about 3.0-fold as compared to the number of cells in the cell composition prior to the culture. In some aspects, the number of cells having a stem-like phenotype in the cell composition is increased at least about 3.5-fold as compared to the number of cells in the cell composition prior to the culture. In some aspects, the number of cells having a stem-like phenotype in the cell composition is increased at least about 4.0-fold as compared to the number of cells in the cell composition prior to the culture. In some aspects, the number of cells having a stem-like phenotype in the cell composition is increased at least about 4.5-fold as compared to the number of cells in the cell composition prior to the culture. In some aspects, the number of cells having a stem-like phenotype in the cell composition is increased at least about 5.0-fold as compared to the number of cells in the cell composition prior to the culture. In some aspects, the number of cells having a stem-like phenotype in the cell composition is increased at least about 5.5-fold as compared to the number of cells in the cell composition prior to the culture. In some aspects, the number of cells having a stem-like phenotype in the cell composition is increased at least about 6.0-fold as compared to the number of cells in the cell composition prior to the culture. In some aspects, the number of cells having a stem-like phenotype in the cell composition is increased at least about 6.5-fold as compared to the number of cells in the cell composition prior to the culture. In some aspects, the number of cells having a stem-like phenotype in the cell composition is increased at least about 7.0-fold as compared to the number of cells in the cell composition prior to the culture. In some aspects, the number of cells having a stem-like phenotype in the cell composition is increased at least about 7.5-fold as compared to the number of cells in the cell composition prior to the culture. In some aspects, the number of cells having a stem-like phenotype in the cell composition is increased at least about 8.0-fold as compared to the number of cells in the cell composition prior to the culture. In some aspects, the number of cells having a stem-like phenotype in the cell composition is increased at least about 9.0-fold as compared to the number of cells in the cell composition prior to the culture. In some aspects, the number of cells having a stem-like phenotype in the cell composition is increased at least about 10-fold as compared to the number of cells in the cell composition prior to the culture. In some aspects, the number of cells having a stem-like phenotype in the cell composition is increased at least about 15-fold as compared to the number of cells in the cell composition prior to the culture. In some aspects, the number of cells having a stem-like phenotype in the cell composition is increased at least about 20-fold as compared to the number of cells in the cell composition prior to the culture. In some aspects, the number of cells having a stem-like phenotype in the cell composition is increased at least about 30-fold as compared to the number of cells in the cell composition prior to the culture. In some aspects, the number of cells having a stem-like phenotype in the cell composition is increased at least about 40-fold as compared to the number of cells in the cell composition prior to the culture. In some aspects, the number of cells having a stem-like phenotype in the cell composition is increased at least about 50-fold as compared to the number of cells in the cell composition prior to the culture. In some aspects, the number of cells having a stem-like phenotype in the cell composition is increased at least about 75-fold as compared to the number of cells in the cell composition prior to the culture. In some aspects, the number of cells having a stem-like phenotype in the cell composition is increased at least about 100-fold as compared to the number of cells in the cell composition prior to the culture. In some aspects, the number of cells having a stem-like phenotype in the cell composition is increased at least about 500-fold as compared to the number of cells in the cell composition prior to the culture. In some aspects, the number of cells having a stem-like phenotype in the cell composition is increased at least about 1000-fold as compared to the number of cells in the cell composition prior to the culture.

In some aspects, following culture of T cells according to the methods disclosed herein, at least about 10% to at least about 70% of the total number of T cells in the culture are CD39⁻/TCF7⁺ T cells. In some aspects, following culture of T cells according to the methods disclosed herein, at least about 10%, at least about 15%, at least about 20%, at least about 25%, at least about 30%, at least about 35%. or at least about 40% of the total number of T cells in the culture are CD39⁻/TCF7⁺ T cells. In some aspects the T cells are CD4+ T cells. In some aspects the T cells are CD8⁺ T cells.

In some aspects, the cell composition comprises immune cells, e.g., T cells and/or NK cells. In some aspects, the cell composition comprises an increase percent of immune cells, e.g., T cells and/or NK cells, which express CD95. In some aspects, the cell composition comprises an increase in the percent of immune cells, e.g., T cells and/or NK cells, which do not express CD45R0. In some aspects, the cell composition comprises an increase in the percent of immune cells, e.g., T cells and/or NK cells, which express CD45RA. In some aspects, the cell composition comprises an increase in the percent of immune cells, e.g., T cells and/or NK cells, which express CCR7. In some aspects, the cell composition comprises an increase in the percent of immune cells, e.g., T cells and/or NK cells, which express CD62L. In some aspects, the cell composition comprises an increase in the percent of immune cells, e.g., T cells and/or NK cells, which express TCF7. In some aspects, the cell composition comprises an increase in the percent of immune cells, e.g., T cells and/or NK cells, which express CD3. In some aspects, the cell composition comprises an increase in the percent of immune cells, e.g., T cells and/or NK cells, which express CD27. In some aspects, the cell composition comprises an increase in the percent of immune cells, e.g., T cells and/or NK cells, which express CD95 and CD45RA. In some aspects, the cell composition comprises an increase in the percent of immune cells, e.g., T cells and/or NK cells, which express CD45RA and CCR7. In some aspects, the cell composition comprises an increase in the percent of immune cells, e.g., T cells and/or NK cells, which express CD95, CD45RA, and CCR7. In some aspects, the cell composition comprises an increase in the percent of immune cells, e.g., T cells and/or NK cells, which express CD45RA, CCR7, and CD62L. In some aspects, the cell composition comprises an increase in the percent of immune cells, e.g., T cells and/or NK cells, which express CD95, CD45RA, CCR7, and CD62L. In some aspects, the cell composition comprises an increase in the percent of immune cells, e.g., T cells and/or NK cells, which express CD45RA, CCR7, CD62L, and TCF7. In some aspects, the cell composition comprises an increase in the percent of immune cells, e.g., T cells and/or NK cells, which express CD95, CD45RA, CCR7, CD62L, and TCF7. In some aspects, the cell composition comprises an increase in the percent of immune cells, e.g., T cells and/or NK cells, which express CD45RA, CCR7, CD62L, TCF7, and CD27. In some aspects, the cell composition comprises an increase in the percent of immune cells, e.g., T cells and/or NK cells, which express CD95, CD45RA, CCR7, CD62L, TCF7, and CD27. In some aspects, the cell composition comprises an increase in the percent of immune cells, e.g., T cells and/or NK cells, which express, CD45RA, CCR7, CD62L, TCF7, and CD27, and which do not express CD45RO or which are CD45RO^{low}. In some aspects, the cell composition comprises an increase in the percent of immune cells, e.g., T cells and/or NK cells, which express CD95, CD45RA, CCR7, CD62L, TCF7, and CD27, and which do not express CD45RO or which are CD45RO^{low}.

In some aspects, the cell composition comprises an increase in the percent of immune cells, e.g., T cells and/or NK cells, which do not express CD39 and CD69. In some aspects, the cell composition comprises an increase in the percent of immune cells, e.g., T cells and/or NK cells, which express CD8, and which do not express CD39 and CD69. In some aspects, following culture of T cells according to the methods disclosed herein, at least about 10% to at least about 40% of the total number of T cells in the culture are CD39⁻/CD69⁻ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39-/CD69⁻ T cells.

In some aspects, the cell composition comprises an increased percentage of immune cells, e.g., T cells and/or NK cells, which express both (i) one or more stem-like markers and (ii) one or more effector-like markers. In some aspects, the cell composition comprises an increased percentage of immune cells, e.g., T cells and/or NK cells, which express at least two stem-like markers and one or more effector-like markers. In some aspects, the cell composition comprises an increase in the percent of immune cells, e.g., T cells and/or NK cells, which express at least three stem-like markers and one or more effector-like markers. In some aspects, the cell composition comprises an increased percentage of immune cells, e.g., T cells and/or NK cells, which express at least four stem-like markers and one or more effector-like markers. In some aspects, the cell composition comprises an increased percentage of immune cells, e.g., T cells and/or NK cells, which express one or more stem-like markers and at least two effector-like markers.

In some aspects, the stem-like markers are selected from CD45RA+, CD62L+, CCR7+, CD27+, CD28+, BACH2+, LEF1+, TCF7+, and any combination thereof. In some aspects the stem-like markers comprise CD45RA+, CD62L+, CCR7+, and TCF7+, or any combination thereof. In some aspects, the immune cell expresses CD45RO^{low}. In some aspects, the stem-like markers comprise one or more genes listed herein as part of a gene-signature (see supra; see, e.g., Gattinoni, L., et al., Nat Med 17(10): 1290-1297 (2011) or Galletti et al. Nat Immunol 21, 1552-1562 (2020)).

In some aspects, the effector-like markers are selected from pSTAT5⁺, STAT5⁺, pSTAT3⁺, STAT3⁺, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL11RA, IL12A, IL12B, IL12RB1, IL12RB2, IL13, IL13RA1, IL13RA2, IL 15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, ILS, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof.

In some aspects, the stem-like markers comprise a gene expressed in the WNT signaling pathway. In some aspects, the stem-like markers comprise one or more genes selected from GNG2, PSMC3, PSMB10, PSMC5, PSMB8, PSMB9, AKT1, MYC, CLTB, PSME1, DVL2, PFN1, H2AFJ, LEF1, CTBP1, MOV10, HIST1H2BD, FZD3, ITPR3, PARD6A, LRP5, HIST2H4A, HIST2H3C, HIST1H2AD, HIST2H2BE, HIST3H2BB, DACT1, and any combination thereof. In some aspects, the stem-like markers comprise one or more genes selected from MYC, AKT1, LEF1, and any combination thereof.

In some aspects, the effector-like markers are effector memory-associated genes that comprise one or more genes selected from TBCD, ARL4C, KLF6, LPGAT1, LPIN2, WDFY1, PCBP4, PIK343, FAS, LLGL2, PPP2R2B, TTC39C, GGA2, LRP8, PMAIP1, MVD, IL15RA, FHOD1, EML4, PEA15, PLEKHA5, WSB2, PAM, CD68, MSC, TLR3, S1PR5, KLRB1, CYTH3, RAB27B, SCD5, and any combination thereof. In some aspects, the effector-like markers comprise one or more genes selected from KLF6, FAS, KLRB1, TLR3, and any combination thereof.

In some aspects, the cell composition comprises an increase in the percent of immune cells that are CD45RA+, STAT5+, and STAT3+. In some aspects, the cell composition comprises an increase in the percent of immune cells that are CD62L+, STAT5+, and STAT3+. In some aspects, the cell composition comprises an increase in the percent of immune cells that are TCF7+, STAT5+, and STAT3+. In some aspects, the cell composition comprises an increase in the percent of immune cells that are CD45RA+, CD62L+, CCR7+, CD27+, CD28+, BACH2+, LEF1+, TCF7+, STAT5+, and STAT3+. In some aspects, the cell composition comprises an increase in the percent of immune cells that are CD45RA+, CD62L+, CCR7+, CD27+, CD28+, BACH2+, LEF1+, TCF7+, pSTAT5+, STAT5+, pSTAT3+, and STAT3+. In some aspects, the cell composition comprises an increase in the percent of immune cells that are CD45RA+, CD45RO⁻, CD62L+, CCR7+, CD27+, CD28+, BACH2+, LEF1+, TCF7+, pSTAT5+, STAT5+, pSTAT3+, and STAT3+.

In some aspects, an immune cell comprises one or more markers selected from CD45RA+, CD62L+, CCR7+, CD27+, CD28+, BACH2+, LEF1+, TCF7+, and any combination thereof and one or more markers selected from pSTAT5+, STAT5+, pSTAT3+, STAT3+, and any combination thereof. In some aspects, the immune cell expresses CD45RO^{low}. In some aspects, an immune cell comprises one or more markers selected from CD45RA+, CD62L+, CCR7+, CD27+, CD28+, BACH2+, LEF1+, TCF7+, and any combination thereof and one or more effector-like markers. In some aspects, an immune cell comprises one or more stem-like markers and one or more markers selected from pSTAT5+, STAT5+, pSTAT3+, STAT3+, and any combination thereof. In some aspects, the immune cell expresses CD45RO^{low}.

Some aspects of the present disclosure are directed to an immune cell, e.g., T cell, NK cell, or TIL, which expresses one or more stem-like markers and one or more effector-like marker. In some aspects, the immune cell, e.g., T cell, NK cell, or TIL, expresses at least two stem-like markers and one or more effector-like markers. In some aspects, the immune cell, e.g., T cell, NK cell, or TIL, expresses at least three stem-like markers and one or more effector-like markers. In some aspects, the immune cell, e.g., T cell, NK cell, or TIL, expresses at least four stem-like markers and one or more effector-like markers. In some aspects, the immune cell, e.g., T cell, NK cell, or TIL, expresses one or more stem-like markers and at least two effector-like markers. In some aspects, the stem-like markers are selected from CD45RA+, CD62L+, CCR7+, CD27+, CD28+, BACH2+, LEF1+, TCF7+, and any combination thereof. In some aspects the stem-like markers comprise CD45RA+, CD62L+, CCR7+, and TCF7+, or any combination thereof. In some aspects, the immune cell expresses CD45RO^{low}. In some aspects, the stem-like markers comprise one or more genes listed herein as part of a gene-signature (see supra; see, e.g., Gattinoni, L., et al., Nat Med 17(10): 1290-1297 (2011) or Galletti et al. Nat Immunol 21, 1552-1562 (2020)). In some aspects, the effector-like markers are selected from pSTAT5+, STAT5+, pSTAT3+, STAT3+, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL1IRA, IL12A, IL12B, IL12RB1, IL12RB2, IL 13, IL13RA1, IL 13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, ILS, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof.

In some aspects, the immune cell, e.g., T cell, NK cell, or TIL, expresses CD45RA+, STAT5+, and STAT3+. In some aspects, the immune cell, e.g., T cell, NK cell, or TIL, expresses CD62L+, STAT5+, and STAT3+. In some aspects, the immune cell, e.g., T cell, NK cell, or TIL, expresses TCF7+, STAT5+, and STAT3+. In some aspects, the immune cell, e.g., T cell, NK cell, or TIL, expresses CD45RA+, CD62L+, CCR7+, CD27+, CD28+, BACH2+, LEF1+, TCF7+, STAT5+, and STAT3+. In some aspects, the cell composition comprises an increase in the percent of immune cells that are CD45RA+, CD62L+, CCR7+, CD27+, CD28+, BACH2+, LEF1+, TCF7+, pSTAT5+, STAT5+, pSTAT3+, and STAT3+. In some aspects, the immune cell, e.g., T cell, NK cell, or TIL, expresses CD45RA+, CD45RO^{low}, CD62L+, CCR7+, CD27+, CD28+, BACH2+, LEF1+, TCF7+, pSTAT5+, STAT5+, pSTAT3+, and STAT3+.

Some aspects of the present disclosure are directed to a cell composition comprising a population of immune cells, e.g., T cells and/or NK cells, wherein the population of immune cells comprises (i) a first sub-population of immune cells expressing one or more stem-like markers (e.g., stem-like immune cells) and (ii) a second sub-population of immune cells expressing one or more effector-like marker (e.g., effector-like immune cells), wherein the population of immune cells comprises a higher percentage (i.e., the number of stem-like immune cells/the total number of immune cells) of the first sub-population of immune cells expressing one or more stem-like markers, as compared to a population of immune cells cultured in a control media. In some aspects, the immune cells, e.g., T cells and/or NK cells, cultured according to the methods disclosed herein result in these cell compositions. In some aspects, immune cells, e.g., T cells and/or NK cells, cultured according to the methods disclosed herein have increased expression, e.g., a higher percentage of immune cells, e.g., T cells and/or NK cells, that express, GZMB, MHC-II, LAG3, TIGIT, and/or NKG7, and decreased expression, e.g., a lower percentage of immune cells, e.g., T cells and/or NK cells, that express, IL-32. Cells highest for NKG7 have been shown to be better killers (Malarkannan et al. 2020 Nat. Immuno.), whereas cells higher in IL-32 have been shown to have activation-induced cell death (Goda et al., 2006 Int. Immunol). In some aspects the immune cells, e.g., T cells and/or NK cells, with higher expression of GZMB, MHC-II, LAG3, TIGIT, and/or NKG7 are CD8+ T cells expressing effector-like markers. In some aspects the immune cells, e.g., T cells and/or NK cells, with lower expression of IL-32 are CD8+ T cells expressing effector-like markers.

Some aspects of the present disclosure are directed to a cell composition comprising the immune cell (e.g., T cell, NK cell, or TIL) or the engineered immune cell. Some aspects of the present disclosure are directed to a population of cells comprising the immune cell (e.g., T cell, NK cell, or TIL) or engineered immune cell. In some aspects, at least about 40%, at least about 50%, at least about 60%, at least about 70%, at least about 80%, at least about 85%, at least about 90%, at least about 95%, at least about 99%, or about 100% of the engineered cells comprise the immune cell.

In some aspects, the immune cell is an engineered immune cell. As used herein, an “engineered” immune cell refers to an immune cell that has been manipulated in a way, e.g., according to the methods disclosed herein, that confers on the cell one or more physical and/or functional properties that are not characteristic of a naturally occurring immune cell. For example, in some aspects, an engineered immune cell can be generated by modifying an immune cell to express one or more proteins heterologous to the cell (e.g., chimeric antigen receptor or T cell receptor) so that the engineered immune cell is not naturally occurring. In some aspects, an engineered immune cell can be generated by culturing an immune cell in a particular way, e.g., culturing in hypotonic and hyperkalemic medium, wherein the resulting engineered immune cell has one or more physical and/or functional properties that are not shown in naturally occurring cells. In some aspects, the immune cell, e.g., engineered cell, that expresses one or more stem-like markers and one or more effector-like markers, e.g., an engineered cell of the disclosure, is a T stem/effector (TSE) cell. In some aspects, the TSE cell retains a less differentiated state (e.g., expresses one or more stem-like markers, is capable of proliferation, is capable of differentiation, or any combination thereof) and the cell has effector function (e.g., expresses one or more effector-like markers, is capable of targeting and/or killing tumor cells, exhibits polyfunctionality, or a combination thereof). In some aspects, a TSE cell disclosed herein expresses CD45RA+, CD62L+, CCR7+, CD27+, CD28+, BACH2+, LEF1+, TCF7+, pSTAT5+, STAT5+, pSTAT3+, and STAT3+. In some aspects, a TSE cell disclosed herein expresses CD45RA+, CD62L+, CCR7+, TCF7+, pSTAT5+, STAT5+, pSTAT3+, and STAT3+. In some aspects, the TSE cell is CD45RO^{low}. In some aspects, the TSE cell is an expresses a recombinant receptor, e.g., as disclosed herein. Some aspects of the present disclosure are directed to a cell composition comprising one or more TSE cell. Some aspects of the present disclosure are directed to a population of engineered immune cells comprising one or more TSE cell. In some aspects, at least about 40%, at least about 50%, at least about 60%, at least about 70%, at least about 80%, at least about 85%, at least about 90%, at least about 95%, at least about 99%, or about 100% of the engineered immune cells in the population are TSE cells. In some aspects, at least about 40% of the engineered immune cells in the population are TSE cells. In some aspects, at least about 50% of the engineered immune cells in the population are TSE cells. In some aspects, at least about 60% of the engineered immune cells in the population are TSE cells. In some aspects, at least about 70% of the engineered immune cells in the population are TSE cells. In some aspects, at least about 75% of the engineered immune cells in the population are TSE cells. In some aspects, at least about 80% of the engineered immune cells in the population are TSE cells. In some aspects, at least about 85% of the engineered immune cells in the population are TSE cells. In some aspects, at least about 90% of the engineered immune cells in the population are TSE cells. In some aspects, at least about 95% of the engineered immune cells in the population are TSE cells. In some aspects, at least about 98% of the engineered immune cells in the population are TSE cells. In some aspects, at least about 99% of the engineered immune cells in the population are TSE cells. In some aspects, about 100% of the engineered immune cells in the population are TSE cells.

In some aspects, the cell composition comprises one or more immune cell, e.g., T cells and/or NK cells, which is genetically engineered. In some aspects, the cell composition comprises one or more immune cell, e.g., T cells and/or NK cells, which is engineered to express a chimeric antigen receptor (CAR). Any CAR disclosed herein, e.g., in section II.G.1., above, can be used in the cells of the cell composition.

In some aspects, the cell composition comprises one or more immune cell, e.g., T cells and/or NK cells, which is engineered to express a T cell receptor (TCR), e.g., an engineered TCR. Any TCR disclosed herein, e.g., in section II.G.2., above, can be used in the cells of the cell composition.

In some aspects, the cell composition comprises one or more immune cell, e.g., T cells and/or NK cells, which is engineered to express a TCRm. Any TCRm disclosed herein, e.g., in section II.G.3., above, can be used in the cells of the cell composition.

In some aspects, the cell composition, obtained by any method described herein (e.g., the yield of the final cell product for use as a therapy), comprises at least about 1×10⁵, 5×10⁵, 1×10⁶, 5×10⁶, 1×10⁷, 5×10⁷, 1×10⁸, 5×10⁸, 1×10⁹, or 5×10⁹ cells. In some aspects, the cell composition, obtained by any method described herein, comprises at least about 1×10³, 5×10³, 1×10⁴, 5×10⁴, 1×10⁵, 5×10⁵, 1×10⁶,5×10⁷, 5×10⁷, 1×10⁸, 5×10⁸, 1×10⁹, or 5×10⁹ stem-like cells. In some aspects, the cell composition, obtained by any method described herein, comprises at least about 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 10×10¹⁰, 11×10¹⁰, 12×10¹⁰, 13×10¹⁰, 14×10¹⁰, or 15×10¹⁰ cells. In some aspects, the cell composition, obtained by any method described herein, comprises at least about 1×10⁶ cells. In some aspects, the cell composition, obtained by any method described herein, comprises at least about 1×10⁶ stem-like cells. In some aspects, the cell composition, obtained by any method described herein, comprises at least about 1×10¹⁰ cells. In some aspects, the cell composition, obtained by any method described herein, comprises at least about 2×10¹⁰ cells. In some aspects, the cell composition, obtained by any method described herein, comprises at least about 3×10¹⁰ cells. In some aspects, the cell composition, obtained by any method described herein, comprises at least about 4×10¹⁰ cells. In some aspects, the cell composition, obtained by any method described herein, comprises at least about 5×10¹⁰ cells. In some aspects, the cell composition, obtained by any method described herein, comprises at least about 6×10¹⁰ cells. In some aspects, the cell composition, obtained by any method described herein, comprises at least about 7×10¹⁰ cells. In some aspects, the cell composition, obtained by any method described herein, comprises at least about 8×10¹⁰ cells. In some aspects, the cell composition, obtained by any method described herein, comprises at least about 9×10¹⁰ cells. In some aspects, the cell composition, obtained by any method described herein, comprises at least about 10×10¹⁰ cells. In some aspects, the cell composition, obtained by any method described herein, comprises at least about 11×10¹⁰ cells. In some aspects, the cell composition, obtained by any method described herein, comprises at least about 12×10¹⁰ cells. In some aspects, the cell composition, obtained by any method described herein, comprises at least about 13×10¹⁰ cells. In some aspects, the cell composition, obtained by any method described herein, comprises at least about 14×10¹⁰ cells. In some aspects, the cell composition, obtained by any method described herein, comprises at least about 15×10¹⁰ cells. In some aspects, cell yield represents the total number of CD3⁺ cells. In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ expanded cells by at least about day 10 of culturing in the presently disclosed medium. In some aspects, the methods disclosed herein yield a composition comprising at least about 1.8×10¹⁰ expanded immune cells by at least about day 10 of culturing in the presently disclosed medium.

In some aspects, the cell composition comprises at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells. In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells by at least about day 10 of culture. In some aspects, the methods disclosed herein yield a composition comprising at least about 1.8×10¹⁰ stem-like cells by at least about day 10 of culturing in the presently disclosed medium.

In some aspects, the methods disclosed herein yield a composition comprising immune cells that are at least about 80%, at least about 85%, at least about 90%, at least about 94%, at least about 95%, at least about 96%, at least about 97%, at least about 98%, or at least about 99% viable. In some aspects, the methods disclosed herein yield a composition comprising at least about 1.8×10¹⁰ stem-like cells with at least about 94% cell viability.

Some aspects of the present disclosure are directed to a composition comprising a population of TILs, which is enriched in CD8⁺ TILs. In some aspects, the composition comprises a population of TILs cultured according to any method disclosed herein. In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the TILs are CD8⁺ TILs. In some aspects, at least about 20% of the TILs are CD8⁺ TILs. In some aspects, at least about 30% of TILs are CD8⁺ TILs. In some aspects, at least about 40% of the TILs are CD8⁺ TILs. In some aspects, at least about 50% of the TILs are CD8⁺ TILs. In some aspects, at least about 60% of the TILs are CD8⁺ TILs. In some aspects, at least about 70% of the TILs are CD8⁺ TILs. In some aspects, at least about 80% of the TILs are CD8⁺ TILs. In some aspects, at least about 90% of the TILs are CD8⁺ TILs. In some aspects, at least about 95% of TILs are CD8⁺ TILs.

In some aspects, the TILs exhibit increased expression of one or more biomarker indicative of a less-differentiated phenotype. In some aspects, the TILs exhibit increased expression of TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold increase in the expression of TCF7. In some aspects, the TILs (e.g., the CD8⁺ TILs) cultured according to the methods disclosed herein exhibit an at least about 40-fold increase in the expression of TCF7. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ TCF7T TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are TCF7⁺.

In some aspects, the TILs exhibit increased expression of CD45RO. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD45RO. In some aspects, the TILs (e.g., the CD8⁺ TILs) cultured according to the methods disclosed herein exhibit an at least about 10-fold increase in the expression of CD45RO. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD45RO⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD45RO⁺.

In some aspects, the TILs exhibit increased expression of CD62L. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD62L. In some aspects, the TILs (e.g., the CD8⁺ TILs) cultured according to the methods disclosed herein exhibit an at least about 10-fold increase in the expression of CD62L. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD62L⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD62L⁺.

In some aspects, the TILs exhibit increased expression of CD27. In some aspects, the TILs (e.g., the CD8⁺ TILs) cultured according to the methods disclosed herein, exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27. In some aspects, the TILs (e.g., the CD8⁺ TILs) cultured according to the methods disclosed herein exhibit an at least about 10-fold increase in the expression of CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺.

In some aspects, the TILs exhibit increased expression of CD62L and CD27. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD62L and CD27. In some aspects, the TILs (e.g., the CD8⁺ TILs) cultured according to the methods disclosed herein exhibit an at least about 10-fold increase in the expression of CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺/CD62L⁺/CD27⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD62L⁺ CD27⁺.

In some aspects, the TILs exhibit increased expression of CD28. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD28. In some aspects, the TILs (e.g., the CD8⁺ TILs) cultured according to the methods disclosed herein exhibit an at least about 10-fold increase in the expression of CD28. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/CD28⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TTLs are CD28⁺.

In some aspects, the TILs exhibit increased expression of CD27 and CD28. In some aspects, the TTLs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27 and CD28. In some aspects, the TILs (e.g., the CD8⁺ TILs) cultured according to the methods disclosed herein exhibit an at least about 10-fold increase in the expression of CD27 and CD28. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺.

In some aspects, the TILs exhibit increased expression of CD27, CD28, PD1, and CD103. In some aspects, the TTLs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, and CD103. In some aspects, the TILs (e.g., the CD8⁺ TILs) cultured according to the methods disclosed herein exhibit an at least about 10-fold increase in the expression of CD27, CD28, PD1, and CD103. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺PD1⁺ CD103⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺ PD1⁺ CD103+.

In some aspects, the TILs exhibit increased expression of CD27, CD28, PD1, and TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, and TCF7. In some aspects, the TILs (e.g., the CD8⁺ TILs) cultured according to the methods disclosed herein exhibit an at least about 10-fold increase in the expression of CD27, CD28, PD1, and TCF7. In some aspects, at least about 5%, at least about 10%, at least about 15%, at least about 20%, at least about 25%, at least about 30%, at least about 35%, at least ab out 40%, at least about 45%, at least about 50%, at least about 55%, at least about 60%, at least about 65%, at least about 70%, or at least about 75% of the TILs are CD8⁺ CD27⁺ CD28⁺ PD1⁺ TCF7⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺ PD1⁺ TCF7+.

In some aspects, the TILs exhibit increased expression of CD27, CD28, PD1, CD103, and TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, CD103, and TCF7. In some aspects, the TILs (e.g., the CD8⁺ TILs) cultured according to the methods disclosed herein exhibit an at least about 10-fold increase in the expression of CD27, CD28, PD1, CD103, and TCF7. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺ PD1⁺ CD103⁺ TCF7⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺TILs are CD27⁺ CD28⁺ PD1⁺ CD103⁺ TCF7+.

In some aspects, the TILs exhibit increased expression of CD39. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD39. In some aspects, the TILs (e.g., the CD8⁺ TILs) cultured according to the methods disclosed herein exhibit an at least about 10-fold increase in the expression of CD39. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD39⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD39⁺.

In some aspects, the TILs exhibit increased expression of CD39 and PD1. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD39 and PD1. In some aspects, the e TILs (e.g., the CD8⁺ TILs) cultured according to the methods disclosed herein exhibit an at least about 10-fold increase in the expression of CD39 and PD1. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD39⁺ PD1⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD39⁺ PD1+.

In some aspects, the TILs exhibit increased expression of PD1. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of PD1. In some aspects, the TTLs (e.g., the CD8⁺ TILs) cultured according to the methods disclosed herein exhibit an at least about 10-fold increase in the expression of PD1. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/PD1⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TTLs are PD1.

In some aspects, the TTLs exhibit increased expression of PD1 and CD27. In some aspects, the TTLs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of PD1 and CD27. In some aspects, the TILs (e.g., the CD8⁺ TILs) cultured according to the methods disclosed herein exhibit an at least about 10-fold increase in the expression of PD1 and CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ PD1⁺ CD27⁺. TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are PD1V CD27⁺.

In some aspects, the TILs exhibit increased expression of CD103. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD103. In some aspects, the TILs (e.g., the CD8⁺ TILs) cultured according to the methods disclosed herein exhibit an at least about 10-fold increase in the expression of CD103. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺/CD103⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD103⁺.

In some aspects, the TILs (e.g., CD8⁺ TILs) cultured according to the methods and/or in the medium disclosed herein have an increased number of less-differentiated cells as compared to comparable immune cells cultured according to conventional methods. In some aspects, the TILs (e.g., CD8⁺ TILs) cultured according to the methods disclosed herein exhibit increased expression of one or more marker typical of a stem-like phenotype. In some aspects, the TILs (e.g., CD8⁺ TILs) cultured according to the methods disclosed herein exhibit increased in vivo viability upon transplantation in a subject. In some aspects, the TILs (e.g., CD8⁺ TILs) cultured according to the methods disclosed herein exhibit increased cell potency. In some aspects, the TILs (e.g., CD8⁺ TILs) cultured according to the methods disclosed herein exhibit decreased cell exhaustion. In some aspects, the TILs (e.g., CD8⁺ TILs) cultured according to the methods disclosed herein exhibit increased in vivo persistence upon transplantation in a subject. In some aspects, the TILs (e.g., CD8⁺ TILs) cultured according to the methods disclosed herein exhibit increased in vivo activity upon transplantation in a subject. In some aspects, the TILs (e.g., CD8⁺ TILs) cultured according to the methods disclosed herein exhibit a more durable in vivo response upon transplantation in a subject. In some aspects, the subject is a human.

In some aspects, at least about 5% of the TILs (e.g., CD8⁺ TILs) in the composition have a stem-like phenotype. In some aspects, at least about 10% of the TILs (e.g., CD8⁺ TILs) in the composition have a stem-like phenotype. In some aspects, at least about 15% of the TILs (e.g., CD8⁺ TILs) in the composition have a stem-like phenotype. In some aspects, at least about 20% of the TILs (e.g., CD8⁺ TILs) in the composition have a stem-like phenotype. In some aspects, at least about 25% of the TILs (e.g., CD8⁺ TILs) in the composition have a stem-like phenotype. In some aspects, at least about 30% of the TILs (e.g., CD8⁺ TILs) in the composition have a stem-like phenotype. In some aspects, at least about 35% of the TILs (e.g., CD8⁺ TILs) in the composition have a stem-like phenotype. In some aspects, at least about 40% of the TILs (e.g., CD8⁺ TILs) in the composition have a stem-like phenotype. In some aspects, at least about 45% of the TILs (e.g., CD8⁺ TILs) in the composition have a stem-like phenotype. In some aspects, at least about 50% of the TILs (e.g., CD8⁺ TILs) in the composition have a stem-like phenotype.

In some aspects, the number of TILs (e.g., CD8⁺ TILs) having a stem-like phenotype in the composition is increased at least about 1.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 2.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 2.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 3.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 3.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 4.0-fold as compared to the number of TILs (e.g., CD8⁺ TILs) in the composition prior to the culture. In some aspects, the number of cells having a stem-like phenotype in the composition is increased at least about 4.5-fold as compared to the number of TILs (e.g., CD8⁺TILs) in the composition prior to the culture. In some aspects, the number of TILs (e.g., CD8⁺TILs) having a stem-like phenotype in the composition is increased at least about 5.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 5.5-fold as compared to the number of TILs (e.g., CD8⁺TILs) in the composition prior to the culture. In some aspects, the number of TILs (e.g., CD8⁺TILs) having a stem-like phenotype in the composition is increased at least about 6.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 6.5-fold as compared to the number of TILs (e.g., CD8⁺TILs) in the composition prior to the culture. In some aspects, the number of TILs (e.g., CD8⁺ TILs) having a stem-like phenotype in the composition is increased at least about 7.0-fold as compared to the number of cells in the composition prior to the culture. In some aspects, the number of TILs (e.g., CD8⁺ TILs), having a stem-like phenotype in the composition is increased at least about 7.5-fold as compared to the number of cells in the composition prior to the culture. In some aspects, the number of TILs (e.g., CD8⁺ TILs) having a stem-like phenotype in the composition is increased at least about 8.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 9.0-fold as compared to the number of TILs (e.g., CD8⁺ TILs) in the composition prior to the culture. In some aspects, the number of cells having a stem-like phenotype in the composition is increased at least about 10-fold as compared to the number of TILs (e.g., CD8⁺TILs) in the composition prior to the culture. In some aspects, the number of cells having a stem-like phenotype in the composition is increased at least about 15-fold as compared to the number of TILs (e.g., CD8⁺ TILs) in the composition prior to the culture. In some aspects, the number of cells having a stem-like phenotype in the composition is increased at least about 20-fold as compared to the number of TILs (e.g., CD8⁺ TILs) in the composition prior to the culture. In some aspects, the number of TILs having a stem-like phenotype in the composition is increased at least about 30-fold as compared to the number of TILs in the composition prior to the culture. In some aspects, the number of TILs having a stem-like phenotype in the composition is increased at least about 40-fold as compared to the number of cells in the composition prior to the culture. In some aspects, the number of TILs having a stem-like phenotype in the composition is increased at least about 50-fold as compared to the number of TILs in the composition prior to the culture. In some aspects, the number of TILs having a stem-like phenotype in the composition is increased at least about 75-fold as compared to the number of TILs in the composition prior to the culture. In some aspects, the number of TILs having a stem-like phenotype in the composition is increased at least about 100-fold as compared to the number of TILs in the composition prior to the culture. In some aspects, the number of TILs having a stem-like phenotype in the composition is increased at least about 500-fold as compared to the number of TILs in the composition prior to the culture. In some aspects, the number of TILs having a stem-like phenotype in the composition is increased at least about 1000-fold as compared to the number of TILs in the composition prior to the culture.

In some aspects, the cell composition comprises an increase in the percent of TILs which express CD95. In some aspects, the cell composition comprises an increase in the percent of TILs which do not express CD45R0. In some aspects, the cell composition comprises an increase in the percent of TILs which express CD45RA. In some aspects, the cell composition comprises an increase in the percent of TILs which express CCR7. In some aspects, the cell composition comprises an increase in the percent of TILs which express CD62L. In some aspects, the cell composition comprises an increase in the percent of TILs which express TCF7. In some aspects, the cell composition comprises an increase in the percent of TILs which express CD3. In some aspects, the cell composition comprises an increase in the percent of TILs which express CD27. In some aspects, the cell composition comprises an increase in the percent of TILs which express CD95 and CD45RA. In some aspects, the cell composition comprises an increase in the percent of TILs which express CD95, CD45RA, and CCR7. In some aspects, the cell composition comprises an increase in the percent of TILs which express CD95, CD45RA, CCR7, and CD62L. In some aspects, the cell composition comprises an increase in the percent of TILs which express CD95, CD45RA, CCR7, CD62L, and TCF7. In some aspects, the cell composition comprises an increase in the percent of TILs which express CD95, CD45RA, CCR7, CD62L, TCF7, and CD27. In some aspects, the cell composition comprises an increase in the percent of TILs which express CD95, CD45RA, CCR7, CD62L, TCF7, and CD27, and which do not express CD45R0.

In some aspects, the cell composition after the initial culture comprises at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶ cells (e.g., TILs). In some aspects, the cell composition after the initial culture comprises about 2×10⁶ to about 10×10⁶, e.g., about 2×10⁶, about 3×10⁶, about 4×10⁶, about 5×10⁶, about 6×10⁶, about 7×10⁶, about 8×10⁶, about 9×10⁶, or about 10×10⁶, cells (e.g., TILs). In some aspects, the composition after the initial culture comprises about 2×10⁶ cells (e.g., TILs) to about 3×10⁶ cells (e.g., TILs). In some aspects, the composition after the initial culture comprises about 3×10⁶ cells (e.g., TILs) to about 4×10⁶ cells (e.g., TILs). In some aspects, the composition after the initial culture comprises about 4×10⁶ cells (e.g., TILs) to about 5×10⁶ cells (e.g., TILs). In some aspects, the composition after the initial culture comprises about 5×10⁶ cells (e.g., TILs) to about 6×10⁶ cells (e.g., TILs). In some aspects, the composition after the initial culture comprises about 6×10⁶ cells (e.g., TILs) to about 7×10⁶ cells (e.g., TILs). In some aspects, the composition after the initial culture comprises about 7×10⁶ cells (e.g., TILs) to about 8×10⁶ cells (e.g., TILs). In some aspects, the composition after the initial culture comprises about 8×10⁶ cells (e.g., TILs) to about 9×10⁶ cells (e.g., TILs). In some aspects, the composition after the initial culture comprises about 9×10⁶ cells (e.g., TILs) to about 10×10⁶ cells (e.g., TILs).

In some aspects, the cell composition after the second TIL expansion comprises at least about 5×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells (e.g., TILs). In some aspects, the cell composition after the second expansion comprises about 5×10⁷ to about 20×10⁷, e.g., about 5×10⁷, about 6×10⁷, about 7×10⁷, about 8×10⁷, about 9×10⁷, about 10×10⁷, about 11×10⁷, about 12×10⁷, about 13×10⁷, about 14×10⁷, about 15×10⁷, about 16×10⁷, about 17×10⁷, about 18×10⁷, about 19×10⁷, or about 20×10⁷ cells (e.g., TILs). In some aspects, the composition after the second expansion comprises about 5×10⁷ to about 6×10⁷ cells (e.g., TILs), about 6×10⁷ to about 7×10⁷ cells (e.g., TILs), about 7×10⁷ to about 8×10⁷ cells (e.g., TILs), about 8×10⁷ to about 9×10⁷ cells (e.g., TILs), about 9×10⁷ to about 10×10⁷ cells (e.g., TILs), about 10×10⁷ to about 11×10⁷ cells (e.g., TILs), about 11×10⁷ to about 12×10⁷ cells (e.g., TILs), about 12×10⁷ to about 13×10⁷ cells (e.g., TILs), about 13×10⁷ to about 14×10⁷ cells (e.g., TILs), about 14×10⁷ to about 15×10⁷ cells (e.g., TILs), about 15×10⁷ to about 16×10⁷ cells (e.g., TILs), about 16×10⁷ to about 17×10⁷ cells (e.g., TILs), about 17×10⁷ to about 18×10⁷ cells (e.g., TILs), about 18×10⁷ to about 19×10⁷ cells (e.g., TILs), or about 19×10⁷ to about 20×10⁷ cells (e.g., TILs). In some aspects, the composition after the second expansion comprises about 5×10⁷ to about 6×10⁷ cells (e.g., TILs). In some aspects, the composition after the second expansion comprises about 6×10⁷ to about 7×10⁷ cells (e.g., TILs). In some aspects, the composition after the second expansion comprises about 7×10⁷ to about 8×10⁷ cells (e.g., TILs). In some aspects, the composition after the second expansion comprises about 8×10⁷ to about 9×10⁷ cells (e.g., TILs). In some aspects, the composition after the second expansion comprises about 9×10⁷ to about 10×10⁷ cells (e.g., TILs). In some aspects, the composition after the second expansion comprises about 10×10⁷ to about 11×10⁷ cells (e.g., TILs). In some aspects, the composition after the second expansion comprises about 11×10⁷ to about 12×10⁷ cells (e.g., TILs). In some aspects, the composition after the second expansion comprises about 12×10⁷ to about 13×10⁷ cells (e.g., TILs). In some aspects, the composition after the second expansion comprises about 13×10⁷ to about 14×10⁷ cells (e.g., TILs). In some aspects, the composition after the second expansion comprises about 14×10⁷ to about 15×10⁷ cells (e.g., TILs). In some aspects, the composition after the second expansion comprises about 15×10⁷ to about 16×10⁷ cells (e.g., TILs). In some aspects, the composition after the second expansion comprises about 16×10⁷ to about 17×10⁷ cells (e.g., TILs). In some aspects, the composition after the second expansion comprises about 17×10⁷ to about 18×10⁷ cells (e.g., TILs). In some aspects, the composition after the second expansion comprises about 18×10⁷ to about 19×10⁷ cells (e.g., TILs). In some aspects, the composition after the second expansion comprises about 19×10⁷ to about 20×10⁷ cells (e.g., TILs).

In some aspects, the cell composition after the final TIL expansion comprises at least about 10×10⁹, at least about 20×10⁹, at least about 30×10⁹, at least about 40×10⁹, at least about 50×10⁹, at least about 60×10⁹, at least about 70×10⁹, at least about 80×10⁹, at least about 90×10⁹, or at least about 100×10⁹ cells (e.g., TILs). In some aspects, the cell composition after the final expansion comprises about 10×10⁹ to about 100×10⁹, e.g., about 10×10⁹, about 20×10⁹, about 30×10⁹, about 40×10⁹, about 50×10⁹, about 60×10⁹, about 70×10⁹, about 80×10⁹, about 90×10⁹, or about 100×10⁹, cells (e.g., TILs). In some aspects, the composition after the final expansion comprises about 10×10⁹ to about 20×10⁹ cells (e.g., TILs), about 20×10⁹ to about 30×10⁹ cells (e.g., TILs), about 30×10⁹ to about 40×10⁹ cells (e.g., TILs), about 40×10⁹ to about 50×10⁹ cells (e.g., TILs), about 50×10⁹ to about 60×10⁹ cells (e.g., TILs), about 60×10⁹ to about 70×10⁹ cells (e.g., TILs), about 70×10⁹ to about 80×10⁹ cells (e.g., TILs), about 80×10⁹ to about 90×10⁹ cells (e.g., TILs), or about 90×10⁹ to about 100×10⁹ cells (e.g., TILs). In some aspects, the composition after the final expansion comprises about 10×10⁹ to about 20×10⁹ cells (e.g., TILs). In some aspects, the composition after the final expansion comprises about 20×10⁹ to about 30×10⁹ cells (e.g., TILs). In some aspects, the composition after the final expansion comprises about 30×10⁹ to about 40×10⁹ cells (e.g., TILs). In some aspects, the composition after the final expansion comprises about 40×10⁹ to about 50×10⁹ cells (e.g., TILs). In some aspects, the composition after the final expansion comprises about 50×10⁹ to about 60×10⁹ cells (e.g., TILs). In some aspects, the composition after the final expansion comprises about 60×10⁹ to about 70×10⁹ cells (e.g., TILs). In some aspects, the composition after the final expansion comprises about 70×10⁹ to about 80×10⁹ cells (e.g., TILs). In some aspects, the composition after the final expansion comprises about 80×10⁹ to about 90×10⁹ cells (e.g., TILs). In some aspects, the composition after the final expansion comprises about 90×10⁹ to about 100×10⁹ cells (e.g., TILs).

In some aspects, the cell compositions suitable for administration to a subject comprises at least about 2×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10¹⁰, or at least about 10×10¹⁰, or at least about 15×10¹⁰, or at least about 20×10¹⁰, or at least about 25×10¹⁰, or at least about 30×10¹⁰ CD8⁺ TILs. In some aspects, the cell compositions suitable for administration to a subject comprises at least about 2×10⁹ CD8⁺ TILs. In some aspects, the cell compositions suitable for administration to a subject comprises at least about 5×10⁹ CD8⁺ TILs. In some aspects, the cell compositions suitable for administration to a subject comprises at least about 9×10⁹ CD8⁺ TILs. In some aspects, the cell compositions comprises at least about 1×10¹⁰ CD8⁺ TILs. In some aspects, the cell compositions suitable for administration to a subject comprises at least about 10×10¹⁰ CD8⁺TILs. In some aspects, the cell compositions comprises at least about 20×10¹⁰ CD8⁺ TILs. In some aspects, the cell compositions suitable for administration to a subject comprises at least about 30×10¹⁰ CD8⁺ TILs.

IV. Methods of Preparing a Cell Culture Medium

Certain aspects of the present disclosure are directed to methods of making a cell culture medium disclosed herein. Some aspects of the present disclosure are directed to methods of making a hypotonic or isotonic cell culture medium comprising at least about 5 mM potassium ion. Some aspects of the present disclosure are directed to methods of making a cell culture medium comprising at least about 50 mM potassium ion. Some aspects of the present disclosure are directed to methods of making a cell culture medium comprising at least about 50 mM potassium ion and less than 90 mM NaCl. In some aspects, the medium is prepared by modifying a commercially available medium, e.g., a basal medium disclosed herein, to potassium ion at a concentration of at least about 5 mM and one or more of a sodium ion, a calcium ion, a saccharide (e.g., glucose), and a cytokine (e.g., IL-2, IL-7, IL-15, and/or IL-21).

In some aspects, the basal medium is any basal medium disclosed herein, e.g., a basal medium described in section II.F., above. In some aspects, the basal medium comprises potassium ion at a concentration less than 5 mM. In some aspects, the basal medium comprises potassium ion at a concentration less than 50 mM. In some aspects, the basal medium comprises one or more of a sodium ion, a calcium ion, a saccharide (e.g., glucose), and a cytokine (e.g., IL-2, IL-7, IL-15, and/or IL-21).

In some aspects, the basal medium is selected from a balanced salt solution (e.g., PBS, DPBS, HBSS, EBSS), Dulbecco's Modified Eagle's Medium (DMEM), Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™ XF, AIM V, TEXMACS™ medium, and any combination thereof. In some aspects, the basal media comprises PRIME-XV T cell CDM. In some aspects, the basal media comprises OPTMIZER™. In some aspects, the basal media comprises OPTMIZER™ Pro. In some aspects, the basal media comprises X-VIVO™ 15 (LONZA). In some aspects, the basal media comprises IMMUNOCULT™. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal media comprises OPTMIZER™ complete.

In some aspects, suitable basal medium includes Click's medium, OPTIMIZER® (CTS®) medium, STEMLINE® T cell expansion medium (Sigma-Aldrich), AIM V® medium (CTS®), TEXMACS® medium (Miltenyi Biotech), IMMUNOCULT® medium (Stem Cell Technologies), PRIME-XV® T-Cell Expansion XSFM (Irvine Scientific), Iscoves medium, and/or RPMI-1640 medium.

In some aspects, the present disclosure comprises a hyperkalemic medium, e.g., metabolic reprogramming media, comprising basal media, NaCl, KCl, Calcium, and glucose, wherein the concentration of NaCl is between about 40 mM and about 80 mM, the concentration of KCl is between 40 and 90 mM, the concentration of calcium is between about 0.5 mM and about 2.8 mM, and the concentration of Glucose between about 10 mM and about 24 mM. In some aspects, the osmolality of the hyperkalemic medium, e.g., metabolic reprogramming media, is between about 250 mOsmol and about 340mOsmol.

In some aspects, the hyperkalemic medium, e.g., metabolic reprogramming media, further comprises immune cells. In some aspects, the immune cells comprises TILs.

In some aspects, the hyperkalemic medium, e.g., metabolic reprogramming media, further comprises IL-2, IL-7, IL-15, IL-21, or any combination thereof. In some aspects, the hyperkalemic medium, e.g., metabolic reprogramming media, further comprises IL-2 and IL21. In some aspects, the concentration of IL-2 is about 200 ng/ml to about 400 ng/ml (e.g., about 200 ng/ml, about 300 ng/ml, or about 400 ng/ml). In some aspects, the concentration of IL-21 is about 20 ng/ml to about 40 ng/ml, (e.g., about 20 ng/ml, about 30 ng/ml, or about 40 ng/ml).

In some aspects, the hyperkalemic medium, e.g., metabolic reprogramming media, further comprises about 2.5% T cell supplement, about 2.5% serum replacement, about 2 mM L-glutamine, about immune Cell Serum Replacement, about 2 mM L-glutamine, about 2 mM Glutamax, MEM Non-Essential Amino Acids Solution, Pen strep, about 20 μg/ml FUNGIN™, Sodium pyruvate, or any combination thereof. In some aspects, the hyperkalemic medium, e.g., metabolic reprogramming media, further comprises O-Acetyl-L-carnitine hydrochloride. In some aspects, the hyperkalemic medium, e.g., metabolic reprogramming media, further comprises a kinase inhibitor.

In some aspects, the hyperkalemic medium, e.g., metabolic reprogramming media, further comprises a CD3 agonist. In some aspects, the CD3 agonist is an anti-CD3 antibody. In some aspects, the anti-CD3 antibody comprises OKT-3.

In some aspects, the hyperkalemic medium, e.g., metabolic reprogramming media, further comprises a CD28 agonist. In some aspects, the CD28 agonist is an anti-CD28 antibody. In some aspects, the hyperkalemic medium, e.g., metabolic reprogramming media, further comprises a CD27 ligand CD27L). In some aspects, the hyperkalemic medium, e.g., metabolic reprogramming media, further comprises a 4-1BB ligand (4-1BBL).

In some aspects, the present disclosure includes a cell culture comprising the hyperkalemic medium, e.g., metabolic reprogramming media, disclosed herein; a cell bag comprising the hyperkalemic medium, e.g., metabolic reprogramming media, disclosed herein; or a bioreactor comprising the hyperkalemic medium, e.g., metabolic reprogramming media, disclosed herein.

In some aspects, the method of preparing the cell culture medium described herein comprises adding potassium ion to a basal medium until the desired concentration of potassium ion is reached. In some aspects, the potassium ion is added to the basal medium by adding a sufficient amount of a potassium salt in a medium. In some aspects, non-limiting examples of potassium salt include potassium aminetrichloroplatinate, potassium aquapentachlororuthenate, potassium bis(oxalato)platinate(II) dihydrate, potassium bisulfate, potassium borohydride, potassium bromide, potassium carbonate, potassium chloride, potassium chromate, potassium dichromate, potassium dicyanoargentate, potassium dicyanoaurate, potassium fluoride, potassium fluorosulfate, potassium hexachloroiridate, potassium hexachloroosmate, potassium hexachloropalladate, potassium hexachloroplatinate, potassium hexachlororhenate, potassium hexacyanochromate, potassium hexacyanoferrate, potassium hexacyanoruthenate(II) hydrate, potassium hexafluoroantimonate, potassium hexafluoronickelate, potassium hexafluorophosphate, potassium hexafluorotitanate, potassium hexafluorozirconate, potassium hexahydroxoantimonate, potassium hexaiodoplatinate, potassium hexaiodorhenate, potassium hydroxide, potassium iodate, potassium iodide, potassium manganate, potassium metavanadate, potassium molybdate, potassium nitrate, potassium nitrosodisulfonate, potassium osmate(VI) dihydrate, potassium pentachloronitrosylruthenate, potassium perchlorate, potassium perrhenate, potassium perruthenate, potassium persulfate, potassium phosphate dibasic, potassium phosphate monobasic, potassium pyrophosphate, potassium selenocyanate, potassium selenocyanate, potassium stannate trihydrate, potassium sulfate, potassium tellurate hydrate, potassium tellurite, potassium tetraborate tetrahydrate, potassium tetrabromoaurate, potassium tetrabromopalladate, potassium tetrachloropalladate, potassium tetrachloroplatinate, potassium tetracyanopalladate, potassium tetracyanoplatinate, potassium tetrafluoroborate, potassium tetranitroplatinate, potassium tetrathionate, potassium p-toluenethiosulfonate, and otassium hydroxycitrate tribasic monohydrate. In certain aspects, the potassium salt comprises potassium chloride (KCl). In certain aspects, the potassium salt comprises potassium citrate. In certain aspects, the potassium salt comprises potassium hydroxycitrate.

In some aspects of the present disclosure, the cell culture medium comprises potassium ion at a concentration of at least about 5 mM and sodium ion, wherein the medium is hypotonic or isotonic. In some aspects of the present disclosure, the cell culture medium comprises potassium ion at a concentration of at least about 50 mM and sodium ion. In some aspects, the method of preparing the cell culture medium described herein comprises adding sodium ion to a basal medium until the desired concentration of sodium ion is reached. In some aspects, the sodium ion is added to the basal medium adding one or more sodium salts. Non-limiting examples of sodium salts include sodium (meta)periodate, sodium arsenyl tartrate hydrate, sodium azide, sodium benzyloxide, sodium bromide, sodium carbonate, sodium chloride, sodium chromate, sodium cyclohexanebutyrate, sodium ethanethiolate, sodium fluoride, sodium fluorophosphate, sodium formate, sodium hexachloroiridate(III) hydrate, sodium hexachloroiridate(IV) hexahydrate, sodium hexachloroplatinate(IV) hexahydrate, sodium hexachlororhodate(III), sodium hexafluoroaluminate, sodium hexafluoroantimonate(V), sodium hexafluoroarsenate(V), sodium hexafluoroferrate(III), sodium hexafluorophosphate, sodium hexafluorosilicate, sodium hexahydroxyplatinate(IV), sodium hexametaphosphate, sodium hydrogen difluoride, sodium hydrogen sulfate, sodium hydrogencyanamide, sodium hydroxide, sodium iodide, sodium metaborate tetrahydrate, sodium metasilicate nonahydrate, sodium metavanadate, sodium molybdate, sodium nitrate, sodium nitrite, sodium oxalate, sodium perborate monohydrate, sodium percarbonate, sodium perchlorate, sodium periodate, sodium permanganate, sodium perrhenate, sodium phosphate, sodium pyrophosphate, sodium selenate, sodium selenite, sodium stannate, sodium sulfate, sodium tellurite, sodium tetraborate, sodium tetrachloroaluminate, sodium tetrachloroaurate(III), sodium tetrachloropalladate(II), sodium tetrachloroplatinate(II), sodium thiophosphate tribasic, sodium thiosulfate, sodium thiosulfate pentahydrate, sodium yttrium oxyfluoride, Trisodium trimetaphosphate, and any combination thereof. In certain aspect, the sodium salt comprises sodium chloride (NaCl). In certain aspects, the sodium salt comprises sodium bicarbonate. In certain aspects, the sodium salt comprises sodium hydroxycitrate. In certain aspects, the sodium salt comprises sodium phosphate. In some aspects of the present disclosure, the cell culture medium comprises potassium ion at a concentration of at least about 50 mM and NaCl at a concentration of less than 90 mM. In some aspects, the method of preparing the cell culture medium described herein comprises adding NaCl to a basal medium until the desired concentration of sodium ion is reached, wherein the total concentration of potassium and NaCl is more than 110 mM and less than 140 mM.

In some aspects, the desired concentration of sodium ion is lower than that of the basal medium. Therefore, in some aspects, the desired concentration of sodium ion is reached by diluting the basal medium with a suitable solution lacking sodium ion or having a lower concentration of sodium ion.

In some aspects of the present disclosure, the cell culture medium comprises potassium ion at a concentration of at least about 5 mM and calcium ion, wherein the medium is hypotonic or isotonic. In some aspects of the present disclosure, the cell culture medium comprises potassium ion at a concentration of at least about 50 mM and calcium ion. In some aspects of the present disclosure, the cell culture medium comprises potassium ion at a concentration of at least about 50 mM, NaCl at a concentration of less than 90 mM and calcium ion. In some aspects, the method of preparing the cell culture medium described herein comprises adding calcium ion to a basal medium until the desired concentration of calcium ion is reached. In some aspects, the calcium ion is added to the basal medium adding one or more calcium salts. Non-limiting examples of calcium salts include calcium bromide, calcium carbonate, calcium chloride, calcium cyanamide, calcium fluoride, calcium hydride, calcium hydroxide, calcium iodate, calcium iodide, calcium nitrate, calcium nitrite, calcium oxalate, calcium perchlorate tetrahydrate, calcium phosphate monobasic, calcium phosphate tribasic, calcium sulfate, calcium thiocyanate tetrahydrate, Hydroxyapatite, or any combination thereof. In some aspects, the calcium salt comprises calcium chloride (CaCl₂)).

In some aspects, the desired concentration of calcium ion is lower than that of the basal medium. Therefore, in some aspects, the desired concentration of calcium ion is reached by diluting the basal medium with a suitable solution lacking calcium ion or having a lower concentration of calcium ion.

In some aspects of the present disclosure, the cell culture medium comprises potassium ion at a concentration of at least about 5 mM and a saccharide, e.g., glucose, wherein the medium is hypotonic or isotonic. In some aspects of the present disclosure, the cell culture medium comprises potassium ion at a concentration of at least about 50 mM and a saccharide, e.g., glucose. In some aspects, the method of preparing the cell culture medium described herein comprises adding a saccharide, e.g., glucose, to a basal medium until the desired concentration of the saccharide, e.g., glucose, is reached. Non-limiting examples of saccharides include glucose, fructose, galactose, mannose, maltose, sucrose, lactose, trehalose, or any combination thereof. In some aspects, the saccharide comprises glucose.

In some aspects, the desired concentration of the saccharide, e.g., glucose, is lower than that of the basal medium. Therefore, in some aspects, the desired concentration of the saccharide, e.g., glucose, is reached by diluting the basal medium with a suitable solution lacking the saccharide, e.g., glucose, or having a lower concentration of the saccharide, e.g., glucose.

In some aspects, the tonicity of the medium is adjusted to reach a target osmolality. In some aspects, the target osmolality of the medium is lower than that of the basal medium. In some aspects, the target osmolality of the medium is higher than that of the basal medium. In some aspect, the target osmolality of the medium is the same as that of the basal medium.

The medium can be hypotonic, isotonic, or hypertonic. The tonicity of the medium can be affected by a number of factors, including the concentration of potassium ion in the media. In some aspects, increased potassium ion concentration is paired with an increase or a decrease in the concentration of one or more other factors. In some aspects, this pairing affects the tonicity of the medium. In some aspects, the concentration of potassium ion is increased while the concentration of sodium ion is decreased. In some aspects, the concentration of potassium ion is increased while the concentration of a saccharide, e.g., glucose, is decreased. In some aspects, the concentration of potassium ion is increased while the concentration of calcium ion is decreased. In some aspects, the concentration of potassium ion is increased while the concentrations of sodium ion and saccharide, e.g., glucose, are decreased. In some aspects, the concentration of potassium ion is increased while the concentrations of sodium ion and calcium ion are decreased. In some aspects, the concentration of potassium ion is increased while the concentrations of sodium ion, saccharide (e.g., glucose), and calcium ion are decreased. In some aspects, the concentration of potassium ion, sodium ion, and/or saccharide (e.g., glucose) are adjusted in order to obtain a target tonicity.

IV. Methods Treatment

Certain aspects of the present disclosure are directed to a population of cells, e.g., pluripotent, multipotent, and/or immune cells (e.g., T cells and/or NK cells), cultured according to any of the methods disclosed herein. In certain aspects, the cells are immune cells. In some aspects, the cells are T cells. In some aspects, the cells are NK cells. In some aspects, the immune cells, e.g., T cells and/or NK cells, are isolated from a human subject. In some aspects, the immune cells are tumor-infiltrating T cells or tumor-infiltrating NK cells. In certain aspects, the immune cells, e.g., T cells and/or NK cells, are engineered. In some aspects, the immune cells, e.g., T cells and/or NK cells, are engineered to comprise a chimeric antigen receptor (CAR). In some aspects, the immune cells, e.g., T cells and/or NK cells, are engineered to comprise an engineered T cell receptor (TCR). In some aspects, the TCR recognizes a neoantigen identified in a cancer patient.

In some aspects, the immune cell, e.g., T cells and/or NK cells, comprises a CAR. In some aspects, the T cell is a CD8+ T cell or CD4+ T cell. In some aspects, the T cell is a Th1, Th2, Th17, or Tc17 cell. In some aspects, a CAR-expressing cell is a CAR T cell, e.g., a mono CAR T cell, a genome-edited CAR T cell, a dual CAR T cell, or a tandem CAR T cell. Examples of such CAR T cells are provided in International Application No. PCT/US2019/044195.

In some aspects, the TILs are tumor-infiltrating T cells. In some aspects, the TILs comprise both CD4⁺ T cells and CD8⁺ T cells. In some aspects, the TILs comprise CD8⁺ T cells. In some aspects, the TILs are enriched for tumor specific TILs. In some aspects, the TILs are enriched for stem-like TILs.

In some aspects, the cell comprises construct expressing an antigen receptor and/or another additional polypeptide. In some aspects, the antigen receptor comprises an antibody, an engineered antibody such as scFv, a CAR, an engineered TCR, a TCR mimic (e.g., an antibody-T cell receptor (abTCR) or a chimeric antibody-T cell receptor (caTCR)), or a chimeric signaling receptor (CSR). By way of example, a TCR may comprise an engineered TCR in which the antigen-binding domain of a TCR (e.g., an alpha/beta TCR or a gamma/delta TCR) has been replaced by that of an antibody (with or without the antibody's constant domains); the engineered TCR then becomes specific for the antibody's antigen while retaining the TCR's signaling functions. A chimeric signaling receptor may comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ). See, e.g., EP340793B1, WO 2017/070608, WO 2018/200582, WO 2018/200583, WO 2018/200585, and Xu et al., Cell Discovery (2018) 4:62.

In some aspects, the antigen receptor targets an antigen of interest (e.g., a tumor antigen or an antigen of a pathogen). The antigens may include, without limitation, AFP (alpha-fetoprotein), αvβ6 or another integrin, BCMA, Braf, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein a), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In certain aspects, the antigen receptor targets hTERT. In some aspects, the antigen receptor targets KRAS. In some aspects, the antigen receptor targets Braf In some aspects, the antigen receptor targets TGFβRII. In some aspects, the antigen receptor targets MAGE A10/A4. In some aspects, the antigen receptor targets AFP. In some aspects, the antigen receptor targets PRAME. In some aspects, the antigen receptor targets MAGE A1. In some aspects, the antigen receptor targets WT-1. In some aspects, the antigen receptor targets NY-ESO. In some aspects, the antigen receptor targets PRAME. In some aspects, the antigen receptor targets NY-ESO. In some aspects, the antigen receptor targets CD19.

In some aspects, the antigen receptor targets BCMA. In some aspects, the antigen receptor targets CD147. In some aspects, the antigen receptor targets CD19. In some aspects, the antigen receptor targets CD19 and CD22. In some aspects, the antigen receptor targets CD19 and CD28. In some aspects, the antigen receptor targets CD20. In some aspects, the antigen receptor targets CD20 and CD19. In some aspects, the antigen receptor targets CD22. In some aspects, the antigen receptor targets CD30. In some aspects, the antigen receptor targets CEA. In some aspects, the antigen receptor targets DLL3. In some aspects, the antigen receptor targets EGFRvIII. In some aspects, the antigen receptor targets GD2. In some aspects, the antigen receptor targets HER2. In some aspects, the antigen receptor targets IL-1RAP. In some aspects, the antigen receptor targets mesothelin. In some aspects, the antigen receptor targets methothelin. In some aspects, the antigen receptor targets NKG2D. In some aspects, the antigen receptor targets PSMA. In some aspects, the antigen receptor targets TnMUC1.

In some aspects, the CAR specifically binds (i.e., target) one or more antigens expressed on a tumor cell, such as a malignant B cell, a malignant T cell, or a malignant plasma cell. In some aspects, the CAR specifically binds to (i.e., targets) an antigen selected from the group consisting of CD19, TRAC, TCRβ, BCMA, CLL-1, CS1, CD38, CD19, TSHR, CD123, CD22, CD30, CD70, CDM71, CD33, EGFRvIII, GD2, GFD3, Tn Ag, PSMA, ROR1, ROR2, GPC1, GPC2, FLT3, FAR, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EpbA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-1R-1, UPK2, HAVCR1, ADRB3, PANX3, GPR20, LY6K, OR51E2, TARP, WTI, NY-ESO-1, LAGE-1a, MAGE-A1, legumain, HPV E6,E7 MAGE A1, EVT6-;AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant., hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, or any combinations thereof.

In some aspects, the CAR targets BCMA. In some aspects, the CAR targets CD147. In some aspects, the CAR targets CD19. In some aspects, the CAR targets CD19 and CD22. In some aspects, the CAR targets CD19 and CD28. In some aspects, the CAR targets CD20. In some aspects, the CAR targets CD20 and CD19. In some aspects, the CAR targets CD22. In some aspects, the CAR targets CD30. In some aspects, the CAR targets CEA. In some aspects, the CAR targets DLL3. In some aspects, the CAR targets EGFRvIII. In some aspects, the CAR targets GD2. In some aspects, the CAR targets HER2. In some aspects, the CAR targets IL-1RAP. In some aspects, the CAR targets mesothelin. In some aspects, the CAR targets methothelin. In some aspects, the CAR targets NKG2D. In some aspects, the CAR targets PSMA. In some aspects, the CAR targets TnMUC1.

In some aspects, an immune cell, e.g., T cells and/or NK cells, disclosed herein comprises a T cell receptor (TCR), e.g., an engineered TCR. In some aspects, the engineered TCR specifically binds to a tumor antigen. As used herein, the term “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of T-cells.

In some aspects, the TCR specifically binds (i.e., target) one or more antigens expressed on a tumor cell, such as a malignant B cell, a malignant T cell, or a malignant plasma cell.

In certain aspects, an engineered cell of the present disclosure expresses a T cell receptor (TCR) targeting an antigen. In some aspects, the TCR engineered cells target shared tumor-associated antigens (shared TAAs). In some aspects, the TCR engineered cells target unique tumor-associated antigens (unique TAAs). In some aspects, the TCR engineered cells target tumor-specific antigens. In some aspects, the TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the TCR engineered cells can target glycoprotein (gp100), melanoma antigen recognized by T cells (MART-1), and/or tyrosinase, which are mainly found in melanomas and normal melanocytes. In some aspects, the TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the TCR engineered cells can target mesothelin, another kind of overexpressed antigen that is highly expressed in mesothelioma but is also present on mesothelial cells of several tissues, including trachea.

In some aspects, the TCR engineered cells can target any neoantigen, which can be formed by random somatic mutations specific to individual tumors. In some aspects, the TCR specifically binds to (i.e., targets) a cancer antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CA, EPCAM, B7H3, KIT, IL-13Ra2, mesothelin, IL-1ARa, PSCA, PRSS21, VEGFR2, LewisY, CD241, PDGFR-beta, SSEA-4, CD20, folate receptor alpha, ERBB2(Her2/neu), MUC1, M-UC 6, EGFR, NCAM, prostase, PAP, ELF2M, Ephrin 132, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-I/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYPI1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3E, CD4, CD5, CD7, the extracellular portion of the APRIL protein, or any combinations thereof.

In certain aspects, the TCR specifically binds (i.e., targets) hTERT. In some aspects, the TCR specifically binds (i.e., targets) KRAS. In some aspects, the TCR specifically binds (i.e., targets) Braf In some aspects, the TCR specifically binds (i.e., targets) TGFβRII. In some aspects, the TCR specifically binds (i.e., targets) MAGE A10/A4. In some aspects, the TCR specifically binds (i.e., targets) AFP. In some aspects, the TCR specifically binds (i.e., targets) PRAME. In some aspects, the TCR specifically binds (i.e., targets) MAGE A1. In some aspects, the TCR specifically binds (i.e., targets) WT-1. In some aspects, the TCR specifically binds (i.e., targets) NY-ESO. In some aspects, the TCR specifically binds (i.e., targets) PRAME. In some aspects, the TCR specifically binds (i.e., targets) NY-ESO. In some aspects, the TCR specifically binds (i.e., targets) CD19. In some aspects, the TCR specifically binds a neoantigen identified in a cancer patient.

In some aspects, the cell, e.g., a pluripotent cell, a multipotent cell, or an immune cell (e.g., a T cell and/or an NK cell), is administered to a subject in need thereof. In some aspects the cell, e.g., a pluripotent cell, a multipotent cell, or an immune cell (e.g., a T cell and/or an NK cell), prepared using the methods disclose herein is administered to a subject to treat a cancer, e.g., a tumor. In some aspects, the method of treating comprises administering to the subject an effective amount of a cell composition of the disclosure, e.g., a cell prepared according to the methods disclosed herein, e.g., a T cell expressing a chimeric polypeptide or TCR disclosed herein. In some aspects, the method of treating comprises administering to the subject an effective amount of a TIL composition of the disclosure, e.g., a composition comprising a population of TILs prepared according to the methods disclosed herein, e.g., a population of TILs enriched for CD8+ TILs, tumor-specific TILs, and/or stem-like TILs.

The present disclosure also provides a method of stimulating a T cell-mediated immune response to a target cell population or tissue in a subject, comprising administering an effective amount of a cell composition of the disclosure, e.g., a cell prepared according to the methods disclosed herein, e.g., a T cell expressing a chimeric polypeptide or TCR disclosed herein.

The present disclosure also provides a method of stimulating a T cell-mediated immune response to a target cell population or tissue in a subject, comprising administering an effective amount of a TIL composition of the disclosure, e.g., a population of TILs prepared according to the methods disclosed herein, e.g., a population of TILs enriched for CD8⁺ TILs.

The present disclosure also provides a method of providing an anti-tumor immunity in a subject in need thereof, the method comprising administering a cell composition of the disclosure, e.g., a cell prepared according to the methods disclosed herein, e.g., a T cell expressing a chimeric polypeptide or TCR disclosed herein to the subject.

In some aspects, the cell administered in the cell composition of the disclosure is a T cell. In some aspects, the cell is an autologous T cell. In some aspects, the population of cells administered in the cell composition of the disclosure comprises TILs. In some aspects, the population of cells administered in the cell composition of the disclosure comprises autologous TILs.

In some aspects, the method comprises administering at least about 1×10⁴, at least about 5×10⁴, at least about 1×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 5×10⁷, at least about 1×10⁸ cells, e.g., TILs, to the subject. In some aspects, the method comprises administering at least about 1×10⁴, at least about 5×10⁴, at least about 1×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 5×10⁷, at least about 1×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 10×10¹⁰, at least about 15×10¹⁰, at least about 20×10¹⁰, at least about 25×10¹⁰, or at least about 30×10¹⁰ cells, e.g., TILs, to the subject. In some aspects, the cells are TILs. In some aspects, the TILs are CD8⁺ TILs.

In some aspects, the method comprises administering at least about 10×10⁹, at least about 20×10⁹, at least about 30×10⁹, at least about 40×10⁹, at least about 50×10⁹, at least about 60×10⁹, at least about 70×10⁹, at least about 80×10⁹, at least about 90×10⁹, or at least about 100×10⁹ cells (e.g., TILs). In some aspects, the method comprises administering about 10×10⁹ to about 100×10⁹, e.g., about 10×10⁹, about 20×10⁹, about 30×10⁹, about 40×10⁹, about 50×10⁹, about 60×10⁹, about 70×10⁹, about 80×10⁹, about 90×10⁹, or about 100×10⁹, cells (e.g., TILs). In some aspects, the method comprises administering about 10×10⁹ to about 20×10⁹ cells (e.g., TILs), about 20×10⁹ to about 30×10⁹ cells (e.g., TILs), about 30×10⁹ to about 40×10⁹ cells (e.g., TILs), about 40×10⁹ to about 50×10⁹ cells (e.g., TILs), about 50×10⁹ to about 60×10⁹ cells (e.g., TILs), about 60×10⁹ to about 70×10⁹ cells (e.g., TILs), about 70×10⁹ to about 80×10⁹ cells (e.g., TILs), about 80×10⁹ to about 90×10⁹ cells (e.g., TILs), or about 90×10⁹ to about 100×10⁹ cells (e.g., TILs). In some aspects, the method comprises administering about 10×10⁹ to about 20×10⁹ cells (e.g., TILs). In some aspects, the method comprises administering about 20×10⁹ to about 30×10⁹ cells (e.g., TILs). In some aspects, the method comprises administering about 30×10⁹ to about 40×10⁹ cells (e.g., TILs). In some aspects, the method comprises administering about 40×10⁹ to about 50×10⁹ cells (e.g., TILs). In some aspects, the method comprises administering about 50×10⁹ to about 60×10⁹ cells (e.g., TILs). In some aspects, the method comprises administering about 60×10⁹ to about 70×10⁹ cells (e.g., TILs). In some aspects, the method comprises administering about 70×10⁹ to about 80×10⁹ cells (e.g., TILs). In some aspects, the method comprises administering about 80×10⁹ to about 90×10⁹ cells (e.g., TILs). In some aspects, the method comprises administering about 90×10⁹ to about 100×10⁹ cells (e.g., TILs). In some aspects, the TILs are CD8⁺ TILs.

In some aspects, administering the cell composition of the disclosure (e.g., an immune cell such as a T cell expressing a CAR or TCR prepared according to the methods disclosed herein) reduces a tumor volume in the subject compared to a reference tumor volume. In some aspects, administering the cell composition of the disclosure (e.g., comprising a population of TILs prepared according to the methods disclosed herein (e.g., enriched for CD8+ TILs, tumor-specific TILs, and/or stem-like TILs)) reduces a tumor volume in the subject compared to a reference tumor volume. In some aspects, the reference tumor volume is the tumor volume in the subject prior to the administration of the engineered cell. In further aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In certain aspects, administering the cell composition of the disclosure (e.g., an immune cell such as a T cell expressing a CAR or TCR prepared according to the methods disclosed herein) can reduce the tumor weight in a subject when administered to the subject. In some aspects, administering the cell composition of the disclosure (e.g., comprising a population of TILs prepared according to the methods disclosed herein (e.g., enriched for CD8+ TILs, tumor-specific TILs, and/or stem-like TILs)) can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the cell composition of the disclosure. In further aspects, the reference tumor weight is the tumor weight in a corresponding subject that did not receive the administration.

In some aspects, administering the cell composition of the disclosure (e.g., an immune cell such as a T cell expressing a CAR or TCR prepared according to the methods disclosed herein) to a subject, e.g., suffering from a tumor, can increase the number and/or percentage of TILs (e.g., CD4⁺ or CD8⁺) in a tumor and/or a tumor microenvironment (TME) of the subject. In some aspects, administering the cell composition of the disclosure (e.g., comprising a population of TILs prepared according to the methods disclosed herein (e.g., enriched for CD8⁺ TILs, tumor-specific TILs, and/or stem-like TILs)) to a subject, e.g., suffering from a tumor, can increase the number and/or percentage of TILs (e.g., CD8⁺ TILs) in a tumor and/or a tumor microenvironment (TME) of the subject. In certain aspects, the number and/or percentage of TILs (e.g., CD8⁺) in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the cell composition of the present disclosure or the same subject prior to the administration of the cell composition of the present disclosure).

In some aspects, administering the cell composition of the disclosure (e.g., an immune cell such as a T cell expressing a CAR or TCR prepared according to the methods disclosed herein) to a subject, e.g., suffering from a tumor, can increase the duration of an immune response in a subject relative to the duration of an immune response in a subject administered a similar cell therapy comprising cells prepared according to conventional methods, e.g., cultured in a medium not comprising a potassium ion concentration of at least 50 mM. In some aspects, administering the cell composition of the disclosure (e.g., comprising a population of TILs prepared according to the methods disclosed herein (e.g., enriched for CD8⁺TILs, tumor-specific TILs, and/or stem-like TILs)) to a subject, e.g., suffering from a tumor, can increase the duration of an immune response in a subject relative to the duration of an immune response in a subject administered a similar cell therapy comprising cells prepared according to conventional methods, e.g., cultured in a medium not comprising a potassium ion concentration of at least about 40 mM to at least about 90 mM, e.g., at least 50 mM. In certain aspects, the duration of the immune response is increased by at least about 10%, at least about 20%, at least about 30%, at least about 40%, at least about 50%, at least about 75%, at least about 100%, at least about 150%, at least about 200%, at least about 300%, at least about 400%, at least about 500%, or at least about 1000% or more compared to a reference (e.g., a subject administered a similar cell therapy comprising cells prepared according to conventional methods, e.g., cultured in a medium not comprising a potassium ion concentration of at least 50 mM). In certain aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., a subject administered a similar cell therapy comprising cells prepared according to conventional methods, e.g., cultured in a medium not comprising a potassium ion concentration of at least 50 mM). In some aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., a subject administered a similar cell therapy comprising cells prepared according to conventional methods, e.g., cultured in a medium not comprising a potassium ion concentration of at least about 40 mM to at least about 90 mM, e.g., at least 50 mM).

In addition to the above, administering the cell composition of the disclosure (e.g., an immune cell such as a T cell expressing a CAR or TCR prepared according to the methods disclosed herein) can have other effects which are conducive for the treatment of a tumor. Further, administering a population of TILs prepared according to the methods disclosed herein (e.g., enriched for CD8+ TILs, tumor-specific TILs, and/or stem-like TILs) can have other effects which are conducive for the treatment of a tumor.

As described herein, the cell composition of the disclosure (e.g., an immune cell such as a T cell expressing a CAR or TCR and/or a TIL prepared according to the methods disclosed herein) can be used to treat variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof. In some aspects, the cancer comprises a solid tumor. In some aspects, the cancer comprises a solid tumor derived from a melanoma, a colon cancer, a lung cancer, a cervical cancer, a gastrointestinal cancer, a breast cancer, a prostate cancer, a liver cancer, bone cancer, a pancreatic cancer, a small cell carcinoma of the head and neck, lung squamous cell carcinoma, lung adenocarcinoma, pancreatic adenocarcinoma, head and neck squamous cell carcinoma, testicular germ cell tumors, stomach adenocarcinoma, skin cutaneous melanoma, mesothelioma, kidney renal clear cell carcinoma, cervical squamous cell carcinoma and endocervical adenocarcinoma, esophageal carcinoma, bladder urothelial carcinoma, breast invasive carcinoma, kidney renal papillary cell carcinoma, colon adenocarcinoma, or any combination thereof.

In some aspects, the cancer comprises a melanoma. In some aspects, the cancer comprises colorectal cancer. In some aspects, the cancer comprises a colon cancer. In some aspects, the cancer comprises pancreatic cancer. In some aspects, the cancer comprises head and neck cancer. In some aspects, the cancer comprises cervical cancer. In some aspects, the cancer comprises ovarian cancer. In some aspects, the cancer comprises a lung cancer. In some aspects, the cancer comprises a gastrointestinal cancer. In some aspects, the cancer comprises a breast cancer. In some aspects, the cancer comprises a prostate cancer. In some aspects, the cancer comprises a liver cancer. In some aspects, the cancer comprises bone cancer. In some aspects, the cancer comprises a small cell carcinoma of the head and neck. In some aspects, the cancer comprises lung squamous cell carcinoma. In some aspects, the cancer comprises lung adenocarcinoma. In some aspects, the cancer comprises pancreatic adenocarcinoma. In some aspects, the cancer comprises head and neck squamous cell carcinoma. In some aspects, the cancer comprises a testicular germ cell tumor. In some aspects, the cancer comprises stomach adenocarcinoma. In some aspects, the cancer comprises skin cutaneous melanoma. In some aspects, the cancer comprises mesothelioma. In some aspects, the cancer comprises kidney renal clear cell carcinoma. In some aspects, the cancer comprises cervical squamous cell carcinoma. In some aspects, the cancer comprises endocervical adenocarcinoma. In some aspects, the cancer comprises esophageal carcinoma. In some aspects, the cancer comprises bladder urothelial carcinoma. In some aspects, the cancer comprises breast invasive carcinoma. In some aspects, the cancer comprises kidney renal papillary cell carcinoma. In some aspects, the cancer comprises colon adenocarcinoma. In some aspects, the cancer comprises a uterine cancer. In some aspects, the cancer comprises a brain. In some aspects, the cancer comprises a thyroid cancer. In some aspects, the cancer comprises an esophageal cancer. In some aspects, the cancer comprises an eye cancer. In some aspects, the cancer comprises a stomach (gastric) cancer. In some aspects, the cancer comprises a gastrointestinal cancer. In some aspects, the cancer comprises a sarcoma. In some aspects, the cancer comprises a leukemia. In some aspects, the cancer comprises a lymphoma. In some aspects, the cancer comprises a myeloma.

As such, some aspects of the present disclosure are directed to methods of treating a melanoma in a subject in need thereof, comprising administering to the subject a cell composition disclosed herein. Some aspects of the present disclosure are directed to methods of treating a colorectal cancer in a subject in need thereof, comprising administering to the subject a cell composition disclosed herein. Some aspects of the present disclosure are directed to methods of treating a colon cancer in a subject in need thereof, comprising administering to the subject a cell composition disclosed herein. Some aspects of the present disclosure are directed to methods of treating pancreatic cancer in a subject in need thereof, comprising administering to the subject a cell composition disclosed herein. Some aspects of the present disclosure are directed to methods of treating head and neck cancer in a subject in need thereof, comprising administering to the subject a cell composition disclosed herein. Some aspects of the present disclosure are directed to methods of treating cervical cancer in a subject in need thereof, comprising administering to the subject a cell composition disclosed herein. Some aspects of the present disclosure are directed to methods of treating ovarian cancer in a subject in need thereof, comprising administering to the subject a cell composition disclosed herein. Some aspects of the present disclosure are directed to methods of treating a lung cancer in a subject in need thereof, comprising administering to the subject a cell composition disclosed herein. Some aspects of the present disclosure are directed to methods of treating a gastrointestinal cancer in a subject in need thereof, comprising administering to the subject a cell composition disclosed herein. Some aspects of the present disclosure are directed to methods of treating a breast cancer in a subject in need thereof, comprising administering to the subject a cell composition disclosed herein. Some aspects of the present disclosure are directed to methods of treating a prostate cancer in a subject in need thereof, comprising administering to the subject a cell composition disclosed herein. Some aspects of the present disclosure are directed to methods of treating a liver cancer in a subject in need thereof, comprising administering to the subject a cell composition disclosed herein. Some aspects of the present disclosure are directed to methods of treating bone cancer in a subject in need thereof, comprising administering to the subject a cell composition disclosed herein. Some aspects of the present disclosure are directed to methods of treating a small cell carcinoma of the head and neck in a subject in need thereof, comprising administering to the subject a cell composition disclosed herein. Some aspects of the present disclosure are directed to methods of treating lung squamous cell carcinoma in a subject in need thereof, comprising administering to the subject a cell composition disclosed herein. Some aspects of the present disclosure are directed to methods of treating lung adenocarcinoma in a subject in need thereof, comprising administering to the subject a cell composition disclosed herein. Some aspects of the present disclosure are directed to methods of treating pancreatic adenocarcinoma in a subject in need thereof, comprising administering to the subject a cell composition disclosed herein. Some aspects of the present disclosure are directed to methods of treating head and neck squamous cell carcinoma in a subject in need thereof, comprising administering to the subject a cell composition disclosed herein. Some aspects of the present disclosure are directed to methods of treating a testicular germ cell tumor in a subject in need thereof, comprising administering to the subject a cell composition disclosed herein. Some aspects of the present disclosure are directed to methods of treating stomach adenocarcinoma in a subject in need thereof, comprising administering to the subject a cell composition disclosed herein. Some aspects of the present disclosure are directed to methods of treating skin cutaneous melanoma in a subject in need thereof, comprising administering to the subject a cell composition disclosed herein. Some aspects of the present disclosure are directed to methods of treating mesothelioma in a subject in need thereof, comprising administering to the subject a cell composition disclosed herein. Some aspects of the present disclosure are directed to methods of treating kidney renal clear cell carcinoma in a subject in need thereof, comprising administering to the subject a cell composition disclosed herein. Some aspects of the present disclosure are directed to methods of treating cervical squamous cell carcinoma in a subject in need thereof, comprising administering to the subject a cell composition disclosed herein. Some aspects of the present disclosure are directed to methods of treating endocervical adenocarcinoma in a subject in need thereof, comprising administering to the subject a cell composition disclosed herein. Some aspects of the present disclosure are directed to methods of treating esophageal carcinoma in a subject in need thereof, comprising administering to the subject a cell composition disclosed herein. Some aspects of the present disclosure are directed to methods of treating bladder urothelial carcinoma in a subject in need thereof, comprising administering to the subject a cell composition disclosed herein. Some aspects of the present disclosure are directed to methods of treating breast invasive carcinoma in a subject in need thereof, comprising administering to the subject a cell composition disclosed herein. Some aspects of the present disclosure are directed to methods of treating kidney renal papillary cell carcinoma in a subject in need thereof, comprising administering to the subject a cell composition disclosed herein. Some aspects of the present disclosure are directed to methods of treating colon adenocarcinoma in a subject in need thereof, comprising administering to the subject a cell composition disclosed herein. Some aspects of the present disclosure are directed to methods of treating a uterine cancer in a subject in need thereof, comprising administering to the subject a cell composition disclosed herein. Some aspects of the present disclosure are directed to methods of treating a brain tumor in a subject in need thereof, comprising administering to the subject a cell composition disclosed herein. Some aspects of the present disclosure are directed to methods of treating an esophageal cancer in a subject in need thereof, comprising administering to the subject a cell composition disclosed herein. Some aspects of the present disclosure are directed to methods of treating a thyroid cancer in a subject in need thereof, comprising administering to the subject a cell composition disclosed herein. Some aspects of the present disclosure are directed to methods of treating an eye cancer in a subject in need thereof, comprising administering to the subject a cell composition disclosed herein. Some aspects of the present disclosure are directed to methods of treating a stomach (gastric) cancer in a subject in need thereof, comprising administering to the subject a cell composition disclosed herein. Some aspects of the present disclosure are directed to methods of treating a gastrointestinal cancer in a subject in need thereof, comprising administering to the subject a cell composition disclosed herein. Some aspects of the present disclosure are directed to methods of treating a sarcoma in a subject in need thereof, comprising administering to the subject a cell composition disclosed herein. Some aspects of the present disclosure are directed to methods of treating a leukemia in a subject in need thereof, comprising administering to the subject a cell composition disclosed herein. Some aspects of the present disclosure are directed to methods of treating a lymphoma in a subject in need thereof, comprising administering to the subject a cell composition disclosed herein. Some aspects of the present disclosure are directed to methods of treating a myeloma in a subject in need thereof, comprising administering to the subject a cell composition disclosed herein.

In some aspects, the cell composition of the disclosure (e.g., an immune cell such as a T cell expressing a CAR or TCR prepared according to the methods disclosed herein) can be used in combination with other therapeutic agents (e.g., anti-cancer agents and/or immunomodulating agents). In some aspects, the cell composition of the disclosure (e.g., comprising a population of TILs prepared according to the methods disclosed herein (e.g., enriched for CD8+ TILs, tumor-specific TILs, and/or naive TILs)) can be used in combination with other therapeutic agents (e.g., anti-cancer agents and/or immunomodulating agents). Accordingly, in certain aspects, a method of treating a tumor disclosed herein comprises administering the cell composition of the disclosure in combination with one or more additional therapeutic agents.

In some aspects, the cell composition of the disclosure (e.g., an immune cell such as a T cell expressing a CAR or TCR prepared according to the methods disclosed herein) can be used in combination with one or more anti-cancer agents, such that multiple elements of the immune pathway can be targeted. In some aspects, the cell composition of the disclosure (e.g., comprising a population of TILs prepared according to the methods disclosed herein (e.g., enriched for CD8⁺ TILs, tumor-specific TILs, and/or naive TILs)) can be used in combination with one or more anti-cancer agents. In some aspects, an anti-cancer agent comprises an immune checkpoint inhibitor (i.e., blocks signaling through the particular immune checkpoint pathway).

Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. In some aspects, the checkpoint inhibitor is a PD-1 antagonist. In some aspects, the checkpoint inhibitor is an anti-PD-1 antibody. In some aspects, the checkpoint inhibitor is an anti-PD-L1 antibody. A comprehensive and non-limiting list of combination treatment is disclosed in detail elsewhere in this application.

In some aspects, the cell composition of the disclosure (e.g., an immune cell such as a T cell expressing a CAR or TCR and/or a population of TILs prepared according to the methods disclosed herein) is administered to the subject prior to or after the administration of the additional therapeutic agent. In other aspects, the cell composition of the disclosure (e.g., an immune cell such as a T cell expressing a CAR or TCR and/or a population of TILs prepared according to the methods disclosed herein) is administered to the subject concurrently with the additional therapeutic agent. In certain aspects, the cell composition of the disclosure (e.g., an immune cell such as a T cell expressing a CAR or TCR and/or a population of TILs prepared according to the methods disclosed herein) and the additional therapeutic agent can be administered concurrently as a single composition in a pharmaceutically acceptable carrier. In other aspects, the cell composition of the disclosure (e.g., an immune cell such as a T cell expressing a CAR or TCR and/or a population of TILs prepared according to the methods disclosed herein) and the additional therapeutic agent are administered concurrently as separate compositions.

In some aspects, the subject is a nonhuman animal such as a rat or a mouse. In some aspects, the subject is a human.

In some aspects, a cell composition disclosed herein (e.g., an immune cell such as a T cell expressing a CAR or TCR and/or a population of TILs prepared according to the methods disclosed herein) can be used in combination with other therapeutic agents (e.g., anti-cancer agents and/or immunomodulating agents). Accordingly, in certain aspects, a method of treating a tumor disclosed herein comprises administering a cell composition of the present disclosure (e.g., an immune cell such as a T cell expressing a CAR or TCR and/or a population of TILs prepared according to the methods disclosed herein) in combination with one or more additional therapeutic agents to a subject. Such agents can include, for example, chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

In some aspects, a cell composition disclosed herein (e.g., an immune cell such as a T cell expressing a CAR or TCR and/or a population of TILs prepared according to the methods disclosed herein) can be used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, a cell composition of the present disclosure (e.g., an immune cell such as a T cell expressing a CAR or TCR and/or a population of TILs prepared according to the methods disclosed herein) can be used in combination with one or more anti-cancer agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically engineered cells, e.g., cells engineered to express a chimeric antigen receptor (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUDA®; MK-3475), pidilizumab (CT-011), PDR001, MEDIO680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; RO5541267), durvalumab (MEDI4736, IMWINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with a PD1 antagonist. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with an anti-PD1 antibody. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with nivolumab. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with pembrolizumab.

In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with a PD-L1 antagonist. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with an anti-PD-L1 antibody. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with atezolizumab. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with durvalumab. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with avelumab.

In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with a CTLA-4 antagonist. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with an anti-CTLA-4 antibody. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with ipilimumab. In some aspects, the cell composition of the present disclosure (e.g., comprising a population of TILs prepared according to the methods disclosed herein (e.g., enriched for CD8⁺ TILs, tumor-specific TILs, and/or naive TILs)) is administered to a subject in combination with tremelimumab.

In some aspects, an anti-cancer agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB CD137) agonist (e.g., anti-CD137 antibody), GITR agonist (e.g., anti-GITR antibody), TIM3 agonist (e.g., anti-TIM3 antibody), or combinations thereof. In some aspects, the additional therapeutic agent comprises a cytokine. In some aspects, the cytokine comprises IL-2, Il-21, Il-7, Il-15, or any combination thereof.

In some aspects, a cell composition disclosed herein (e.g., an immune cell such as a T cell expressing a CAR or TCR and/or a population of TILs prepared according to the methods disclosed herein) is administered to the subject prior to or after the administration of the additional therapeutic agent. In other aspects, cell composition disclosed herein (e.g., an immune cell such as a T cell expressing a CAR or TCR and/or a population of TILs prepared according to the methods disclosed herein) is administered to the subject concurrently with the additional therapeutic agent. In certain aspects, the cell composition disclosed herein (e.g., an immune cell such as a T cell expressing a CAR or TCR and/or a population of TILs prepared according to the methods disclosed herein) and the additional therapeutic agent can be administered concurrently as a single composition in a pharmaceutically acceptable carrier. In other aspects, the cell composition disclosed herein (e.g., an immune cell such as a T cell expressing a CAR or TCR and/or a population of TILs prepared according to the methods disclosed herein) and the additional therapeutic agent are administered concurrently as separate compositions. In some aspects, the additional therapeutic agent and the cell composition disclosed herein (e.g., an immune cell such as a T cell expressing a CAR or TCR and/or a population of TILs prepared according to the methods disclosed herein) are administered sequentially.

Certain aspects of the present disclosure are directed to methods of treating an autoimmune disease, comprising administering a cell, e.g., a Treg cell, cultured according to any of the methods disclosed herein. Other aspects of the present disclosure are directed to methods of treating an inflammatory pathology, comprising administering a cell, e.g., a Treg cell, cultured according to any of the methods disclosed herein. In some aspects, the inflammatory pathology comprises cytokine release syndrome. In some aspects, the inflammatory pathology comprises sepsis. In some aspects, the inflammatory pathology comprises graft-versus host disease. In some aspects, the cell, e.g., the Treg cell, is engineered.

In some aspects, a cell composition disclosed herein (e.g., comprising a population of TILs prepared according to the methods disclosed herein (e.g., enriched for CD8⁺TILs, tumor-specific TILs, and/or naive TILs)) is administered to the subject in combination with a checkpoint inhibitor (e.g., an anti-PD1 antibody). In some aspects, the cell composition is administered before the checkpoint inhibitor (e.g., an anti-PD1 antibody). In some aspects, the cell composition is administered after the checkpoint inhibitor (e.g., an anti-PD1 antibody).

In some aspects, the subject is administered a lymphodepleting therapy prior to receiving the cell composition. Any lymphodepleting therapy can be used in the method disclosed herein. In some aspects, the lymphodepleting therapy comprises a chemotherapy. In some aspects, the lymphodepleting therapy comprises cyclophosphamide. In some aspects, the lymphodepleting therapy comprises fludarabine. In some aspects, the lymphodepleting therapy comprises cyclophosphamide and fludarabine. In some aspects, the lymphodepleting therapy is administered at least about 3 days, at least about 4 days, at least about 5 days, at least about 7 days, at least about 8 days, at least about 9 days, at least about 10 days, at least about 11 days, at least about 12 days, at least about 13 days, or at least about 14 days prior to the cell composition.

### EXAMPLES

### Example 1. Methods

Media preparation: T cell conditioned media (TCM) was supplemented with immune Cell Serum Replacement (Thermo Fisher), 2 mM L-glutamine (Gibco), 2 mM Glutamax (Gibco), MEM Non-Essential Amino Acids Solution (Gibco), Sodium pyruvate (Gibco), IL-2, 200 IU/mL; IL-7,120 IU/ml; IL-15, 20 IU/ml.

For hypotonic conditioning medium, TCM media with varying concentrations of sodium, potassium, glucose and calcium were adjusted by adding NaCl, glucose, and calcium free RPMI. After adding defined NaCl free RPMI to TCM, the final concentrations were in the range of: NaCl (40-80 mM), KCl (40-80 mM), Calcium (0.5-2.8 mM), Glucose (10-24 mM) and osmolality (˜250-260 mOsmol). See Table. 1.

TABLE 1
Hypotonic conditioning medium with varying concentrations
of potassium, sodium, glucose, and calcium

| | K | NaCl | Glucose | Ca | Osmolality | Tonicity* |
|---|---|---|---|---|---|---|
| Media | (mM) | (mM) | (mM) | (mM) | (mOsmol) | (mOsmol) |
| Basal Media | 4 | 118.47 | ~24 mM | ~2.8 mM | 245 | 245 |
| Hyper K | 80 mM | 55.6 mM | 15 mM | 1.2 mM | ~262.26 | 271.2 |
| Hyper K | 75 | 59.3 | 15.4 | 1.3 | ~260 | 268.6 |
| Hyper K | 70 | 63.9 | 15.9 | 1.4 | ~259.7 | 267.8 |
| Hyper K | 65 | 67.6 | 16.3 | 1.5 | ~257.5 | 265.2 |
| Hyper K | 60 | 72.2 | 16.8 | 1.6 | ~257.2 | 264.4 |
| Hyper K | 55 | 76 | 17.2 | 1.7 | ~255.2 | 262 |
| Hyper K | 50 | 80.5 | 17.7 | 1.8 | ~254.7 | 261 |
| RPMI Gibco + | 5.34 | 103 | 11.1 | 0.4 | | 216.7 |
| ICSR | | | | | | |
| RPMI 1640 + 50 | 55.34 | 103 | | | | 316.7 |
| mM K+ | | | | | | |

*Tonicity is calculated based on the following formula: 2 × (concentration of K + concentration of NaCl)

We also tested the effect of tonicity on T cells by maintaining constant tonicity conditions (250 mOsmol—hypotonic, 280 mOsmol—isotonic, 320 mOsmol—hypertonic) with varying potassium concentrations. Final concentrations in hypotonic conditions, NaCl (35-75 mM), KCl (50-90 mM), final concentrations in isotonic conditions NaCl (50-90 mM), KCl (50-90 mM), final concentrations in hypertonic conditions NaCl (70-110 mM), KCl (50-90 mM). See Table. 2.

TABLE 2
Hypotonic, isotonic, hypertonic solutions with
varying concentrations of potassium and NaCl

| Tonicity* | mOsmol | K (mM) | NaCl (mM) |
|---|---|---|---|
| Hypotonic | 250 | 50 | 75 |
| | | 60 | 65 |
| | | 70 | 55 |
| | | 80 | 45 |
| | | 90 | 35 |
| Isotonic | 280 | 50 | 90 |
| | | 60 | 80 |
| | | 70 | 70 |
| | | 80 | 60 |
| | | 90 | 50 |
| Hypertonic | 320 | 50 | 110 |
| | | 60 | 100 |
| | | 70 | 90 |
| | | 80 | 80 |
| | | 90 | 70 |

*Tonicity is calculated according to the formula: Tonicity = ([K] + [NaCl]) × 2 wherein “[K]” is the potassium concentration and “[NaCl]” is the sodium chloride concentration of the media.

Cell culture and Transduction: Healthy donor cryopreserved human CD4 and CD8 cells were activated with TRANSACT™ (Miltenyi) in T cell conditioned media-TCM, basal media, or hypotonic conditioning medium. After 24 hours of activation in the TCM or hypotonic conditioning medium, T cells were transduced with lentiviral particles to introduce chimeric antigen receptor (anti-CD19 CAR) in Grex plates (Wilson Wolf). The following day after transduction, T cells were supplemented with fresh media to dilute the TRANSACT™ and end T-cell activation. Depending on the cell growth and density, T cells were fed with warm 2× cytokine media by aspirating half of the media in the Grex plate. On day 7, cells were harvested, counted and analyzed for the expression of stemness markers by flow cytometry.

Intracellular Cytokine assays: On day 7, T cells were washed and placed in control media and subjected to a 5 hour re-stimulation with phorbol myrystate acetate (PMA) and ionomycin in the presence of brefeldin A to measure intracellular cytokines, IL-2, IFNγ, and TNFα. T cells were stained with surface antibody staining in FACS buffer containing fixable live/dead solution. Cells were stained with respective antibodies for intracellular cytokines following fixation and permeabilization. Quantification of intracellular cytokine expression was assessed using flow cytometry.

Stemness phenotype CAR expression measurement via flow cytometry: On day 7, live T cells from the respective treatments were assessed via flow cytometry. Cells were first washed with cell staining buffer and stained with anti-CCR7 for 15 minutes at 37° C. Following this, a 2× master mix of the antibodies against several other antigens (as detailed below) was added to cells and incubated for 20 minutes at 4° C. Cells were washed with cell staining buffer and permeabilized with the foxp3 staining kit (ebioscience) as per manufacturers' protocol. After fixing, the cells were stained for TCF7 for twenty minutes at 4° C. following which, cells were analyzed by flow cytometry on aurora (cytek). The following are the list of antibodies used for assessing the stemness markers: CD8 (BD-#563795), CD4 (BD-#612936), CD27 (BD-#612829), CD3 (Thermo-#612893), CD28 (Biolegend-#302936), CD62L, CAR-EGFR (Thermo-#352911), CD45RO (BD #564290), CD39 (Biolegend-#328236), TCF7 (Cell signaling-#14456), CCR7 (BD-#562381), CD127 (Bio legend-#351324), CD45RA (BD-#560673).

### Example 2. Results

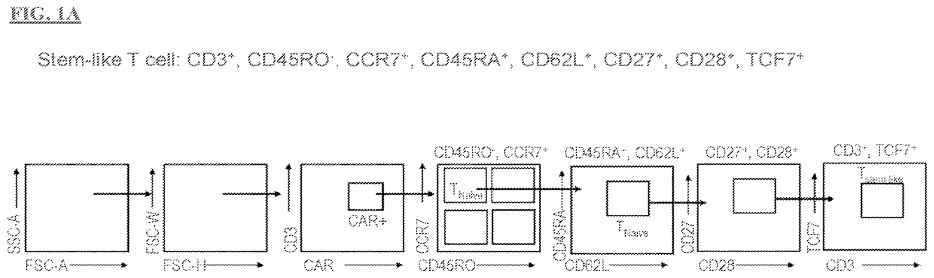

Multiple avenues of manipulation of media components can result in profound alterations to the generated T cell product. In sum we manipulated one or more of the following: tonicity, inorganic salts, nutrients (glucose), and/or cytokine content both in sequence and parallel. We started with manipulation of the diluent tonicity to assess its role in affecting the final T cell stemness, as assessed by multiple surface markers. To summarize the stemness of the final T cell population we depicted the flow cytometry gating schema to quantify the relative population of the desired subpopulation of T cells with the highest amount of relative stemness (FIG. 1A). The depicted flow cytometry schema is a well-accepted surrogate for the anticipated stemness, metabolic function, and in vivo behavior of the resulting T cells (FIG. 1B).

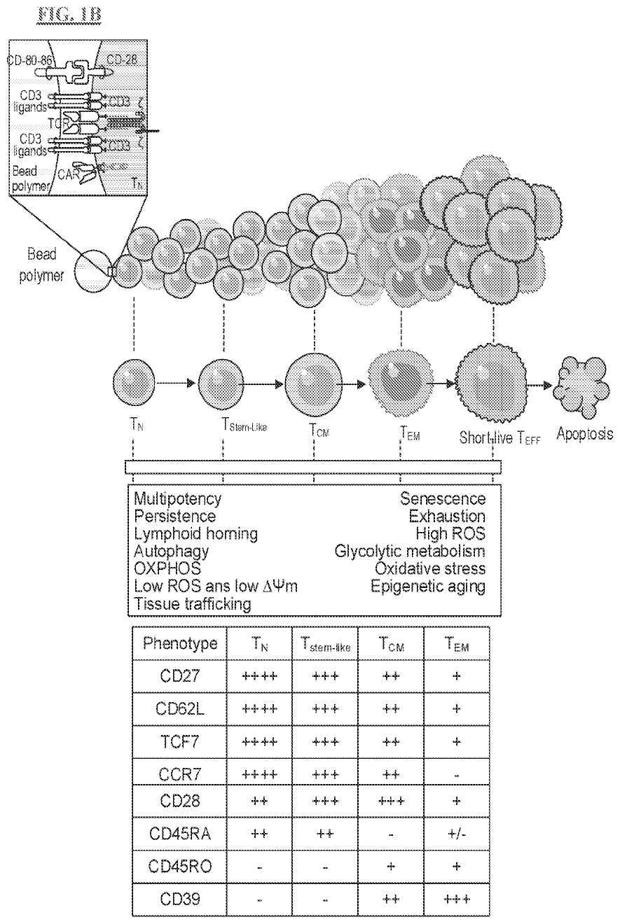

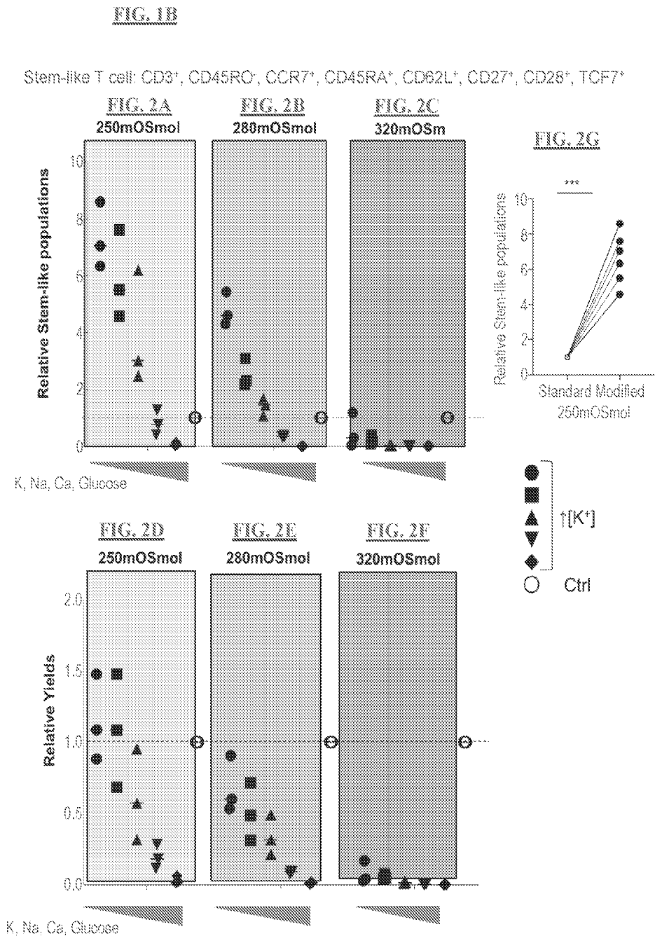

We quantified the effect of these interventions by the relative number of T cells that were CD3⁺, CD45RO⁻, CCR7⁺, CD45RA⁺, CD62L⁺, CD27⁺, CD28⁺, and TCF7⁺ as assessed by cell enumeration and polychromatic flow cytometry (FIGS. 2A-2F). In tandem with tonicity we concurrently altered Na⁺, K⁺, and Ca²⁺ as these have all reported roles in controlling T cell fate and function (Trebak & Kinet, Nature reviews (2019), Verkhratsky et al, Exp. Physio (2019)). Chloride was additionally altered in concert with alterations to the described cationic ions. We found that a relatively “hypotonic” solution with an elevated concentration of potassium, lower concentration of Na, and lowered concentration of Ca resulted in the largest number of stem-like cells (FIG. 2A-2F).

In particular, culture of T cells in hypotonic media (<280 mOsm/L) having 50 mM, 55 mM, or 60 mM potassium had at least a 1-fold increase and up to 2.5-fold increase (typically closer to the higher end of the range) in the relative number of stem-like cells, as compared to T cells cultured in control media. Further, culture of T cells in hypotonic media having 65 mM, 70 mM, 75 mM, 80 mM, 85 mM, or 90 mM potassium had at least a 2.5-fold increase and up to a 15-fold increase (typically at least 4-5-fold increase) in the relative number of stem-like cells, as compared to T cells cultured in control media. Similarly, T cells cultured in isotonic media (˜270-300 mOsm/L) having 50 mM, 55 mM, 60 mM, 65 mM, or 70 mM potassium had at least a 1-fold increase and up to 2.5-fold increase (typically closer to the higher end of the range) in the relative number of stem-like cells, as compared to T cells cultured in control media.

Increased potassium levels generally correlated with acceptable or even improved cell yields, to a certain degree. In particular, T cells cultured in hypotonic media or isotonic media having 50 mM, 55 mM, 60 mM, 65 mM, 70 mM, or 75 mM potassium had therapeutically acceptable cell yields, as compared to T cells cultured in control media. Conversely, an increase of K+ concentration beyond 70-75 mM was reproducibly harmful to cell yield across multiple donors (FIG. 2D-2F).

Glucose concentration was additionally manipulated across these titrations. Nutrient restriction has been reported to promote T cell stemness and longevity, with inhibition of glycolysis promoting stemness. Conversely, higher glucose concentrations promote effector function and coincident loss of stemness. Consistent with this, the media formulation that demonstrated the most desirable T cell profile had a lower glucose concentration. Thus, we conclude that the reformulated media with the most desirable cell product, consisting of the highest number of stem-like T cells is hypotonic, moderately hyperkalemic, hyponatremic, hypocalcemic, and hypoglycemic relative to control media conditions.

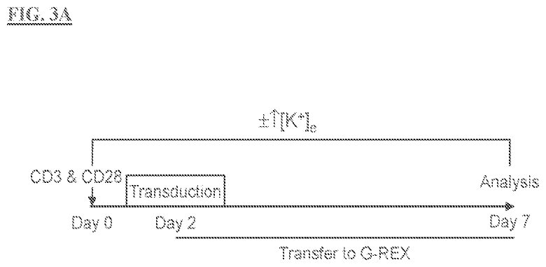

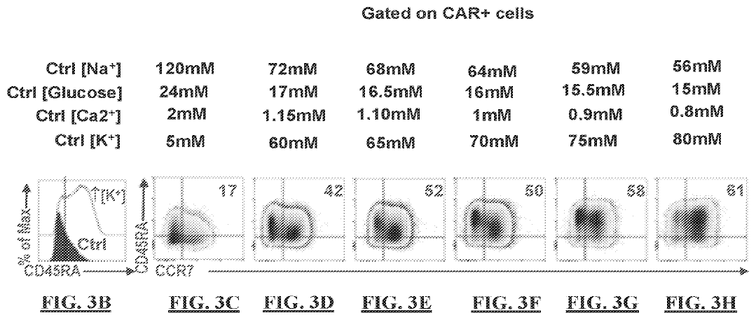

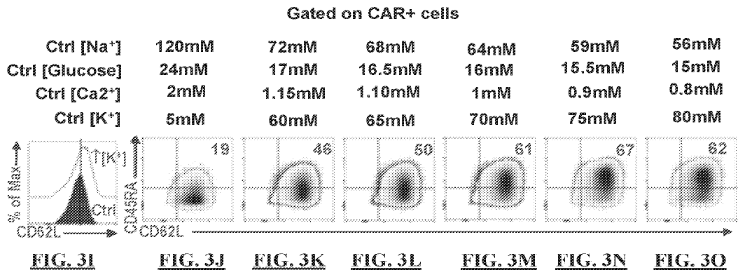

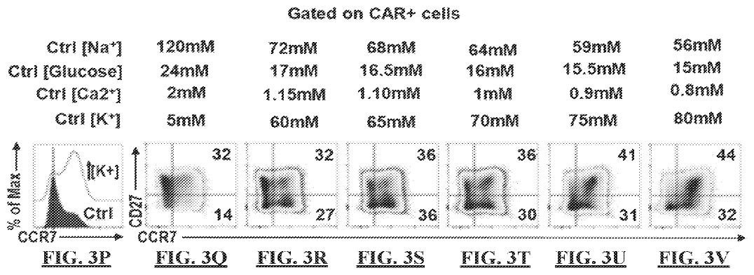

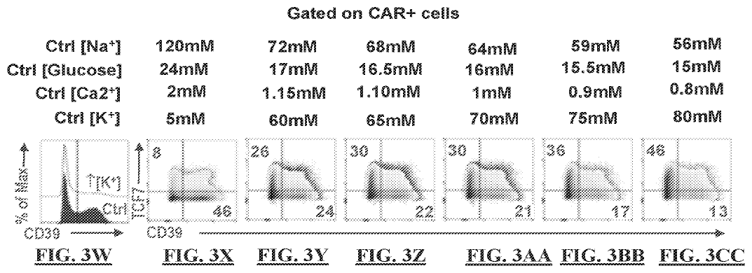

Across, titrations of ionic salts in hypotonic culture conditions (FIG. 3A) we found increased expression of CD45RA⁺ along with the lymph node homing molecules CCR7T, CD62L⁺, and T cell persistence associated marker CD27 (FIGS. 3B-3V). Table 1 shows each medium with potassium (50-90 mM), sodium chloride (50-90 mM), glucose (15-20 mM), calcium (1.2-2.0 mM), osmolality (˜250-280 mOsm), and tonicity. Table 1 also includes control media, i.e., a hypertonic medium with elevated potassium (55.3 mM) in an RPMI basal medium, which contains about 103 mM NaCl (data for this experiment is not shown). Compared to control conditions, hypotonic media with elevated potassium resulted in ˜30-50% increase in CD45RA⁺, CCR7⁺ expression, ˜30-50% increase in CD45RA⁺, CD62L⁺ expression and ˜10-20% increase in CCR7⁺ CD27⁺ cells (FIGS. 3B-3V). CD39 is an ecto-ATP/ADPase expressed on activated T cells and is involved in T cell effector differentiation, exhaustion and apoptosis. Increased expression of CD39 is a measure of accelerated aging and coincidental loss of stem cell-like phenotype. Compared to control conditions, hypotonic conditioned media resulted in ˜20-30% decrease in CD39⁺ expression (FIGS. 3W-3CC). These results indicate that elevated potassium enriches less differentiated cells while preventing exhaustion in T cells. The results also indicate that hypotonic conditioning increased TCF7 and decreased CD39 expression in CAR⁺ T cells (FIGS. 3W-3CC). Transcription factor TCF7 plays a major role in maintenance of T stem cell-like phenotype and ability to self-renew and produce more effector T cells. Compared to control conditions, media with elevated potassium ranging from 60 mM-80 mM resulted in ˜20-40% increase in TCF7⁺ expression (FIG. 3DD-3GG). These results indicate that hypotonic conditioning to CAR-T or T-cell products is associated with the ability to survive and persist following adoptive transfer.

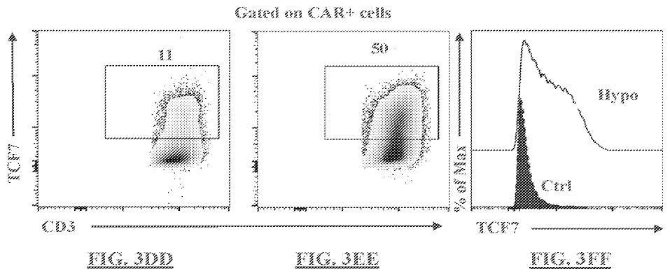

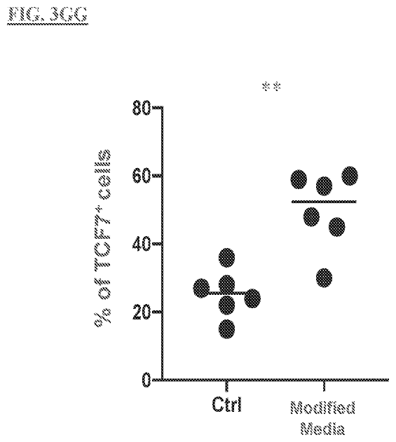

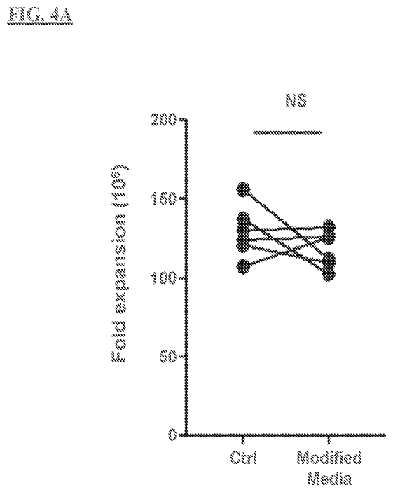

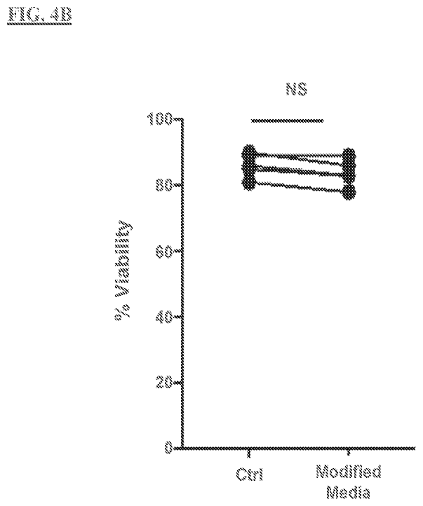

Culturing in hypotonic conditioning did not affect the yield or viability of the T cells during in vitro expansion (FIGS. 4A-4B). T cell yields and viability were consistent across multiple donors demonstrating that hypotonic media can be effectively used to produce cellular products containing less differentiated cells using clinical grade reagents.

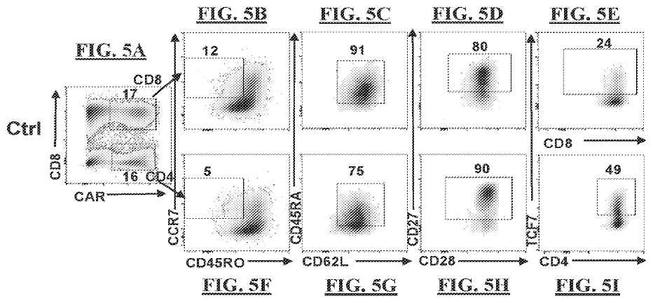

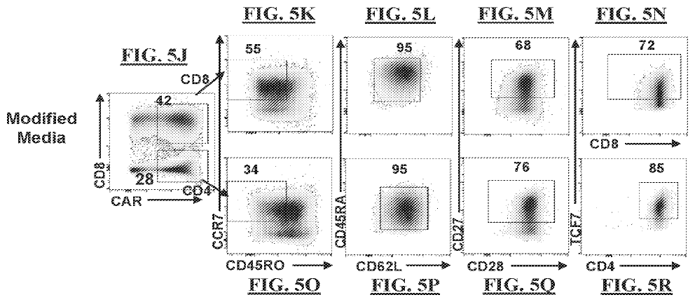

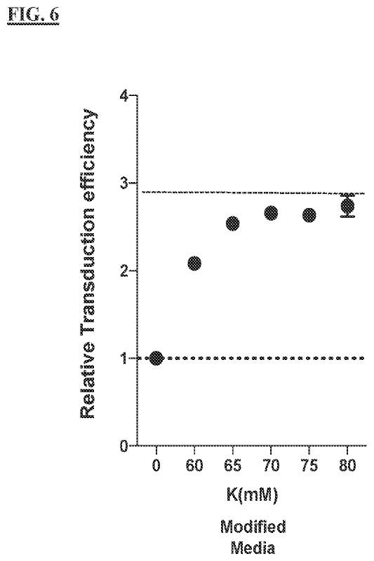

Since elevated potassium conditioning enriched less differentiated cells, we next sought to check T_{SCM} populations using stringent surface marker and transcription factors that accurately define “stem-like” T cells (FIG. 1A) (See, e.g., Gattinoni et al, Nature Medicine 2011). Our results indicated that hypotonic conditioning enriched CD45RO⁻ CCR7⁺ CD45RA⁺CD62L⁺CD27⁺ CD28⁺TCF7⁺ in both CD4 and CD8 CAR⁺ T cells (FIGS. 5A-5R). Compared to control conditions, hypotonic conditioned media increased the transduction efficiency by ˜2-3 fold (FIG. 6). Using these stringent criteria, we found that elevated potassium increases stem-like populations by 3- to 8-fold compared to control conditions (FIG. 7).

To further characterize the differentiation status of the cells, T cells transduced with a recombinant TCR were expanded with control media or metabolic reprogramming media (MRM; NaCl (40-80 mM), KCl (40-90 mM), Calcium (0.5-2.8 mM), Glucose (10-24 mM) and osmolality (˜250-340 mOsmol), see Example 3, below) and the level of protein expression of stemness related genes (BACH2, LEF1 and TCF1) was measured by western blot (FIG. 8A). Additionally, the level of protein expression of STAT3 and STAT5, including their activated phosphorylated forms was also measured (FIG. 8B). These data demonstrate that cells cultured in MRM exhibit significantly higher protein expression of stemness linked transcription factors, meaning the cells have retained an increased capacity for stemness features. Additionally, the increased abundance of phosphorylated STAT3 and STAT5 indicates that these cells also exhibit higher levels of pro-growth and pro-inflammatory signaling pathways that can provide increased anti-tumor effector function.

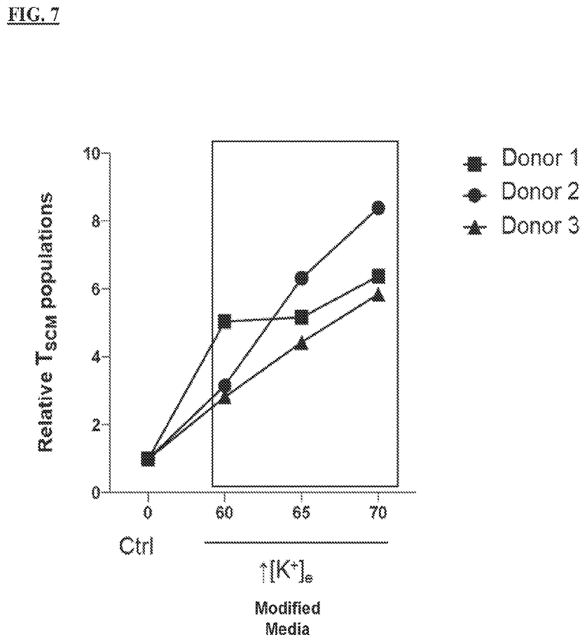

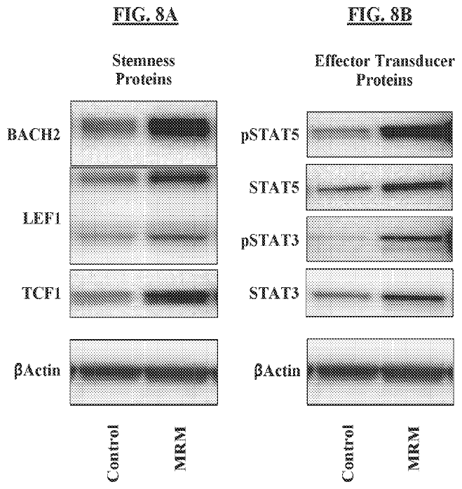

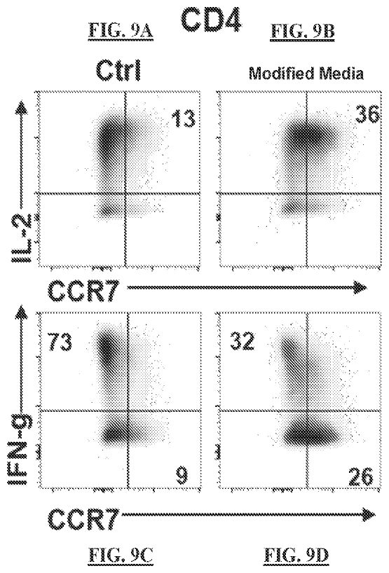

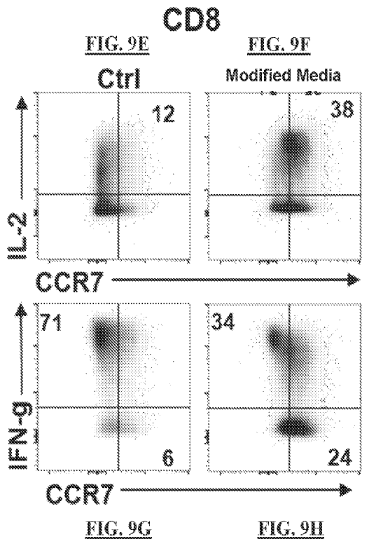

Previous reports indicate that in vitro acquisition of full effector functions impairs the efficacy of adoptively transferred T cells, with per cell production of IFNγ being a reliable surrogate for this acquisition of effector function (Gattinoni et al, JCI 2005). Conversely, those T cells with retained capacity for IL-2 production represent a population with retained capacity for stemness (Gattinoni et al., JCI(2005); Wang et al., STM(2012)). We therefore assessed the fate of effector functions of T cell populations generated using hypotonic conditioned media. Compared to control conditions, hypotonic conditioned media preserved lymphoid homing receptors with enhanced IL-2 expression by 3-fold and reduced IFNγ expression by 3-fold (FIGS. 9A-9J). The opposing effects of IFNγ and IL-2 are crucial to maintain less differentiated state during in vitro expansion of T-cells. This phenomenon is observed in both CD4 and CD8 subsets without the loss of CCR7 expression (FIGS. 9A-911).

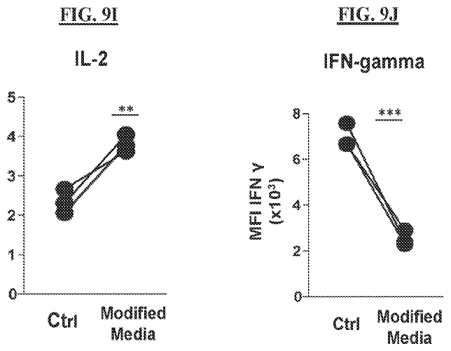

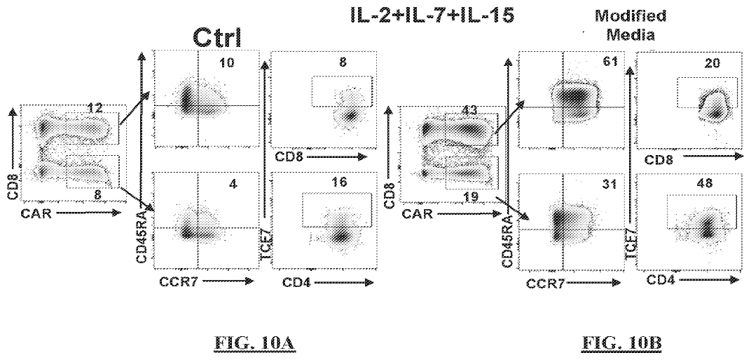

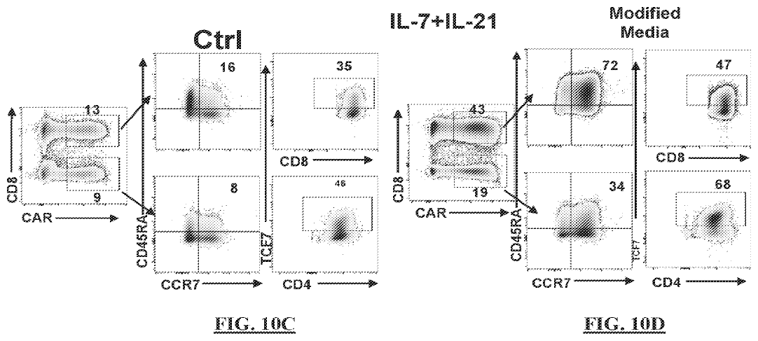

Previous reports indicate that while IL-2 is of great utility for the production of large numbers of activated T cells, a concurrent rapid differentiation and coincident loss of stemness is also observed following in vitro exposure to IL-2 differentiation and proliferation of T cells (Waldmann et al, Nat Rev Immunol, 2006). Conversely, prior reports (Hinrichs et al, Blood, 2008) have indicated that utilization of alternative common gamma chain cytokines can result in similar cell yield with a comparative preservation of T cell stemness. To this end, we tested whether titrated amounts of different combinations of these cytokines could provide an added benefit to our otherwise reformulated media with respect to T cell stemness and total cell yield. Our results indicated that combination of IL-7 and IL-21 was sufficient to enrich less differentiated products (FIGS. 10A-10D). Combinations of these cytokines had very minimal effects in enriching naive-like cells compared to standard culture conditions. However, using stringent markers including CD45RO⁻CCR7⁺ CD45RA⁺CD62L⁺CD27⁺ CD28⁺TCF7⁺, our results indicate that compared to standard culture conditions IL-7, IL-21 with elevated potassium and hypotonic culture conditions resulted in relatively higher number (˜20 fold more) of stem-like cells in CAR engineered products (FIGS. 11A-11B).

The effects of culturing CAR engineered T cells in hypotonic media comprising elevated potassium were also observed in recombinant TCR-modified immune cells. T cells were transfected with an NY-ESO-1 TCR, and cultured in hypotonic media with elevated potassium, as described above. Culture of such TCR-modified immune cells in hypotonic media with elevated potassium resulted in similar cell yield (˜150 million cells), viability (greater than or equal to 90%), and a stem-like phenotype (˜₅₀% of CD3⁺ cells expressed CCR7 and CD45RA) as CAR-T cells cultured under similar hypotonic conditions with elevated potassium (data not show).

### Example 3. Methods of Preparing Media and Culturing TILs

Control Media: Commercially available T cell media (e.g., CTS™ OPTIMIZER™, IMMUNOCULT™ or TEXMACS™)

Metabolic reprogramming media (“MRM”): The inorganic salt ion concentrations of T cell media were adjusted using NaCl free T cell media. The final concentrations of MRM were the following: NaCl (40-80 mM), KCl (40-90 mM), Calcium (0.5-2.8 mM), Glucose (10-24 mM) and osmolality (˜250-340 mOsmol).

TIL media preparation used for initial culture and secondary and final TIL expansions: Either Control media or MRM was supplemented with 2.5% serum supplement (CTS™ Immune Cell SR, Thermo Fisher), 2 mM L-glutamine (Gibco), 2 mM L-glutamax (Gibco), MEM Non-Essential Amino Acids Solution (Gibco), Pen-strep (Gibco), 20 g/ml FUNGIN™ (InvivoGen), Sodium pyruvate (Gibco), and 1 mM of O-Acetyl-L-carnitine hydrochloride (Sigma).

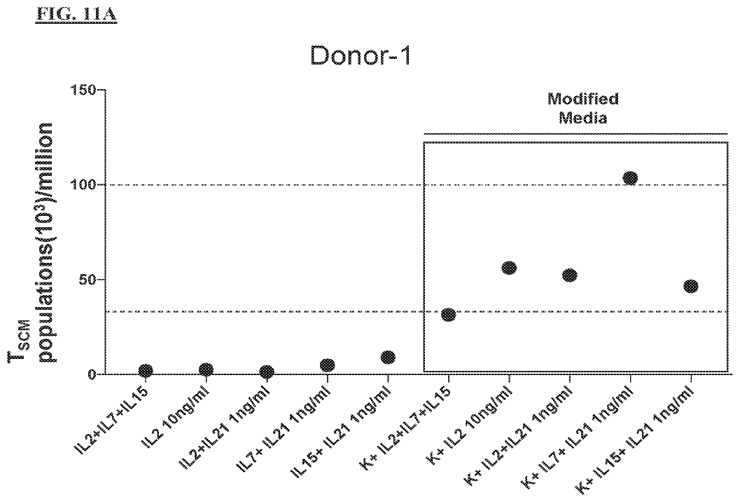

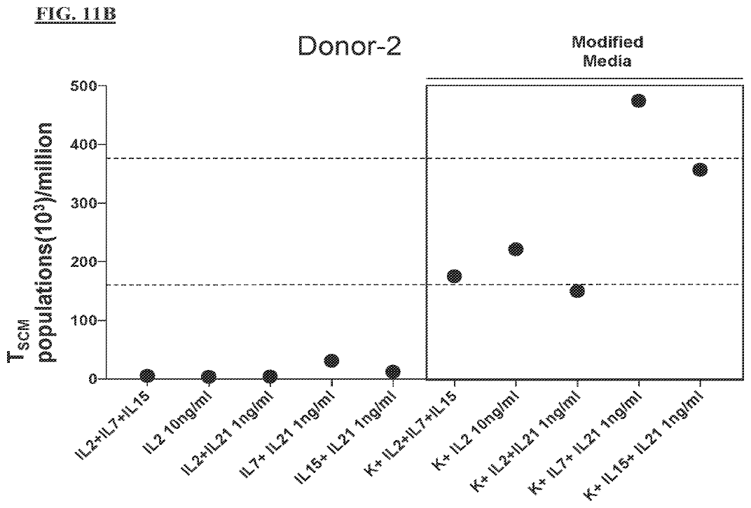

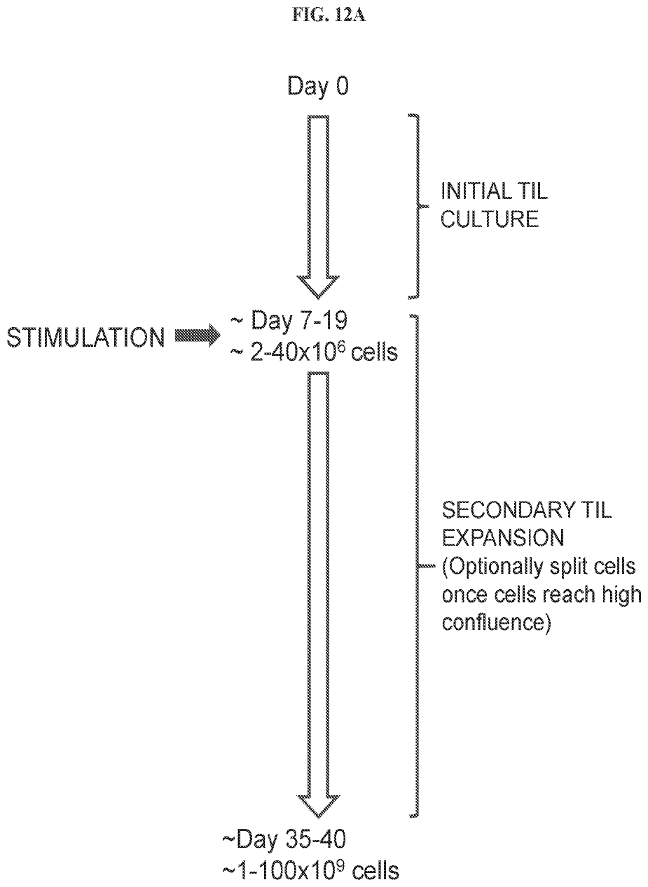

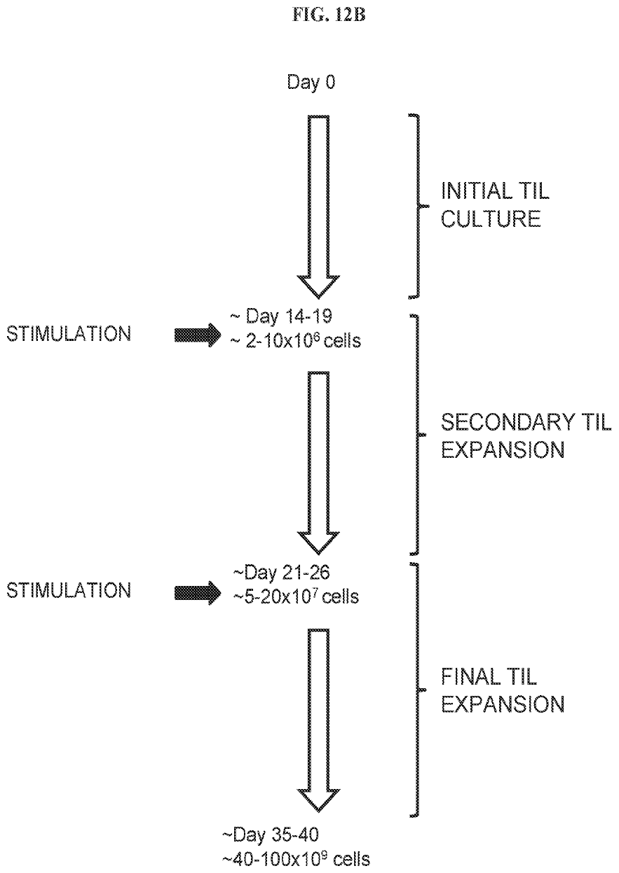

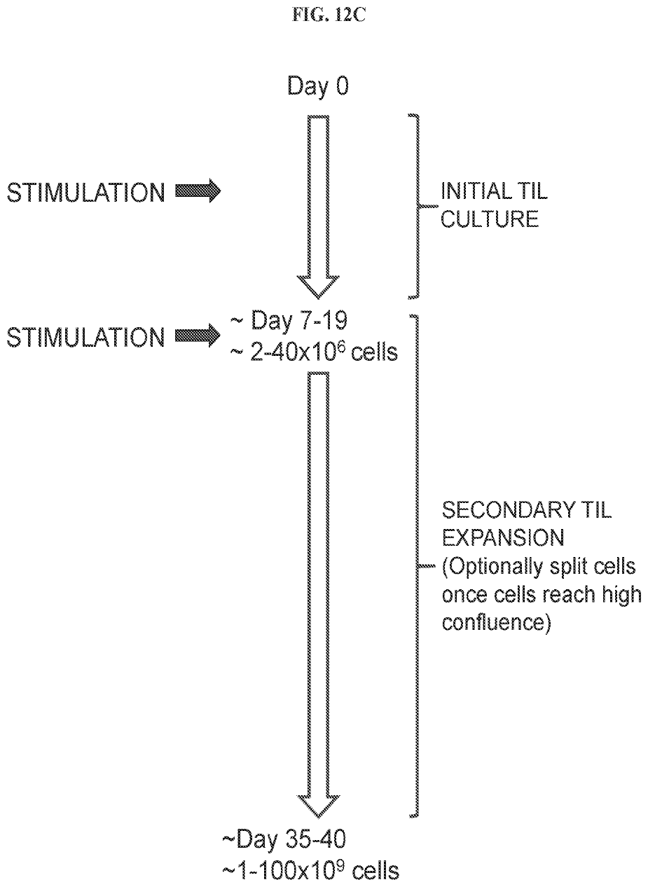

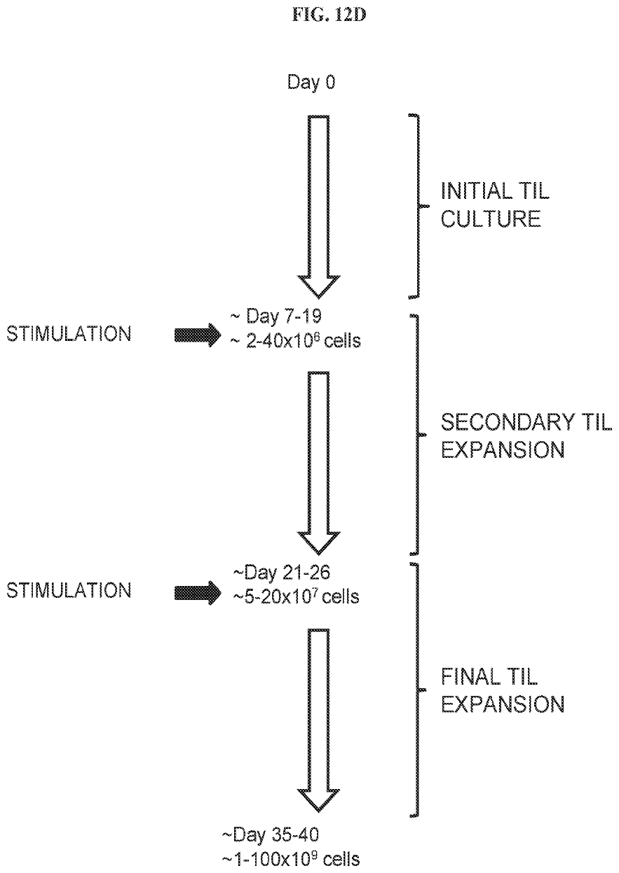

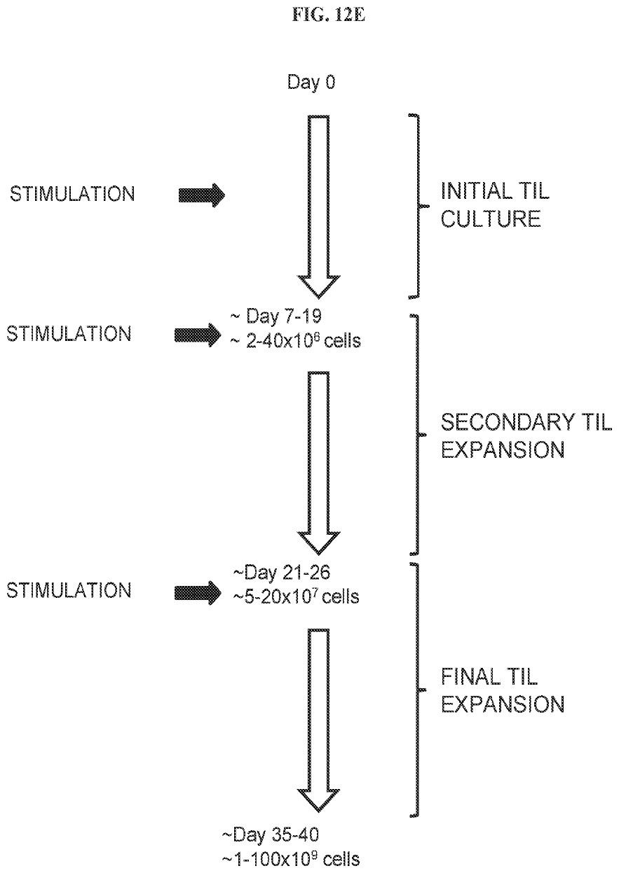

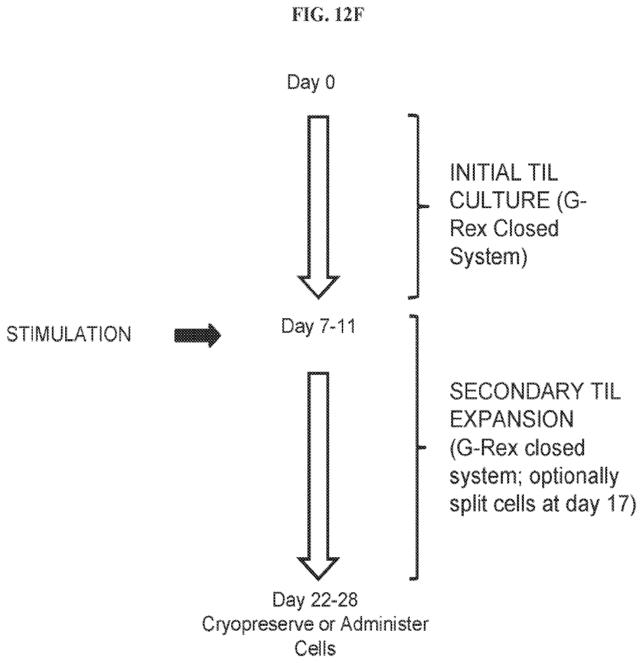

Initial TIL Culture: FIG. 12 is a schematic depicting generally certain aspects of the methods of culturing TILs described herein. Multiple tumors surgically resected from various tumor types (colon, lung, hepatocellular carcinoma, renal, pancreas, breast, melanoma, and prostate) with an average size of 1-10 mm³ were seeded in 24-well plates in 2 ml of either control media or MRM as described above, both supplemented with IL-2 (300 ng/mL) and IL-21 (30 ng/ml). Tumor fragments were cultured in a heat jacketed incubator at 37° C. incubator with 5% CO₂ until colony formation was visible. Fresh media (control or MRM) supplemented with IL-2 (300 ng/mL) and IL-21 (30 ng/ml) were replenished every 3 days depending on the growth of the cells. This method resulted in a yield of about 2×10⁶-10×10⁶ cells per fragment at the end of the initial culture. A subset of cells for analysis were passed through a 40 μm strainer and phenotyped with multi-color flow cytometry using various biomarkers including CD62L, CD27, CD28, CD45RO⁻, CD39, TIM3, CD127, PD1, CD103, CD45RA, and TCF7.

Secondary TIL Expansion: When cell yield from the initial culture reached about 2×10⁶-10×10⁶ cells per cultured fragment (usually at about day 14 to day 19), the TILs cultured in either control media or MRM, both supplemented with IL-2 (73.6 ng/ml), IL-21(10 ng/ml), and IL-15 (0.4 ng/ml), were stimulated by adding 1:100 T cell TRANSACT™ (Miltenyi Biotec), 5 μg/ml recombinant human CD27 ligand (R&D systems), and 1 μg/ml recombinant human 4-1BB ligand/TNFSF9 (R&D systems). Cells were maintained in culture until about 5×10⁷ to 20×10⁷ cells were obtained (about 7 to 11 days post-stimulation). At the end of the secondary expansion period, TILs were analyzed with multicolor flow cytometry using various biomarkers including, CD62L, CD27, CD28, CD45RO⁻, CD39, TIM3, CD127, PD1, CD103, CD45RA and TCF7. Only live and CD3⁺ cells were analyzed.

Final TIL Expansion: When the cultures reached a yield of about 5×10⁷ to 20×10⁷ cells, the TILs cultured in either control media or MRM were transferred to fresh control media supplemented with IL-2 (73.6 ng/ml), IL-21(10 ng/ml), and IL-15 (0.4 ng/ml). TILs were stimulated for a second time with 1:100 TRANSACT™ (Miltenyi Biotec), 5 μg/ml recombinant human CD27 ligand (R&D systems), and 1 g/ml recombinant human 4-1BB ligand/TNFSF9 (R&D systems). The cells were cultured in static GREX or stirred tank until a yield of about 10×10⁹-100×10⁹ cells per fragment was achieved (about 14 days) and analyzed with multi-color flow cytometry for various biomarkers, including CD62L, CD27, CD28, CD45RO⁻, CD39, TIM3, CD127, PD1, CD103, CD45RA, and TCF7. To check for polyfunctionality of the cells, the resultant TILs were stimulated with PMA/ionomycin (1:500) for 4 hours and intracellular staining was performed using the following markers: CD4, CD8, CD27, IL2, IFNγ, TNFα and TCF7. Only live and CD3⁺ cells were analyzed.

### Example 4. MRM results in expansion of CD8⁺, tumor-reactive, less differentiated TILs

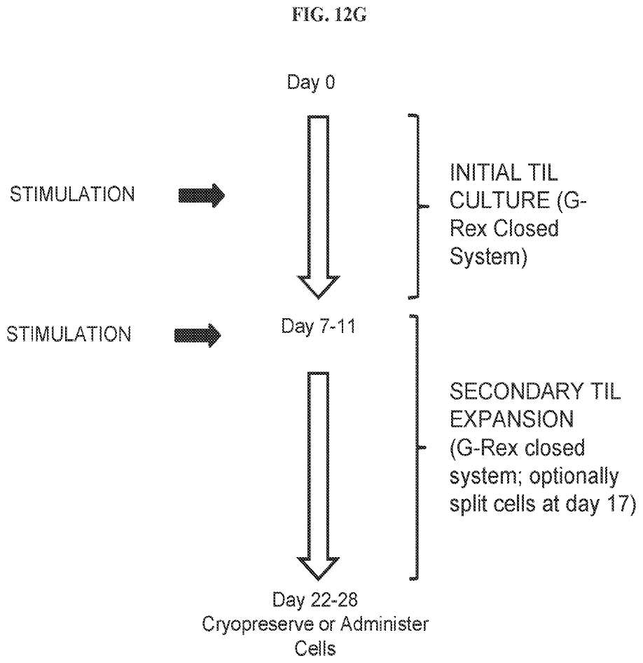

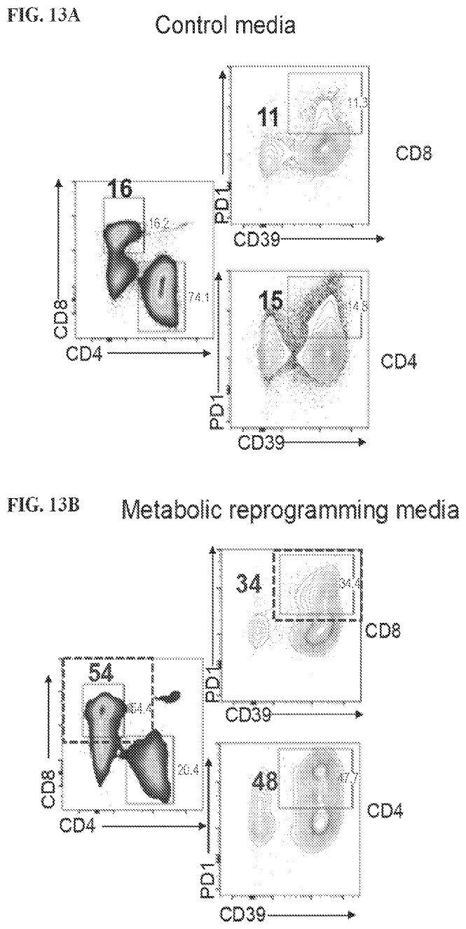

TILs were grown as described in Example 3 (FIG. 12). After the initial TIL culture (i.e., 14 days), multiparameter flow cytometry was performed to quantify the percentages of CD4⁺ and CD8⁺ TILs present in the cell culture. Cells cultured in MRM had significantly enriched CD8⁺ TILs by ˜20-80% as compared to the cells cultured control media (FIGS. 13A-13C and data not shown). Although CD4⁺ TILs are capable of eradicating solid tumors, superior cytolytic activity towards tumors is primarily mediated by CD8⁺ TILs. Tumor cells predominantly express MHC class I associated tumor antigens, which are recognized by CD8⁺ TILs. Thus, having a greater proportion of CD8⁺TLs in the TIL therapy infusion product is therapeutically beneficial. Use of MRM to culture TILs unexpectedly enriched CD8⁺ TILs as compared to TTLs cultured in control media (FIG. 13C).

TTLs obtained at the end of the initial culture in MRM (about day 14) also demonstrated consistent expression of several cell surface markers of tumor-reactive TILs (e.g., CD39, CD103, CD226, and/or PD1). Initial culturing in MRM produced TILs with enhanced expression of CD39 and PD1 (greater than 20%) as compared to TILs cultured in control media (FIGS. 13A-13B). Previously used methods show that PD1 expression is completely lost in the PD1 subsets during initial TIL culture, indicating undesirable loss of tumor-reactive TTLs (see, e.g., Poschke et al, Oncoimmunology 5(12):e1240859 (2016); and Gros et al, JCI 124(5):2246-59 (2014)). We observed that maintenance of PD1 expression after T cell stimulation was dependent on donor (data not shown). Similar results were obtained for expression of other markers of tumor reactivity including CD39 and CD103 (see FIGS. 13A-13B and 13A-13B, and data not shown). However, clonal repertoire observed at day 14 is maintained throughout entire TIL process (data not shown).

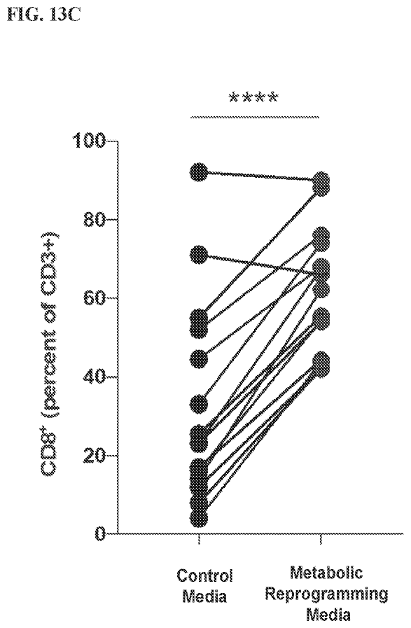

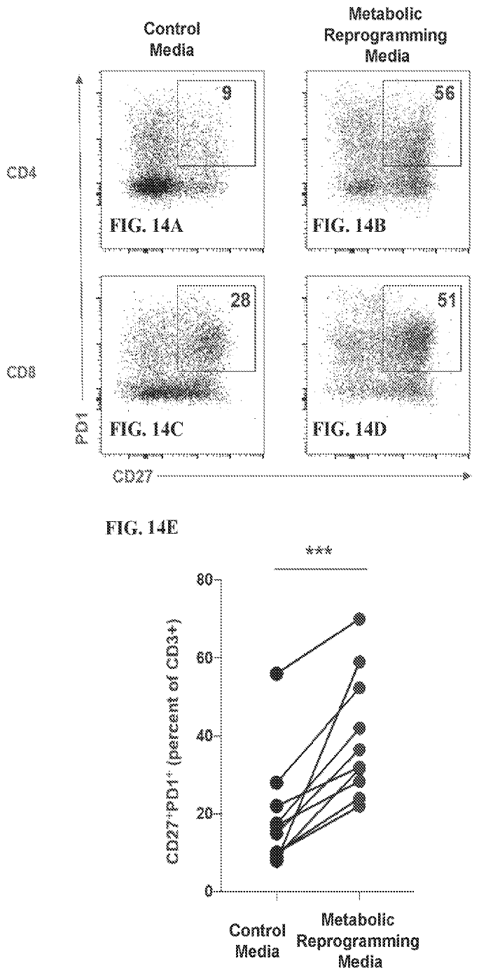

We also observed co-expression of PD1 (indicative of a tumor-reactive metabolic state) and CD27, in both CD4⁺ and CD8⁺ TILs cultured in MRM (FIGS. 14A-14E). CD27 expression, which is constitutively expressed on naive and memory committed T-cells, is indicative of a stem-like phenotype in T cells. Previous reports indicate that CD27 expression is reduced in CD8⁺ T cells during cell expansion following T cell stimulation (see, e.g., Tran et al., J. Immunotherapy 31(8):742-51 (2008); and Rosenberg et al., Clinical Cancer Research 17(13):4550-557 (2011), Huang et al, J. Immunology 176(12):7726-35 (2006)). In contrast, TILs cultured in MRM, disclosed herein, have preserved CD27 expression throughout the culturing process, allowing for selective expansion of stem-like tumor-reactive clones. As expected, not all CD27⁺ cells at day 14 co-expressed PD1, indicating that not all stem-like cells displayed a tumor-reactive metabolic state.

Anti-tumor function and survival of TILs are dependent on the consolidated signals received by the TCR, cytokine, and costimulatory receptors. Inadequate exposure of any of these signals will result in anergy and atrophy of the TILs. Previously used methods of culturing and expanding TILs result in loss of CD27 expression in the TILs. However, TILs that maintain CD27 expression in an infusion product, e.g., in minimally expanded TILs, have been shown to be associated with tumor regression following adoptive T cell therapy (see, e.g., Tran et al., J. Immunotherapy 31(8):742-51 (2008); and Rosenberg et al., Clinical Cancer Research 17(13):4550-557 (2011)). In addition, elevated expression of costimulatory receptors, e.g., CD27 and CD28, is associated with in vivo therapeutic efficacy (Tran et al., J. Immunotherapy 31(8):742-51 (2008), Geltink et al., Cell 171, 385-397 (2017)). Expression of these costimulatory receptors has also been associated with stemness and longer telomere lengths that correlated with young TIL cultures. In contrast to previously used methods, e.g., control media, the use of MRM described herein enriched TILs with CD27 and CD28 expression to about 20% to about 80% of the total number of TILs across several tumor types (FIG. 15 and data not shown). Enrichment of CD27 and CD28 was not unique to the CD8⁺T cell subset but was also observed in the CD4⁺subset (data not shown).

Expression of CD27 and CD62L is correlated with less differentiated T cells and is associated with efficient trafficking of the T cells to tumor tissues and lymph nodes. Expression of CD27, CD28, and CD62L is also linked to longer telomere length, which is indirectly linked to the age of the TIL and in vivo therapeutic efficacy (see, e.g., Tran et al., J. Immunotherapy 31(8):742-51 (2008); and Rosenberg et al., Clinical Cancer Research 17(13):4550-557 (2011)). However, TILs cultured in MRM, as disclosed herein, maintained both CD27 and CD62L expression throughout the process, similar to that of minimally cultured TILs. We observed a ˜50% increase in CD27 and CD62L expression in TILs cultured in MRM as compared to those cultured in control media (data not shown).

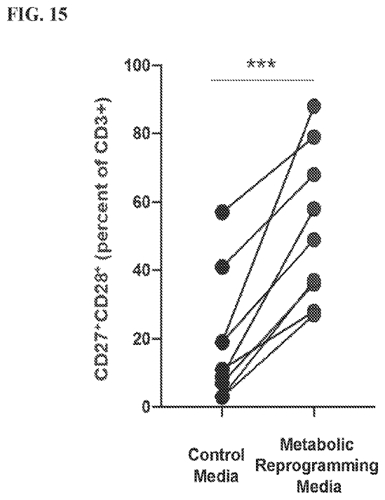

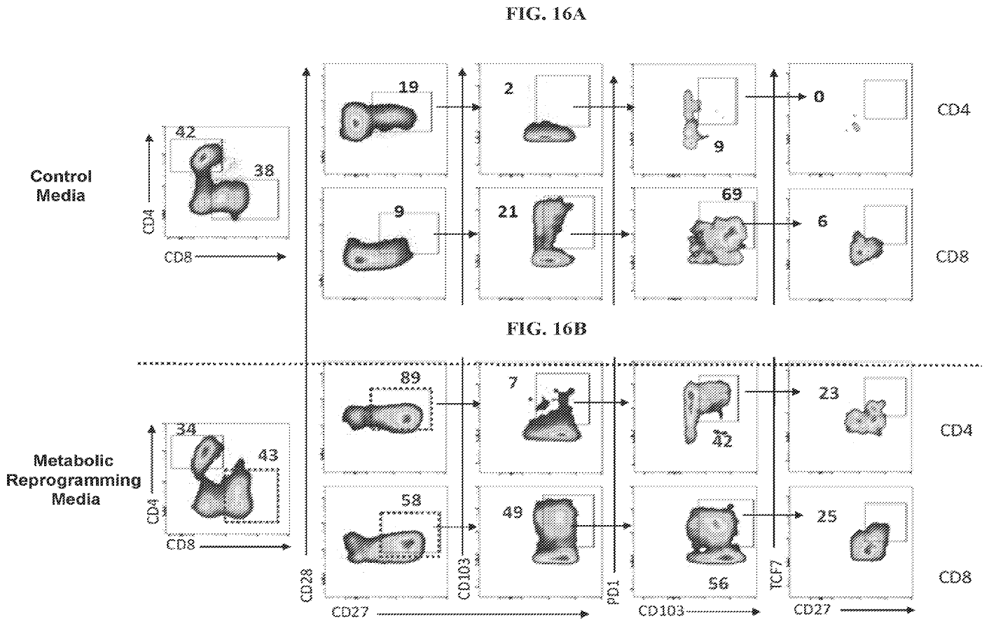

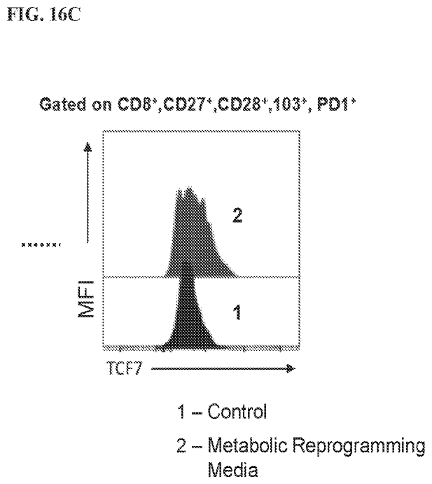

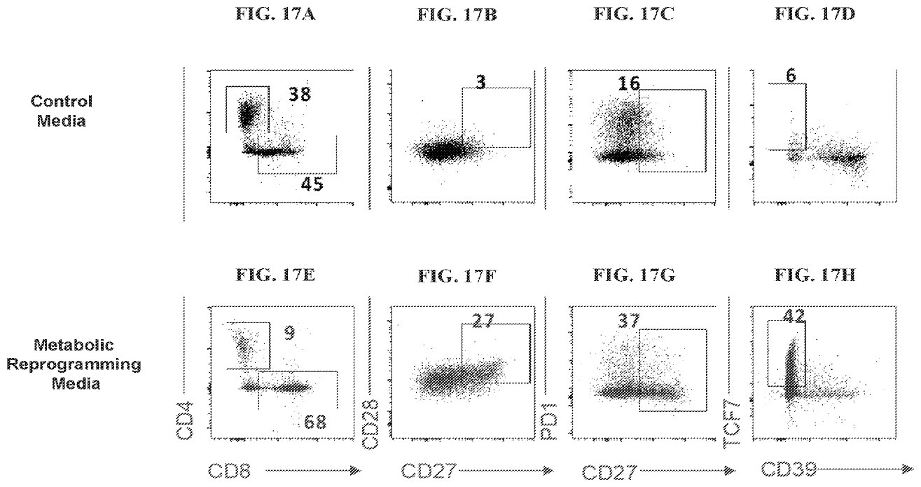

Tumor-reactive clones that mediate tumor regression post immune checkpoint blockade are believed to be derived from the CD8⁺ T cells that expressed PD1 and transcription factor TCF-7 (Im et al., Nature 537:417-21 (2016); and Feldman et al., Cell 175(4):998-1013 (2018)). TILs cultured in MRM disclosed herein displayed enrichment of tumor-reactive TIL biomarkers (PD1 and CD103) with a concurrent 4-fold to 50-fold higher level of TCF7 expression (FIGS. 16A-16C, 17D, 17H, and 21). Expression of TCF7 is indicative of more stem-like cells. Despite the expression of PD1, these cells retained proliferative capacity and maintained less differentiated cells upon further stimulation (FIGS. 17A-17H).

### Example 5: MRM Preserves Tumor Reactivity of TILs

Tumors are heterogeneous in nature and often contain common mutations in genes such as KRAS, P53, and BRAF (public neoantigens). The methods using MRM as disclosed herein enriched for TILs that recognize such neoantigens.

Tumor resections were obtained from a patient with pancreatic adenocarcinoma, a cancer that predominantly has tumor cells with KRAS^{G12V}, KRAS^{G12C}, and KRAS^{G12D} mutations. HPLC-purified 9mer, 10mer, and 25mer peptides of the above-mentioned KRAS hot spot mutations were purified and used to pulse immature dendritic cells (DCs) generated from patient-matched peripheral blood monocytes. These peptide pool-pulsed DCs were co-cultured with TILs obtained from the same pancreatic adenocarcinoma patient and cultured according to the methods described in the Examples above. Culture of the TILs in MRM resulted in ˜30% more CD8⁺ T cells with significant co-expression of CD27, CD28, PD1, and TCF-7 (FIGS. 18A-18H). These results indicate that MRM disclosed herein preserves tumor-reactive TCR clones that recognize public antigens.

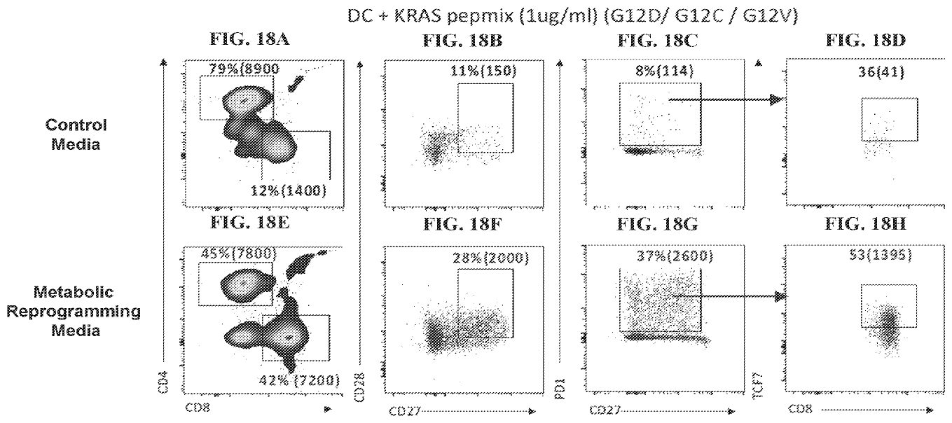

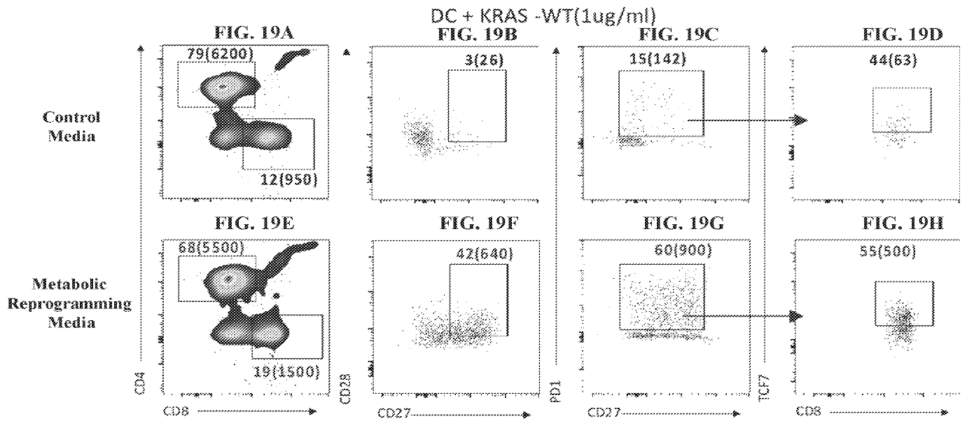

As expected, TILs pulsed with wild type KRAS peptides did not result in specific expansion of KRAS-specific TILs. However, we consistently observed increased expression of CD27, CD28, PD1, and TCF7 expression by TILs cultured in MRM. These results indicate that culturing cells in MRM preserves the growth and proliferation of TILs that recognize rare public neoantigens (FIGS. 19A-19H).

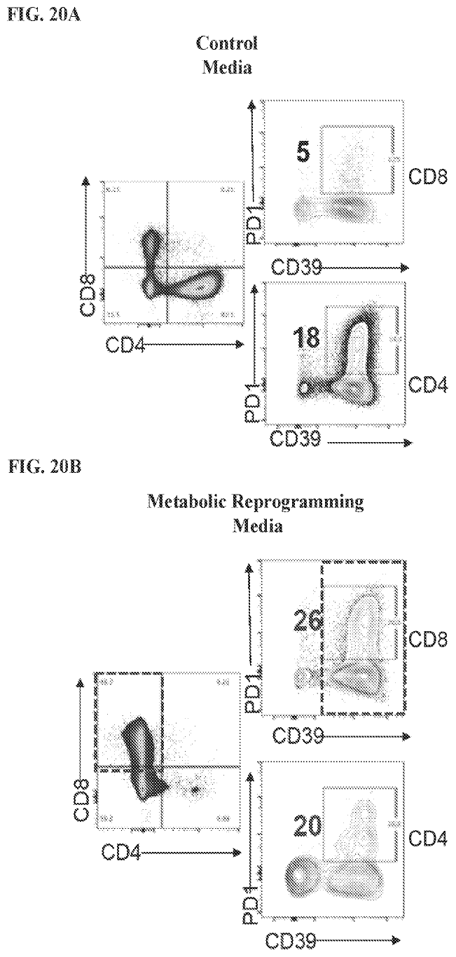

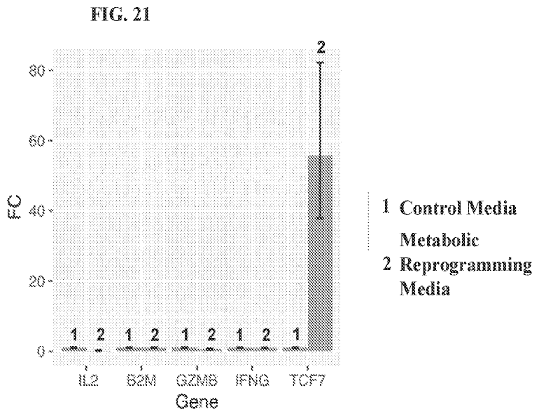

Preferential enrichment of CD8⁺ TILs and markers associated with tumor reactivity CD39 and PD1) were maintained during this expansion process (FIGS. 20A-20B). These results show that MRM further enhances PD1 expression in tumor-reactive TILs even after initial culture. In addition, TILs cultured in MRM according to the methods disclosed herein exhibited a greater than 50-fold increase in TCF7 expression as measured by qPCR (FIG. 21). These data are consistent with the role of TCF7, which is a master transcriptional regulator required for self-renewal and proliferative burst of the TILs post re-stimulation.

Expression of CD103 in TILs is reported to be correlated with TIL infiltration in tumors with high mutation antigen burden. In addition to CD39 and PD1 expression, our data demonstrated that expansion of TILs in MRM increased the number of PD1⁺CD39⁺CD103⁺ cells (FIGS. 22A-22L) while also increasing expression of stemness-associated genes. Therefore, culture of TILs in MRM may be sufficient to increase the number of cells with both tumor-reactivity and higher proportion of stem-like cells in the resulting expanded cell products.

The data presented herein demonstrate that expansion of TILs in MRM generates cell populations with not only increased stemness, but also increased expression of several cell surface markers associated with tumor reactivity. The methods described herein using MRM produce TILs that have characteristics and properties of an improved therapeutic product.

### Example 6. Methods of Treatment

TILs that will be used for infusion will be derived from a patient's own tumor, excised from primary, metastatic, or lymphnodes. TIL cultures will be initiated by plating small tumor fragments (˜1-10 mm³) in 24 well plates containing MRM as described above in Example 1. These fragments will be grown until about 2×10⁶ to about 10×10⁶ cells/tumor fragment are obtained (typically about 2-3 weeks). The resulting cells from all the fragments will be pooled and plated at a density of 2×10⁶/well for T cell stimulation, e.g., by adding TRANSACT™, and optionally CD27 agonist, 41BB agonist, and/or OX-40. Cells will be maintained in culture until about 5×10⁷ to about 20×10⁷ TILs is obtained. These cells will be further stimulated, e.g., using TRANSACT™ and optionally CD27 agonist, 41BB agonist, and/or OX-40 to achieve a 1000-2000 fold increase in the number of cells (˜1-150×10⁹ TILs) for the infusion product.

Prior to administration of the TIL infusion product, patients will be administered a lymphodepletion treatment, e.g., comprising cyclophosphamide and fludarabine. In addition to TIL infusion, patients will also receive an anti-PD1 checkpoint inhibitor (e.g., pembrolizumab or nivolumab) after infusion of TILs cultured as disclosed herein.

### Example 7. Characterization of CAR T cells cultured in MRM

CD4+ and CD8+ donor matched T cells were cultured in control media or metabolic reprogramming media (MRM) as described above, both of which were supplemented with 200 IU/ml IL-2, 1200 IU/ml IL-7, and 200 IU/ml IL-15 (R&D systems) and activated using TRANSACT™ (Miltenyi Biotec). 24 or 48 hours post activation, cells were transduced with lentivirus encoding a ROR1 chimeric antigen receptor (CAR). T cells were resuspended at 2.5e6 live T cells/ml in either control media or MRM, supplemented with 200 IU/ml IL-2, 1200 IU/ml IL-7, and 200 IU/ml IL-15. After overnight incubation cells were transferred to G-REX® plates (Wilson Wolf) for expansion for 7 days post-activation before cryopreservation for subsequent use in the assays described below.

MRM substantially improves retention of a less differentiated T cell during production. Such stem-like T cells also have higher capacity for proliferation upon target stimulation and tumor cell killing capacity (data not shown).

At day 7 post expansion, 1×10⁶ CAR T cells per group were stimulated with a PMA and ionomycin cocktail followed by golgi-inhibitors (Biolegend, per manufacturer's instructions). Cytokine production was assessed via intracellular staining and flow cytometry measured by live/CD3+/CAR+ and then comparing IL-2+, IFNγ+, IL-2+/IFNγ+ double positive, and double negative subsets.

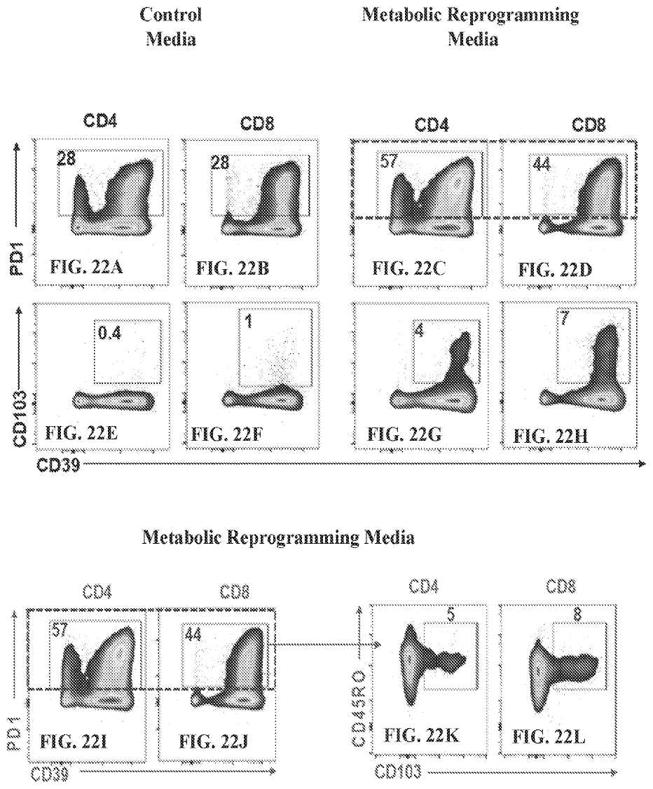

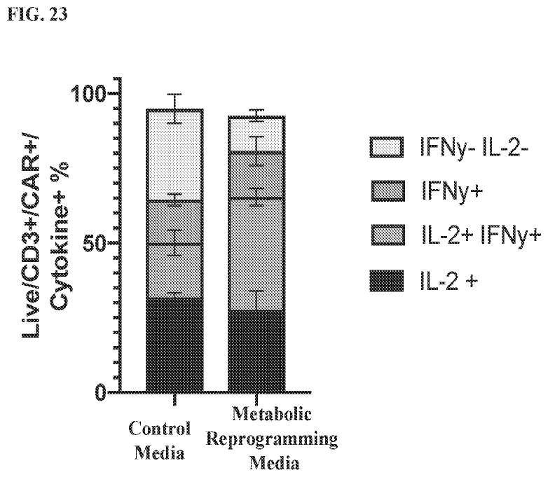

CAR T cells expanded in MRM show enrichment for IL-2+ and IL-2+/IFNγ+double positive T cells compared to CAR T cells expanded in control media (FIG. 23; average of three unique donors). This cytokine profile is consistent with the surface marker analysis, in which culturing of cells in MRM produces a CAR T cell population that has more T cells that are less differentiated. IL-2 production in T cells is indicative of a less differentiated T cell in comparison to solely IFNγ production or no cytokine production. Moreover, increased IL-2 production would support persistence of the CAR T cells and thus a better therapeutic.

To assess T cell cytotoxicity, ROR1 CAR T cells generated as described above were seeded at a 1:5 (E:T) ratio with Nuclight Red labeled (NLR) H1975-ROR1⁺target tumor cells in a 24 well plate with 3× technical replicates per CAR T condition. Tumor cell killing was determined via Incucyte fluorescent measurement over time of mean NLR intensity compared to tumor cells alone. After 3 days of culture one-third of T cells were removed from target cell wells, seeded on fresh target cells, and replaced in the Incucyte. This was repeated for up to 3-4 more times at either 3 or 4 day intervals.

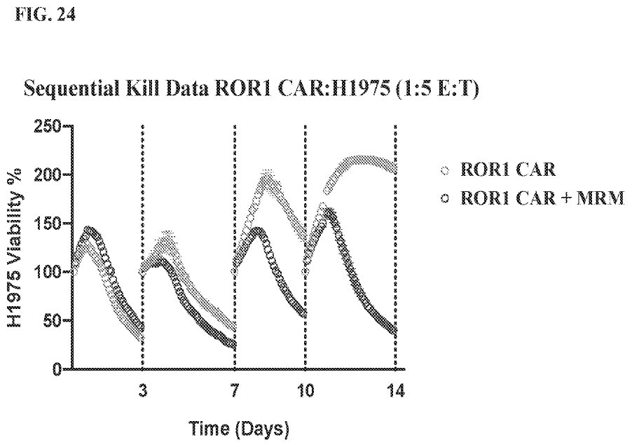

CAR T cells cultured in MRM showed enhanced cytotoxicity as compared to CAR T cells cultured in control media (FIG. 24; y-axis represents target cell density). CAR T cells cultured in control media lose the ability to control target tumor cell growth over time. This increased cytotoxicity indicates that CAR T cells cultured in MRM have a greater and more persistent anti-tumor effect.

Cytokine secretion of the CAR T cells from the sequential killing assays above was assessed. Briefly, after each stimulation (e.g., on days 3, 7, and 10), media supernatant was collected after 24 hours of coculture with target tumor cells. Cytokine secretion was measured using a V-PLEX Proinflammatory Panel 1 Human Kit (MSD).

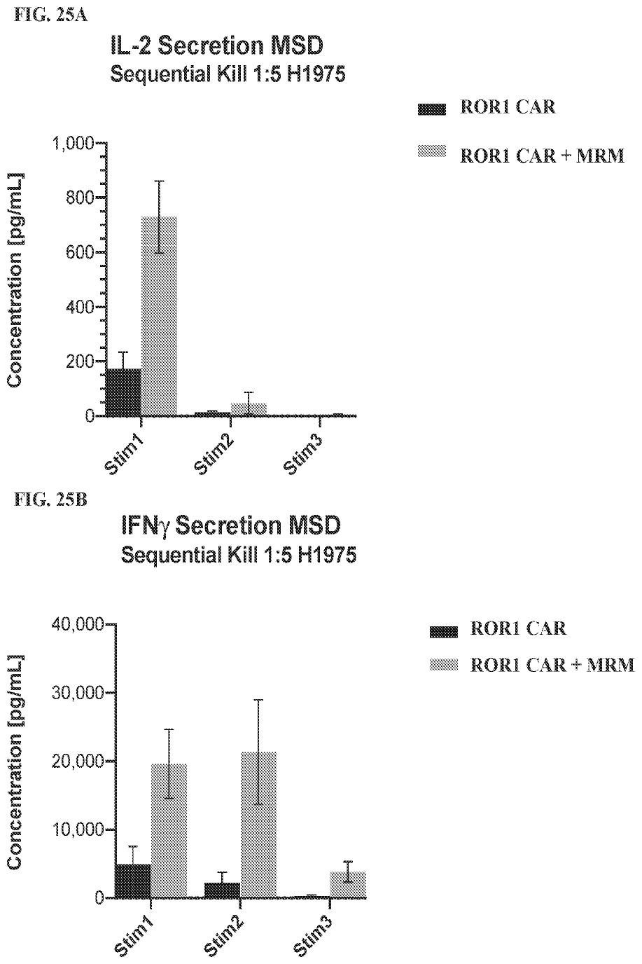

ROR1 CAR T cells cultured in MRM produced significantly more IL-2 (FIG. 25A) and IFNγ (FIG. 25B) in co-culture with H1975 target tumor cells as compared to CAR T cells cultured in control media. Similar to the data obtained with PMA/ionomycin stimulated T cells shown in FIG. 23, CAR T cells cultured in MRM show increased IL-2 (˜4×) and IFNγ (˜4×) secretion when co-cultured with target tumor cells, as assessed via ELISA. Upon repeat stimulation, IL-2 secretion is lost in all samples; however, CAR T cells cultured in MRM showed substantially higher IFNγ throughout three target cell stimulations. The increased magnitude and duration of cytokine secretion with CAR T cells cultured in MRM indicates a stronger anti-tumor response with delayed terminal differentiation of the CAR T cells.

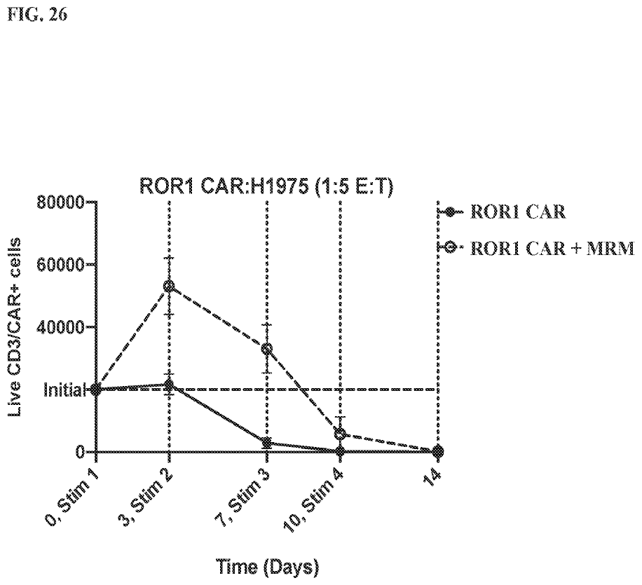

MRM produced CAR T cells also show enhanced proliferation in co-culture with H1975 target tumor cells in the sequential killing assay (FIG. 26). The greater proliferation indicates that MRM produced cells are more stem-like with high proliferative capacity. The increased expansion also supports that CAR T cells produced by culturing in MRM have a superior anti-tumor response.

### Example 8. Cells Cultured in MRM are Transcriptionally Distinct

Recombinant TCR transduced T cells were generated using control media or MRM using the methods described herein using T cells of a healthy donor (H) and two diseased donors (D1, D2). TCR+ cells from the product were sorted and subjected to bulk RNA-seq (performed as described in Galletti et al. Nat. Immunology 21, 1552-1562 (2020)), bulk ATAC-seq (performed as described in Lynn et al. Nature 2019) and single-cell Cellular Indexing of Transcriptomes and Epitopes (CITE)-seq using the 10× genomics platform (Stoeckius et al. Nat. Method 14, 865-868 (2017)). CITE-seq data was processed using the 10× cellranger software (10× Genomics) with standard parameters. Bulk RNA-seq and ATAC-seq data was processed using standard analysis pipelines (as described in Lynn et al. Nature 2019).

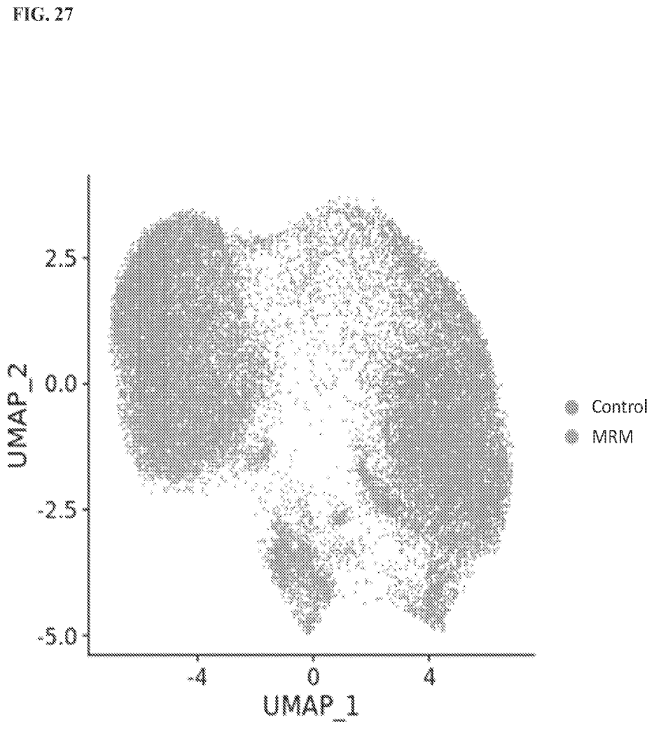

Single-cell gene expression across hundreds of genes from the resulting cells were mapped to two dimensions using Uniform Manifold Approximation and Projection (UMAP) (Becht, E. et al. Nat. Biotech. 2018) with each dot representing a cell (FIG. 27). The UMAP illustrates that cells generated from the culturing in control media (UMAP_1 generally greater than 0) or MRM (UMAP_1 generally less than 0) cluster separately in the two-dimensional space indicating that they differ substantially in their global transcriptome. Cells generated using MRM appear as a distinct and predominantly homogeneous population of cells that is distinctly different than the results generated from cells cultured in control media, however it should be noted that there is a limited degree of overlap as projected as ‘0’ along the abscissa.

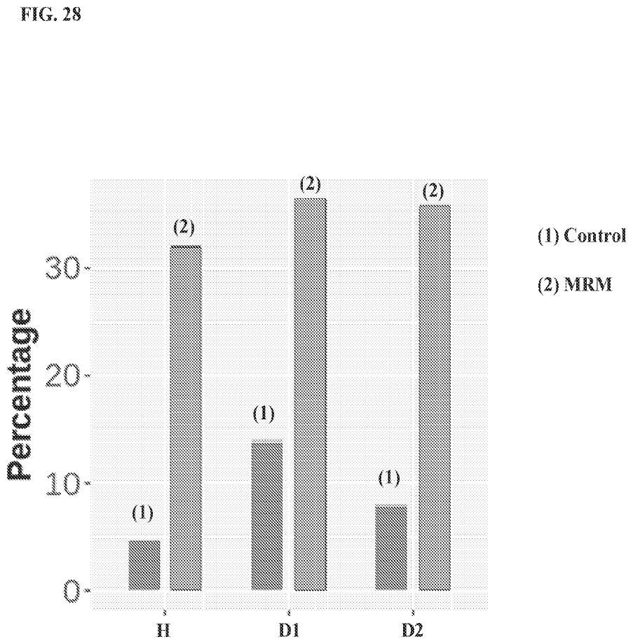

Cells were then subjected to cluster analysis using the FindClusters function in Seurat (Satija et al. Nat. Biotech 2015). Each cell was assigned a score based on the naive-associated geneset from Gattinoni et al. 2009, using the AddModuleScore function in Seurat. To assess the capability to generate cells with less-differentiated phenotypes, percentage of cells in clusters with high naive-associated scores in each donor and process (control media or MRM) were calculated (FIG. 28). Across all three donors, cells generated using MRM show substantially higher proportion of less-differentiated cells compared to cells generated using control media.

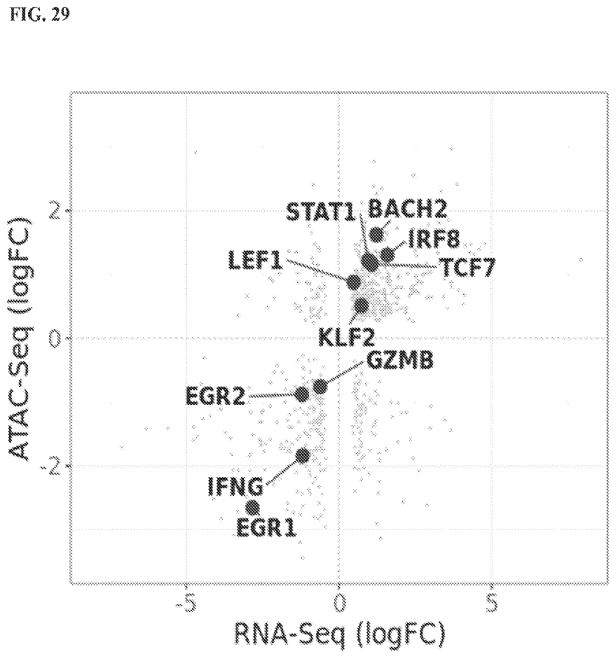

To further understand transcriptional and epigenetic differences between cells generated by culturing in control media versus MRM, differential peak analysis from bulk ATAC-seq and differential gene analysis from bulk RNA-seq were performed (as described in Lynn et al. Nature 2019). Several transcription factors associated with naive-like phenotype such as TCF7, LEF1, KLF2, BACH2, not only have significant differential peaks in their respective promoter regions, but also have significantly higher differential expression in cells produced by culturing in MRM (FIG. 29).

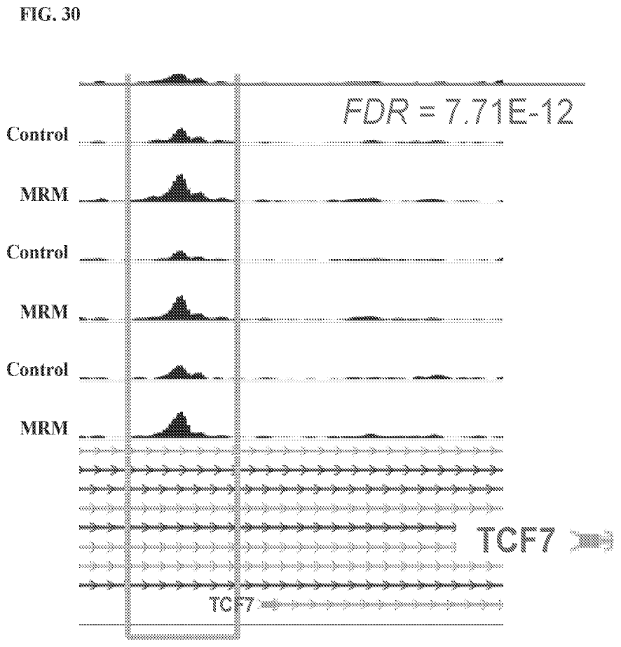

The significant differential peak in the promoter region of TCF7 is shown in FIG. 30. Transcription factors associated with more of an effector/activated phenotype such as STAT1 and IRF8 are also significant both in terms of chromatin accessibility and gene expression in cells produced using MRM (FIG. 29). EGR2, a transcription factor associated with T cell anergy (Mi Oh et al. Nat Comm. 2015) is significantly down-regulated in cells produced using MRM as well, suggesting that these cells are more functional. Hence, at the bulk level, cells produced using MRM are enriched for transcription factors representing both naive-like phenotype and effector/activated phenotype compared to cells cultured in control media.

Single cell trajectory construction and pseudotime calculations were used to further characterize T cells cultured in MRM. Unlike T cells cultured in control media, T cells cultured in MRM are able to both re-populate the stem-like cell population and also to differentiate to produce effector cells, even after prolonged and repetitive challenges with tumor cells. Single cell trajectory construction and pseudotime calculations were performed on T cells using Monocle 3 (Trapnell et al., Nature Biotechnology 2014; Qiu et al., Nature Methods 2017; Cao et al., Nature 2019) implemented in Seurat (Ha, et al., bioRXiv 2020). Pseudotime cell trajectories revealed an abundance of less-differentiated CD8⁺ T cells even after repetitive stimulation with tumor cells using T cells expanded in MRM, whereas the growth of T cells expanded in control media were predominantly more differentiated. The characteristics of the most differentiated cells were also different, with MRM cultured T cells generating cells that are higher for GZMB, MHC-II, LAG3, TIGIT, and NKG7 and lower for IL-32. Cells highest for NKG7 have been shown to be better killers (Malarkannan et al., Nat. Immuno. 2020) whereas cells higher in IL-32 have been shown to have activation-induced cell death (Goda et al., Int. Immunol 2006) illustrating that T cells expanded in MRM generate effector cells resembling highly cytotoxic cells able to engage in effector function.

### Example 9: Analysis of CAR Transduction Efficiency

To assess the effect that metabolic reprogramming media has on CAR transduction efficiency, human CD4⁺ and CD8⁺ T cells were transduced with anti-RORi CAR constructs in either metabolic reprogramming media (MRM) or a T-cell conditioned medium (i.e., TCM). Provided below are exemplary methods used in carrying out the present Example.

### Media Preparation

Media was prepared as described above (see Example 1).

### Lentiviral Vector (LVV) Construction and Lentiviral Production

An anti-RORi CAR construct comprising the following components was generated: (i) anti-RORi CAR (derived from the R12 antibody) (referred to herein as “R12 CAR”), (ii) truncated EGFR (“EGFRt”), and (iii) wild-type c-Jun protein (referred to herein as the “c-Jun-R12 CAR”). The c-Jun-R12 CAR construct was designed, such that when transduced in a cell (e.g., T cell), the transduced cell would exhibit increased c-Jun protein expression along with surface expression of the anti-RORi CAR and EGFRt. As a control, a corresponding anti-RORi CAR construct comprising truncated CD19 (“CD19t”) instead of c-Jun was also generated (referred to herein as the “control CD19t-R12 CAR”). See Terakura, S. et al., Blood 119(1): 72-82 (2012), which is incorporated herein by reference in its entirety.

Lentiviral vectors were pseudotyped with the VSV-G envelope and produced by transient transfection of HEK293T cells. The final bulk was held at 2-8° C. for no longer than 24 hours prior to filling 1 mL aliquots of LVV and stored at −80° C. The LVV aliquots were thawed on ice prior to T cell transduction.

T Cell Isolation

CD4+ and CD8+ T cells were isolated from three healthy donors and frozen using vendors, BloodWorks (Seattle, WA, USA) and AllCells (Alameda, CA, USA). The vendors obtained and maintained all appropriate consent forms from the donors. To isolate the CD4+ and CD8+ T cells, samples were collected samples via apheresis, from which CD4+ and CD8+ cells were isolated separately in order of CD8+ T cells positively selected first followed by positive selection for CD4+ T cells of the flow-through from the CD8 selection. Isolated CD4+ or CD8+ T cells were frozen either at 20E+06 cells (AllCells) or 50E+06 cells (BloodWorks) per vial.

Cell Culture and Transduction

Healthy donor cryopreserved human CD4+ and CD8+ T cells (i.e., from the vendors) were thawed in the appropriate media (i.e., TCM or MRM) and combined at a 1:1 ratio. The combined donor CD4+ and CD8+ T cells were centrifuged at 300×g for 5 minutes and resuspended in appropriate media (i.e., T cell conditioned media or MRM) supplemented with IL-2, IL-7, and IL-15. The T cells were then activated using CD3/CD28 TRANSACT™ (Miltenyi Biotec Inc.). After 24 hours of activation (i.e., day 1) in either TCM or MRM, the T cells were transduced with the above-described LVVs comprising the anti-ROR1 CAR constructs (i.e., “c-Jun-R12 CAR” and “control CD19t-R12 CAR”). Non-transduced T cells were used as control. The following day after transduction (i.e., day 2), fresh media (i.e., TCM or MRM) were added to dilute the TRANSACT™ and end T cell activation. The transduced T cells were allowed to further expand for five additional days (i.e., day 7), and then either subsequently analyzed or cryopreserved in liquid nitrogen for long-term storage.

Transduction Efficiency Analysis

To compare the CAR transduction efficiency from the different groups (see Table 3), the percentage of CD4+ and CD8+ T cells expressing the following was determined using flow cytometry: (i) c-Jun, anti-ROR1 R12 scFv, and EGFRt or (ii) c-Jun, anti-ROR1 R12 scFv, and truncated CD19.

TABLE 3
Experimental Groups

| Group No. | Description |
|---|---|
| 1 | Non-transduced T cells cultured in TCM |
| 2 | T cells transduced with control CD19t-R12 CAR and |
| | cultured in TCM |
| 3 | T cells transduced with c-Jun-R12 CAR and cultured in |
| | TCM |
| 4 | Non-transduced T cells cultured in MRM |
| 5 | T cells transduced with control CD19t-R12 CAR cultured |
| | in MRM |
| 6 | T cells transduced with c-Jun-R12 CAR cultured in MRM |

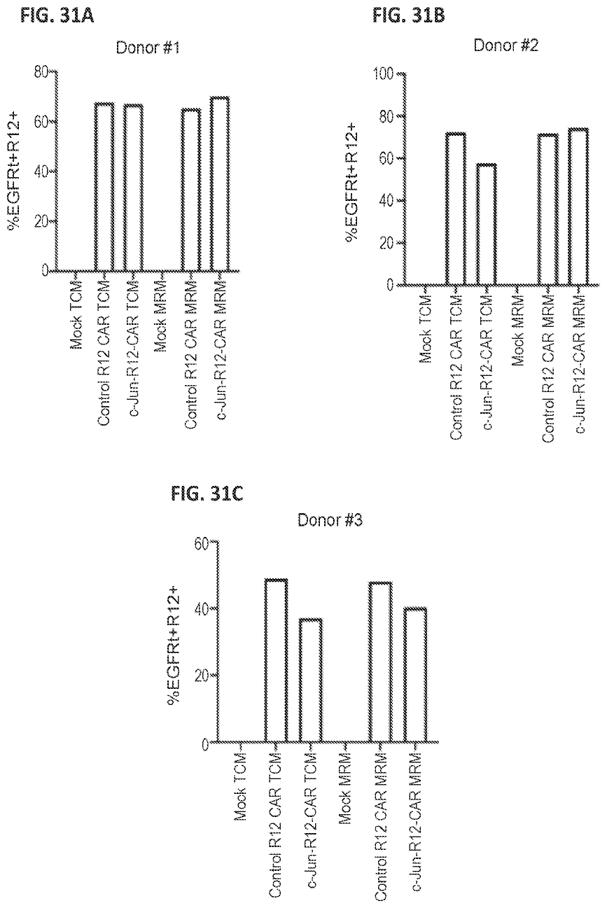

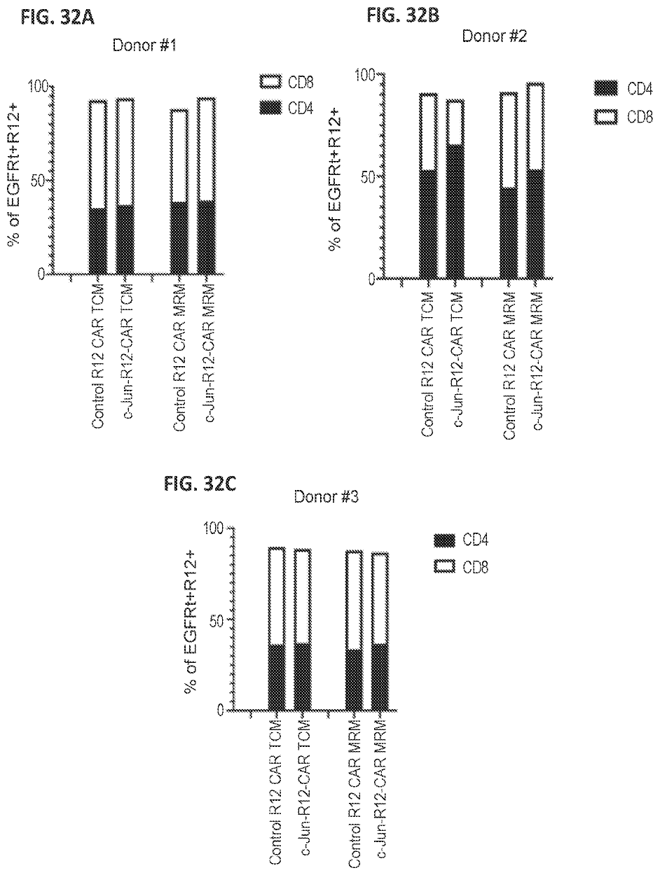

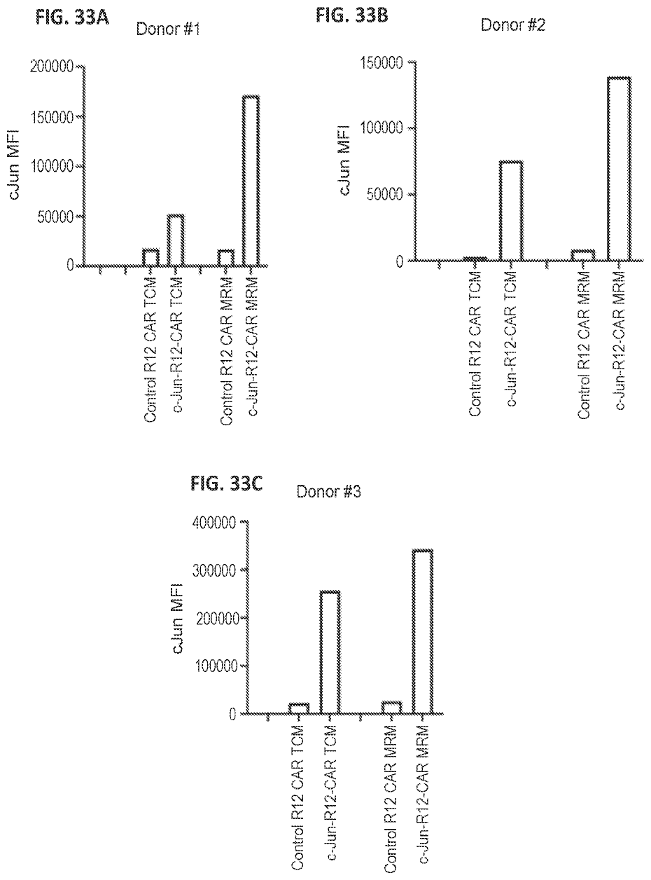

As shown in FIGS. 31A-31C, in both the TCM and MRM groups and within each donor, the transduction efficiency between the control CD19t-R12 CAR and c-Jun-R12 CAR was comparable. Similarly, as between the TCM and MRM groups, the percentage of transduced T cells (i.e., expressing both EGFRt and R12 CAR) was comparable. There was also no significant difference observed in the percentage of CD4+ and CD8+ T cells that were transduced from the different groups within each donor (see FIGS. 32A-32C). Interestingly, T cells transduced with c-Jun-R12 CAR from the MRM group expressed significantly higher levels of c-Jun protein expression compared to the corresponding transduced T cells from the TCM group (see FIGS. 33A-33C). The increased expression was specific to c-Jun protein and was not global to the other transgenes (i.e., R12 CAR and EGFRt).

These results suggest that the anti-ROR1 CAR constructs described herein are capable of being transduced into T cells with similar degree under both culture conditions. The results further suggest that the metabolic reprogramming media condition could be useful in selectively increasing the expression of the c-Jun protein of the anti-ROR1 CAR constructs provided herein.

### Example 10: Analysis of Stem-Like Phenotypic Expression

To assess whether the metabolic reprogramming media has any effect on the stem-like properties of the transduced T cells, human CD4+ and CD8+ T cells were transduced with anti-ROR1 CAR constructs as described in Example 1 (i.e., c-Jun-R12 CAR or control CD19t-R12 CAR). Then, after the cells were allowed to expand for four to five additional days (i.e., day 6 or 7), the stemness of the transduced T cells was assessed using flow cytometry.

Briefly, the T cells were first washed with cell staining buffer and stained with anti-CCR7 for 15 minutes at 37° C. Next, the T cells were washed again and then a master mix of the antibodies against several other antigens (as detailed below) was added to the cells and incubated for 25 minutes in the dark at room temperature. Cells were then washed with cell staining buffer and permeabilized with the FOXP3 staining kit (ebioscience) as per manufacturers' protocol. After fixing, the cells were blocked with pre-diluted normal mouse serum (Jackson ImmunoResearch-#015-000-120) and normal rabbit serum (Jackson ImmunoResearch-#011-000-120) for 15 minutes in the dark at room temperature. The cells were then stained with a 2× antibody cocktail of TCF7 and c-Jun for 30 minutes in the dark at room temperature. After thoroughly washing the cells, they were analyzed by flow cytometry on Cytek Aurora Spectral Flow Cytometer and analyzed using FlowJo software (TreeStar, Ashland, OR).

The following are the list of antibodies used for assessing the sternness markers: CD8 (Thermo-#58-0088-42), CD4 (BD-#612936), CD27 (BD-#612829), CD3 (Thermo-#612896), CD28 (Biolegend-#302936), CD62L (BD-#740301), R12 Anti-Id (Genscript-#48F6H5E1), EGFR (BioLegend-#98812), CD45RO (BioLegend-#566143), CD39 (BioLegend-#328236), TCF7 (Cell Signaling-#9066S), c-Jun (Cell Signaling-#15683S), CCR7 (BD-#562381), CD45RA (BD-#560673), LAG-3 (Thermo-#67-2239-42), TIM-3 (Thermo-#78-3109-42), TIGIT (Thermo-#46-9500-42), PD-1 (Thermo-#25-2799-42). Specifically, as described herein, “stem-like” cells were defined as: CD45RO⁻CCR7⁺ CD45RA⁺CD62L⁺CD27⁺ CD28⁺TCF7+.

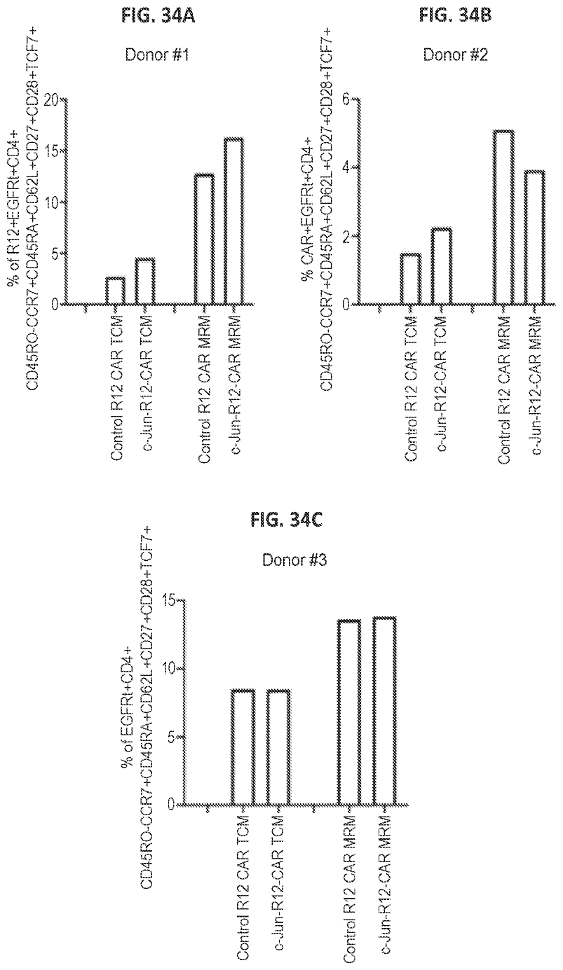

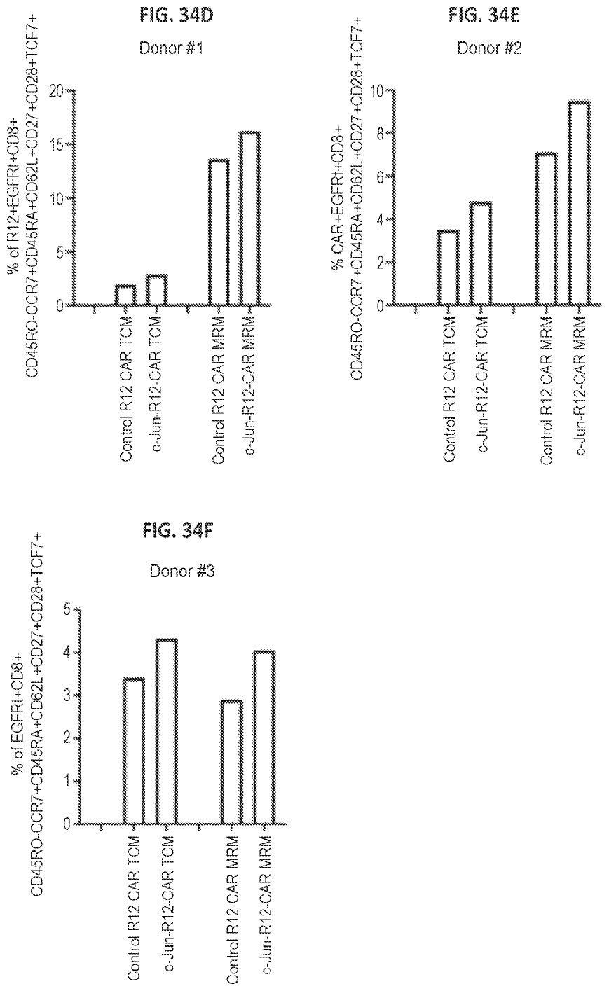

As shown in FIGS. 34A-34C, compared to cells from the TCM groups, CD4+ T cells transduced with an anti-ROR1 CAR construct in MRM were more stem-like as to their phenotypic expression. This was generally true regardless of whether the CD4+ T cells were transduced with the c-Jun-R12 CAR or the control CD19t-R12 CAR (see last two bars in FIGS. 34A-34C). Similarly, CD8+ T cells transduced in MRM were generally more stem-like compared to corresponding cells transduced in TCM (at least for CD8+ T cells derived from donors #1 and #2; see FIGS. 34D and 34E). But, unlike the CD4+ T cells, consistent increase in stem-like cells were observed when CD8+ T cells were transduced with c-Jun-R12 CAR as compared to the control CD19t--R12 CAR. Accordingly, among the CD8+ T cells, the greatest percentage of stem-like cells was observed when CD8+ T cells were transduced with c-Jun-R12 CAR in MRM.

These results highlight the usefulness of the metabolic reprogramming media described herein in producing transduced CD4+ and CD8+ T cells that are less differentiated (i.e., more stem-like). And, at least for CD8+ T cells, the results further suggest that overexpressing transcription factors, such as c-Jun, can further improve the stem-like properties of the transduced T cells.

### Example 11: Phenotypic and Functional Analysis

To further assess the effect that MRM has on anti-ROR1 CAR T cells, human CD4+ and CD8+ T cells were transduced with anti-ROR1 CAR constructs and expanded as described in Example 1. At day 6 or 7, the transduced CD4+ and CD8+ T cells were analyzed both phenotypically (e.g., expression of different surface markers, such as CD39, LAG3, PD1, TIGIT, and TIM3) and functionally (e.g., IL-2 and/or IFN-7 production and in vitro killing after primary and/or chronic antigen stimulation).

Phenotypic Expression

Expression of the surface markers on the transduced CD4+ and CD8+ T cells were assessed using Cytek Aurora Spectral Flow Cytometer and analyzed using FlowJo software (TreeStar, Ashland, OR).

Cytotoxicity and Cytokine Secretion

The cytolytic activity of the transduced T cells was measured using an in vitro killing assay. Briefly, the transduced T cells (“effector”) were co-cultured with target tumor cells (“target) at an effector:target ratios of 1:4, 1:16, 1:64, and 1:128, and scanned at 4× magnification every 6 hours using the IncuCyte (cytolytic activity was measured by tracking the number of red nuclei representing the target tumor cells). After 24 hours of co-culture, supernatant was collected from the different conditions and frozen at −80° C. for later cytokine analysis. The culture plates containing the cells were then returned to the IncuCyte for continued periodic scanning.

For cytokine secretion analysis, the previously frozen supernatant was thawed and the levels of certain cytokines (e.g., IL-2 and IFN-g) was assessed using the MesoScaleDiscovery (MSD) multiplex platform and measured on the MSD Meso Sector S 600 machine according to the manufacturer's protocol.

Serial Restimulation Assay

In this assay, the transduced CD4+ and CD8+ T cells were serially restimulated every three or four days with A549 NLR target cells. The T cells were plated at an E:T ratio of 1:1 for a total of 2 to 4 rounds of stimulation. A density of 3×10⁵ transduced T cells/mL was maintained throughout the study. To set up each round of stimulation, the T cells were stained with the following markers and analyzed using flow cytometry to calculate the proportion of transduced T cell population present in the co-culture: CD45, CD3, CD4, CD8, CAR, and EGFRt. An aliquot of each sample was reserved for a titrated Incucyte killing assay, as described above.

Results

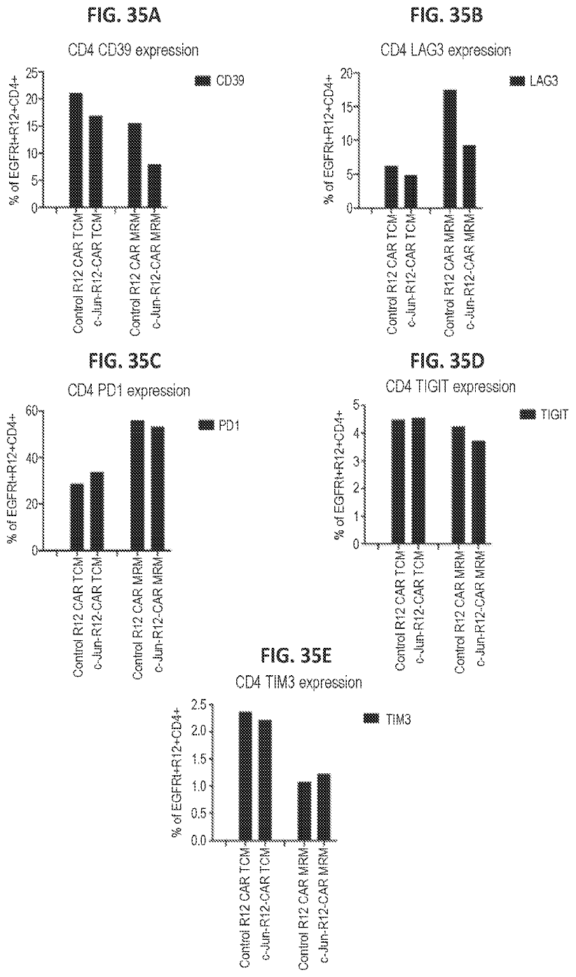

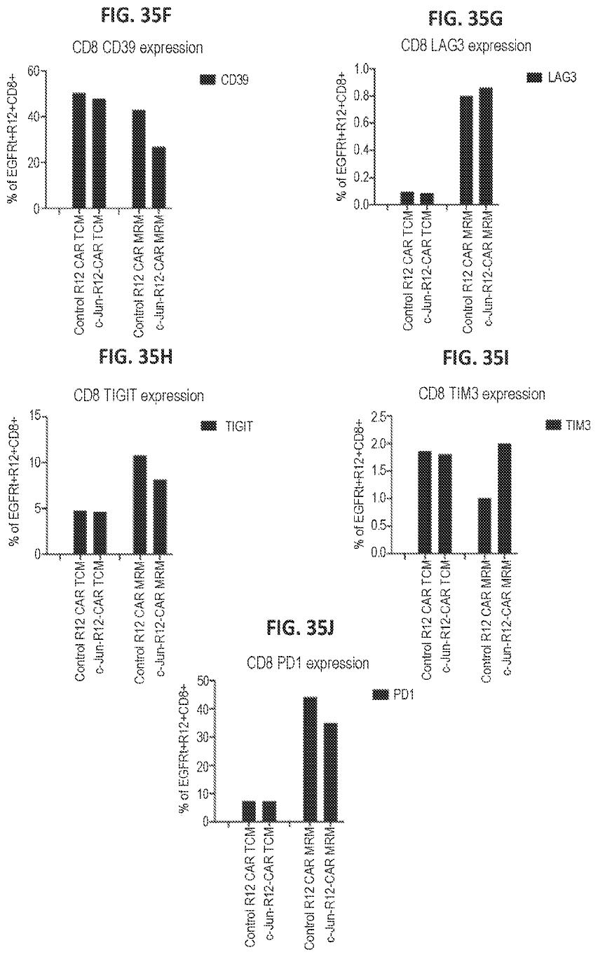

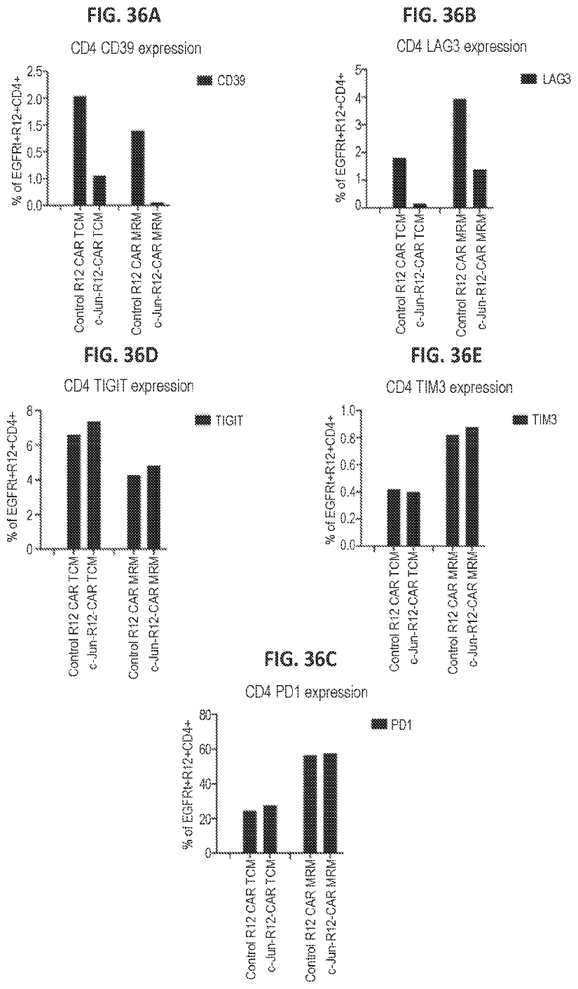

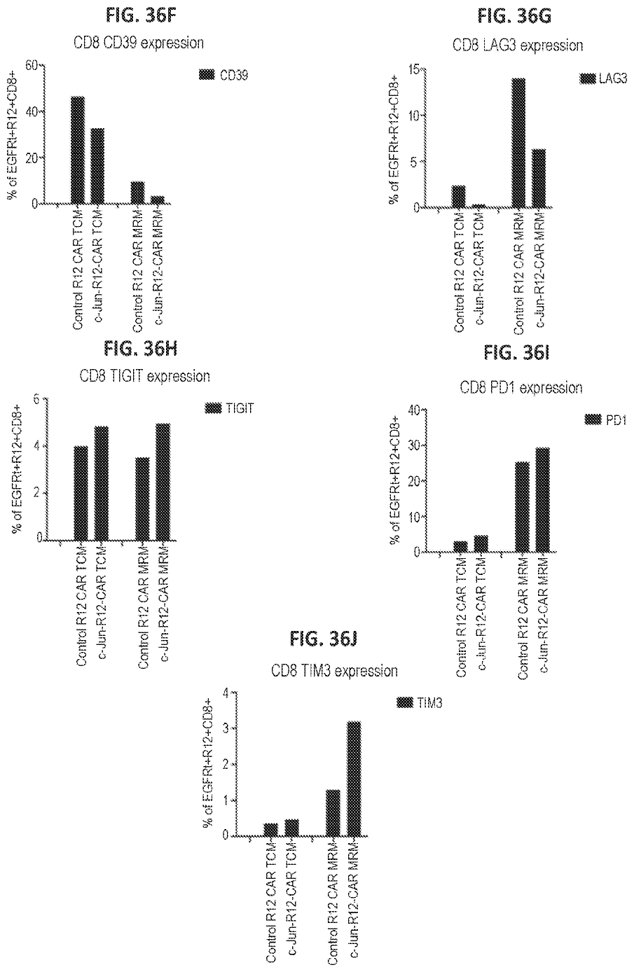

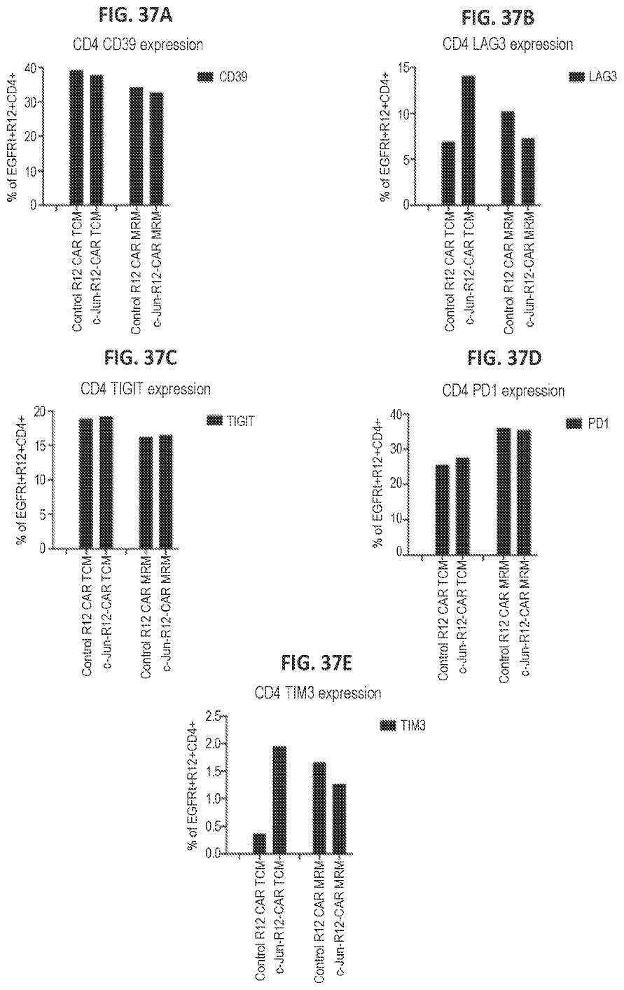

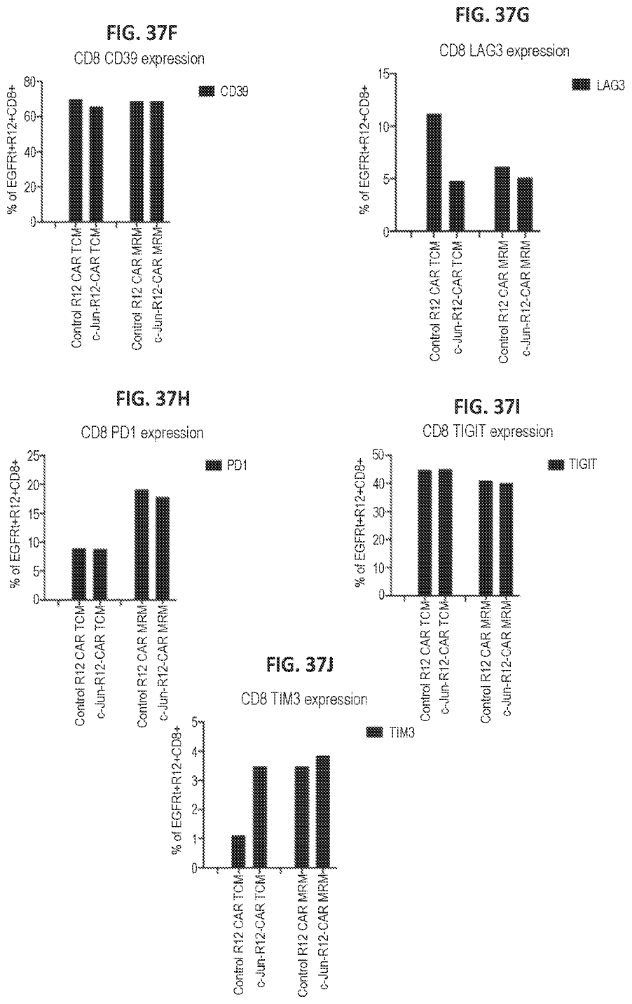

As shown in FIGS. 35A-35E, 36A-36E, and 37A-37E, noticeable differences were observed in various surface marker expression levels between CD4+ T cells transduced with an anti-ROR1 CAR construct in MRM compared to corresponding cells transduced in TCM. Similar results were observed for CD8+ T cells (see FIGS. 35F-35J, 36F-36J, and 37F-37J), further confirming that the potassium concentration present during transduction can have effects on the transduced T cells.

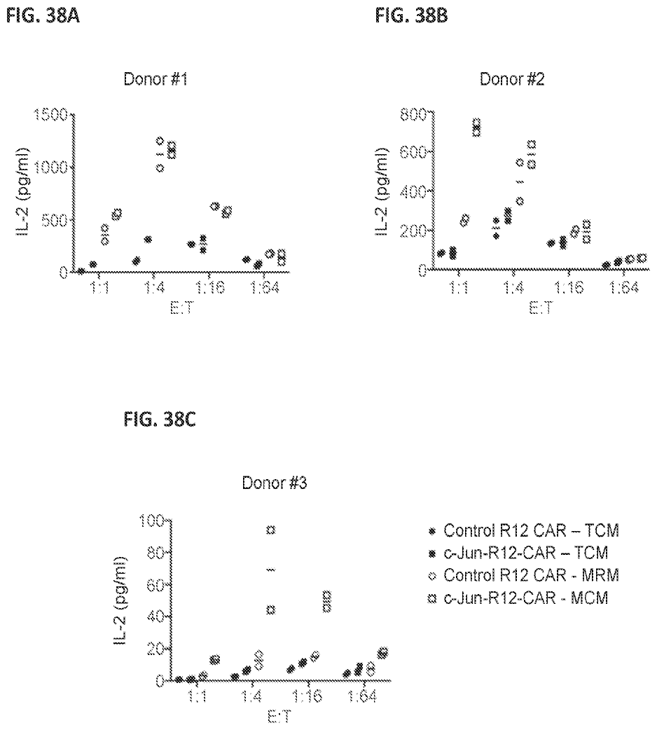

Moreover, in addition to the phenotypic differences, a clear functional difference was also observed in the transduced T cells. As shown in FIGS. 38A-38C, after primary antigen stimulation, T cells transduced and cultured in MRM produced higher amounts of IL-2 compared to the corresponding cells transduced and cultured in TCM. And, as observed earlier in Example 1, the increased c-Jun protein expression in the transduced T cells also resulted in greater IL-2 secretion. For instance, in both the TCM and MRM groups, T cells that were transduced with the c-Jun-R12 CAR produced higher levels of IL-2 after primary stimulation, compared to corresponding cells that were transduced with the control CD19t-R12 CAR. Accordingly, greatest IL-2 production was generally observed in T cells modified to overexpress c-Jun and cultured in MRM.

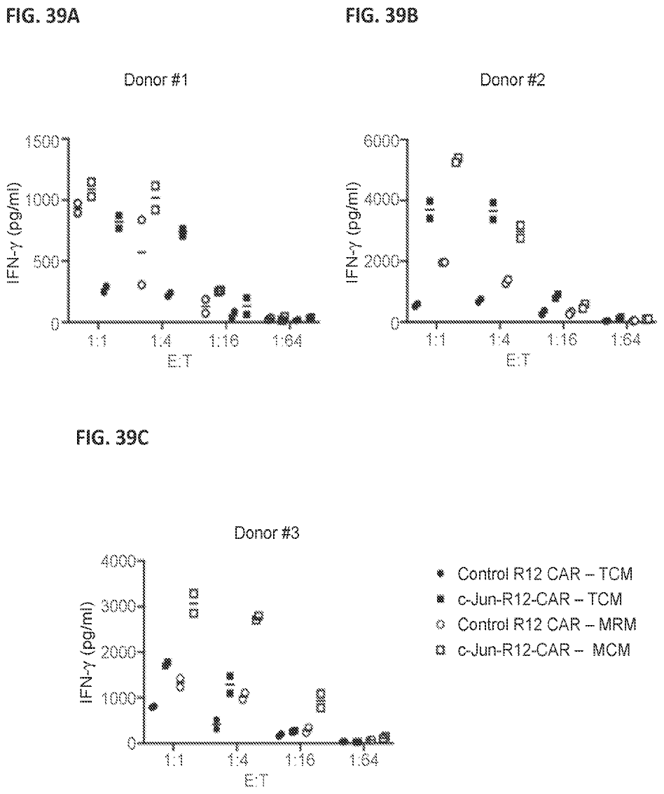

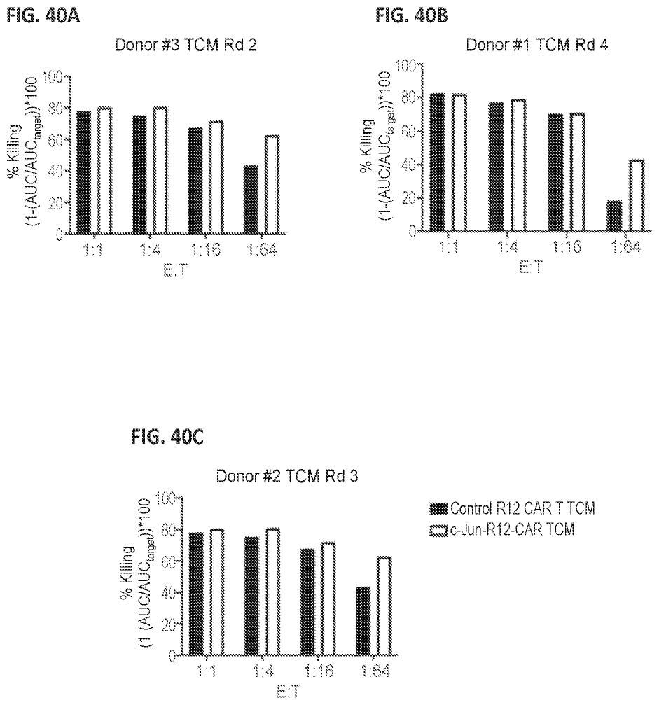

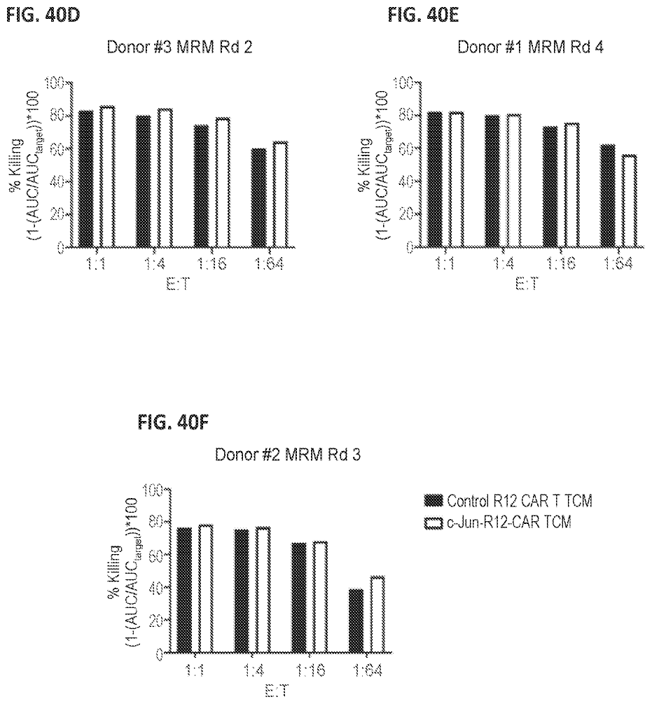

Similar results were observed after serial/chronic antigen stimulation. As shown in FIGS. 39A-39C, following the terminal round of antigen stimulation, T cells transduced and cultured in MRM retained their ability to produce IFN-7 compared to the corresponding cells that were transduced and cultured in TCM. Again, T cells transduced with c-Jun-R12 CAR from the MRM group maintained the ability to produce IFN-7 much longer compared to transduced cells from the other groups. As to the cytotoxicity of the transduced cells after multiple antigen stimulation, T cells transduced and cultured in MRM maintained their ability to kill tumor cells much longer, compared to the corresponding cells from the TCM group (FIGS. 40A-40E).

Collectively, the above results confirm that CAR T cells modified to overexpress c-Jun (e.g., with ROR1 CAR and c-Jun overexpression) cultured in MRM allow for the generation of stem-like transduced T cells that remain functional even after chronic antigen stimulation.

### Example 12. TIL Clearance of Tumor Cells ex vivo

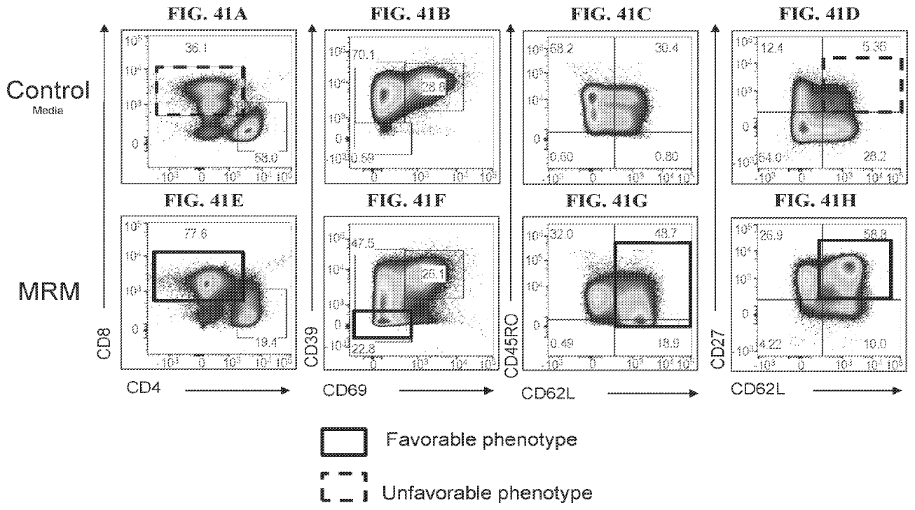

Non-small cell lung cancer (NSCLC) TILs produced following standard TIL expansion methods were compared to TIL produced in MRM, according to the methods disclosed herein. While the control process generated TTLs highly enriched for CD8+ T cells, these cells were not enriched for a stem-like CD8+ CD39−CD69− T cell population and expressed low levels of central memory markers and co-stimulatory receptor, CD27 (FIGS. 41A-41D). The control expansion process generated T cell products with enriched stem-like CD8+ CD39−CD69− T cells and higher expression of co-stimulatory receptor CD27, but resulted in reduced expansion of CD8+ T cells. TILs cultured in MRM yield highly enriched populations of CD8+ T cells with enhanced abundance of CD8+ CD39−CD69− stem-like T cells, central memory markers CD45RO+CD62L+), and markedly higher expression of co-stimulatory receptor CD27 (FIGS. 41E-41H). Collectively these markers demonstrate attributes of stem-like cells that are correlated with clinical responses.

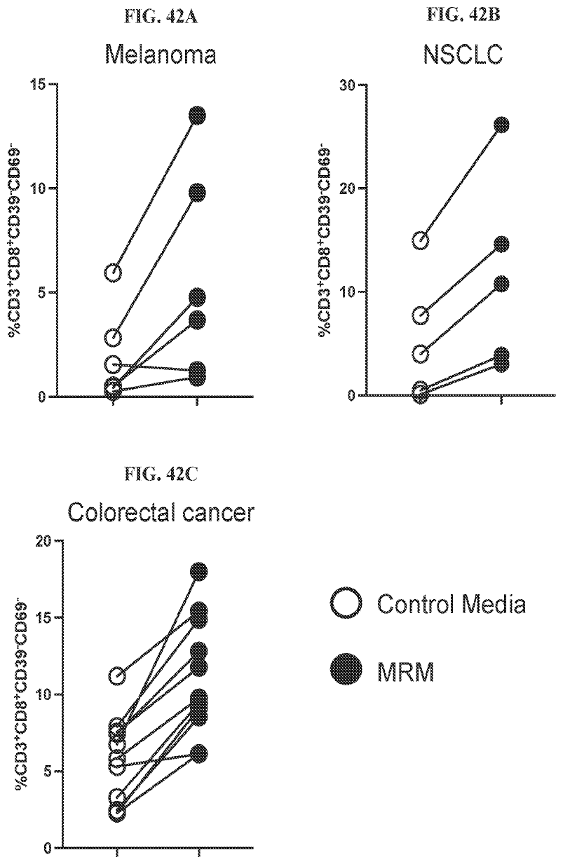

TIL products with enhanced stem-like properties, including retention of key co-stimulatory receptors CD27, elongated telomeres, memory cell phenotypes and the presence of populations of cells enriched with CD39⁻/CD69⁻ CD8⁺ T cells, are associated with improved clinical responses. Additional differentiation state-linked characteristics of T cells, including ability to secrete polyfunctional cytokines, and expression of co-stimulatory receptor CD28 have also been correlated with anti-tumor potency. TILs expanded in MRM exhibit favorable phenotypic attributes, including an increased abundance of CD8⁺ CD39⁻CD69⁻ T cells when compared with control TIL expansion process in melanoma, NSCLC, and colorectal cancers (FIGS. 42A-42C).

### Example 13. TIL culture in MRM

The data presented herein show that culturing TILs in a culture medium comprising increased potassium generates a TIL population that has increased expansion of CD8⁺ TILs and increased stemness, relative to TILs cultured in medium having lower levels of potassium (e.g., less than about 40 mM potassium ion, e.g., 5 mM potassium ion). Further, data suggest that exceedingly high levels of potassium (e.g., greater than about 80 mM, greater than about 90 mM, or greater than about 100 mM potassium ion) in the culture medium can lead to decreased TIL yield, likely due to decreased TIL growth and expansion, data not shown. To further characterize the effects of MRM on TIL culture, (1) TILs will be cultured in control medium (an isotonic RPMI formulation comprising 55 mM potassium ion) during an initial expansion stage followed by culture in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during a final expansion stage; (2) TILs will be cultured in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during an initial expansion stage followed by culture in control medium during a final expansion stage; and (3) TILs will be cultured in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during an initial expansion stage followed by culture in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during a final expansion stage. In all experiments, initial culture conditions will further comprise 6000 IU/mL IL-2, and final culture conditions will further comprise 3000 IU/mL IL-2. TILs cultured according to this method will be characterized for expression of stemness and effector markers, cytotoxicity, and yield.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

What is claimed is:

1. A method of culturing immune cells and/or stem cells ex vivo or in vitro comprising placing immune cells and/or stem cells in a medium comprising potassium ion at a concentration higher than 40 mM, wherein the medium is not hypertonic.

2. A method of preparing a population of immune cells and/or stem cells comprising placing immune cells and/or stem cells into a medium comprising potassium ion at a concentration higher than 40 mM, wherein the medium is not hypertonic.

3. The method of claim 1 or 2, wherein the medium further comprises one or more cytokines.

4. The method of claim 3, wherein the one or more cytokines comprise Interleukin-2 (IL-2), Interleukin 21, Interleukin-15 (IL-15), or any combination thereof.

5. A method of increasing a number or percentage of undifferentiated or less differentiated cells ex vivo or in vitro comprising culturing immune cells and/or stem cells in a medium comprising potassium ion at a concentration of higher than 40 mM, wherein the medium comprises IL-2, but does not comprise IL-7 and IL-15.

6. The method of claim 6, wherein the immune cells and/or stem cells after the culturing comprises a higher number and/or percentage of undifferentiated or less differentiated immune cells and/or stem cells compared to immune cells and/or stem cells that are cultured in a medium comprising IL-2, IL-7, and IL-15.

7. A method of increasing a number and/or percentage of undifferentiated or less differentiated immune cells and/or stem cells ex vivo or in vitro comprising placing immune cells and/or stem cells in a medium comprising potassium ion at a concentration higher than 40 mM, wherein the medium comprises IL-7 and IL-21.

8. A method of increasing a number or percentage of undifferentiated or less differentiated immune cells and/or stem cells ex vivo or in vitro comprising placing the immune cells and/or stem cells in a medium comprising potassium ion at a concentration higher than 40 mM, wherein the medium comprises IL-15 and IL-21.

9. The method of any one of claims 1 to 8, wherein the cells comprise immune cells.

10. The method of claim 8, wherein the immune cells comprise T cells, TILs, NK cells, TILs, Tregs, and or combination thereof.

11. The method of any one of claims 1 to 8, wherein the cells comprise stem cells.

12. The method of any one of claims 1 to 11, wherein the cells express chimeric antigen receptor (CAR).

13. The method of any one of claims 1 to 11, wherein the cells express T cell receptor (TCR).

14. The method of any one of claims 1 to 13, wherein the medium is hypotonic.

15. The method of any one of claims 1 to 14, wherein the medium further comprises sodium ion, calcium ion, glucose, and/or any combination thereof.

16. The method of any one of claims 1 to 15, wherein the medium further comprises a cell expansion agent.

17. The method of claim 16, wherein the cell expansion agent comprises a GSK3B inhibitor, an ACLY inhibitor, a PI3K inhibitor, an AKT inhibitor, or any combination thereof.

18. The method of claim 17, wherein the PI3K inhibitor is selected from LY294002, pictilisib, CAL101, IC87114, or any combination thereof.

19. The method of claim 17, wherein the AKT inhibitor is selected from MK2206, A443654, AKTi-VIII, or any combination thereof.

20. The method of any one of claims 1 to 19, wherein the medium is capable of:
a. increasing the number and/or percentage of less differentiated and/or undifferentiated cells;
b. increasing transduction efficiency;
c. increasing stem-like immune cells;
d. increasing in vivo viability;
e. increasing cell potency;
f. preventing cell exhaustion; or
g. any combination thereof,
in the final cell product as compared to the starting cell population

21. The method of any one of claims 1 to 20, wherein the concentration of potassium ion is at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM.

22. The method of any one of claims 1 to 21, wherein the concentration of potassium ion is about 45 mM to about 110 mM, about 45 mM to about 100 mM, about 45 mM to about 90 mM, about 45 mM to about 80 mM, about 45 mM to about 70 mM, about 45 mM to about 60 mM, about 45 mM to about 50 mM, about 50 mM to about 110 mM, about 50 mM to about 100 mM, about 50 mM to about 90 mM, about 50 mM to about 80 mM, about 50 mM to about 70 mM, about 60 mM to about 110 mM, about 60 mM to about 100 mM, about 60 mM to about 90 mM, about 60 mM to about 80 mM, about 60 mM to about 70 mM, about 70 mM to about 100 mM, about 70 mM to about 90 mM, about 70 mM to about 80 mM, about 80 mM to about 110 mM, about 80 mM to about 100 mM, about 80 mM to about 90 mM, about 90 mM to about 110 mM, about 90 mM to about 100 mM, or about 100 mM to about 110 mM.

23. The method of any one of claims 1 to 22, wherein the concentration of potassium ion is about 50 mM to about 90 mM.

24. The method of any one of claims 1 to 23, wherein the concentration of potassium ion is about 50 mM to about 80 mM.

25. The method of any one of claims 1 to 24, wherein the medium has an osmolality lower than about 280 mOsm/L.

26. The method of any one of claims 1 to 25, wherein the medium has an osmolality between about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L.

27. The method of any one of claims 1 to 26, wherein the medium has an osmolality of about 100 mOsm/L to about 280 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L.

28. The method of any one of claims 1 to 27, wherein the medium has an osmolality of about 250.

29. The method of any one of claims 1 to 28, wherein the medium has an osmolality of about 255.

30. The method of any one of claims 1 to 29, wherein the medium has an osmolality of about 260.

31. The method of any one of claims 1 to 24, wherein the medium is isotonic.

32. The method of claim 31, wherein the medium has an osmolality of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L.

33. The method of claim 31 or 32, wherein the medium has an osmolality of about 285 mOsm/L, about 290 mOsm/L, about 295 mOsm/L, about 300 mOsm/L, about 305 mOsm/L, about 310 mOsm/L, or about 315 mOsm/L.

34. The method of any one of claims 1 to 33, wherein the medium further comprises sodium ion.

35. The method of claim 34, wherein the concentration of the sodium ion is from about 25 mM to about 100 mM.

36. The method of claim 34 or 35, wherein the concentration of the sodium ion is from about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM.

37. The method of any one of claims 34 to 36, wherein the concentration of the sodium ion is about 30 mM, about 35 mM, about 40 mM, about 45 mM, about 50 mM, about 55 mM, about 60 mM, about 65 mM, about 70 mM, about 75 mM, or about 80 mM.

38. The method of any one of claims 34 to 37, wherein the concentration of the sodium ion is about 55 mM.

39. The method of any one of claims 34 to 38, wherein the concentration of the sodium ion is about 60 mM.

40. The method of any one of claims 34 to 39, wherein the concentration of the sodium ion is about 65 mM.

41. The method of any one of claims 1 to 40, wherein the medium further comprises glucose.

42. The method of claim 41, wherein the concentration of glucose is more than about 10 mM.

43. The method of claim 41 or 42, wherein the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM.

44. The method of any one of claims 41 to 43, wherein the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM.

45. The method of any one of claims 41 to 44, wherein the concentration of glucose is about 15.4 mM, about 15.9 mM, about 16.3 mM, about 16.8 mM, about 17.2 mM, or about 17.7 mM.

46. The method of any one of claims 1 to 45, wherein the medium further comprises calcium ion.

47. The method of claim 46, wherein the concentration of calcium ion is more than about 0.4 mM.

48. The method of claim 46 or 47, wherein the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

49. The method of any one of claims 46 to 48, wherein the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

50. The method of any one of claims 1 to 49, wherein the medium comprises about 50 mM potassium ion and
(i) about 80.5 mM sodium ion;
(ii) about 17.7 mM glucose;
(iii) about 1.8 mM calcium ion; or
(iv) any combination of (i)-(iii).

51. The method of claim 50, wherein the medium has an osmolality of about 254.7 mOsmol.

52. The method of any one of claims 1 to 49, wherein the medium comprises about 55 mM potassium ion and
(i) about 76 mM sodium ion;
(ii) about 17.2 mM glucose;
(iii) about 1.7 mM calcium ion; or
(iv) any combination of (i)-(iii).

53. The method of claim 52, wherein the medium has an osmolality of about 255.2 mOsmol.

54. The method of any one of claims 1 to 49, wherein the medium comprises about 60 mM potassium ion and
(i) about 72.2 mM sodium ion;
(ii) about 16.8 mM glucose;
(iii) about 1.6 mM calcium ion; or
(iv) any combination of (i)-(iii).

55. The method of claim 54, wherein the medium has an osmolality of about 257.2 mOsmol.

56. The method of any one of claims 1 to 49, wherein the medium comprises about 65 mM potassium ion and
(i) about 67.6 mM sodium ion;
(ii) about 16.3 mM glucose;
(iii) about 1.5 mM calcium ion; or
(iv) any combination of (i)-(iii).

57. The method of claim 56, wherein the medium has an osmolality of about 257.5 mOsmol.

58. The method of any one of claims 1 to 49, wherein the medium comprises about 70 mM potassium ion and
(i) about 63.9 mM sodium ion;
(ii) about 15.9 mM glucose;
(iii) about 1.4 mM calcium ion; or
(iv) any combination of (i)-(iii).

59. The method of claim 58, wherein the medium has an osmolality of about 259.7 mOsmol.

60. The method of any one of claims 1 to 49, wherein the medium comprises about 75 mM potassium ion and
(i) about 59.3 mM sodium ion;
(ii) about 15.4 mM glucose;
(iii) about 1.3 mM calcium ion; or
(iv) any combination of (i)-(iii).

61. The method of claim 60, wherein the medium has an osmolality of about 260 mOsmol.

62. The method of any one of claims 1 to 49, wherein the medium comprises about 80 mM potassium ion and
(i) about 55.6 mM sodium ion;
(ii) about 15 mM glucose;
(iii) about 1.2 mM calcium ion; or
(iv) any combination of (i)-(iii).

63. The method of claim 62, wherein the medium has an osmolality of about 262.26 mOsmol.

64. The method of any one of claims 9 to 55, wherein the immune cells are CD3+, CD45RO⁻, CCR7+, CD45RA+, CD62L⁺CD27+, CD28+, or TCF7+, or any combination thereof, following culture.

65. The method of any one of claims 4 to 6 and 9 to 64, wherein the concentration of IL-2 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

66. The method of claim 65, wherein the concentration of IL-2 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

67. The method of claim 65 or 66, wherein the concentration of IL-2 is about 1.0 ng/mL.

68. The method of claim 65 or 66, wherein the concentration of IL-2 is about 10 ng/mL.

69. The method of any one of claims 4, 7, and 8 to 68, wherein the concentration of IL-21 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

70. The method of claim 69, wherein the concentration of IL-21 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

71. The method of claim 69 or 70, wherein the concentration of IL-21 is about 1.0 ng/mL.

72. The method of claim 69 or 70, wherein the concentration of IL-21 is about 10 ng/mL.

73. The method of any one of claims 5 to 7 and 9 to 72, wherein the concentration of IL-7 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

74. The method of claim 73, wherein the concentration of IL-7 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

75. The method of claim 73 or 74, wherein the concentration of IL-7 is about 1.0 ng/mL.

76. The method of claim 73 or 74, wherein the concentration of IL-7 is about 10 ng/mL.

77. The method of any one of claims 4-6 and 8 to 76, wherein the concentration of IL-15 is from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL.

78. The method of claim 77, wherein the concentration of IL-15 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL.

79. The method of claim 77 or 78, wherein the concentration of IL-15 is about 1.0 ng/mL.

80. The method of claim 77 or 78, wherein the concentration of IL-15 is about 10 ng/mL.

81. The method of any one of claims 1 to 80, wherein the immune cells and/or stem cells comprises a chimeric antigen receptor, an engineered T cell receptor, or any combination thereof.

82. The method of any one of claims 1 to 81, wherein the immune cells and/or stem cells are administered to a human subject following culture.

83. The method of any one of claims 1 to 82, wherein the cells are further transduced with a vector.

84. The method of claim 83, wherein the vector comprises a transgene encoding a chimeric antigen receptor (CAR), a T cell receptor (TCR), or a TCR mimic.

85. The method of claim 84, wherein the CAR targets CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof.

86. The method of claim 84, wherein the TCR targets AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin Bi, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LTLRA2, CD300LF, CLEC12A, BST2, EMR2, LY75, GPC3, FCRL5, IGLL1, CD2, CD3P, CD4, CD5, CD7, the extracellular portion of the APRIL protein, or any combinations thereof.

87. The method of claim 84, wherein the vector is a retroviral vector, a lentiviral vector, an adeno-associated virus (AAV), an adenovirus, an AAV hybrid virus, a baculovirus, or any combination thereof.

88. A population of cells prepared by the method of any one of claims 1 to 87.

89. A population of cells comprising at least about 5%, at least about 10%, at least about 15%, at least about 20%, at least about 25%, at least about 30%, at least about 35%, at least about 40%, at least about 45%, at least about 50%, at least about 60%, or at least about 70% of the cells are CD3+, CD45RO⁻, CCR7+, CD45RA+, CD62L⁺, CD27+, CD28+, and TCF7+, wherein the cells are cultured according to the method of any one of claims 1 to 87.

90. A cell culture medium useful for the method of any one of claims 1 to 87.

91. A cell culture medium comprising (i) immune cells or stem cells as described in claims 1 to 87 and (ii) a potassium ion at a concentration higher than 40 mM, wherein the culture medium is not hypertonic.

92. The culture medium of claim 91, wherein the concentration of the potassium ion is at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM.

93. The culture medium of claim 91 or 92, wherein the concentration of the potassium ion is about 50 mM, about 60 mM, about 70 mM, or about 80 mM.

94. The culture medium of any one of claims 91 to 93, which further comprises one or more cytokines.

95. The culture medium of claim 94, wherein the one or more cytokines are IL-2, IL-7, IL-15, IL-21, or any combination thereof.

96. A method of treating a disease or condition in a subject in need thereof comprising administering the population of cells of claim 88 or 89 to the subject.

97. The method of claim 96, wherein the disease or condition comprises a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

98. A method of expanding immune cells obtained from a human subject comprising culturing the immune cells in initial expansion media, wherein the initial expansion media are hyperkalemic.

99. The method of claim 98, comprising culturing the immune cells in secondary expansion media, after culturing the cells in the initial expansion media.

100. The method of claim 99, wherein the second expansion media are hyperkalemic.

101. A method of expanding immune cells obtained from a human subject comprising culturing the immune cells (a) in initial expansion media and (b) in secondary expansion media, wherein the initial expansion media or the secondary expansion media are hyperkalemic.

102. The method of claim 101, wherein the secondary expansion media are hyperkalemic, and wherein the initial expansion media is not hyperkalemic.

103. The method of any one of claims 98 to 102, further comprising culturing the immune cells in third (or final) expansion media.

104. The method of claim 103, wherein the third expansion media are hyperkalemic.

105. The method of claim 103, wherein the initial expansion media and the secondary expansion media are hyperkalemic and the third expansion media are not hyperkalemic.

106. The method of any one of claims 98 to 105, wherein the initial expansion media further comprises IL-2, IL-21, or both.

107. The method of any one of claims 98 to 106, wherein the initial expansion media further comprises a T cell supplement, a serum replacement, glutamine, a glutamine substitute (e.g., Glutamax (L-alanine-L-glutamine)), non-essential amino acids, an antibiotics (e.g., Penicillin, Streptomycin, or both), an anti-fungal agent (e.g., FUNGIN™), and/or sodium pyruvate.

108. The method of any one of claims 98 to 107, wherein the immune cells are cultured in the initial expansion media for up to about six days, about seven days, about eight days, about nine days, or about 10 days.

109. The method of any one of claims 99 to 108, wherein the immune cells are stimulated with a CD3 agonist, a CD28 agnostic, or both in the secondary expansion media.

110. The method of any claims 99 to 109, wherein the immune cells are further stimulated with a CD27 ligand in the secondary expansion media.

111. The method of any one of claims 99 to 110, wherein the immune cells are further stimulated with a 4-1BB ligand in the secondary expansion media.

112. The method of any one of claims 99 to 111, wherein the immune cells are cultured for at least about 20 days, at least about 21 days, at least about 22 days, at least about 23 days, at least about 24 days, at least about 25 days, or at least about 26 days, after stimulation with a CD3 agonist, a CD28 agonist, and/or a CD27 ligand.

113. The method of any one of claims 103 to 112, wherein the immune cells are stimulated with a CD3 agonist, a CD28 agonist, a CD27 ligand, and/or a 4-1BB ligand in the third expansion media.

114. The method of any one of claims 103 to 113, wherein the third expansion media are not hyperkalemic.

115. The method of claim 113 or 114, wherein the immune cells are cultured for at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, or at least about 43 days.

116. A method of increasing tumor reactive immune cells comprising:
a. culturing immune cells in initial expansion media, which are hyperkalemic and optionally comprise IL-2 and/or IL-21, up to about seven to 14 days;
b. culturing the immune cells in secondary expansion media, which are hyperkalemic after adding a CD3 agonist, a CD28 agonist, a CD27 ligand, a 4-1BB ligand, or any combination thereof for about 20 days to about 25 days, e.g., about 21 days to about 24 days;
c. culturing the immune cells in third expansion media, which are not hyperkalemic, after adding a CD3 agonist, a CD28 agonist, a CD27 ligand, a 4-1BB ligand, or any combination thereof for about 30 days to about 50 days, e.g., about 34 days to about 45 days.

117. The method of any one of claims 1 to 87, wherein the immune cells comprise TILs.

118. The method of any one of claims 98 to 116, wherein the immune cells comprise TILs.

119. The method of claim 117 or 118, wherein the TILs comprise CD8⁺ TILs.

120. The method of any one of claims 117 to 119, wherein the TILs are enriched for CD8⁺ TTLs.

121. The method of any one of claims 117 to 119, wherein the TILs are enriched for tumor-specific TILs.

122. The method of any one of claims 117 to 119, wherein the TILs are enriched for stem-like TILs.

123. An immune cell expressing one or more stem-like markers and one or more effector-like markers.

124. The immune cell of claim 123, wherein the stem-like markers comprise CD45RA+, CD62L⁺, CCR7+, CD27+, CD28+, BACH2+, LEF1+, TCF7+, or any combination thereof.

125. The immune cell of claim 123 or 124, wherein the effector-like markers comprise pSTAT5+, STAT5+, pSTAT3+, STAT3+, or any combination thereof.

126. A population of engineered cells comprising the immune cell of any one of claims 123 to 125.

127. The population of engineered cells, wherein at least about 40%, at least about 50%, at least about 60%, at least about 70%, at least about 80%, at least about 85%, at least about 90%, at least about 95%, at least about 99%, or about 100% of the engineered cells comprise the immune cell of any one of claims 123 to 125.

128. A pharmaceutical composition comprising the immune cell of any one of claims 123 to 125 or the population of engineered cells of claim 126 or 127 and a pharmaceutically acceptable carrier.

129. A method of treating a disease or condition in a subject in need thereof comprising administering the immune cell of any one of claims 123 to 125, the population of engineered cells of claim 126 or 127, or the pharmaceutical composition of claim 128 to the subject.

130. The method of claim 129, wherein the disease or condition is a cancer.

131. A method of preparing the immune cell of any one of claims 123 to 125, comprising culturing the immune cell in culture medium under suitable conditions.

132. A population of human T cells, wherein at least about 5% of the T cells in the population of T cells have a stem-like phenotype.

133. The population of human T cells of claim 132, wherein at least about 10%, at least about 15%, at least about 20%, at least about 25%, at least about 30%, at least about 35%, at least about 40%, at least about 45%, or at least about 50% of the T cells in the population of T cells have a stem-like phenotype.

134. The population of human T cells of claim 132 or 133, wherein the T cells having a stem-like phenotype are TCF7⁺.

135. The population of human T cells of any one of claims 132 to 134, wherein the T cells having a stem-like phenotype are CD3⁺, CD45RO⁻, CCR7⁺, CD45RA⁺, CD62L⁺, CD27⁺, CD28⁺, and TCF7⁺.

136. The population of human T cells of any one of claims 132 to 135, wherein the T cells having a stem-like phenotype are CD39⁻ and CD69⁻.

137. The population of human T cells of any one of claims 132 to 136, wherein the T cells are CD8⁺ T cells.

138. A pharmaceutical composition comprising the population of human T cells of any one of claims 132 to 137, and a pharmaceutically acceptable carrier.

139. A method of killing target cells, comprising contacting the target cells with the population of human T cells of any one of claims 132 to 137 or the pharmaceutical composition of claim 138 under conditions that allow killing of the target cells by the T cells.

140. A method of treating a patient in need thereof, comprising administering the population of human T cells of any one of claims 132 to 137 or the pharmaceutical composition of claim 138 to the patient.

141. A method of inducing an anti-tumor immune response in a patient, comprising administering the population of T cells of any one of claims 132 to 137 or the pharmaceutical composition of claim 138 to the patient.

142. Use of the population of human T cells of any one of claims 132 to 137, or the pharmaceutical composition of claim 138 for the manufacture of a medicament for treating a patient in need thereof in the method of any one of claims 139 to 141.

143. The population of human T cells of any one of claims 132 to 137 or the pharmaceutical composition of claim 138 for use in a method of treating a cancer in a patient in need thereof.

144. The method of any one of claims 96, 97, and 139 to 141 wherein after the administration the size of a tumor (tumor size) in the patient is decreased compared to a reference tumor size.

145. The method of claim 144, wherein the reference tumor size comprises: (i) the tumor size before the administration, (ii) the tumor size in a corresponding subject that did not receive the administration, or (iii) both (i) and (ii).

146. The method of claim 144 or 145, wherein, compared to the reference tumor size, the tumor size is decreased by at least about 5%, at least about 10%, at least about 20%, at least about 30%, at least about 40%, at least about 50%, at least about 60%, at least about 70%, at least about 80%, at least about 90% or about 100%.

147. The method of any one of claims 96, 97, 139 to 141, and 144 to 146, wherein after the administration the duration of survival of the subject is increased compared to a reference duration of survival.

148. The method of claim 147, wherein the reference duration of survival comprises the duration of survival of a corresponding subject that did not receive the administration.

149. The method of claim 147 or 148, wherein, compared to the reference duration of survival, the duration of survival is by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold or more.

150. The method of any one of claims 1 to 87 and 98 to 122, wherein the immune cells express increased IL-2 following culture, as compared to immune cells cultured in control medium.

151. The method of claim 150, wherein the immune cells express at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold more IL-2 as compared to immune cells cultured in control medium.

152. The method of claim 150 or 151, wherein the immune cells express at least about 20 μg/ml, at least about 25 μg/ml, at least about 50 μg/ml, at least about 75 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, at least about 600 μg/ml, at least about 700 μg/ml, at least about 800 μg/ml, at least about 900 μg/ml, at least about 1000 μg/ml, at least about 1250 μg/ml, at least about 1500 μg/ml, at least about 1750 μg/ml, at least about 2000 μg/ml, at least about 2500 μg/ml, at least about 3000 μg/ml, at least about 3500 μg/ml, at least about 4000 μg/ml, at least about 4500 μg/ml, or at least about 5000 μg/ml IL-2.

153. The method of any one of claims 150 to 152, wherein the immune cells express increased IL-2 following a single stimulation.

154. The method of any one of claims 150 to 153, wherein the immune cells express increased IL-2 following at least two serial stimulations, and least three serial stimulations, and least four serial stimulations, or at least five serial stimulations.

155. The method of any one of claims 150 to 154, wherein the immune cells further express c-Jun.

156. The method of any one of claims 1 to 87, 98 to 122, and 150 to 155, wherein the immune cells exhibit increased cytotoxic activity as compared to immune cells cultured in control medium.

157. The method of claim 156, wherein immune cell cytotoxicity is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, at least about 100-fold, at least about 200-fold, at least about 300-fold, at least about 400-fold, at least about 500-fold, at least about 750-fold, or at least about 1,000-fold more IL-2 as compared to immune cells cultured in control medium.

158. The method of any one of claims 1 to 87, 98 to 122, and 150 to 157, wherein the immune cells exhibit increased persistence as compared to immune cells cultured in control medium.

159. The method of claim 158, wherein the immune cells exhibit cytotoxic activity following repeated contact with target cells for at least about 5 days, at least about 7 days, at least about 10 days, at least about 12 days, or at least about 14 days.
